(19) United States
(12) Patent Application Publication
(10) Pub. No.: US 2022/0049273 A1
(43) Pub. Date: Feb. 17, 2022

(54) NOVEL CRISPR DNA TARGETING ENZYMES AND SYSTEMS

(71) Applicant: ARBOR BIOTECHNOLOGIES, INC., Cambridge, MA (US)

(72) Inventors: David R. Cheng, Boston, MA (US); David A. Scott, Cambridge, MA (US); Winston X. Yan, Boston, MA (US); Jason Carte, Boston, MA (US); Elise Keston-Smith, Cambridge, MA (US); Pratyusha Hunnewell, Needham, MA (US); Shaorong Chong, Cambridge, MA (US)

(21) Appl. No.: 17/435,563

(22) PCT Filed: Feb. 28, 2020
(86) PCT No.: PCT/US2020/020426

§ 371 Date: Sep. 1, 2021

Related U.S. Application Data
(60) Provisional application No. 62/812,919, filed on Mar. 1, 2019.
(60) Provisional application No. 62/869,454, filed on Jul. 1, 2019.

(51) Int. Cl. C12N 15/90; C12N 9/22; C12N 15/11; C12N 15/86

(52) U.S. Cl. CPC C12N 15/902; C12N 9/22; C12N 15/111; C12N 2750/14143; C07K 2319/09; C12N 2310/20; C12N 15/86

(57) ABSTRACT

The disclosure describes novel systems, methods, and compositions for the manipulation of nucleic acids in a targeted fashion. The disclosure describes non-naturally occurring, engineered CRISPR-Cas systems, components, and methods for targeted modification of nucleic acids such as DNA. Each system includes one or more protein components and one or more nucleic acid components that together target nucleic acids.

### CROSS REFERENCE TO RELATED APPLICATIONS

This application claims priority to U.S. Ser. No. 62/812,919, filed Mar. 1, 2019, and U.S. Ser. No. 62/869,454, filed Jul. 1, 2019. The content of each of the foregoing applications is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Feb. 27, 2020, is named A2186-7015WO_SL.txt and is 1,436,701 bytes in size.

### FIELD OF THE INVENTION

The present disclosure relates to novel CRISPR-Cas system compositions and methods of using the compositions, for example, nucleic acid targeting.

### BACKGROUND

Recent application of advances in genome sequencing technologies and analysis have yielded significant insights into the genetic underpinning of biological activities in many diverse areas of nature, ranging from prokaryotic biosynthetic pathways to human pathologies. To fully understand and evaluate the vast quantities of information produced by genetic sequencing technologies, equivalent increases in the scale, efficacy, and ease of technologies for genome and epigenome manipulation are needed. These novel genome and epigenome engineering technologies will accelerate the development of novel applications in numerous areas, including biotechnology, agriculture, and human therapeutics.

Clustered Regularly Interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The CRISPR-Cas systems of prokaryotic adaptive immunity are an extremely, diverse group of proteins effectors, non-coding elements, as well as loci architectures, some examples of which have been engineered and adapted to produce important biotechnologies.

The components of the system involved in host defense include one or more effector proteins capable of modifying DNA or RNA and an RNA guide element that is responsible to targeting these protein activities to a specific sequence on the phage DNA or RNA. The RNA guide is composed of a CRISPR RNA (crRNA) and may require an additional trans-activating RNA (tracrRNA) to enable targeted nucleic acid manipulation by the effector protein(s). The crRNA consists of a direct repeat responsible for protein binding to the crRNA and a spacer sequence that is complementary to the desired nucleic acid target sequence. CRISPR-Cas systems can be reprogrammed to target alternative DNA or RNA targets by modifying the spacer sequence of the crRNA.

CRISPR-Cas systems can be broadly classified into two classes: Class 1 systems are composed of multiple effector proteins that together form a complex around a crRNA, and Class 2 systems consist of a single effector protein that complexes with the crRNA to target DNA or RNA substrates. The single-subunit effector composition of the Class 2 systems provides a simpler component set for engineering and application translation and have thus far been an important source of programmable effectors. Thus, the discovery, engineering, and optimization of novel Class 2 systems may lead to widespread and powerful programmable technologies for genome engineering and beyond.

The characterization and engineering of Class 2 CRISPR-Cas systems, exemplified by CRISPR-Cas9, have paved the way for a diverse array of biotechnology applications in genome editing and beyond. For example, the effector proteins Cas12a (Cpf1) and Cas13a (C2c2) possess non-target-specific “collateral” single-stranded-nuclease cleavage activities, which may be harnessed to create novel diagnostics, methods, and other applications. Nevertheless, there remains a need for additional programmable effectors and systems for modifying nucleic acids and polynucleotides (i.e., DNA, RNA, or any hybrid, derivative, or modification) beyond the current CRISPR-Cas systems that enable novel applications through their unique properties.

### SUMMARY

It is against the above background that the present invention provides certain advantages and advancements over the prior art.

Although this invention disclosed herein is not limited to specific advantages or functionalities, the invention provides a composition comprising a CRISPR-Cas effector protein or a nucleic acid encoding the CRISPR-Cas effector protein, wherein the CRISPR-Cas effector protein has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identity to an amino acid sequence set forth in any one of SEQ ID NOs: 1057-1066.

In one aspect of the composition, the CRISPR-Cas effector protein has an amino acid sequence set forth in any one of SEQ ID NOs: 1057-1066.

The invention further provides a composition comprising a CRISPR-Cas effector protein or a nucleic acid encoding the CRISPR-Cas effector protein, wherein the CRISPR-Cas effector protein comprises a mutation in a RuvC motif.

In one aspect of a composition of invention, the CRISPR-Cas effector protein comprises a mutation in a catalytic residue of a RuvC motif.

In one aspect of a composition of invention, the RuvC motif is a RuvC I, RuvC II, and/or RuvC III motif.

In one aspect of a composition of invention, the CRISPR-Cas effector protein comprises at least 10% (e.g., 10%, 20%, 30%, 40%, 50%, 60%, 70%, 80%, 90%, or 100%) less nuclease activity than a reference composition.

In one aspect of a composition of invention, the CRISPR-Cas effector protein lacks nuclease activity.

In one aspect of a composition of invention, the CRISPR-Cas effector protein comprises at least 10% (e.g., 10%, 20%, 30%, 40%, 50%, 60%, 70%, 80%, 90%, or 100%) greater nuclease activity than a reference composition.

In one aspect of a composition of invention, the composition further comprises an RNA guide or a nucleic acid encoding the RNA guide, wherein the RNA guide comprises a direct repeat sequence and a spacer sequence, wherein the CRISPR-Cas effector protein binds to the RNA guide, and wherein the spacer sequence binds to a target nucleic acid.

In one aspect of a composition of invention, the spacer sequence comprises between 15 and 24 nucleotides in length. In one aspect of a composition of invention, the spacer sequence comprises between 16 and 22 nucleotides in length.

In one aspect of a composition of invention, the target nucleic acid comprises a sequence complementary to a nucleotide sequence in the spacer sequence.

In one aspect of a composition of invention, the CRISPR-Cas effector protein recognizes a protospacer adjacent motif (PAM) sequence in the target nucleic acid, wherein the PAM sequence comprises a nucleotide sequence set forth as 5′-TTN-3′ or 5′-YTN-3′, wherein N is any nucleotide and Y is cytosine or thymine.

In one aspect of a composition of invention, the target nucleic acid is DNA. In one aspect of a composition of invention, the target nucleic acid is supercoiled (e.g., plasmid) DNA. In one aspect of a composition of invention, the CRISPR-Cas effector protein further comprises at least one nuclear localization signal (NLS), at least one nuclear export signal (NES), or at least one NLS and at least one NES.

In one aspect of a composition of invention, the nucleic acid encoding the CRISPR-Cas effector protein is codon-optimized for expression in a cell.

In one aspect of a composition of invention, the nucleic acid encoding the CRISPR-Cas effector protein is operably linked to a promoter.

In one aspect of a composition of invention, the nucleic acid encoding the CRISPR-Cas effector protein is in a vector. In one aspect of a composition of invention, the vector comprises a retroviral vector, a lentiviral vector, a phage vector, an adenoviral vector, an adeno-associated vector, or a herpes simplex vector.

In one aspect of a composition of invention, the composition is present in a delivery system comprising a nanoparticle, a liposome, an exosome, a microvesicle, or a gene-gun.

The invention further provides a cell comprising a composition of the invention. In one aspect of the cell, the cell is a eukaryotic cell. In one aspect of the cell, the cell is a prokaryotic cell.

The invention further provides a method of expressing a composition of the invention, wherein the method comprises providing a composition of the invention and delivering the composition to the cell.

The present disclosure further provides non-naturally-occurring, engineered systems and compositions for new single-effector Class 2 CRISPR-Cas systems, together with methods for computational identification of new CRISPR-Cas systems from genomic databases, together with the development of the natural loci into engineered systems, and experimental validation and application translation. These new effectors are divergent in sequence to orthologs and homologs of existing Class 2 CRISPR effectors, and also have unique domain organizations. They provide additional features that include, but are not limited to, 1) novel DNA/RNA editing properties and control mechanisms, 2) smaller size for greater versatility in delivery strategies, 3) genotype triggered cellular processes such as cell death, and 4) programmable RNA-guided DNA insertion, excision, and mobilization. Adding the novel DNA-targeting systems described herein to the toolbox of techniques for genome and epigenome manipulation enables broad applications for specific, programmed perturbations.

This disclosure relates to new CRISPR-Cas systems including newly discovered enzymes and other components used to create minimal systems that can be used in non-natural environments, e.g., in bacteria other than those in which the system was initially discovered or in mammalian cells.

As used herein, the term “catalytic residue” refers to an amino acid that activates catalysis. A catalytic residue is an amino acid that is involved (e.g., directly involved) in catalysis. In some embodiments, a catalytic residue is a histidine, an aspartic acid, or a glutamic acid residue.

The term “cleavage event,” as used herein, refers to a DNA break in a target nucleic acid created by a nuclease of a CRISPR-Cas system described herein. In some embodiments, the cleavage event is a double-stranded DNA break. In some embodiments, the cleavage event is a single-stranded DNA break.

The term “CRISPR-Cas system” as used herein refers to nucleic acids and/or proteins involved in the expression of, or directing the activity of, CRISPR-Cas effectors, including sequences encoding CRISPR-Cas effectors, RNA guides, and other sequences and transcripts from a CRISPR locus.

The term “CRISPR array” as used herein refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The term “CRISPR repeat,” or “CRISPR direct repeat,” or “direct repeat,” as used herein, refers to multiple short direct repeating sequences, which show very little or no sequence variation within a CRISPR array.

The term “CRISPR RNA” or “crRNA” as used herein refers to an RNA molecule comprising a guide sequence used by a CRISPR effector to specifically target a nucleic acid sequence. Typically, crRNAs contain a sequence that mediates target recognition and a sequence that forms a duplex with a tracrRNA. The crRNA: tracrRNA duplex binds to a CRISPR effector. The term “donor template nucleic acid,” as used herein refers to a nucleic acid molecule that can be used by one or more cellular proteins to alter the structure of a target nucleic acid after a CRISPR enzyme described herein has altered a target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome).

The term “CRISPR-Cas effector,” “CRISPR effector,” “effector,” “CRISPR-associated protein,” or “CRISPR enzyme” as used herein refers to a protein that carries out an enzymatic activity or that binds to a target site on a nucleic acid specified by an RNA guide. In some embodiments, a CRISPR effector is a nuclease. In some embodiments, a CRISPR effector has endonuclease activity, nickase activity, exonuclease activity, and/or excision activity.

As used herein, the terms “domain” and “protein domain” refer to a distinct functional and/or structural unit of a protein. In some embodiments, a domain may comprise a conserved amino acid sequence.

As used herein, the term “enzymatic activity” refers to the catalytic ability of an enzyme. For example, enzymatic activity may include nuclease activity.

As used herein, the terms “engineered,” “genetically-engineered,” “genetically-modified,” “recombinant,” and “modified” are used interchangeably and indicate intentional human manipulation to create, or cause a change in, a sequence, combination of sequences, or composition such that the sequence, combination of sequences, or composition does not exist in nature. In some embodiments, a composition of the invention is a genetically-engineered composition.

As used herein, the term “nuclease” refers to an enzyme capable of cleaving a phosphodiester bond. A nuclease hydrolyzes phosphodiester bonds in a nucleic acid backbone.

As used herein the term “operably linked” refers to nucleic acid sequences or amino acid sequences placed into a functional relationship with one another. For instance, a promoter or enhancer is operably linked to a coding sequence if it affects the modulation of the transcription of the coding sequence. Operably linked DNA sequences encoding regulatory sequences are typically contiguous to the coding sequence. However, enhancers can be functional when separated from a promoter, e.g., by up to several kilobases or more. Accordingly, some nucleic acid molecules may be operably linked, but not contiguous.

As used herein, the terms “protospacer adjacent motif” and “PAM sequence” refer to a sequence located near or adjacent to a target sequence. As used herein, a PAM sequence is required for cleavage by a nuclease described herein.

As used herein, the terms “parent,” “nuclease parent,” and “parent sequence” refer to a nuclease to which an alteration is made to produce a variant nuclease of the present invention.

In some embodiments, the parent is a nuclease having an identical amino acid sequence of the variant at one or more of specified positions. The parent may be a naturally occurring (wild-type) polypeptide. In a particular embodiment, the parent is a nuclease with at least 60%, at least 61%, at least 62%, at least 63%, at least 64%, at least 65%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% identity to a polypeptide of SEQ ID NO: 1 or SEQ ID NO: 20.

As used herein, the terms “reference composition,” “reference sequence,” and “reference” refer to a control, such as a negative control or a parent (e.g., a parent sequence, a parent protein, or a wild-type protein). In some embodiments, a reference sequence is set forth in SEQ ID NO: 1 or SEQ ID NO: 20.

As used herein, the terms “RNA guide” or “RNA guide sequence” refer to a molecule that recognizes (e.g., binds to) a target nucleic acid. An RNA guide may be designed to be complementary to a specific nucleic acid sequence. An RNA guide comprises a spacer sequence and a direct repeat (DR) sequence. The terms CRISPR RNA (crRNA), pre-crRNA, mature crRNA, and CRISPR array are also used herein to refer to an RNA guide.

As used herein, the term “RuvC domain” refers to a conserved domain or motif of amino acids having nuclease (e.g., endonuclease) activity. As used herein, a protein having a split RuvC domain refers to a protein having two or more RuvC motifs, at sequentially disparate sites within a sequence, that interact in a tertiary structure to form a RuvC domain.

As used herein, the terms “target nucleic acid” and “target sequence” refer to a nucleic acid that is specifically bound by a targeting moiety. In some embodiments, the spacer sequence of an RNA guide binds to the target nucleic acid.

As used herein, the terms “trans-activating crRNA” and “tracrRNA” refer to an RNA molecule involved in or required for the binding of an RNA guide to a target nucleic acid.

As used herein, the terms “variant” and “mutant” refer to a protein comprising an alteration, e.g., a substitution, insertion, deletion and/or fusion, at one or more (or one or several) positions, compared to its parent sequence. In some embodiments, the variant is a CRISPR-Cas effector protein variant. In some embodiments, the variant has an amino acid sequence set forth in any one of SEQ ID NOs: 1057-1066.

As used herein, the term “subject,” refers to any mammals, including, without limitation, humans and other primates, including rhesus macaques, chimpanzees and other monkey and ape species; farm animals, such as cattle, sheep, pigs, goats, and horses; domestic mammals, such as dogs and cats; laboratory animals, including rabbits, mice, rats, and guinea pigs; as well as birds, including domestic, wild, and game birds, such as chickens, turkeys, ducks, and geese; and the like. The term includes adult, young, and newborn individuals as well as male and female subjects. In some embodiments, a host cell is derived from a subject (e.g., stem cells, progenitor cells, or tissue-specific cells). In some embodiments, the subject is a non-human subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF FIGURE DESCRIPTION

FIGS. 1A and 1B are a group of schematic sequence representations that together show conserved CLUST.018837 effectors and CRISPR array elements for representative loci.

FIG. 2A is a series of sequences that show the multiple sequence alignment of examples of CRISPR direct repeat elements for CLUST.018837.

FIG. 2B is a depiction of the 3′ end of a multiple sequence alignment of CLUST.018837 direct repeat sequences.

FIGS. 3A and 3B are a group of schematic diagrams that together show predicted secondary structure of the RNA transcript of examples of CLUST.018837 direct repeats.

FIGS. 4A, 4B, 4C, 4D, 4E, and 4F are schematic representations that together show a phylogenetic tree of CLUST.018837 effector proteins.

FIG. 5A shows PFAM domain mapping results for CLUST.018837 effector proteins.

FIG. 5B is a schematic representation of a multiple sequence alignment of CLUST.018837 effector proteins, with the locations of the conserved catalytic residues of the RuvC domain indicated by the short bars and RuvC-I/II/III annotations above the alignment.

FIGS. 6A, 6B, 6C, and 6D are a series of schematic representations that together show an example of an engineered, non-naturally occurring construct for the CLUST.018837 CRISPR-Cas system containing the NZ_LDOS01000005 effector protein and CRISPR array, both expressed separately from artificial promoters.

FIGS. 7A, 7B, 7C, 7D, and 7E are graphs that show the degree of depletion activity of the engineered constructs for CRISPR-Cas systems NZ_LDOS01000005, 3300009004, APMI01033782. NZ_LVXZ1000012, and ADIG01000806, respectively.

FIGS. 8A, 8B, 8C, 8D, and 8E are graphic representations that show the location of strongly depleted targets on the pACYC184 plasmid for the engineered CLUST.018837 CRISPR-Cas systems NZ_LDOS01000005, 3300009004, APMI01033782, NZ_LVXZ01000012, and ADIG01000806, respectively. Depleted targets on the top strand and bottom strand are shown separately, and in relation to the orientation of the annotated genes. Depleted targets are depicted by gray bars, with the length of the bar corresponding to the length of the matching spacer, and the shade corresponding to the magnitude of depletion (darker shades corresponding to more depletion). The light gray line indicates the total number of screened spacers targeting each nucleotide position, and the vertical gray lines delineate the boundaries between two features (e.g. tetracycline-resistance gene and the adjacent non-coding region).

FIGS. 9A, 9B, 9C, 9D, and 9E are graphic representations that show the locations of strongly depleted targets relative to the targeted E. coli essential genes for the engineered CLUST.018837 CRISPR-Cas systems NZ_LDOS01000005, 3300009004, APMI01033782, NZ_LVXZ01000012, and ADIG01000806, respectively.

FIGS. 10A, 10B, 10C, and 10D are graphic representations that show the locations of strongly depleted targets on the pACYC184 plasmid for the “effector deletion” (negative control) CLUST.018837 CRISPR-Cas constructs for 3300009004, APMI01033782, NZ_LVXZ01000012, and ADIG01000806, respectively.

FIGS. 11A, 11B, 11C, and 11D are graphic representations that show the locations of strongly depleted targets relative to the targeted E. coli essential genes for the “effector deletion” (negative control) CLUST.018837 CRISPR-Cas constructs for 3300009004, APMI01033782, NZ_LVXZ01000012, and ADIG01000806, respectively.

FIGS. 12A, 12B, 12C, 12D, and 12E show sequences flanking the sites of strongly depleted targets for the engineered CLUST.018837 CRISPR-Cas systems NZ_LDOS01000005, 3300009004, APMI01033782, NZ_LVXZ01000012, and ADIG01000806, respectively.

FIGS. 13A, 13B, and 13C show the mature crRNA (comprising a direct repeat and a spacer) for exemplary CLUST.018837 CRISPR-Cas systems NZ_LDOS01000005, 3300009004, and ADIG01000806, respectively. FIGS. 13A, 13B, and 13C also show sequence alignments of RNA-sequenced transcripts including the processed form of the direct repeat and the orientation of the spacer with regard to the direct repeat on the mature crRNA, the processed crRNA sequence, and the secondary structure of a mature crRNA for exemplary CLUST.018837 CRISPR-Cas systems, NZ_LDOS01000005, 3300009004, and ADIG01000806, respectively.

FIG. 14 is an image of a gel that shows processing of the pre-crRNA into a mature crRNA by the NZ_LDOS01000005 effector protein in a dose-dependent manner. Pre-crRNA processing in the presence of EDTA suggests that magnesium is not required.

FIGS. 15A and 15B are graphs that show depletion of GFP fluorescent signal by CLUST.018837 CRISPR-Cas systems NZ_LDOS01000005 (SEQ ID NO: 1) and ADIG01000806 (SEQ ID NO: 20), respectively, by in vitro transcription-translation assays. Effectors were targeted against linear or plasmid GFP, with “TS” and “BS” indicating whether the top strand or bottom strand of the GFP was targeted, respectively. “Apo” indicates that the effector was not complexed with pre-crRNA to form a ribonucleoprotein (RNP).

FIGS. 16A-F are graphs that show the effects of amino acid substitutions in putative RuvCI, RuvCII, and RuvCIII domains of CLUST.018837 CRISPR-Cas system NZ_LDOS01000005 on GFP fluorescent signal. Effectors were targeted against supercoiled plasmid GFP, with “TS” and “BS” indicating whether the bottom strand or top strand of GFP was targeted, respectively. “Apo” indicates that the effector was not complexed with pre-crRNA to form an RNP. The wild-type NZ_LDOS01000005 (SEQ ID NO: 1) is shown in FIG. 16A, and the NZ_LDOS01000005 mutants (SEQ ID NOs: 1057-1061) are shown in FIGS. 16B-F.

FIGS. 16G-L are graphs that show the effects of amino acid substitutions in putative RuvCI, RuvCII, and RuvCIII domains of CLUST.018837 CRISPR-Cas system ADIG01000806 on GFP fluorescent signal. Effectors were targeted against supercoiled plasmid GFP, with “TS” and “BS” indicating whether the bottom strand or top strand of GFP was targeted, respectively. “Apo” indicates that the effector was not complexed with pre-crRNA to form an RNP. The wild-type ADIG01000806 (SEQ ID NO: 20) is shown in FIG. 16G, and the ADIG01000806 mutants (SEQ ID NOs: 1062-1066) are shown in FIGS. 16H-L.

FIGS. 17A-D are graphs that show in vitro cleavage of supercoiled plasmid GFP by CLUST.018837 CRISPR-Cas system NZ_LDOS01000005 (SEQ ID NO: 1). The target region of the guide is indicated by a box, and arrows point to specific cleavage by the effector. Reads were binned by nucleotide position along the GFP plasmid and normalized to the total number of reads. 5′ and 3′ graphs indicate whether the reads were aligned to the 5′ or 3′ end of the GFP sequence. Data from two biological replicates are shown (BR1 and BR2).

FIGS. 17E-H are graphs that show in vitro cleavage of supercoiled plasmid GFP by CLUST.018837 CRISPR-Cas systems ADIG01000806 (SEQ ID NO: 20). The target region of the guide is indicated by a box, and arrows point to specific cleavage by the effector. Reads were binned by nucleotide position along the GFP plasmid and normalized to the total number of reads. 5′ and 3′ graphs indicate whether the reads were aligned to the 5′ or 3′ end of the GFP sequence. Data from two biological replicates are shown (BR1 and BR2).

### DETAILED DESCRIPTION

The present disclosure relates to novel compositions comprising CRISPR-Cas effector proteins and methods of use thereof. In some aspects, a composition comprising a CRISPR-Cas effector protein having one or more characteristics is described herein. In some aspects, a method of producing the composition is described. In some aspects, a method of delivering the composition is described.

### Class 2 CRISPR-Cas Effectors

In one aspect, the disclosure provides Class 2 CRISPR-Cas systems referred to herein as CLUST.018837. These Class 2 CRISPR-Cas systems contain an isolated CRISPR-associated protein having a RuvC domain.

In some embodiments, the CRISPR-associated protein and the RNA guide form a “binary” complex that may include other components. The binary complex is activated upon binding to a nucleic acid substrate that is complementary to a spacer sequence in the RNA guide (i.e., a sequence-specific substrate or target nucleic acid). In some embodiments, the sequence-specific substrate is a double-stranded DNA. In some embodiments, the sequence-specific substrate is a single-stranded DNA. In some embodiments, the sequence-specific substrate is a single-stranded RNA. In some embodiments, the sequence-specific substrate is a double-stranded RNA. In some embodiments, the sequence-specificity requires a complete match of the spacer sequence in the RNA guide (e.g., crRNA) to the target substrate. In other embodiments, the sequence specificity requires a partial (contiguous or non-contiguous) match of the spacer sequence in the RNA guide (e.g., crRNA) to the target substrate.

In some embodiments, the binary complex becomes activated upon binding to the target substrate. In some embodiments, the activated complex exhibits “multiple turnover” activity, whereby upon acting on (e.g., cleaving) the target substrate the activated complex remains in an activated state. In some embodiments, the activated binary complex exhibits “single turnover” activity, whereby upon acting on the target substrate the binary complex reverts to an inactive state. In some embodiments, the activated binary complex exhibits non-specific (i.e., “collateral”) cleavage activity whereby the complex cleaves non-target nucleic acids. In some embodiments, the non-target nucleic acid is a DNA (e.g., a single-stranded or a double-stranded DNA). In some embodiments, the non-target nucleic acid is an RNA (e.g., a single-stranded or a double-stranded RNA).

In some embodiments, the composition of the present invention includes a CRISPR-Cas effector protein described herein. A nucleic acid sequence encoding the CRISPR-Cas effector protein described herein may be substantially identical to a reference nucleic acid sequence if the nucleic acid encoding the CRISPR-Cas effector protein comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the CRISPR-Cas effector protein is encoded by a nucleic acid sequence having at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a reference nucleic acid sequence.

The CRISPR-Cas effector protein described herein may substantially identical to a reference polypeptide if the CRISPR-Cas effector protein comprises an amino acid sequence having at least about 60%, least about 65%, least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the reference polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 20. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 20.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1057. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1057.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1058. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1058.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1059. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1059.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1060. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1060.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1061. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1061.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1062. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1062.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1063. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1063.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1064. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1064.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1065. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1065.

In some embodiments, the CRISPR-Cas effector protein of the present invention comprises a polypeptide sequence having 50, 60, 65, 70, 75, 80, 85, 90, 91, 92, 93, 94, 95, 96, 97, 98, 99 or 100% identity to SEQ ID NO: 1066. In some embodiments, the CRISPR-Cas effector protein of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1066.

In some embodiments, the CRISPR-Cas effector protein of the present invention is a CRISPR-Cas effector protein having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to the amino acid sequence of SEQ ID NO: 1 or SEQ ID NO: 20. Homology or identity can be determined by amino acid sequence alignment, e.g., using a program such as BLAST, ALIGN, or CLUSTAL, as described herein.

In some embodiments, the CRISPR-Cas effector protein comprises a protein with an amino acid sequence with at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference amino acid sequence.

Also provided is a CRISPR-Cas effector protein of the present invention and comprising an amino acid sequence which differs from the amino acid sequences of any one of SEQ ID NO: 1, SEQ ID NO: 20, SEQ ID NO: 1057, SEQ ID NO: 1058, SEQ ID NO: 1059, SEQ ID NO: 1060, SEQ ID NO: 1061, SEQ ID NO: 1062, SEQ ID NO: 1063, SEQ ID NO: 1064, SEQ ID NO: 1065, or SEQ ID NO: 1066 by no more than 50, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), when aligned using any of the previously described alignment methods.

In some embodiments, the CRISPR-Cas effector protein comprises a RuvC domain. In some embodiments, the CRISPR-Cas effector protein comprises a split RuvC domain or two or more (e.g., 3) partial RuvC domains. For example, the CRISPR-Cas effector protein comprises RuvC motifs that are not contiguous with respect to the primary amino acid sequence of the CRISPR-Cas effector protein but form a RuvC domain once the protein folds. In some embodiments, the catalytic residue of a RuvC motif is a histidine, glutamic acid residue, and/or an aspartic acid residue, including H297, D303, E311, E504, or D559 according to the numbering of SEQ ID NO: 1 or H300, D306, E332, E516, or D569 according to the numbering of SEQ ID NO: 20.

In some embodiments, the invention includes an isolated, recombinant, substantially pure, or non-naturally occurring CRISPR-Cas effector protein comprising a RuvC domain, wherein the CRISPR-Cas effector protein has enzymatic activity, e.g., nuclease or endonuclease activity, wherein the CRISPR-Cas effector protein comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1, SEQ ID NO: 20, SEQ ID NO: 1057, SEQ ID NO: 1058, SEQ ID NO: 1059, SEQ ID NO: 1060, SEQ ID NO: 1061, SEQ ID NO: 1062, SEQ ID NO: 1063, SEQ ID NO: 1064, SEQ ID NO: 1065, or SEQ ID NO: 1066.

In some embodiments, the invention includes a CRISPR-Cas effector protein comprising a mutated RuvC domain, wherein the CRISPR-Cas effector protein has modified enzymatic activity, e.g., nuclease or endonuclease activity, wherein the CRISPR-Cas effector protein comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1057, SEQ ID NO: 1058, SEQ ID NO: 1059, SEQ ID NO: 1060, SEQ ID NO: 1061, SEQ ID NO: 1062, SEQ ID NO: 1063, SEQ ID NO: 1064, SEQ ID NO: 1065, or SEQ ID NO: 1066.

### CRISPR Enzyme Modifications

Modified CRISPR Enzyme Activity

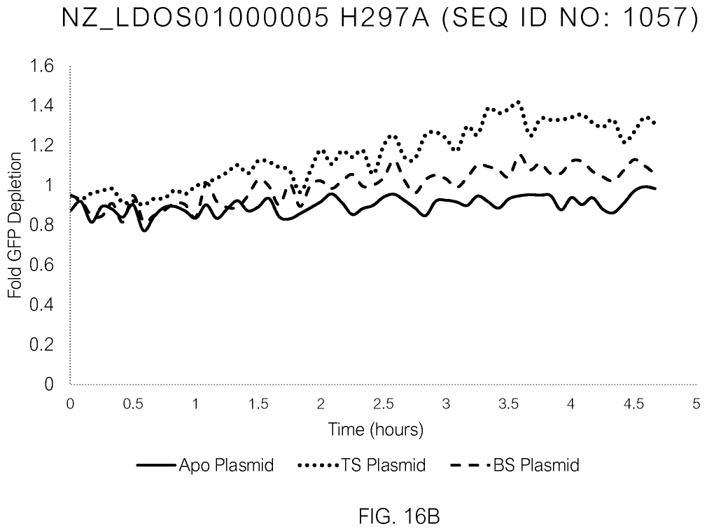

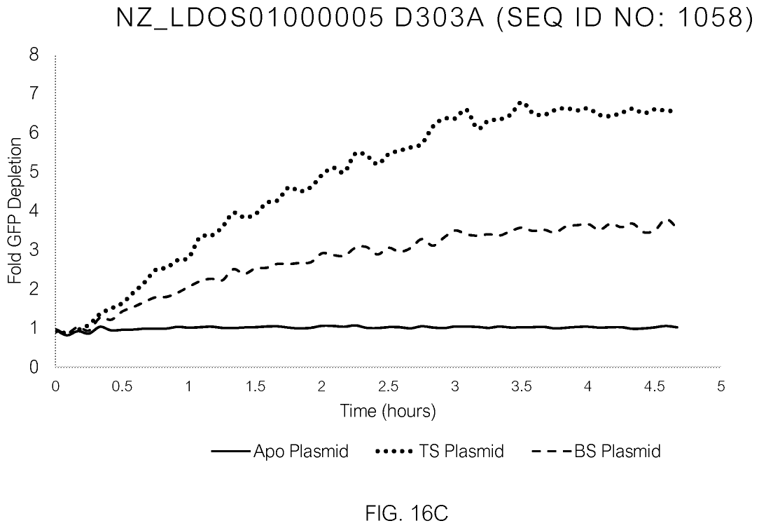

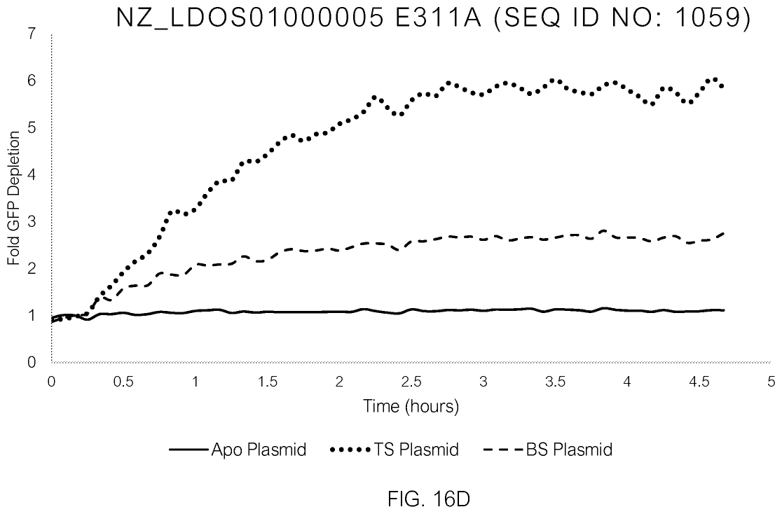

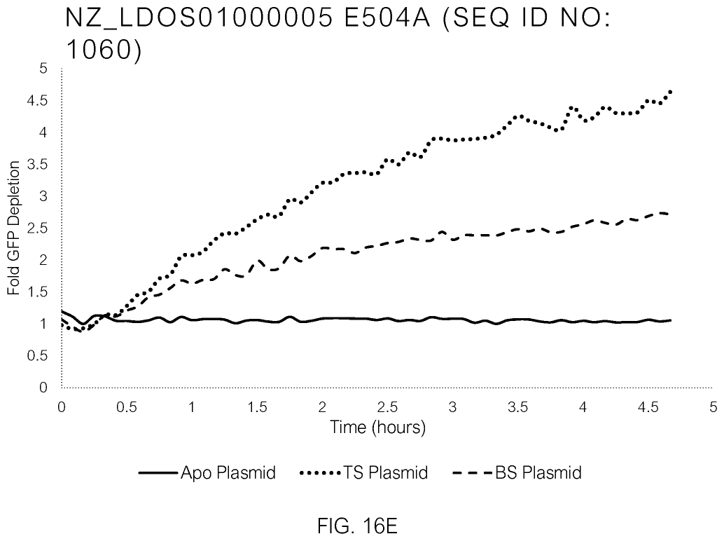

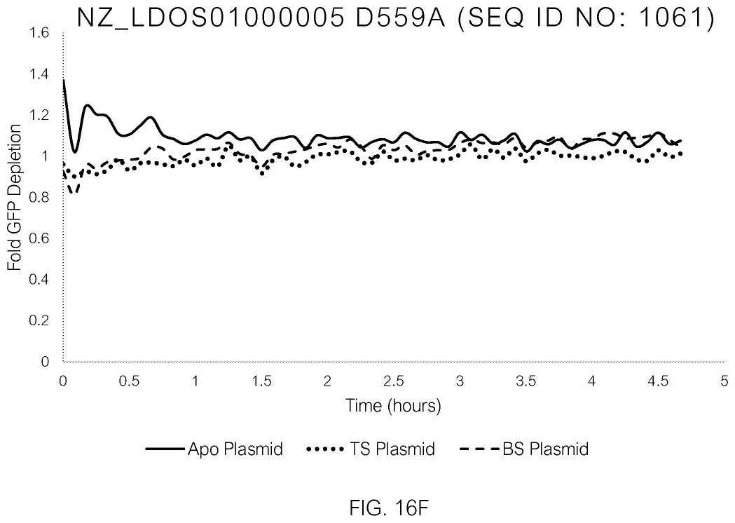

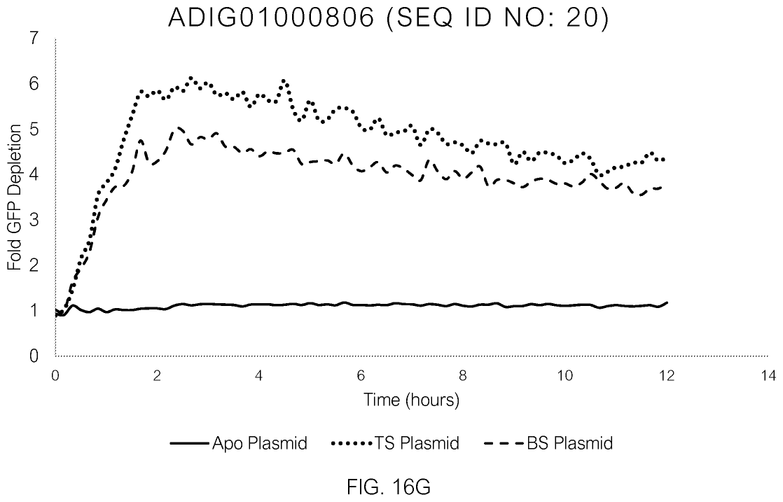

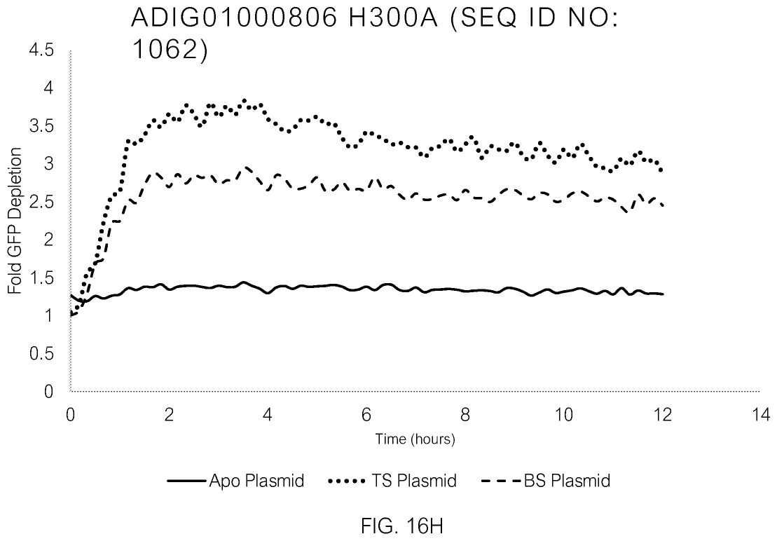

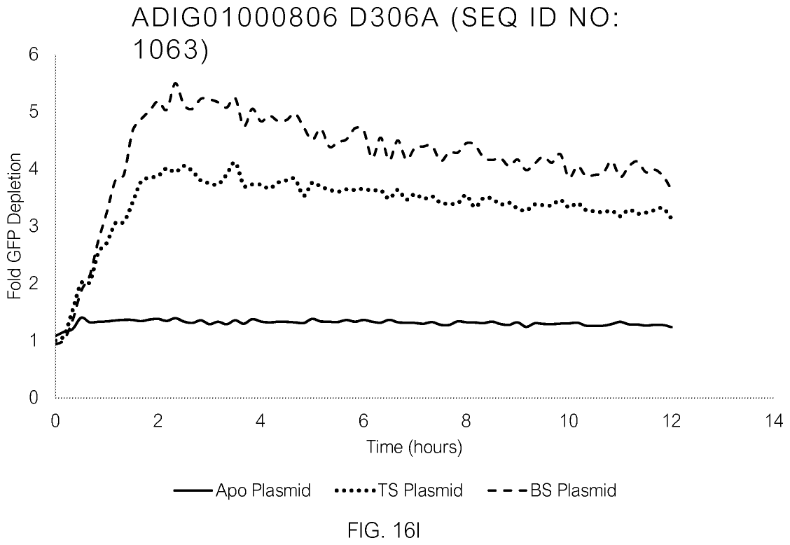

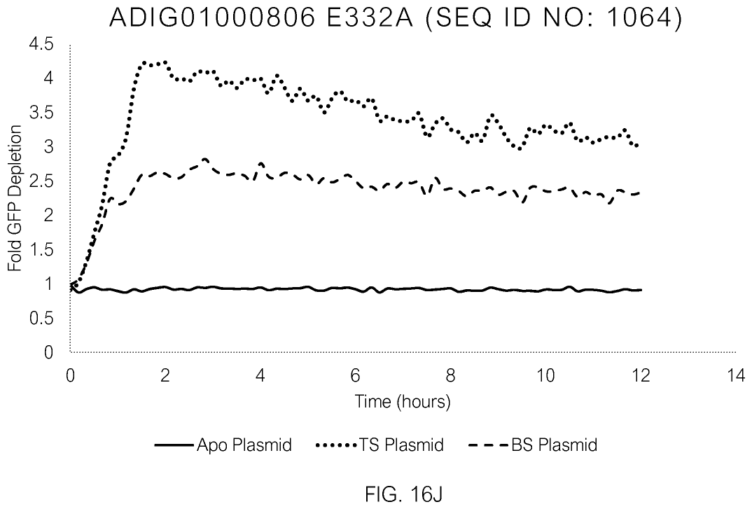

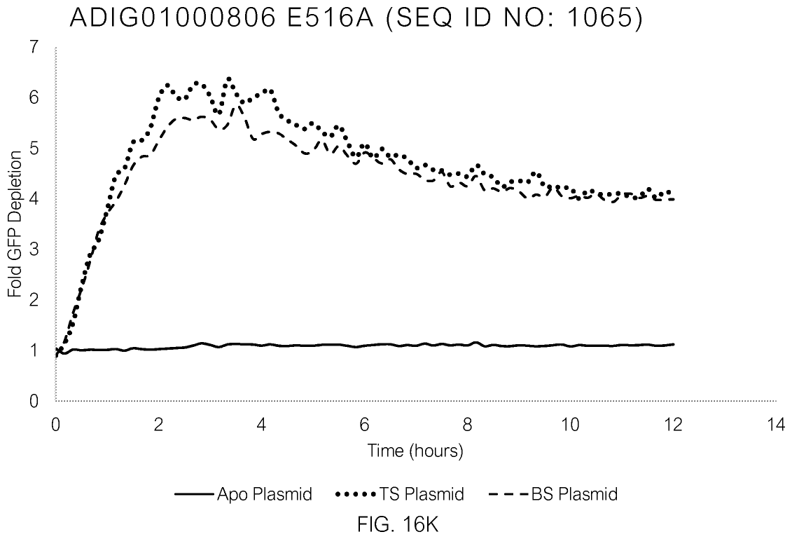

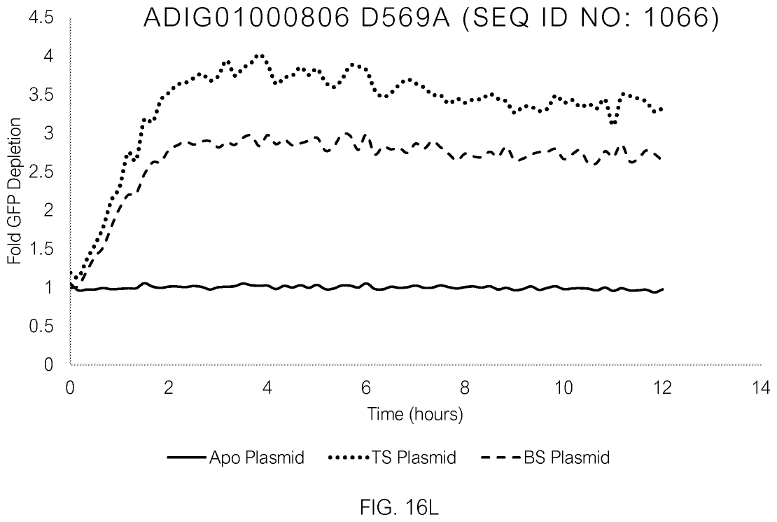

In some embodiments, the present invention includes variants of the nuclease described herein. In some embodiments, the nuclease described herein can be mutated at one or more amino acid residues to modify one or more functional activities. For example, in some embodiments, the nuclease is mutated at one or more amino acid residues to modify its nuclease activity (e.g., cleavage activity). For example, in some embodiments, the nuclease may comprise one or more mutations that increase the ability of the nuclease to cleave a target nucleic acid. In some embodiments, the nuclease is mutated at one or more amino acid residues to modify its ability to functionally associate with an RNA guide. In some embodiments, the nuclease is mutated at one or more amino acid residues to modify its ability to functionally associate with a target nucleic acid.

Where the CRISPR enzymes described herein have nuclease activity, the CRISPR enzymes can be modified to have diminished nuclease activity, e.g., nuclease inactivation of at least 50%, at least 60%, at least 70%, at least 80%, at least 90%, at least 95%, at least 97%, or 100% as compared with the wild type CRISPR enzymes. The nuclease activity can be diminished by several methods known in the art, e.g., introducing mutations into the nuclease domains of the proteins. In some embodiments, catalytic residues for the nuclease activities are identified, and these amino acid residues can be substituted by different amino acid residues (e.g., glycine or alanine) to diminish the nuclease activity. In some embodiments, a catalytic residue of a RuvC motif (RuvC I, RuvC II, or RuvC III) is mutated to decrease or inactivate nuclease activity. See, e.g., FIGS. 16B, 16F. In some embodiments, these nucleases are referred to as “RuvC inactivated,” RuvC diminished,” or “nuclease dead” proteins.

Where the CRISPR enzymes described herein have nuclease activity, the CRISPR enzymes can be modified to have increased nuclease activity, e.g., nuclease activation of at least 50%, at least 60%, at least 70%, at least 80%, at least 90%, at least 95%, at least 97%, or 100% as compared with the wild type CRISPR enzymes. The nuclease activity can be increased by several methods known in the art, e.g., introducing mutations into the nuclease domains of the proteins. In some embodiments, catalytic residues for the nuclease activities are identified, and these amino acid residues can be substituted by different amino acid residues (e.g., glycine or alanine) to increase or activate the nuclease activity. See, e.g., FIGS. 16C, 16I.

As used herein, a “biologically active portion” is a portion that maintains the function (e.g. completely, partially, minimally) of the nuclease (e.g., a “minimal” or “core” domain). In some embodiments, a nuclease fusion protein is useful in the methods described herein. Accordingly, in some embodiments, a nucleic acid encoding the fusion nuclease is described herein. In some embodiments, all or a portion of one or more components of the nuclease fusion protein are encoded in a single nucleic acid sequence.

In some embodiments, a variant nuclease has a conservative or non-conservative amino acid substitution, deletion or addition. In some embodiments, the variant nuclease has a silent substitution, deletion or addition, or a conservative substitution, none of which alter the polypeptide activity of the present invention. Typical examples of the conservative substitution include substitution whereby one amino acid is exchanged for another, such as exchange among aliphatic amino acids Ala, Val, Lu and Ile, exchange between hydroxyl residues Ser and Thr, exchange between acidic residues Asp and Glu, substitution between amide residues Asn and Gln, exchange between basic residues Lys and Arg, and substitution between aromatic residues Phe and Tyr. In some embodiments, one or more residues of a nuclease disclosed herein are mutated to an Arg residue. In some embodiments, one or more residues of a nuclease disclosed herein are mutated to a Gly residue.

A variety of methods are known in the art that are suitable for generating modified polynucleotides that encode variant nucleases of the invention, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., nucleases) include DNA shuffling methodologies, methods based on non-homologous recombination of genes, such as ITCHY (See, Ostermeier et al., 7:2139-44 [1999]), SCRACHY (See, Lutz et al. 98:11248-53 [2001]), SHIPREC (See, Sieber et al., 19:456-60 [2001]), and NRR (See, Bittker et al., 20:1024-9 [2001]; Bittker et al., 101:7011-6 [2004]), and methods that rely on the use of oligonucleotides to insert random and targeted mutations, deletions and/or insertions (See, Ness et al., 20:1251-5 [2002]; Coco et al., 20:1246-50 [2002]; Zha et al., 4:34-9 [2003]; Glaser et al., 149:3903-13 [1992]).

Generation of Fusion Proteins

Additionally, nuclease dead CRISPR enzymes, whether in their native form or with mutations to modulate their nuclease activity, can provide a foundation from which fusion proteins with additional functional proteins can be created. The nuclease dead CRISPR enzymes can comprise or be associated (e.g., via fusion protein, linker peptides, and “GS” linkers) with one or more functional domains. These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). In some embodiments, the functional domains are Krüppel associated box (KRAB), VP64, VP16, FokI, P65, HSF1, MyoD1, and biotin-APEX.

The positioning of the one or more functional domains on the nuclease dead CRISPR enzymes is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., FokI) is positioned to cleave or partially cleave the target. In some embodiments, the functional domain is positioned at the N-terminus of the CRISPR enzyme. In some embodiments, the functional domain is positioned at the C-terminus of the CRISPR enzyme. In some embodiments, the inactivated CRISPR enzyme is modified to comprise a first functional domain at the N-terminus and a second functional domain at the C-terminus.

The addition of functional domains to the CRISPR enzymes or onto other effector proteins in the complex may provide an ability for the CRISPR-Cas system to modify the physical DNA (e.g., methylation, etc.) or its regulation (e.g., transcriptional or repression) in situ.

Split Enzymes

The present disclosure also provides a split version of the CRISPR enzymes described herein. The split version of the CRISPR enzymes may be advantageous for delivery. In some embodiments, the CRISPR enzymes are split to two parts of the enzymes, which together substantially comprises a functioning CRISPR enzyme.

The split can be done in a way that the catalytic domain(s) are unaffected. The CRISPR enzymes may function as a nuclease or may be inactivated enzymes, which are essentially RNA-binding proteins with very little or no catalytic activity (e.g., due to mutation(s) in its catalytic domains).

In some embodiments, the nuclease lobe and α-helical lobe are expressed as separate polypeptides. Although the lobes do not interact on their own, the RNA guide recruits them into a ternary complex that recapitulates the activity of full-length CRISPR enzymes and catalyzes site-specific DNA cleavage. The use of a modified RNA guide abrogates split-enzyme activity by preventing dimerization, allowing for the development of an inducible dimerization system. The split enzyme is described, e.g., in Wright, Addison V., et al. “Rational design of a split-Cas9 enzyme complex,” Proc. Nat'l. Acad. Sci., 112.10 (2015): 2984-2989, which is incorporated herein by reference in its entirety.

In some embodiments, the split enzyme can be fused to a dimerization partner, e.g., by employing rapamycin sensitive dimerization domains. This allows the generation of a chemically inducible CRISPR enzyme for temporal control of CRISPR enzyme activity. The CRISPR enzymes can thus be rendered chemically inducible by being split into two fragments and rapamycin-sensitive dimerization domains can be used for controlled reassembly of the CRISPR enzymes.

The split point is typically designed in silico and cloned into the constructs. During this process, mutations can be introduced to the split enzyme and non-functional domains can be removed. In some embodiments, the two parts or fragments of the split CRISPR enzyme (i.e., the N-terminal and C-terminal fragments), can form a full CRISPR enzyme, comprising, e.g., at least 70%, at least 80%, at least 90%, at least 95%, or at least 99% of the sequence of the wild-type CRISPR enzyme.

Self-Activating or Inactivating Enzymes

The CRISPR enzymes described herein can be designed to be self-activating or self-inactivating. In some embodiments, the CRISPR enzymes are self-inactivating. For example, the target sequence can be introduced into the CRISPR enzyme coding constructs. Thus, the CRISPR enzymes can modify, e.g., cleave, the target sequence, as well as the construct encoding the enzyme thereby self-inactivating their expression. Methods of constructing a self-inactivating CRISPR-Cas system is described, e.g., in Epstein, Benjamin E., and David V. Schaffer. “Engineering a Self-Inactivating CRISPR-Cas System for AAV Vectors,” Mol. Ther., 24 (2016): S50, which is incorporated herein by reference in its entirety.

In some other embodiments, an additional RNA guide, expressed under the control of a weak promoter (e.g., 7SK promoter), can target the nucleic acid sequence encoding the CRISPR enzyme to prevent and/or block its expression (e.g., by preventing the transcription and/or translation of the nucleic acid). The transfection of cells with vectors expressing the CRISPR enzyme, and RNA guides that target the nucleic acid encoding the CRISPR enzyme can lead to efficient disruption of the nucleic acid encoding the CRISPR enzyme and decrease the levels of CRISPR enzyme, thereby limiting the genome editing activity.

In some embodiments, the genome editing activity of the CRISPR enzymes can be modulated through endogenous RNA signatures (e.g., miRNA) in mammalian cells. The CRISPR enzyme switch can be made by using a miRNA-complementary sequence in the 5′-UTR of mRNA encoding the CRISPR enzyme. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa, Moe et al. “Cell-type-specific genome editing with a microRNA-responsive CRISPR-Cas9 switch,” Nucl. Acids Res., 2017 Jul. 27; 45(13): e118).

Inducible CRISPR Enzymes

The CRISPR enzymes can be inducible, e.g., light inducible or chemically inducible. This mechanism allows for activation of the functional domain in the CRISPR enzymes. Light inducibility can be achieved by various methods known in the art, e.g., by designing a fusion complex wherein CRY2PHR/CIBN pairing is used in split CRISPR Enzymes (see, e.g., Konermann et al. “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split CRISPR Enzymes. Rapamycin is required for forming the fusion complex, thereby activating the CRISPR enzymes (see, e.g., Zetsche, Volz, and Zhang, “A split-Cas9 architecture for inducible genome editing and transcription modulation,” Nature Biotech., 33.2 (2015): 139-142).

Furthermore, expression of the CRISPR enzymes can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless, Stephen J. et al. “Direct and specific chemical control of eukaryotic translation with a synthetic RNA-protein interaction,” Nuc. Acids Res., 40.9 (2012): e64-e64).

Various embodiments of inducible CRISPR enzymes and inducible CRISPR-Cas systems are described, e.g., in U.S. Pat. No. 8,871,445, US20160208243, and WO2016205764, each of which is incorporated herein by reference in its entirety.

Functional Mutations

Various mutations or modifications can be introduced into CRISPR enzymes as described herein to improve specificity and/or robustness. In some embodiments, the amino acid residues that recognize the Protospacer Adjacent Motif (PAM) are identified. The CRISPR enzymes described herein can be modified further to recognize different PAMs, e.g., by substituting the amino acid residues that recognize PAM with other amino acid residues. In some embodiments, the CRISPR enzymes can recognize a PAM, e.g., 5′-TTN-3′ or 5′-YTN-3′, wherein N is any nucleobase and Y is cytosine or thymine.

In some embodiments, at least one Nuclear Localization Signal (NLS) is attached to the nucleic acid sequences encoding the CRISPR enzyme. In some embodiments, at least one Nuclear Export Signal (NES) is attached to the nucleic acid sequences encoding the CRISPR enzyme. In a preferred embodiment a C-terminal and/or N-terminal NLS or NES is attached for optimal expression and nuclear targeting in eukaryotic cells, e.g., human cells.

In some embodiments, the CRISPR enzyme is mutated at one or more amino acid residues to alter its ability to functionally associate with an RNA guide. In some embodiments, the CRISPR enzyme is mutated at one or more amino acid residues to alter its ability to functionally associate with a target nucleic acid.

In some embodiments, the CRISPR enzymes described herein are capable of binding to or modifying a target nucleic acid molecule. In some embodiments, the CRISPR enzyme modifies both strands of the target nucleic acid molecule. However, in some embodiments, the CRISPR enzyme is mutated at one or more amino acid residues to alter its nucleic acid manipulation activity. For example, in some embodiments, the CRISPR enzyme may comprise one or more mutations which render the enzyme incapable of cleaving a target nucleic acid. In other embodiments, the CRISPR enzyme may comprise one or more mutations such that the enzyme is capable of cleaving a single strand of the target nucleic acid (i.e., nickase activity). In some embodiments, the CRISPR enzyme is capable of cleaving the strand of the target nucleic acid that is complementary to the strand to which the RNA guide hybridizes. In some embodiments, the CRISPR enzyme is capable of cleaving the strand of the target nucleic acid to which the RNA guide hybridizes.

In some embodiments, a CRISPR enzyme described herein may be engineered to comprise a deletion in one or more amino acid residues to reduce the size of the enzyme while retaining one or more desired functional activities (e.g., nuclease activity and the ability to interact functionally with an RNA guide). The truncated CRISPR enzyme may be advantageously used in combination with delivery systems having load limitations.

Nucleic Acids Encoding the CRISPR-Associated Proteins

Nucleic acids encoding the proteins (e.g., a CRISPR-associated protein) and RNA guides (e.g., a crRNA) described herein are also provided. In some embodiments, the nucleic acid is a synthetic nucleic acid. In some embodiments, the nucleic acid is a DNA molecule. In some embodiments, the nucleic acid is an RNA molecule (e.g., an mRNA molecule). In some embodiments, the nucleic acid is an mRNA. In some embodiments, the mRNA is capped, polyadenylated, substituted with 5-methylcytidine, substituted with pseudouridine, or a combination thereof. In some embodiments, the nucleic acid (e.g., DNA) is operably-linked to a regulatory element (e.g., a promoter) to control the expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter. Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a β-actin promoter. For example, a U6 promoter can be used to regulate the expression of an RNA guide molecule described herein.

In some embodiments, the nucleic acids are modified, e.g., optimized, e.g., codon-optimized, for expression in a eukaryotic cell, e.g., a mammalian cell, such as a human cell.

In some embodiments, the nucleic acid(s) are present in a vector (e.g., a viral vector or a phage). The vectors can include one or more regulatory elements that allow for the propagation of the vector in a cell of interest (e.g., a bacterial cell or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

### RNA Guide Modifications

Spacer Lengths

The spacer length of RNA guides can range from about 15 to 50 nucleotides. In some embodiments, the spacer length of an RNA guide is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer. In some embodiments, the direct repeat length of the RNA guide is at least 16 nucleotides, or is from 16 to 20 nucleotides (e.g., 16, 17, 18, 19, or 20 nucleotides). In some embodiments, the direct repeat length of the RNA guide is 19 nucleotides.

Exemplary RNA guide direct repeat sequences and effector protein pairs are provided in Table 3. In some embodiments, the RNA guide includes a direct repeat sequence comprising or consisting of a nucleic acid sequence listed in Table 3 (e.g., SEQ ID Nos: 27-47, 263-440).

The RNA guide sequences can be modified in a manner that allows for formation of the CRISPR complex and successful binding to the target, while at the same time not allowing for successful effector activity (i.e., without nuclease activity/without causing indels). These modified guide sequences are referred to as “dead guides” or “dead guide sequences.” These dead guides or dead guide sequences may be catalytically inactive or conformationally inactive with regard to nuclease activity. Dead guide sequences are typically shorter than respective guide sequences that result in active DNA modification. In some embodiments, dead guides are 5%, 10%, 20%, 30%, 40%, or 50%, shorter than respective RNA guides that have nuclease activity. Dead guide sequences of RNA guides can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR-Cas systems including a functional CRISPR enzyme as described herein, and an RNA guide wherein the RNA guide includes a dead guide sequence whereby the RNA guide is capable of hybridizing to a target sequence such that the CRISPR-Cas system is directed to a genomic locus of interest in a cell without detectable nucleic acid modification activity.

A detailed description of dead guides is described, e.g., in WO 2016094872, which is incorporated herein by reference in its entirety.

Inducible Guides

RNA guides can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy.

In some embodiments, the transcription of RNA guides can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in their entirety.

Chemical Modifications

Chemical modifications can be applied to the RNA guide's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized RNA guide molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the RNA guide includes one or more phosphorothioate modifications. In some embodiments, the RNA guide includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

Sequence Modifications

The sequences and the lengths of the RNA guides described herein can be optimized. In some embodiments, the optimized length of RNA guide can be determined by identifying the processed form of tracrRNA and/or crRNA, or by empirical length studies for guide RNAs, tracrRNAs, crRNAs, and the tracrRNA tetraloops.

The RNA guides can also include one or more aptamer sequences. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the RNA guide has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, φCb5, φCb8r, φCb12r, φCb23r, 7s, and PRR1. Accordingly, in some embodiments, the aptamer is selected from binding proteins specifically binding any one of the adaptor proteins as described herein. In some embodiments, the aptamer sequence is a MS2 loop. A detailed description of aptamers can be found, e.g., in Nowak et al., “Guide RNA engineering for versatile Cas9 functionality,” Nucl. Acid. Res., 2016 Nov. 16; 44(20):9555-9564; and WO 2016205764, which are incorporated herein by reference in their entirety.

Guide: Target Sequence Matching Requirements

In classic CRISPR-Cas systems, the degree of complementarity between a guide sequence and its corresponding target sequence can be about 50%, 60%, 75%, 80%, 85%, 90%, 95%, 97.5%, 99%, or 100%. In some embodiments, the degree of complementarity is 100%. The RNA guides can be about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

To reduce off-target interactions, e.g., to reduce the guide interacting with a target sequence having low complementarity, mutations can be introduced to the CRISPR-Cas systems so that the CRISPR-Cas systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required provided that there is sufficient complementarity to be functional. For CRISPR nucleases, modulation of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

### Methods of Using CRISPR-Cas Systems

The CRISPR-Cas systems described herein have a wide variety of utilities including modifying (e.g., deleting, inserting, translocating, inactivating, or activating) a target polynucleotide in a multiplicity of cell types. The CRISPR-Cas systems have a broad spectrum of applications in, e.g., DNA/RNA detection (e.g., specific high sensitivity enzymatic reporter unlocking (SHERLOCK)), tracking and labeling of nucleic acids, enrichment assays (extracting desired sequence from background), detecting circulating tumor DNA, preparing next generation library, drug screening, disease diagnosis and prognosis, and treating various genetic diseases or disorders, and treating various non-genetic diseases or disorders, or augmenting health via manipulation of the genome.

DNA/RNA Detection

In one aspect, the CRISPR-Cas systems described herein can be used in DNA/RNA detection. Single effector RNA-guided DNases can be reprogrammed with CRISPR RNAs (crRNAs) to provide a platform for specific single-stranded DNA (ssDNA) sensing. Upon recognition of its DNA target, activated Type V single effector DNA-guided DNases engage in “collateral” cleavage of nearby non-targeted ssDNAs. This crRNA-programmed collateral cleavage activity allows the CRISPR-Cas systems to detect the presence of a specific DNA by nonspecific degradation of labeled ssDNA.

The collateral ssDNA activity can be combined with a reporter in DNA detection applications such as a method called the DNA Endonuclease-Targeted CRISPR trans reporter (DETECTR) method, which achieves attomolar sensitivity for DNA detection (see, e.g., Chen et al., Science, 360(6387):436-439, 2018), which is incorporated herein by reference in its entirety. One application of using the enzymes described herein is to degrade non-specific ssDNA in an in vitro environment. A “reporter” ssDNA molecule linking a fluorophore and a quencher can also be added to the in vitro system, along with an unknown sample of DNA (either single-stranded or double-stranded). Upon recognizing the target sequence in the unknown piece of DNA, the effector complex cleaves the reporter ssDNA resulting in a fluorescent readout.

In other embodiments, the SHERLOCK method (Specific High Sensitivity Enzymatic Reporter UnLOCKing) also provides an in vitro nucleic acid detection platform with attomolar (or single-molecule) sensitivity based on nucleic acid amplification and collateral cleavage of a reporter ssDNA, allowing for real-time detection of the target. Methods of using CRISPR in SHERLOCK are described in detail, e.g., in Gootenberg, et al. “Nucleic acid detection with CRISPR-Cas13a/C2c2,” Science, 356(6336):438-442 (2017), which is incorporated herein by reference in its entirety.

In some embodiments, the CRISPR-Cas systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference in its entirety.

Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The RNA targeting effector proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965; WO 2016205764; and WO 2017070605; each of which is incorporated herein by reference in its entirety.

High-Throughput Screening

The CRISPR-Cas systems described herein can be used for preparing next generation sequencing (NGS) libraries. For example, to create a cost-effective NGS library, the CRISPR-Cas systems can be used to disrupt the coding sequence of a target gene, and the CRISPR enzyme transfected clones can be screened simultaneously by next-generation sequencing (e.g., on an Illumina system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

Engineered Microorganisms

Microorganisms (e.g., E. coli, yeast, and microalgae) are widely used for synthetic biology. The development of synthetic biology has a wide utility, including various clinical applications. For example, the programmable CRISPR-Cas systems can be used to split proteins of toxic domains for targeted cell death, e.g., using cancer-linked RNA as target transcript. Further, pathways involving protein-protein interactions can be influenced in synthetic biological systems with e.g. fusion complexes with the appropriate effectors such as kinases or enzymes.

In some embodiments, RNA guide sequences that target phage sequences can be introduced into the microorganism. Thus, the disclosure also provides methods of vaccinating a microorganism (e.g., a production strain) against phage infection.

In some embodiments, the CRISPR-Cas systems provided herein can be used to engineer microorganisms, e.g., to improve yield or improve fermentation efficiency. For example, the CRISPR-Cas systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast, 2017 Sep. 8. doi: 10.1002/yea.3278; and Hlavova et al., “Improving microalgae for biotechnology—from genetics to synthetic biology,” Biotechnol. Adv., 2015 Nov. 1; 33:1194-203, both of which are incorporated herein by reference in their entirety.

Application in Plants

The CRISPR-Cas systems described herein have a wide variety of utility in plants. In some embodiments, the CRISPR-Cas systems can be used to engineer genomes of plants (e.g., improving production, making products with desired post-translational modifications, or introducing genes for producing industrial products). In some embodiments, the CRISPR-Cas systems can be used to introduce a desired trait to a plant (e.g., with or without heritable modifications to the genome), or regulate expression of endogenous genes in plant cells or whole plants.

In some embodiments, the CRISPR-Cas systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol., 11(3):222-8 (2011), and WO 2016205764 A1; both of which are incorporated herein by reference in their entirety.

Gene Drives

Gene drive is the phenomenon in which the inheritance of a particular gene or set of genes is favorably biased. The CRISPR-Cas systems described herein can be used to build gene drives. For example, the CRISPR-Cas systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR-Cas systems described herein to build gene drives is described, e.g., in Hammond et al., “A CRISPR-Cas9 gene drive system targeting female reproduction in the malaria mosquito vector Anopheles gambiae,” Nat. Biotechnol., 2016 January; 34(1):78-83, which is incorporated herein by reference in its entirety.

Pooled-Screening

As described herein, pooled CRISPR screening is a powerful tool for identifying genes involved in biological mechanisms such as cell proliferation, drug resistance, and viral infection. Cells are transduced in bulk with a library of RNA guide-encoding vectors described herein, and the distribution of RNA guides is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR-Cas systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods., 2017 March; 14(3):297-301, which is incorporated herein by reference in its entirety.

Saturadon Mutagenesis (“Bashing”)

The CRISPR-Cas systems described herein can be used for in situ saturating mutagenesis. In some embodiments, a pooled RNA guide library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature, 2015 Nov. 12; 527(7577):192-7, which is incorporated herein by reference in its entirety.

Quantitative Trait Mapping (crisprQTL)

The CRISPR-Cas systems described herein can be used for mapping coding and non-coding regions of a genome that influence gene expression. For example, in some embodiments, a population of cells may be transduced with multiple random, barcoded, CRISPR guide RNA-programmed perturbations in each cell. Single-cell RNA-sequencing may then be used to profile gene expression and the collection of RNA guides in each cell. The generated data can then be used to identify associations between RNA guides and quantitative changes in gene expression, which facilitates the analysis of the cis-regulatory architecture of the cells. These methods are described, for example, in Gasperini et al., “crisprQTL mapping as a genome-wide association framework for cellular genetic screens,” bioRxiv 314344, posted May 4, 2018, doi: doi.org/10.1101/314344, which is incorporated herein by reference in its entirety.

Therapeutic Applications

The CRISPR-Cas systems described herein can have various therapeutic applications. In some embodiments, the new CRISPR-Cas systems can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting), and various cancers, etc.

In some embodiments, the CRISPR-Cas systems described herein can be used to edit a target nucleic acid to modify the target nucleic acid (e.g., by inserting, deleting, or mutating one or more amino acid residues). For example, in some embodiments the CRISPR-Cas systems described herein comprise an exogenous donor template nucleic acid (e.g., a DNA molecule or an RNA molecule), which comprises a desirable nucleic acid sequence. Upon resolution of a cleavage event induced with the CRISPR-Cas system described herein, the molecular machinery of the cell utilizes the exogenous donor template nucleic acid in repairing and/or resolving the cleavage event. Alternatively, the molecular machinery of the cell can utilize an endogenous template in repairing and/or resolving the cleavage event. In some embodiments, the CRISPR-Cas systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). Methods of designing exogenous donor template nucleic acids are described, for example, in PCT Publication No. WO 2016094874 A1, the entire contents of which are expressly incorporated herein by reference.

In one aspect, the CRISPR-Cas systems described herein can be used for treating a disease caused by overexpression of RNAs, toxic RNAs, and/or mutated RNAs (e.g., splicing defects or truncations). For example, expression of the toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR-Cas systems as described herein can target overexpressed RNA or toxic RNA, e.g., the DMPK gene or any of the mis-regulated alternative splicing in DM1 skeletal muscle, heart, or brain.

The CRISPR-Cas systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR-Cas systems described herein is summarized in Cooper et al., “RNA and disease,” Cell, 136.4 (2009): 777-793, and WO 2016205764 A1, both of which are incorporated herein by reference in their entirety. Those of skill in this field will understand how to use the new CRISPR-Cas systems to treat these diseases.

The CRISPR-Cas systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR-Cas systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR-Cas systems described herein can further be used for antiviral activity, in particular against RNA viruses. The effector proteins can target the viral RNAs using suitable RNA guides selected to target viral RNA sequences.

Furthermore, in vitro RNA sensing assays can be used to detect specific RNA substrates. The RNA targeting effector proteins can be used for RNA-based sensing in living cells. Examples of applications are diagnostics by sensing of, for examples, disease-specific RNAs.

A detailed description of therapeutic applications of the CRISPR-Cas systems described herein can be found, e.g., in U.S. Pat. No. 8,795,965, EP 3009511, WO 2016205764, and WO 2017070605; each of which is incorporated herein by reference in its entirety.

Delivery of CRISPR-Cas Systems

Through this disclosure and the knowledge in the art, the CRISPR-Cas systems described herein, or components thereof, nucleic acid molecules thereof, or nucleic acid molecules encoding or providing components thereof, can be delivered by various delivery systems such as vectors, e.g., plasmids, viral delivery vectors. The new CRISPR enzymes and/or any of the RNAs (e.g., RNA guides) can be delivered using suitable vectors, e.g., plasmids or viral vectors, such as adeno-associated viruses (AAV), lentiviruses, adenoviruses, and other viral vectors, or combinations thereof. The proteins and one or more RNA guides can be packaged into one or more vectors, e.g., plasmids or viral vectors.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in their entirety.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AV2, AAV5, AAV6, AAV8, AAV 8.2, AAV9, AAV rhlO, modified AAV vectors (e.g., modified AAV2, modified AAV3, modified AAV6) and pseudotyped AAV (e.g., AAV2/8, AAV2/5 and AAV2/6). Exemplary AAV vectors and techniques that may be used to produce rAAV particles are known in the art (see, e.g., Aponte-Ubillus et al. (2018) Appl. Microbiol. Biotechnol. 102(3): 1045-54; Zhong et al. (2012) J. Genet. Syndr. Gene Ther. S1: 008; West et al. (1987) Virology 160: 38-47 (1987); Tratschin et al. (1985) Mol. Cell. Biol. 5: 3251-60); U.S. Pat. Nos. 4,797,368 and 5,173,414; and International Publication Nos. WO 2015/054653 and WO 93/24641, each of which is incorporated herein by reference in its entirety).

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids generally include (i) a promoter, (ii) a sequence encoding a nucleic acid-targeting CRISPR enzymes, operably linked to the promoter; (iii) a selectable marker, (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofectin formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR-Cas systems to the cell is by using cell penetrating peptides (CPP). In some embodiments, a cell penetrating peptide is linked to the CRISPR enzymes. In some embodiments, the CRISPR enzymes and/or RNA guides are coupled to one or more CPPs to transport them inside cells effectively (e.g., plant protoplasts). In some embodiments, the CRISPR enzymes and/or RNA guide(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hallbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR-Cas systems described herein are also described, e.g., in U.S. Pat. No. 8,795,965, EP 3009511, WO 2016205764, and WO 2017070605; each of which is incorporated herein by reference in its entirety.

ADDITIONAL SEQUENCES

NZ_LDOS01000005 H297A (SEQ ID NO: 1057)
MKLSPALPPTGDVLIYEYGARVDGDCLPAVGDQIAKARRLYNDLVAVIRGIVDEMRGFVLKH
AGSEALALQARIDGLSEAFDAARAANDEDRMKQIAGERRALWAELGEQVKAVRKAHRAEIQE
LFLSRIGKKSTCDTYQMRCKAVGDGLGWATANQVLDAALQAFKTSFQRGQAPRFARGEEKIQ
DTLTLQFTAAGGVPVAALLSGDHSELSMVSSCGRRKYGSFSFRLGSASADTYANGTWQYHRP
LPDGATVGLARLVRRSVGKDFKWALQLMVKRPATEPAMMEGRKPLVAVAFGWAGDASGRRVA
GITDGADPGVARVLQLPVEVEDGIRRAAEFQSARDEARDVIMTTIKNIAWGDAVACLGESSQ
FMHGSEPWLRARLSEELSTIRRLPAQHVAPRRLHRLCGLLRATNQMHDELEAWRKQDRLAWQ
ASAHMARRARNLRKDFYRRVAIDLARRYSAIVLEPLDLAAAALKVNEITGEKTEFAKKARSG
RVVAAIYELESSIRWAAAKSGTALLDLSGAETAARCGICGGASQSDESNSQVLHCVECGAEL
DRKKNGAAIAWQFAHENLDEAVTDFWAAVIAQRCEHAEKTREKKAKMAEGRRLARTLSAGVS
AVGSRNV
NZ_LDOS01000005 D303A (SEQ ID NO: 1058)
MKLSPALPPTGDVLIYEYGARVDGDCLPAVGDQIAKARRLYNDLVAVIRGIVDEMRGFVLKH
AGSEALALQARIDGLSEAFDAARAANDEDRMKQIAGERRALWAELGEQVKAVRKAHRAEIQE
LFLSRIGKKSTCDTYQMRCKAVGDGLGWATANQVLDAALQAFKTSFQRGQAPRFARGEEKIQ
DTLTLQFTAAGGVPVAALLSGDHSELSMVSSCGRRKYGSFSFRLGSASADTYANGTWQYHRP
LPDGATVGLARLVRRSVGKDFKWALQLMVKRPATEPAMMEGRKPLVAVHFGWAGAASGRRVA
GITDGADPGVARVLQLPVEVEDGIRRAAEFQSARDEARDVIMTTIKNIAWGDAVACLGESSQ
FMHGSEPWLRARLSEELSTIRRLPAQHVAPRRLHRLCGLLRATNQMHDELEAWRKQDRLAWQ
ASAHMARRARNLRKDFYRRVAIDLARRYSAIVLEPLDLAAAALKVNEITGEKTEFAKKARSG
RVVAAIYELESSIRWAAAKSGTALLDLSGAETAARCGICGGASQSDESNSQVLHCVECGAEL
DRKKNGAAIAWQFAHENLDEAVTDFWAAVIAQRCEHAEKTREKKAKMAEGRRLARTLSAGVS
AVGSRNV
NZ_LDOS01000005 E311A (SEQ ID NO: 1059)
MKLSPALPPTGDVLIYEYGARVDGDCLPAVGDQIAKARRLYNDLVAVIRGIVDEMRGFVLKH
AGSEALALQARIDGLSEAFDAARAANDEDRMKQIAGERRALWAELGEQVKAVRKAHRAEIQE
LFLSRIGKKSTCDTYQMRCKAVGDGLGWATANQVLDAALQAFKTSFQRGQAPRFARGEEKIQ
DTLTLQFTAAGGVPVAALLSGDHSELSMVSSCGRRKYGSFSFRLGSASADTYANGTWQYHRP
LPDGATVGLARLVRRSVGKDFKWALQLMVKRPATEPAMMEGRKPLVAVHFGWAGDASGRRVA
AITDGADPGVARVLQLPVEVEDGIRRAAEFQSARDEARDVIMTTIKNIAWGDAVACLGESSQ
FMHGSEPWLRARLSEELSTIRRLPAQHVAPRRLHRLCGLLRATNQMHDELEAWRKQDRLAWQ
ASAHMARRARNLRKDFYRRVAIDLARRYSAIVLEPLDLAAAALKVNEITGEKTEFAKKARSG
RVVAAIYELESSIRWAAAKSGTALLDLSGAETAARCGICGGASQSDESNSQVLHCVECGAEL
DRKKNGAAIAWQFAHENLDEAVTDFWAAVIAQRCEHAEKTREKKAKMAEGRRLARTLSAGVS
AVGSRNV
NZ_LDOS01000005 D504A (SEQ ID NO: 1060)
MKLSPALPPTGDVLIYEYGARVDGDCLPAVGDQIAKARRLYNDLVAVIRGIVDEMRGFVLKH
AGSEALALQARIDGLSEAFDAARAANDEDRMKQIAGERRALWAELGEQVKAVRKAHRAEIQE
LFLSRIGKKSTCDTYQMRCKAVGDGLGWATANQVLDAALQAFKTSFQRGQAPRFARGEEKIQ
DTLTLQFTAAGGVPVAALLSGDHSELSMVSSCGRRKYGSFSFRLGSASADTYANGTWQYHRP
LPDGATVGLARLVRRSVGKDFKWALQLMVKRPATEPAMMEGRKPLVAVHFGWAGDASGRRVA
GITDGADPGVARVLQLPVEVEDGIRRAAEFQSARDEARDVIMTTIKNIAWGDAVACLGESSQ
FMHGSEPWLRARLSEELSTIRRLPAQHVAPRRLHRLCGLLRATNQMHDELEAWRKQDRLAWQ
ASAHMARRARNLRKDFYRRVAIDLARRYSAIVLEPLDLAAAALKVNEITGEKTEFAKKARSG
RVVAAIYALESSIRWAAAKSGTALLDLSGAETAARCGICGGASQSDESNSQVLHCVECGAEL
DRKKNGAAIAWQFAHENLDEAVTDFWAAVIAQRCEHAEKTREKKAKMAEGRRLARTLSAGVS
AVGSRNV
NZ_LDOS01000005 D559A(SEQ ID NO: 1061)
MKLSPALPPTGDVLIYEYGARVDGDCLPAVGDQIAKARRLYNDLVAVIRGIVDEMRGFVLKH
AGSEALALQARIDGLSEAFDAARAANDEDRMKQIAGERRALWAELGEQVKAVRKAHRAEIQE
LFLSRIGKKSTCDTYQMRCKAVGDGLGWATANQVLDAALQAFKTSFQRGQAPRFARGEEKIQ
DTLTLQFTAAGGVPVAALLSGDHSELSMVSSCGRRKYGSFSFRLGSASADTYANGTWQYHRP
LPDGATVGLARLVRRSVGKDFKWALQLMVKRPATEPAMMEGRKPLVAVHFGWAGDASGRRVA
GITDGADPGVARVLQLPVEVEDGIRRAAEFQSARDEARDVIMTTIKNIAWGDAVACLGESSQ
FMHGSEPWLRARLSEELSTIRRLPAQHVAPRRLHRLCGLLRATNQMHDELEAWRKQDRLAWQ
ASAHMARRARNLRKDFYRRVAIDLARRYSAIVLEPLDLAAAALKVNEITGEKTEFAKKARSG
RVVAAIYELESSIRWAAAKSGTALLDLSGAETAARCGICGGASQSDESNSQVLHCVECGAEL
ARKKNGAAIAWQFAHENLDEAVTDFWAAVIAQRCEHAEKTREKKAKMAEGRRLARTLSAGVS
AVGSRNV
ADIG01000806 H300A (SEQ ID NO: 1062)
MIVQITPAPLPQGDVRIYEFGARLDHDCVRTVDEQIFKAHQLYNQLVACMQTTVRDMQAYLL
DHAGPDAHAAKARVDGLNEAFNAARAANDENRMTTVATERREAWRALAAVLRIARKEHRTAM
QETFLSRIGKKSACETYQLRCKAVADGLGWATANATLDAALIAFKKSFALGRAPRFARIADS
IQDTLTLQFTAAGGINIERLLDGKHTELALKPPAVCGKRGYGTFAFRLGAASAETQATGTWQ
YHRPLPPGGTVGLARLVRRRIGPKTTWSLQLQVRSPLPEREHEDRRPLVTVAPGWAADLSGR
RIAGIADAADPGLATVLQLPPDIEHGLQRAAELESTRSQARDALTPMLKVHPWPQELLNAAT
PEEDASASGDSGPMAPERIMCRKVADEILALRRLPAQHIAIRRLHRLARWLRLAEVDVPDWL
ETWRKEDKLRWQASAAAAKRARNRRRGFYRETALRLASQYQAIVIEPLNLADAAKKIDEATG
ERSDFAKKARAGRVVAAIFELDSAIRWAATKCGTAVLDLTGETAQHCAICGGHSLKADDEDS
QCLRCSDCGADIDRKRNGAALAWQAAAAHLETHLEDFWRLTLENRASAAAKRDEKKTKLQEG
RRAAMRETLET
ADI001000806 D306A (SEQ ID NO: 1063)
MIVQITPAPLPQGDVRIYEFGARLDHDCVRTVDEQIFKAHQLYNQLVACMQTTVRDMQAYLL
DHAGPDAHAAKARVDGLNEAFNAARAANDENRMTTVATERREAWRALAAVLRIARKEHRTAM
QETFLSRIGKKSACETYQLRCKAVADGLGWATANATLDAALIAFKKSFALGRAPRFARIADS
IQDTLTLQFTAAGGINIERLLDGKHTELALKPPAVCGKRGYGTFAFRLGAASAETQATGTWQ
YHRPLPPGGTVGLARLVRRRIGPKTTWSLQLQVRSPLPEREHEDRRPLVTVHPGWAAALSGR
RIAGIADAADPGLATVLQLPPDIEHGLQRAAELESTRSQARDALTPMLKVHPWPQELLNAAT
PEEDASASGDSGPMAPERIMCRKVADEILALRRLPAQHIAIRRLHRLARWLRLAEVDVPDWL
ETWRKEDKLRWQASAAAAKRARNRRRGFYRETALRLASQYQAIVIEPLNLADAAKKIDEATG
ERSDFAKKARAGRVVAAIFELDSAIRWAATKCGTAVLDLTGETAQHCAICGGHSLKADDEDS
QCLRCSDCGADIDRKRNGAALAWQAAAAHLETHLEDFWRLTLENRASAAAKRDEKKTKLQEG
RRAAMRETLET
ADIG01000806 E332A (SEQ ID NO: 1064)
MIVQITPAPLPQGDVRIYEFGARLDHDCVRTVDEQIFKAHQLYNQLVACMQTTVRDMQAYLL
DHAGPDAHAAKARVDGLNEAFNAARAANDENRMTTVATERREAWRALAAVLRIARKEHRTAM
QETFLSRIGKKSACETYQLRCKAVADGLGWATANATLDAALIAFKKSFALGRAPRFARIADS
IQDTLTLQFTAAGGINIERLLDGKHTELALKPPAVCGKRGYGTFAFRLGAASAETQATGTWQ
YHRPLPPGGTVGLARLVRRRIGPKTTWSLQLQVRSPLPEREHEDRRPLVTVHPGWAADLSGR
RIAGIADAADPGLATVLQLPPAIEHGLQRAAELESTRSQARDALTPMLKVHPWPQELLNAAT
PEEDASASGDSGPMAPERIMCRKVADEILALRRLPAQHIAIRRLHRLARWLRLAEVDVPDWL
ETWRKEDKLRWQASAAAAKRARNRRRGFYRETALRLASQYQAIVIEPLNLADAAKKIDEATG
ERSDFAKKARAGRVVAAIFELDSAIRWAATKCGTAVLDLTGETAQHCAICGGHSLKADDEDS
QCLRCSDCGADIDRKRNGAALAWQAAAAHLETHLEDFWRLTLENRASAAAKRDEKKTKLQEG
RRAAMRETLET
ADIG01000806 E516A (SEQ ID NO: 10651
MIVQITPAPLPQGDVRIYEFGARLDHDCVRTVDEQIFKAHQLYNQLVACMQTTVRDMQAYLL
DHAGPDAHAAKARVDGLNEAFNAARAANDENRMTTVATERREAWRALAAVLRIARKEHRTAM
QETFLSRIGKKSACETYQLRCKAVADGLGWATANATLDAALIAFKKSFALGRAPRFARIADS
IQDTLTLQFTAAGGINIERLLDGKHTELALKPPAVCGKRGYGTFAFRLGAASAETQATGTWQ
YHRPLPPGGTVGLARLVRRRIGPKTTWSLQLQVRSPLPEREHEDRRPLVTVHPGWAADLSGR
RIAGIADAADPGLATVLQLPPDIEHGLQRAAELESTRSQARDALTPMLKVHPWPQELLNAAT
PEEDASASGDSGPMAPERIMCRKVADEILALRRLPAQHIAIRRLHRLARWLRLAEVDVPDWL
ETWRKEDKLRWQASAAAAKRARNRRRGFYRETALRLASQYQAIVIEPLNLADAAKKIDEATG
ERSDFAKKARAGRVVAAIFALDSAIRWAATKCGTAVLDLTGETAQHCAICGGHSLKADDEDS
QCLRCSDCGADIDRKRNGAALAWQAAAAHLETHLEDFWRLTLENRASAAAKRDEKKTKLQEG
RRAAMRETLET
ADIG01000806 D569A (SEQ ID NO: 1066)
MIVQITPAPLPQGDVRIYEFGARLDHDCVRTVDEQIFKAHQLYNQLVACMQTTVRDMQAYLL
DHAGPDAHAAKARVDGLNEAFNAARAANDENRMTTVATERREAWRALAAVLRIARKEHRTAM
QETFLSRIGKKSACETYQLRCKAVADGLGWATANATLDAALIAFKKSFALGRAPRFARIADS
IQDTLTLQFTAAGGINIERLLDGKHTELALKPPAVCGKRGYGTFAFRLGAASAETQATGTWQ
YHRPLPPGGTVGLARLVRRRIGPKTTWSLQLQVRSPLPEREHEDRRPLVTVHPGWAADLSGR
RIAGIADAADPGLATVLQLPPDIEHGLQRAAELESTRSQARDALTPMLKVHPWPQELLNAAT
PEEDASASGDSGPMAPERIMCRKVADEILALRRLPAQHIAIRRLHRLARWLRLAEVDVPDWL
ETWRKEDKLRWQASAAAAKRARNRRRGFYRETALRLASQYQAIVIEPLNLADAAKKIDEATG
ERSDFAKKARAGRVVAAIFELDSAIRWAATKCGTAVLDLTGETAQHCAICGGHSLKADDEDS
QCLRCSDCGAAIDRKRNGAALAWQAAAAHLETHLEDFWRLTLENRASAAAKRDEKKTKLQEG
RRAAMRETLET

### EXAMPLES

The invention is further described in the following examples, which do not limit the scope of the invention described in the claims.

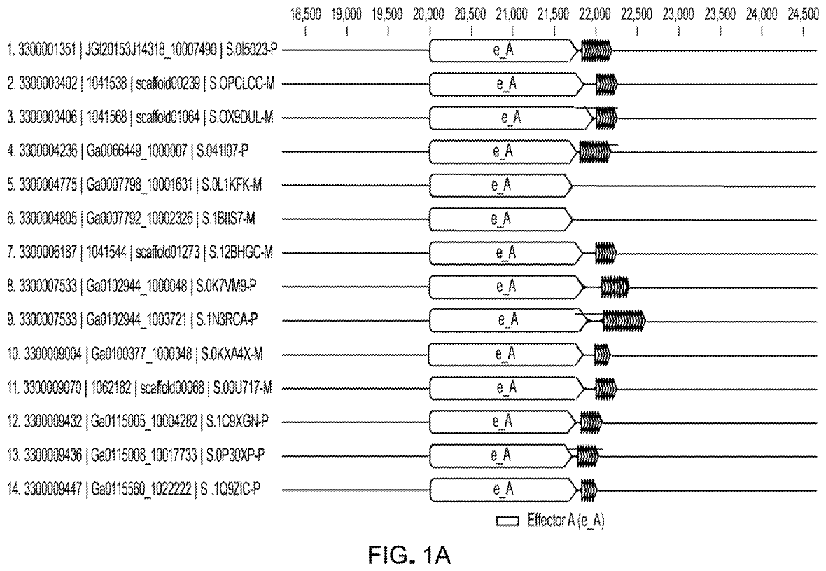

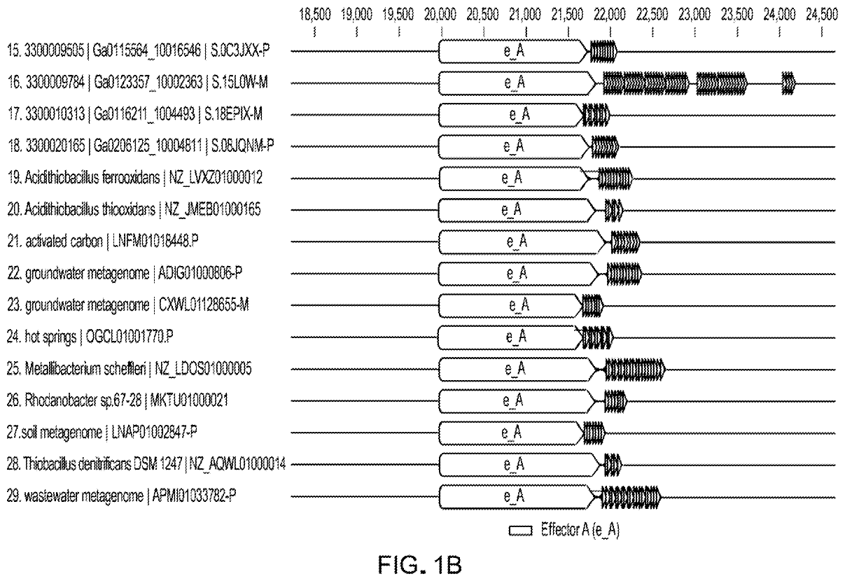

### Example 1—Identification of Minimal Components for CLUST.018837 CRISPR-Cas System (FIGS. 1-5)

Genome and metagenome sequences were downloaded from NCBI (Benson et al., 2013; Pruitt et al., 2012), NCBI whole genome sequencing (WGS), and DOE JGI Integrated Microbial Genomes (Markowitz et al., 2012) and processed as described in the Detailed Description of this disclosure.

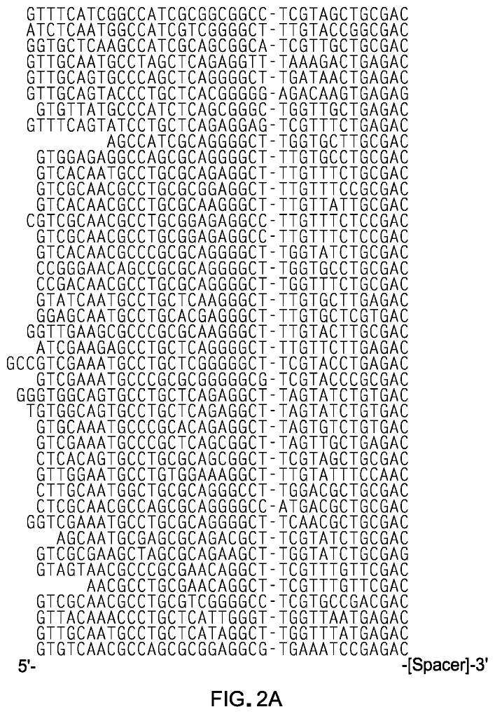

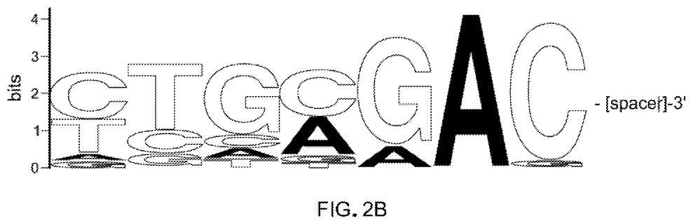

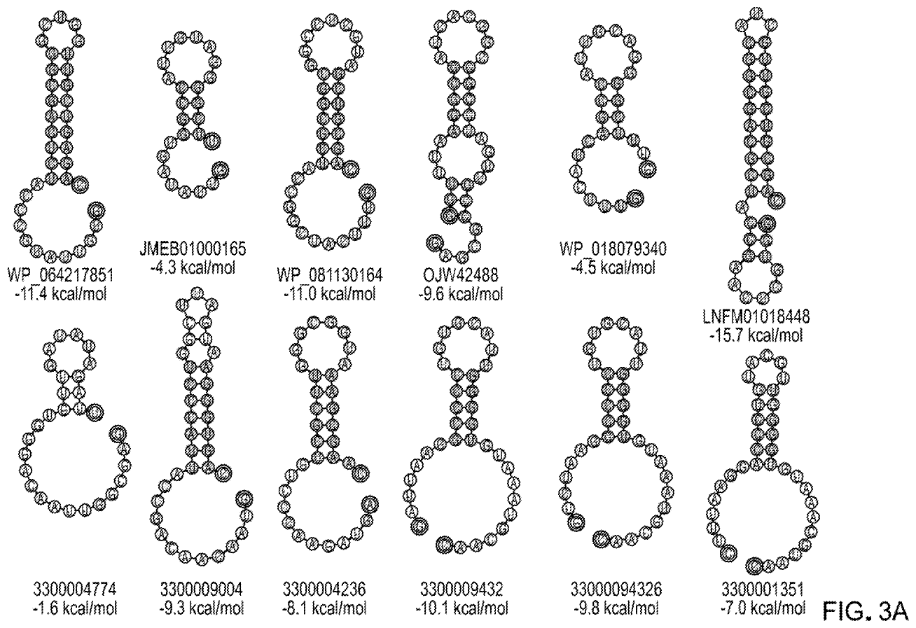

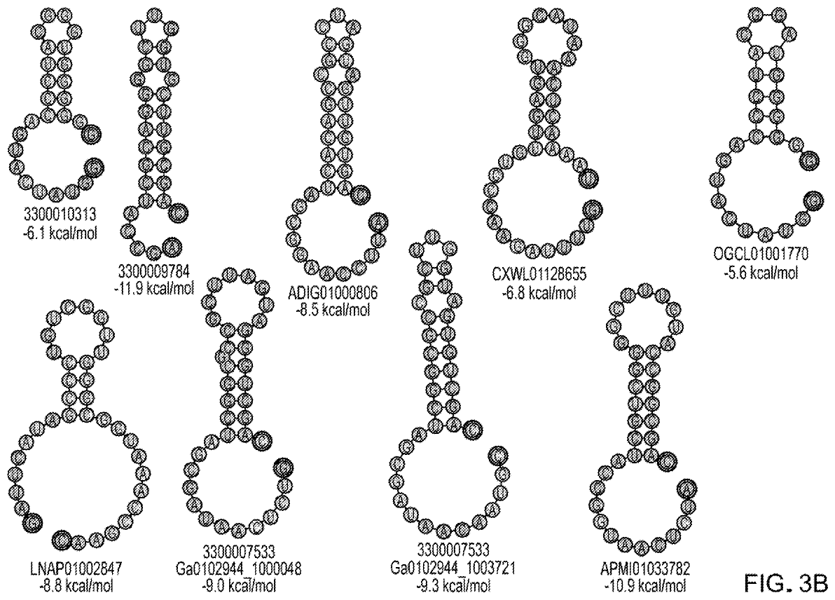

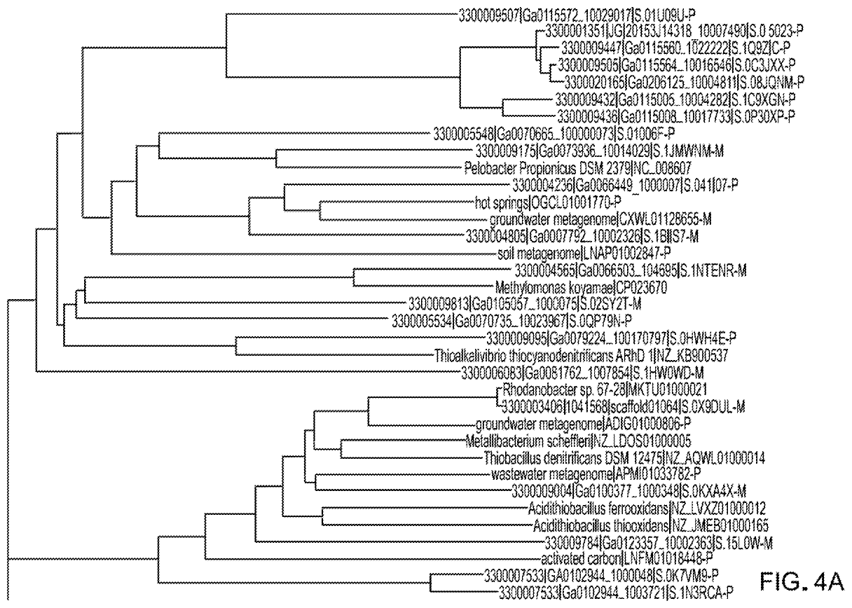

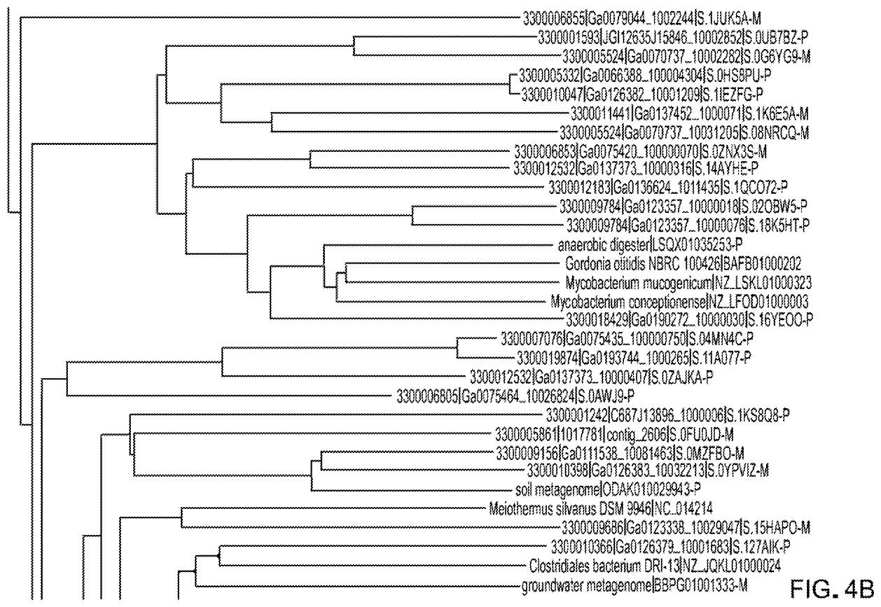

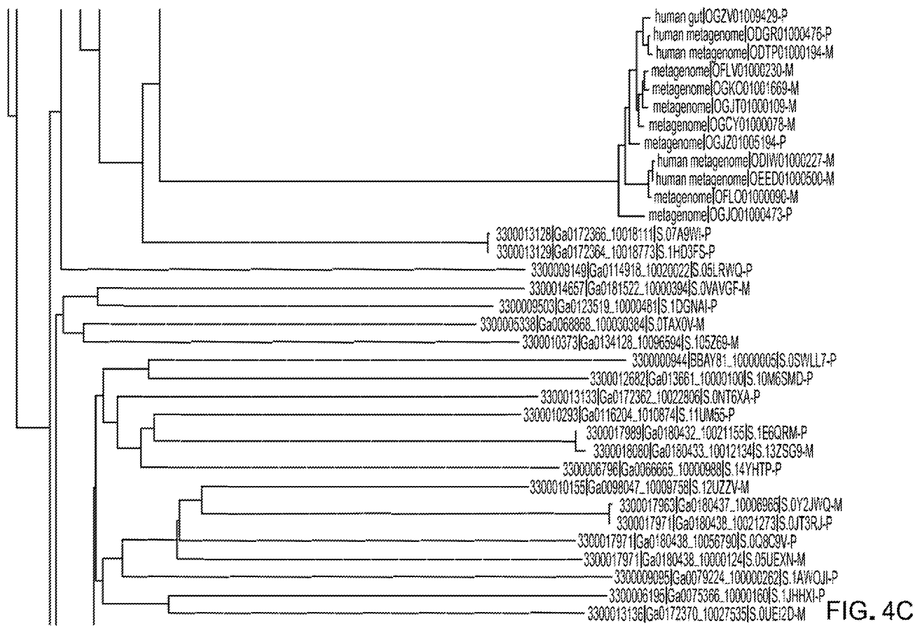

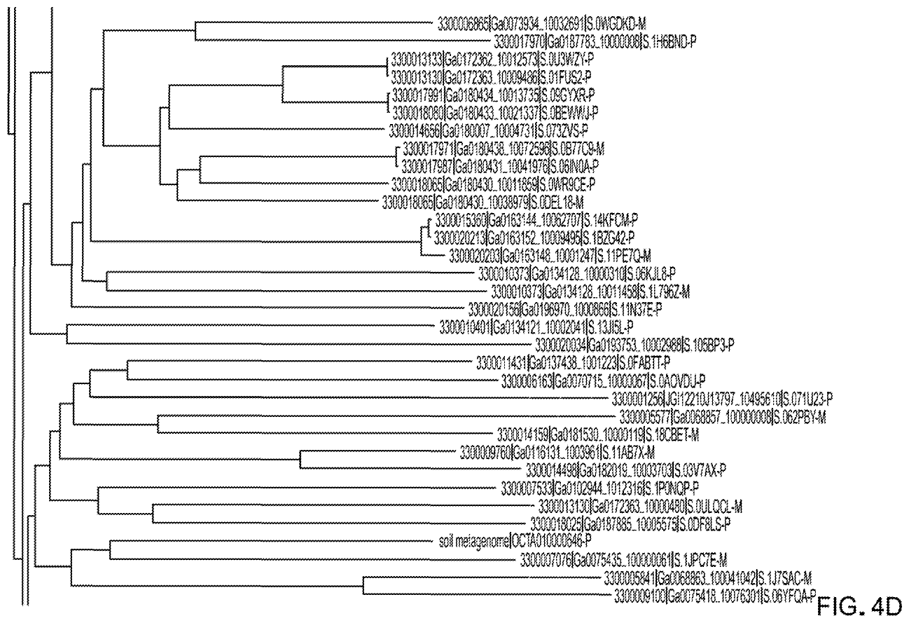

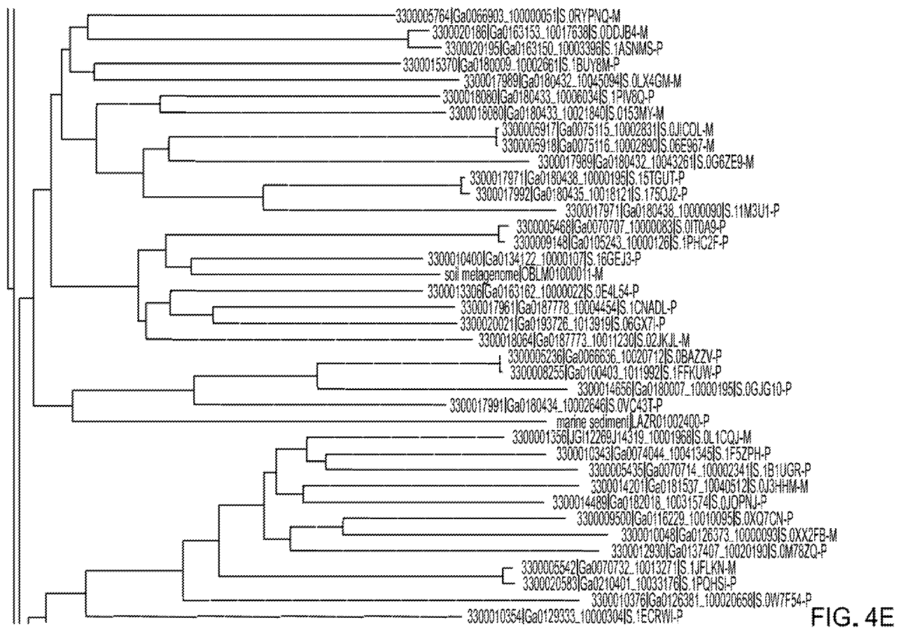

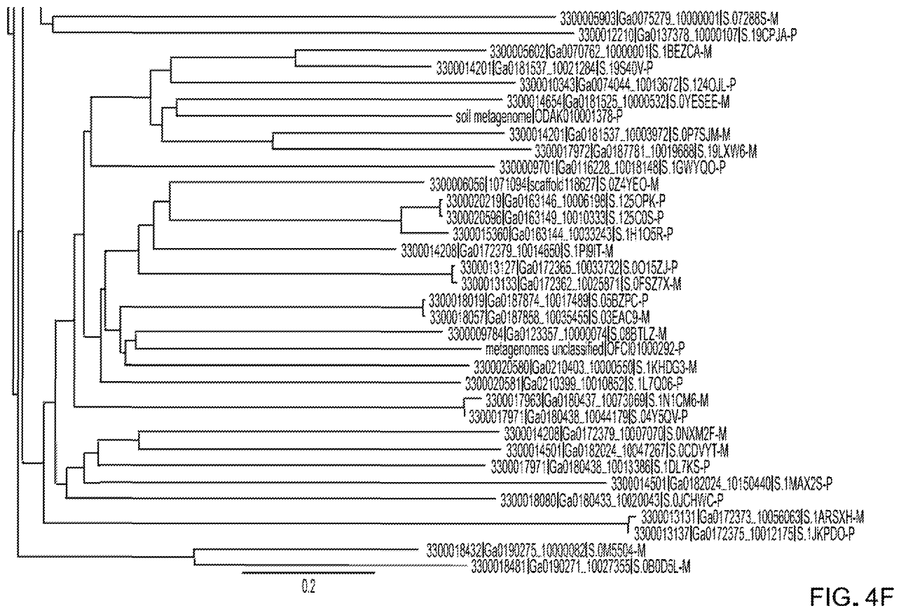

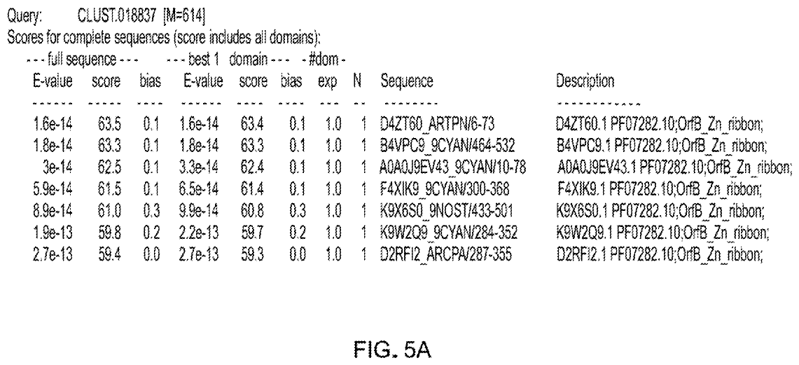

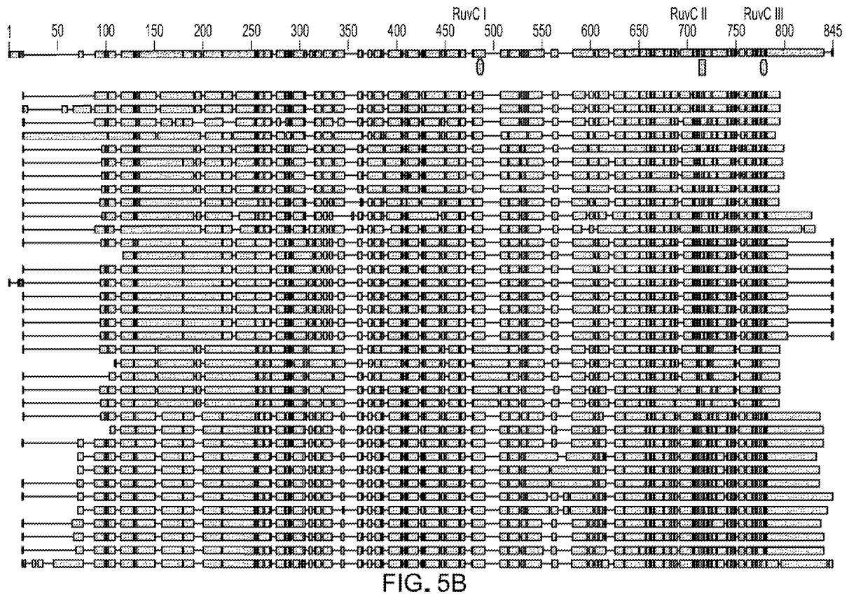

The identified CRISPR-Cas system described herein, designated CLUST.018837, contains a large single effector associated with CRISPR arrays found in Acidithiobacillus, Clostridiales, Gordonia, Metallibacterium, Mycobacterium, Pelobacter, Rhodanobacter, Thioalkalivibrio, and Thiobacillus bacteria, as well as uncultured metagenomic sequences collected from a range of environments, including termite gut, soil, ground water, waste water, marine, and hot springs environments (TABLE 1). CLUST.018837 effectors include the exemplary proteins detailed in TABLES 1 and 2. Exemplary direct repeat sequences for these systems are shown in TABLE 3.
Examples of naturally occurring loci containing this effector complex are depicted in FIGS. 1A-B, indicating that for loci containing the CLUST.018837 CRISPR-Cas system, the effector protein co-occurs with a CRISPR array. No other families of large proteins were identified within a bi-directional 15 kb window that co-occur with the effector protein or CRISPR array.
The direct repeat sequences for CLUST.018837 CRISPR-Cas systems show a consensus 5′-YBVMRAC-3′ (wherein Y is C or T; B is T, C, or G; V is G, C, or A; M is A or C; and R is A or G) nucleotide sequence at the 3′ terminal end (FIG. 2B).
The predicted secondary structure of direct repeat sequences for example CLUST.018837 CRISPR-Cas systems is depicted in FIGS. 3A-B, indicating a high prevalence of predicted stem loop structures.
FIGS. 4A-F, combined, show a phylogenetic tree of CLUST.018837 effectors, showing that the family exhibits sequence diversity and at a top level comprises three sub-families.
An HMM profile search of the multiple sequence alignment of CLUST.018837 effectors against the PFAM database indicates the presence of the OrfB_Zn_ribbon domain (FIG. 5A). Manual inspection of the multiple sequence alignment reveals the locations of the conserved catalytic residues of the RuvC domain, indicated in FIG. 5B. Notably, the RuvC I domain does not contain any highly conserviced residues across this family.

TABLE 1
Representative CLUST.018837 Effector Proteins

| | | | | | effec- |
|---|---|---|---|---|---|
| | | # | | | tor |
| Species | effector accession | spacers | cas1 | cas2 | size |
| Metallibacterium scheffleri | WP_081130164.1 | 9 | N | N | 627 |
| (NZ_LDOS01000005) | | | | | |
| Thiobacillus denitrificans DSM 12475 | WP_018079340.1 | 2 | N | N | 633 |
| (NZ_AQWL01000014) | | | | | |
| Acidithiobacillus ferrooxidans | WP_064217851.1 | 5 | N | N | 596 |
| (NZ_LVXZ01000012) | | | | | |
| Acidithiobacillus thiooxidans | JMEB01000165_11 | 2 | N | N | 593 |
| (JMEB01000165) | | | | | |
| Acidithiobacillus thiooxidans | WP_051690567.1 | 2 | N | N | 615 |
| (JMEB01000165) | | | | | |
| Rhodanobacter sp. 67-28 | OJW42488.1 | 3 | N | N | 617 |
| (MKTU01000021) | | | | | |
| activated carbon metagenome | LNFM01018448_6 | 4 | N | N | 655 |
| (LNFM01018448) | | | | | |
| aquatic-freshwater | 3300004774\|Ga0007794_10001723_8 | 2 | N | N | 573 |
| (3300004774\|Ga0007794_10001723) | | | | | |
| aquatic-freshwater | 3300004776\|Ga0007800_10001775_2 | 2 | N | N | 573 |
| (3300004776\|Ga0007800_10001775) | | | | | |
| aquatic-freshwater-aquifer | 3300009004\|Ga0100377_1000348_44 | 2 | N | N | 614 |
| (3300009004\|Ga0100377_1000348) | | | | | |
| aquatic-freshwater-freshwater sediment | 3300004236\|Ga0066449_1000007_83 | 5 | N | N | 582 |
| (3300004236\|Ga0066449_1000007) | | | | | |
| aquatic-marine | 3300009432\|Ga0115005_10004282_5 | 3 | N | N | 585 |
| (3300009432\|Ga0115005_10004282) | | | | | |
| aquatic-marine | 3300009436\|Ga0115008_10017733_3 | 3 | N | N | 587 |
| (3300009436\|Ga0115008_10017733) | | | | | |
| aquatic-marine | 3300009436\|Ga0115008_10017733_4 | 3 | N | N | 569 |
| (3300009436\|Ga0115008_10017733) | | | | | |
| aquatic-marine-pelagic marine | 3300001351\|JGI20153J14318_100074 | 5 | N | N | 585 |
| (3300001351\|JGI20153J14318_1000749 | 90_6 | | | | |
| 0) | | | | | |
| aquatic-marine-pelagic marine | 3300009447\|Ga0115560_1022222_2 | 2 | N | N | 585 |
| (3300009447\|Ga0115560_1022222) | | | | | |
| aquatic-marine-pelagic marine | 3300009505\|Ga0115564_10016546_3 | 4 | N | N | 586 |
| (3300009505\|Ga0115564_10016546) | | | | | |
| aquatic-marine-seawater | 3300020165\|Ga0206125_10004811_3 | 4 | N | N | 592 |
| (3300020165\|Ga0206125_10004811) | | | | | |
| aquatic-thermal springs-hot spring | 3300010313\|Ga0116211_1004493_2 | 4 | N | N | 577 |
| (3300010313\|Ga0116211_1004493) | | | | | |
| arthropoda-digestive system-termite gut | 3300009784\|Ga0123357_10002363_9 | 24 | N | N | 614 |
| (3300009784\|Ga0123357_10002363) | | | | | |
| groundwater metagenome | ADIG01000806_20 | 5 | N | N | 631 |
| (ADIG01000806) | | | | | |
| groundwater metagenome | CXWL01128655_18 | 3 | N | N | 575 |
| (CXWL01128655) | | | | | |
| hot springs metagenome | OGCL01001770_13 | 5 | N | N | 577 |
| (OGCL01001770) | | | | | |
| soil metagenome (LNAP01002847) | LNAP01002847_16 | 3 | N | N | 579 |
| terrestrial-soil-pond soil | 3300007533\|Ga0102944_1000048_72 | 4 | N | N | 621 |
| (3300007533\|Ga0102944_1000048) | | | | | |
| terrestrial-soil-pond soil | 3300007533\|Ga0102944_1003721_10 | 6 | N | N | 632 |
| (3300007533\|Ga0102944_1003721) | | | | | |
| terrestrial-soil-pond soil | 3300007533\|Ga0102944_1003721_8 | 6 | N | N | 621 |
| (3300007533\|Ga0102944_1003721) | | | | | |
| wastewater metagenome | APMI01033782_24 | 9 | N | N | 612 |
| (APMI01033782) | | | | | |
| Clostridiales bacterium DRI-13 | NZ_JQKL01000024_23 | 14 | N | N | 567 |
| (NZ_JQKL01000024) | | | | | |
| Clostridiales bacterium DRI-13 | WP_081908191.1 | 14 | N | N | 594 |
| (NZ_JQKL01000024) | | | | | |
| Gordonia otitidis NBRC 100426 | GAB36148.1 | 5 | N | N | 607 |
| (BAFB01000202) | | | | | |
| Gordonia otitidis NBRC 100426 | BAFB01000202_4 | 5 | N | N | 591 |
| (BAFB01000202) | | | | | |
| Gordonia otitidis NBRC 100426 | WP_039994403.1 | 5 | N | N | 597 |
| (NZ_BAFB01000202) | | | | | |
| Meiothermus silvanus DSM 9946 | WP_013159911.1 | 3 | N | N | 536 |
| (NC_014214) | | | | | |
| Methylomonas koyamae (NZ_CP023670) | WP_096876841.1 | 4 | N | N | 589 |
| Mycobacterium conceptionense | WP_048895525.1 | 9 | N | N | 603 |
| (NZ_LF0D01000003) | | | | | |
| Mycobacterium mucogenicum | WP_061006603.1 | 14 | N | N | 596 |
| (LSKL01000323) | | | | | |
| Pelobacter propionicus DSM 2379 | WP_011733919.1 | 3 | N | N | 664 |
| (CP000483) | | | | | |
| Thioalkalivibrio thiocyanodenitrificans | WP_018234394.1 | 3 | N | N | 599 |
| ARhD 1 (NZ_KB900537) | | | | | |
| algae-green algae-macroalgal surface- | 33000009441BBAY81_10000005_89 | 6 | N | N | 636 |
| ecklonia radiata 2 | | | | | |
| (33000009441BBAY81_10000005) | | | | | |
| anaerobic digester metagenome | LSQX01035253_23 | 5 | N | N | 592 |
| (LSQX01035253) | | | | | |
| aquatic-freshwater | 3300013131\|Ga0172373_10056063_2 | 2 | N | N | 696 |
| (3300013131\|Ga0172373_10056063) | | | | | |
| aquatic-freshwater | 3300013136\|Ga0172370_10027535_4 | 5 | N | N | 670 |
| (3300013136\|Ga0172370_10027535) | | | | | |
| aquatic-freshwater | 3300013137\|Ga0172375_10012175_6 | 10 | N | N | 655 |
| (3300013137\|Ga0172375_10012175) | | | | | |
| aquatic-freshwater-anoxic lake water | 3300010293\|Ga0116204_1010874_1 | 3 | N | N | 601 |
| (3300010293\|Ga0116204_1010874) | | | | | |
| aquatic-freshwater-anoxic lake water | 3300010293\|Ga0116204_1010874_2 | 3 | N | N | 620 |
| (3300010293\|Ga0116204_1010874) | | | | | |
| aquatic-freshwater-aquifer | 3300008255\|Ga0100403_1011992_3 | 7 | N | N | 588 |
| (3300008255\|Ga0100403_1011992) | | | | | |
| aquatic-freshwater-bog | 3300014155\|Ga0181524_10003409_2 | 3 | N | N | 685 |
| (3300014155\|Ga0181524_10003409) | 3 | | | | |
| aquatic-freshwater-bog | 3300014156\|Ga0181518_10000096_2 | 12 | N | N | 685 |
| (3300014156\|Ga0181518_10000096) | 8 | | | | |
| aquatic-freshwater-bog | 3300014158\|Ga0181521_10000063_9 | 11 | N | N | 685 |
| (3300014158\|Ga0181521_10000063) | 2 | | | | |
| aquatic-freshwater-bog | 3300014159\|Ga0181530_10000119_9 | 11 | N | N | 685 |
| (3300014159\|Ga0181530_10000119) | 8 | | | | |
| aquatic-freshwater-bog | 3300014201\|Ga0181537_10003972_1 | 2 | N | N | 702 |
| (3300014201\|Ga0181537_10003972) | 3 | | | | |
| aquatic-freshwater-bog | 3300014201\|Ga0181537_10021284_1 | 31 | N | N | 629 |
| (3300014201\|Ga0181537_10021284) | | | | | |
| aquatic-freshwater-bog | 3300014201\|Ga0181537_10040512_3 | 9 | N | N | 560 |
| (3300014201\|Ga0181537_10040512) | | | | | |
| aquatic-freshwater-bog | 3300014654\|Ga0181525_10000532_4 | 7 | N | N | 618 |
| (3300014654\|Ga0181525_10000532) | | | | | |
| aquatic-freshwater-bog | 3300014657\|Ga0181522_10000394_5 | 23 | N | N | 591 |
| (3300014657\|Ga0181522_10000394) | 2 | | | | |
| aquatic-freshwater-bog | 3300014657\|Ga0181522_10000394_5 | 23 | N | N | 610 |
| (3300014657\|Ga0181522_10000394) | 3 | | | | |
| aquatic-freshwater-freshwater lake | 3300009175\|Ga0073936_10014029_2 | 5 | N | N | 717 |
| hypolimnion | | | | | |
| (3300009175\|Ga0073936_10014029) | | | | | |
| aquatic-freshwater-freshwater microbial | 3300015360\|Ga0163144_10020017_5 | 5 | N | N | 611 |
| mat | | | | | |
| (3300015360\|Ga0163144_10020017) | | | | | |
| aquatic-freshwater-freshwater microbial | 3300015360\|Ga0163144_10020017_4 | 5 | N | N | 588 |
| mat | | | | | |
| (3300015360\|Ga0163144_10020017) | | | | | |
| aquatic-freshwater-freshwater microbial | 3300015360\|Ga0163144_10033243_8 | 2 | N | N | 603 |
| mat | | | | | |
| (3300015360\|Ga0163144_10033243) | | | | | |
| aquatic-freshwater-freshwater microbial | 3300015360\|Ga0163144_10033243_7 | 2 | N | N | 555 |
| mat | | | | | |
| (3300015360\|Ga0163144_10033243) | | | | | |
| aquatic-freshwater-freshwater microbial | 3300015360\|Ga0163144_10062707_6 | 24 | N | N | 571 |
| mat | | | | | |
| (3300015360\|Ga0163144_10062707) | | | | | |
| aquatic-freshwater-freshwater microbial | 3300015360\|Ga0163144_10062707_6 | 24 | N | N | 562 |
| mat | | | | | |
| (3300015360\|Ga0163144_10062707) | | | | | |
| aquatic-freshwater-freshwater microbial | 3300020057\|Ga0163151_10006104_16 | 5 | N | N | 611 |
| mat | | | | | |
| (3300020057\|Ga0163151_10006104) | | | | | |
| aquatic-freshwater-freshwater microbial | 3300020186\|Ga0163153_10017638_7 | 6 | N | N | 561 |
| mat | | | | | |
| (3300020186\|Ga0163153_10017638) | | | | | |
| aquatic-freshwater-freshwater microbial | 3300020195\|Ga0163150_10003396_1 | 19 | N | N | 570 |
| mat | 4 | | | | |
| (3300020195\|Ga0163150_10003396) | | | | | |
| aquatic-freshwater-freshwater microbial | 3300020203\|Ga0163148_10001247_2 | 13 | N | N | 565 |
| mat | | | | | |
| (3300020203\|Ga0163148_10001247) | | | | | |
| aquatic-freshwater-freshwater microbial | 3300020203\|Ga0163148_10001247_2 | 13 | N | N | 574 |
| mat | | | | | |
| (3300020203\|Ga0163148_10001247) | | | | | |
| aquatic-freshwater-freshwater microbial | 3300020213\|Ga0163152_10009495_1 | 15 | N | N | 571 |
| mat | 4 | | | | |
| (3300020213\|Ga0163152_10009495) | | | | | |
| aquatic-freshwater-freshwater microbial | 3300020213\|Ga0163152_10009495_1 | 15 | N | N | 562 |
| mat | 4 | | | | |
| (3300020213\|Ga0163152_10009495) | | | | | |
| aquatic-freshwater-freshwater microbial | 3300020219\|Ga0163146_10006198_1 | 5 | N | N | 611 |
| mat | 8 | | | | |
| (3300020219\|Ga0163146_10006198) | | | | | |
| aquatic-freshwater-freshwater microbial | 3300020596\|Ga0163149_10010333_1 | 8 | N | N | 611 |
| mat | 3 | | | | |
| (3300020596\|Ga0163149_10010333) | | | | | |
| aquatic-freshwater-freshwater microbial | 3300020596\|Ga0163149_10010333_1 | 8 | N | N | 588 |
| mat | 2 | | | | |
| (3300020596\|Ga0163149_10010333) | | | | | |
| aquatic-freshwater-freshwater sediment | 3300004174\|Ga0066406_1000030_21 | 6 | N | N | 593 |
| (3300004174\|Ga0066406_1000030) | | | | | |
| aquatic-freshwater-freshwater sediment | 3300004200\|Ga0066422_1000628_7 | 6 | N | N | 593 |
| (3300004200\|Ga0066422_1000628) | | | | | |
| aquatic-freshwater-freshwater sediment | 3300004205\|Ga0066415_1000057_23 | 6 | N | N | 593 |
| (3300004205\|Ga0066415_1000057) | | | | | |
| aquatic-freshwater-freshwater sediment | 3300004565\|Ga0066503_104695_4 | 6 | N | N | 593 |
| (3300004565\|Ga0066503_104695) | | | | | |
| aquatic-freshwater-glacier valley | 3300009686\|Ga0123338_10029047_2 | 4 | N | N | 535 |
| (3300009686\|Ga0123338_10029047) | | | | | |
| aquatic-freshwater-groundwater | 330000124210687J13896_1000006_1 | 24 | N | N | 599 |
| (330000124210687J13896_1000006) | 34 | | | | |
| aquatic-freshwater-groundwater | 3300005236\|Ga0066636_10020712_3 | 8 | N | N | 588 |
| (3300005236\|Ga0066636_10020712) | | | | | |
| aquatic-freshwater-groundwater | 3300014208\|Ga0172379_10007070_1 | 3 | N | N | 623 |
| (3300014208\|Ga0172379_10007070) | 5 | | | | |
| aquatic-freshwater-groundwater | 3300014208\|Ga0172379_10014650_2 | 5 | N | N | 612 |
| (3300014208\|Ga0172379_10014650) | | | | | |
| aquatic-freshwater-groundwater | 3300014613\|Ga0180008_1000021_8 | 6 | N | N | 627 |
| (3300014613\|Ga0180008_1000021) | | | | | |
| aquatic-freshwater-groundwater | 3300014613\|Ga0180008_1000021_9 | 6 | N | N | 658 |
| (3300014613\|Ga0180008_1000021) | | | | | |
| aquatic-freshwater-groundwater | 3300014656\|Ga0180007_10000195_4 | 3 | N | N | 627 |
| (3300014656\|Ga0180007_10000195) | 4 | | | | |
| aquatic-freshwater-groundwater | 3300014656\|Ga0180007_10000195_4 | 3 | N | N | 658 |
| (3300014656\|Ga0180007_10000195) | 8 | | | | |
| aquatic-freshwater-groundwater | 3300014656\|Ga0180007_10004731_7 | 3 | N | N | 560 |
| (3300014656\|Ga0180007_10004731) | | | | | |
| aquatic-freshwater-groundwater | 3300014656\|Ga0180007_10004731_5 | 3 | N | N | 561 |
| (3300014656\|Ga0180007_10004731) | | | | | |
| aquatic-freshwater-groundwater | 3300015370\|Ga0180009_10002661_7 | 8 | N | N | 589 |
| (3300015370\|Ga0180009_10002661) | | | | | |
| aquatic-freshwater-peatland | 3300009760\|Ga0116131_1003961_2 | 5 | N | N | 606 |
| (3300009760\|Ga0116131_1003961) | | | | | |
| aquatic-freshwater-peatland | 3300018019\|Ga0187874_10017489_1 | 4 | N | N | 623 |
| (3300018019\|Ga0187874_10017489) | | | | | |
| aquatic-freshwater-peatland | 3300018025\|Ga0187885_10005575_2 | 6 | N | N | 619 |
| (3300018025\|Ga0187885_10005575) | | | | | |
| aquatic-freshwater-peatland | 3300018025\|Ga0187885_10005575_1 | 6 | N | N | 642 |
| (3300018025\|Ga0187885_10005575) | | | | | |
| aquatic-freshwater-peatland | 3300018057\|Ga0187858_10035455_2 | 2 | N | N | 623 |
| (3300018057\|Ga0187858_10035455) | | | | | |
| aquatic-freshwater-polar desert sand | 3300012183\|Ga0136624_1011435_1 | 3 | N | N | 556 |
| (3300012183\|Ga0136624_1011435) | | | | | |
| aquatic-freshwater-polar desert sand | 3300012682\|Ga0136611_10000100_4 | 12 | N | N | 582 |
| (3300012682\|Ga0136611_10000100) | | | | | |
| aquatic-freshwater-sediment | 3300013127\|Ga0172365_10004082_5 | 2 | N | N | 547 |
| (3300013127\|Ga0172365_10004082) | | | | | |
| aquatic-freshwater-sediment | 3300013127\|Ga0172365_10004082_3 | 2 | N | N | 538 |
| (3300013127\|Ga0172365_10004082) | | | | | |
| aquatic-freshwater-sediment | 3300013127\|Ga0172365_10033732_1 | 2 | N | N | 610 |
| (3300013127\|Ga0172365_10033732) | | | | | |
| aquatic-freshwater-sediment | 3300013128\|Ga0172366_10016188_4 | 2 | N | N | 547 |
| (3300013128\|Ga0172366_10016188) | | | | | |
| aquatic-freshwater-sediment | 3300013128\|Ga0172366_10018111_5 | 6 | N | N | 543 |
| (3300013128\|Ga0172366_10018111) | | | | | |
| aquatic-freshwater-sediment | 3300013129\|Ga0172364_10001281_2 | 16 | N | N | 593 |
| (3300013129\|Ga0172364_10001281) | 6 | | | | |
| aquatic-freshwater-sediment | 3300013129\|Ga0172364_10017363_4 | 2 | N | N | 547 |
| (3300013129\|Ga0172364_10017363) | | | | | |
| aquatic-freshwater-sediment | 3300013129\|Ga0172364_10018773_2 | 7 | N | N | 543 |
| (3300013129\|Ga0172364_10018773) | | | | | |
| aquatic-freshwater-sediment | 3300013129\|Ga0172364_10045136_2 | 2 | N | N | 610 |
| (3300013129\|Ga0172364_10045136) | | | | | |
| aquatic-freshwater-sediment | 3300013130\|Ga0172363_10000480_2 | 4 | N | N | 593 |
| (3300013130\|Ga0172363_10000480) | 2 | | | | |
| aquatic-freshwater-sediment | 3300013130\|Ga0172363_10009486_8 | 2 | N | N | 547 |
| (3300013130\|Ga0172363_10009486) | | | | | |
| aquatic-freshwater-sediment | 3300013130\|Ga0172363_10014785_2 | 2 | N | N | 566 |
| (3300013130\|Ga0172363_10014785) | | | | | |
| aquatic-freshwater-sediment | 3300013133\|Ga0172362_10012573_3 | 2 | N | N | 547 |
| (3300013133\|Ga0172362_10012573) | | | | | |
| aquatic-freshwater-sediment | 3300013133\|Ga0172362_10022806_8 | 2 | N | N | 566 |
| (3300013133\|Ga0172362_10022806) | | | | | |
| aquatic-freshwater-sediment | 3300013133\|Ga0172362_10025871_2 | 2 | N | N | 610 |
| (3300013133\|Ga0172362_10025871) | | | | | |
| aquatic-marine | 3300010155\|Ga0098047_10009758_2 | 2 | N | N | 620 |
| (3300010155\|Ga0098047_10009758) | | | | | |
| aquatic-marine-aqueous | 3300006805\|Ga0075464_10026824_2 | 10 | N | N | 479 |
| (3300006805\|Ga0075464_10026824) | | | | | |
| aquatic-marine-aqueous | 3300006805\|Ga0075464_10026824_2 | 10 | N | N | 481 |
| (3300006805\|Ga0075464_10026824) | | | | | |
| aquatic-marine-deep subsurface | 3300009149\|Ga0114918_10020022_2 | 5 | N | N | 664 |
| (3300009149\|Ga0114918_10020022) | | | | | |
| aquatic-marine-diffuse hydrothermal | 3300006083\|Ga0081762_1007854_6 | 8 | N | N | 572 |
| flow volcanic vent | | | | | |
| (3300006083\|Ga0081762_1007854) | | | | | |
| aquatic-marine-freshwater to marine | 3300010354\|Ga0129333_10000304_8 | 6 | N | N | 551 |
| saline gradient | | | | | |
| (3300010354\|Ga0129333_10000304) | | | | | |
| aquatic-marine-freshwater to marine | 3300010354\|Ga0129333_10000304_1 | 6 | N | N | 574 |
| saline gradient | 0 | | | | |
| (3300010354\|Ga0129333_10000304) | | | | | |
| aquatic-marine-pelagic marine | 3300009507\|Ga0115572_10029017_2 | 4 | N | N | 600 |
| (3300009507\|Ga0115572_10029017) | | | | | |
| aquatic-non marine saline and alkaline- | 3300017963\|Ga0180437_10000100_1 | 17 | N | N | 642 |
| hypersaline lake sediment | 51 | | | | |
| (3300017963\|Ga0180437_10000100) | | | | | |
| aquatic-non marine saline and alkaline- | 3300017963\|Ga0180437_10000153_2 | 10 | N | N | 732 |
| hypersaline lake sediment | 5 | | | | |
| (3300017963\|Ga0180437_10000153) | | | | | |
| aquatic-non marine saline and alkaline- | 3300017963\|Ga0180437_10000488_7 | 6 | N | N | 584 |
| hypersaline lake sediment | 8 | | | | |
| (3300017963\|Ga0180437_10000488) | | | | | |
| aquatic-non marine saline and alkaline- | 3300017963\|Ga0180437_10000692_1 | 5 | N | N | 654 |
| hypersaline lake sediment | 3 | | | | |
| (3300017963\|Ga0180437_10000692) | | | | | |
| aquatic-non marine saline and alkaline- | 3300017963\|Ga0180437_10006965_2 | 6 | N | N | 670 |
| hypersaline lake sediment | 0 | | | | |
| (3300017963\|Ga0180437_10006965) | | | | | |
| aquatic-non marine saline and alkaline- | 3300017963\|Ga0180437_10006965_2 | 6 | N | N | 645 |
| hypersaline lake sediment | 0 | | | | |
| (3300017963\|Ga0180437_10006965) | | | | | |
| aquatic-non marine saline and alkaline- | 3300017963\|Ga0180437_10073069_2 | 7 | N | N | 625 |
| hypersaline lake sediment | | | | | |
| (3300017963\|Ga0180437_10073069) | | | | | |
| aquatic-non marine saline and alkaline- | 3300017971\|Ga0180438_10000090_9 | 10 | N | N | 732 |
| hypersaline lake sediment | 1 | | | | |
| (3300017971\|Ga0180438_10000090) | | | | | |
| aquatic-non marine saline and alkaline- | 3300017971\|Ga0180438_10000124_1 | 5 | N | N | 654 |
| hypersaline lake sediment | 14 | | | | |
| (3300017971\|Ga0180438_10000124) | | | | | |
| aquatic-non marine saline and alkaline- | 3300017971\|Ga0180438_10000195_1 | 17 | N | N | 642 |
| hypersaline lake sediment | 44 | | | | |
| (3300017971\|Ga0180438_10000195) | | | | | |
| aquatic-non marine saline and alkaline- | 3300017971\|Ga0180438_10013386_7 | 8 | N | N | 584 |
| hypersaline lake sediment | | | | | |
| (3300017971\|Ga0180438_10013386) | | | | | |
| aquatic-non marine saline and alkaline- | 3300017971\|Ga0180438_10021273_1 | 6 | N | N | 645 |
| hypersaline lake sediment | | | | | |
| (3300017971\|Ga0180438_10021273) | | | | | |
| aquatic-non marine saline and alkaline- | 3300017971\|Ga0180438_10044179_5 | 3 | N | N | 674 |
| hypersaline lake sediment | | | | | |
| (3300017971\|Ga0180438_10044179) | | | | | |
| aquatic-non marine saline and alkaline- | 3300017971\|Ga0180438_10056790_2 | 6 | N | N | 645 |
| hypersaline lake sediment | | | | | |
| (3300017971\|Ga0180438_10056790) | | | | | |
| aquatic-non marine saline and alkaline- | 3300017971\|Ga0180438_10072596_2 | 3 | N | N | 556 |
| hypersaline lake sediment | | | | | |
| (3300017971\|Ga0180438_10072596) | | | | | |
| aquatic-non marine saline and alkaline- | 3300017987\|Ga0180431_10022214_3 | 6 | N | N | 572 |
| hypersaline lake sediment | | | | | |
| (3300017987\|Ga0180431_10022214) | | | | | |
| aquatic-non marine saline and alkaline- | 3300017987\|Ga0180431_10041976_5 | 11 | N | N | 556 |
| hypersaline lake sediment | | | | | |
| (3300017987\|Ga0180431_10041976) | | | | | |
| aquatic-non marine saline and alkaline- | 3300017989\|Ga0180432_10002388_5 | 6 | N | N | 572 |
| hypersaline lake sediment | | | | | |
| (3300017989\|Ga0180432_10002388) | | | | | |
| aquatic-non marine saline and alkaline- | 3300017989\|Ga0180432_10021155_3 | 20 | N | N | 630 |
| hypersaline lake sediment | | | | | |
| (3300017989\|Ga0180432_10021155) | | | | | |
| aquatic-non marine saline and alkaline- | 3300017989\|Ga0180432_10021155_5 | 20 | N | N | 643 |
| hypersaline lake sediment | | | | | |
| (3300017989\|Ga0180432_10021155) | | | | | |
| aquatic-non marine saline and alkaline- | 3300017989\|Ga0180432_10043261_1 | 2 | N | N | 651 |
| hypersaline lake sediment | | | | | |
| (3300017989\|Ga0180432_10043261) | | | | | |
| aquatic-non marine saline and alkaline- | 3300017989\|Ga0180432_10045094_6 | 4 | N | N | 633 |
| hypersaline lake sediment | | | | | |
| (3300017989\|Ga0180432_10045094) | | | | | |
| aquatic-non marine saline and alkaline- | 3300017991\|Ga0180434_10002646_1 | 6 | N | N | 572 |
| hypersaline lake sediment | | | | | |
| (3300017991\|Ga0180434_10002646) | | | | | |
| aquatic-non marine saline and alkaline- | 3300017991\|Ga0180434_10013735_9 | 9 | N | N | 549 |
| hypersaline lake sediment | | | | | |
| (3300017991\|Ga0180434_10013735) | | | | | |
| aquatic-non marine saline and alkaline- | 3300017992\|Ga0180435_10018121_1 | 6 | N | N | 642 |
| hypersaline lake sediment | 1 | | | | |
| (3300017992\|Ga0180435_10018121) | | | | | |
| aquatic-non marine saline and alkaline- | 3300018065\|Ga0180430_10011859_2 | 11 | N | N | 560 |
| hypersaline lake sediment | | | | | |
| (3300018065\|Ga0180430_10011859) | | | | | |
| aquatic-non marine saline and alkaline- | 3300018065\|Ga0180430_10038979_3 | 7 | N | N | 567 |
| hypersaline lake sediment | | | | | |
| (3300018065\|Ga0180430_10038979) | | | | | |
| aquatic-non marine saline and alkaline- | 3300018080\|Ga0180433_10006034_1 | 13 | N | N | 575 |
| hypersaline lake sediment | 7 | | | | |
| (3300018080\|Ga0180433_10006034) | | | | | |
| aquatic-non marine saline and alkaline- | 3300018080\|Ga0180433_10006034_1 | 13 | N | N | 598 |
| hypersaline lake sediment | 8 | | | | |
| (3300018080\|Ga0180433_10006034) | | | | | |
| aquatic-non marine saline and alkaline- | 3300018080\|Ga0180433_10012134_6 | 13 | N | N | 610 |
| hypersaline lake sediment | | | | | |
| (3300018080\|Ga0180433_10012134) | | | | | |
| aquatic-non marine saline and alkaline- | 3300018080\|Ga0180433_10012134_6 | 13 | N | N | 642 |
| hypersaline lake sediment | | | | | |
| (3300018080\|Ga0180433_10012134) | | | | | |
| aquatic-non marine saline and alkaline- | 3300018080\|Ga0180433_10020043_6 | 12 | N | N | 640 |
| hypersaline lake sediment | | | | | |
| (3300018080\|Ga0180433_10020043) | | | | | |
| aquatic-non marine saline and alkaline- | 3300018080\|Ga0180433_10021337_5 | 10 | N | N | 549 |
| hypersaline lake sediment | | | | | |
| (3300018080\|Ga0180433_10021337) | | | | | |
| aquatic-non marine saline and alkaline- | 3300018080\|Ga0180433_10021840_7 | 5 | N | N | 584 |
| hypersaline lake sediment | | | | | |
| (3300018080\|Ga0180433_10021840) | | | | | |
| aquatic-non marine saline and alkaline- | 3300018080\|Ga0180433_10021840_7 | 5 | N | N | 601 |
| hypersaline lake sediment | | | | | |
| (3300018080\|Ga0180433_10021840) | | | | | |
| aquatic-non marine saline and alkaline- | 3300001256\|JGI12210J13797_104956 | 5 | N | N | 580 |
| hypersaline mat | 08_9 | | | | |
| (3300001256IJG112210J13797_1049560 | | | | | |
| 8) | | | | | |
| aquatic-non marine saline and alkaline- | 3300001256\|JGI12210J13797_104956 | 7 | N | N | 580 |
| hypersaline mat | 10_14 | | | | |
| (3300001256IJG112210J13797_1049561 | | | | | |
| 0) | | | | | |
| aquatic-non marine saline and alkaline- | 3300005917\|Ga0075115_10002831_4 | 14 | N | N | 635 |
| saline lake | | | | | |
| (3300005917\|Ga0075115_10002831) | | | | | |
| aquatic-non marine saline and alkaline- | 3300005918\|Ga0075116_10002890_7 | 3 | N | N | 635 |
| saline lake | | | | | |
| (3300005918\|Ga0075116_10002890) | | | | | |
| aquatic-sediment-groundwater | 3300011414\|Ga0137442_1000121_10 | 17 | N | N | 631 |
| sediment | | | | | |
| (3300011414\|Ga0137442_1000121) | | | | | |
| aquatic-sediment-groundwater | 3300011431\|Ga0137438_1001223_2 | 8 | N | N | 631 |
| sediment | | | | | |
| (3300011431\|Ga0137438_1001223) | | | | | |
| aquatic-sediment-groundwater | 3300011441\|Ga0137452_1000071_9 | 7 | N | N | 553 |
| sediment | | | | | |
| (3300011441\|Ga0137452_1000071) | | | | | |
| aquatic-thermal springs-hot spring | 3300006855\|Ga0079044_1002244_2 | 3 | N | N | 625 |
| (3300006855\|Ga0079044_1002244) | | | | | |
| aquatic-thermal springs-hot spring | 3300006855\|Ga0079044_1002244_2 | 3 | N | N | 649 |
| (3300006855\|Ga0079044_1002244) | | | | | |
| aquatic-thermal springs-hot spring | 3300009503\|Ga0123519_10000481_1 | 8 | N | N | 598 |
| (3300009503\|Ga0123519_10000481) | 9 | | | | |
| aquatic-thermal springs-hot spring | 3300009503\|Ga0123519_10000481_2 | 8 | N | N | 618 |
| (3300009503\|Ga0123519_10000481) | 2 | | | | |
| aquatic-thermal springs-hot spring | 3300006865\|Ga0073934_10032691_1 | 2 | N | N | 572 |
| sediment | | | | | |
| (3300006865\|Ga0073934_10032691) | | | | | |
| aquatic-thermal springs-hypersaline mat | 3300001340\|JGI20133J14441_100260 | 11 | N | N | 580 |
| (3300001340IJGI20133J14441_1002607) | 7_2 | | | | |
| arthropoda-digestive system-termite gut | 3300009784\|Ga0123357_10000018_1 | 2 | N | N | 619 |
| (3300009784\|Ga0123357_10000018) | 05 | | | | |
| arthropoda-digestive system-termite gut | 3300009784\|Ga0123357_10000074_4 | 2 | N | N | 667 |
| (3300009784\|Ga0123357_10000074) | 2 | | | | |
| arthropoda-digestive system-termite gut | 3300009784\|Ga0123357_10000076_3 | 2 | N | N | 618 |
| (3300009784\|Ga0123357_10000076) | 2 | | | | |
| groundwater metagenome | BBPF01004549_6 | 9 | N | N | 584 |
| (BBPF01004549) | | | | | |
| groundwater metagenome | BBPG01001333_4 | 8 | N | N | 584 |
| (BBPG01001333) | | | | | |
| human gut metagenome | OGZV01009429_1 | 3 | N | N | 567 |
| (OGZV01009429) | | | | | |
| human gut metagenome | OKWZ01000119_10 | 4 | N | N | 563 |
| (OKWZ01000119) | | | | | |
| human metagenome (ODGR01000476) | ODGR01000476_16 | 2 | N | N | 567 |
| human metagenome (ODIG01000268) | ODIG01000268_14 | 4 | N | N | 563 |
| human metagenome (ODIP01002140) | ODIP01002140_2 | 4 | N | N | 567 |
| human metagenome (ODIW01000227) | ODIW01000227_18 | 4 | N | N | 567 |
| human metagenome (ODJA01000260) | ODJA01000260_38 | 4 | N | N | 563 |
| human metagenome (ODJP01000229) | ODJP01000229_55 | 4 | N | N | 563 |
| human metagenome (ODKZ01007116) | ODKZ01007116_1 | 3 | N | N | 567 |
| human metagenome (ODM001000523) | ODM001000523_12 | 4 | N | N | 563 |
| human metagenome (ODTN01000195) | ODTN01000195_35 | 4 | N | N | 563 |
| human metagenome (ODTP01000194) | ODTP01000194_18 | 4 | N | N | 567 |
| human metagenome (ODWI01002981) | ODWI01002981_3 | 2 | N | N | 563 |
| human metagenome (ODZZ01005262) | ODZZ01005262_2 | 4 | N | N | 563 |
| human metagenome (OEED01000500) | OEED01000500_25 | 4 | N | N | 567 |
| human metagenome (OEFT01000529) | OEFT01000529_3 | 4 | N | N | 563 |
| marine sediment metagenome | LAZR01002400_15 | 20 | N | N | 492 |
| (LAZR01002400) | | | | | |
| marine sediment metagenome | LAZR01002400_19 | 20 | N | N | 511 |
| (LAZR01002400) | | | | | |
| metagenome (FLSK01003024) | FLSK01003024_2 | 4 | N | N | 563 |
| metagenome (OFLM01000072) | OFLM01000072_9 | 4 | N | N | 567 |
| metagenome (OFLO01000090) | OFLO01000090_50 | 4 | N | N | 567 |
| metagenome (OFLU01000140) | OFLU01000140_22 | 3 | N | N | 567 |
| metagenome (OFLV01000230) | OFLV01000230_3 | 3 | N | N | 567 |
| metagenome (OGCY01000078) | OGCY01000078_30 | 3 | N | N | 567 |
| metagenome (OGJ001000473) | OGJ001000473_2 | 4 | N | N | 563 |
| metagenome (OGJT01000109) | OGJT01000109_37 | 3 | N | N | 567 |
| metagenome (OGJZ01005194) | OGJZ01005194_5 | 2 | N | N | 567 |
| metagenome (OGK001001669) | OGK001001669_8 | 4 | N | N | 567 |
| metagenomes unclassified sequences. | OFC101000292_37 | 5 | N | N | 582 |
| (OFC101000292) | | | | | |
| plants-endosphere-populus endosphere | 3300006048\|Ga0075363_100000001_ | 4 | N | N | 634 |
| (3300006048\|Ga0075363_100000001) | 25 | | | | |
| plants-endosphere-populus endosphere | 3300006048\|Ga0075363_100000001_ | 4 | N | N | 648 |
| (3300006048\|Ga0075363_100000001) | 20 | | | | |
| plants-endosphere-populus endosphere | 3300006048\|Ga0075363_100000020_ | 18 | N | N | 488 |
| (3300006048\|Ga0075363_100000020) | 49 | | | | |
| plants-endosphere-populus endosphere | 3300006178\|Ga0075367_10000108_6 | 4 | N | N | 634 |
| (3300006178\|Ga0075367_10000108) | | | | | |
| plants-endosphere-populus endosphere | 3300006178\|Ga0075367_10000108_6 | 4 | N | N | 648 |
| (3300006178\|Ga0075367_10000108) | | | | | |
| plants-endosphere-populus endosphere | 3300006195\|Ga0075366_10000160_1 | 4 | N | N | 634 |
| (3300006195\|Ga0075366_10000160) | 3 | | | | |
| plants-peat moss-host associated | 3300009500\|Ga0116229_10010095_9 | 21 | N | N | 604 |
| (3300009500\|Ga0116229_10010095) | | | | | |
| plants-peat moss-host associated | 3300009701\|Ga0116228_10018148_5 | 4 | N | N | 683 |
| (3300009701\|Ga0116228_10018148) | | | | | |
| plants-rhizoplane-corn rhizosphere | 3300005577\|Ga0068857_100000008_ | 15 | N | N | 698 |
| (3300005577\|Ga0068857_100000008) | 197 | | | | |
| plants-rhizoplane-miscanthus | 3300005338\|Ga0068868_100030384_ | 5 | N | N | 637 |
| rhizosphere | 5 | | | | |
| (3300005338\|Ga0068868_100030384) | | | | | |
| plants-rhizoplane-switchgrass | 3300005841\|Ga0068863_100041042_ | 13 | N | N | 693 |
| rhizosphere | 2 | | | | |
| (3300005841\|Ga0068863_100041042) | | | | | |
| plants-rhizoplane-switchgrass | 3300013306\|Ga0163162_10000022_1 | 20 | N | N | 586 |
| rhizosphere | 53 | | | | |
| (3300013306\|Ga0163162_10000022) | | | | | |
| plants-rhizosphere-miscanthus | 3300009148\|Ga0105243_10000126_6 | 10 | N | N | 626 |
| rhizosphere | 0 | | | | |
| (3300009148\|Ga0105243_10000126) | | | | | |
| plants-rhizosphere-populus rhizosphere | 3300006846\|Ga0075430_100000057_ | 3 | N | N | 617 |
| (3300006846\|Ga0075430_100000057) | 67 | | | | |
| plants-rhizosphere-populus rhizosphere | 3300006853\|Ga0075420_100000070_ | 3 | N | N | 617 |
| (3300006853\|Ga0075420_100000070) | 3 | | | | |
| plants-rhizosphere-populus rhizosphere | 3300006854\|Ga0075425_100000037_ | 22 | N | N | 488 |
| (3300006854\|Ga0075425_100000037) | 57 | | | | |
| plants-rhizosphere-populus rhizosphere | 3300006903\|Ga0075426_10000611_2 | 2 | N | N | 646 |
| (3300006903\|Ga0075426_10000611) | 8 | | | | |
| plants-rhizosphere-populus rhizosphere | 3300006914\|Ga0075436_100000782_ | 2 | N | N | 646 |
| (3300006914\|Ga0075436_100000782) | 9 | | | | |
| plants-rhizosphere-populus rhizosphere | 3300007076\|Ga0075435_100000061_ | 2 | N | N | 646 |
| (3300007076\|Ga0075435_100000061) | 47 | | | | |
| plants-rhizosphere-populus rhizosphere | 3300007076\|Ga0075435_100000750_ | 22 | N | N | 488 |
| (3300007076\|Ga0075435_100000750) | 29 | | | | |
| plants-rhizosphere-populus rhizosphere | 3300009100\|Ga0075418_10076301_2 | 6 | N | N | 710 |
| (3300009100\|Ga0075418_10076301) | | | | | |
| plants-rhizosphere-populus rhizosphere | 3300009100\|Ga0075418_10076301_2 | 6 | N | N | 713 |
| (3300009100\|Ga0075418_10076301) | | | | | |
| plants-rhizosphere-populus rhizosphere | 3300009156\|Ga0111538_10081463_8 | 3 | N | N | 558 |
| (3300009156\|Ga0111538_10081463) | | | | | |
| plants-rhizosphere-switchgrass | 3300005548\|Ga0070665_100000073_ | 7 | N | N | 597 |
| rhizosphere | 173 | | | | |
| (3300005548\|Ga0070665_100000073) | | | | | |
| soil metagenome (OBLM01000011) | OBLM01000011_1 | 2 | N | N | 635 |
| soil metagenome (OCTA010000646) | OCTA010000646_37 | 6 | N | N | 628 |
| soil metagenome (ODAK010001378) | ODAK010001378_33 | 5 | N | N | 617 |
| soil metagenome (ODAK010029943) | ODAK010029943_5 | 11 | N | N | 595 |
| soil metagenome (ODAK010029943) | ODAK010029943_6 | 11 | N | N | 638 |
| terrestrial-soil | 3300005602\|Ga0070762_10000001_3 | 50 | N | N | 628 |
| (3300005602\|Ga0070762_10000001) | 4 | | | | |
| terrestrial-soil | 3300005602\|Ga0070762_10000001_3 | 50 | N | N | 660 |
| (3300005602\|Ga0070762_10000001) | 2 | | | | |
| terrestrial-soil | 3300006796\|Ga0066665_10000988_1 | 2 | N | N | 628 |
| (3300006796\|Ga0066665_10000988) | 5 | | | | |
| terrestrial-soil | 3300018429\|Ga0190272_10000030_1 | 4 | N | N | 622 |
| (3300018429\|Ga0190272_10000030) | 13 | | | | |
| terrestrial-soil | 3300018432\|Ga0190275_10000082_1 | 10 | N | N | 605 |
| (3300018432\|Ga0190275_10000082) | 54 | | | | |
| terrestrial-soil | 3300018481\|Ga0190271_10027355_3 | 7 | N | N | 596 |
| (3300018481\|Ga0190271_10027355) | | | | | |
| terrestrial-soil | 3300019874\|Ga0193744_1000265_21 | 4 | N | N | 488 |
| (3300019874\|Ga0193744_1000265) | | | | | |
| terrestrial-soil | 3300020021\|Ga0193726_1013919_1 | 3 | N | N | 711 |
| (3300020021\|Ga0193726_1013919) | | | | | |
| terrestrial-soil | 3300020021\|Ga0193726_1013919_1 | 3 | N | N | 745 |
| (3300020021\|Ga0193726_1013919) | | | | | |
| terrestrial-soil | 3300020034\|Ga0193753_10002988_1 | 2 | N | N | 630 |
| (3300020034\|Ga0193753_10002988) | 0 | | | | |
| terrestrial-soil | 3300020034\|Ga0193753_10002988_9 | 2 | N | N | 669 |
| (3300020034\|Ga0193753_10002988) | | | | | |
| terrestrial-soil | 3300020156\|Ga0196970_1000866_40 | 6 | N | N | 559 |
| (3300020156\|Ga0196970_1000866) | | | | | |
| terrestrial-soil | 3300020579\|Ga0210407_10000200_1 | 8 | N | N | 621 |
| (3300020579\|Ga0210407_10000200) | 4 | | | | |
| terrestrial-soil | 3300020580\|Ga0210403_10000550_3 | 8 | N | N | 621 |
| (3300020580\|Ga0210403_10000550) | 5 | | | | |
| terrestrial-soil | 3300020580\|Ga0210403_10001296_1 | 5 | N | N | 518 |
| (3300020580\|Ga0210403_10001296) | 7 | | | | |
| terrestrial-soil | 3300020581\|Ga0210399_10010852_9 | 9 | N | N | 596 |
| (3300020581\|Ga0210399_10010852) | | | | | |
| terrestrial-soil | 3300020583\|Ga0210401_10033176_5 | 3 | N | N | 518 |
| (3300020583\|Ga0210401_10033176) | | | | | |
| terrestrial-soil-agricultural soil | 3300005435\|Ga0070714_100002341_ | 11 | N | N | 521 |
| (3300005435\|Ga0070714_100002341) | 12 | | | | |
| terrestrial-soil-agricultural soil | 3300009095\|Ga0079224_100000262_ | 6 | N | N | 573 |
| (3300009095\|Ga0079224_100000262) | 28 | | | | |
| terrestrial-soil-agricultural soil | 3300009095\|Ga0079224_100170797_ | 3 | N | N | 618 |
| (3300009095\|Ga0079224_100170797) | 3 | | | | |
| terrestrial-soil-bog forest soil | 3300010343\|Ga0074044_10013672_1 | 9 | N | N | 672 |
| (3300010343\|Ga0074044_10013672) | | | | | |
| terrestrial-soil-bog forest soil | 3300010343\|Ga0074044_10041345_4 | 3 | N | N | 561 |
| (3300010343\|Ga0074044_10041345) | | | | | |
| terrestrial-soil-corn, switchgrass and | 3300005468\|Ga0070707_100000083_ | 3 | N | N | 628 |
| miscanthus rhizosphere | 12 | | | | |
| (3300005468\|Ga0070707_100000083) | | | | | |
| terrestrial-soil-corn, switchgrass and | 3300006163\|Ga0070715_10000067_4 | 42 | N | N | 690 |
| miscanthus rhizosphere | 4 | | | | |
| (3300006163\|Ga0070715_10000067) | | | | | |
| terrestrial-soil-fen | 3300014498\|Ga0182019_10003703_1 | 4 | N | N | 630 |
| (3300014498\|Ga0182019_10003703) | | | | | |
| terrestrial-soil-forest soil | 3300001131\|JGI12631J13338_100029 | 22 | N | N | 674 |
| (3300001131\|JGI12631J13338_1000296) | 6_13 | | | | |
| terrestrial-soil-forest soil | 3300001593\|JGI12635J15846_100028 | 22 | N | N | 674 |
| (3300001593\|JGI12635J15846_1000285 | 52_1 | | | | |
| 2) | | | | | |
| terrestrial-soil-groundwater sand | 3300009813\|Ga0105057_1000075_5 | 8 | N | N | 600 |
| (3300009813\|Ga0105057_1000075) | | | | | |
| terrestrial-soil-groundwater sand | 3300009813\|Ga0105057_1000075_5 | 8 | N | N | 604 |
| (3300009813\|Ga0105057_1000075) | | | | | |
| terrestrial-soil-palsa | 3300014489\|Ga0182018_10031574_1 | 4 | N | N | 525 |
| (3300014489\|Ga0182018_10031574) | | | | | |
| terrestrial-soil-palsa | 3300014501\|Ga0182024_10047267_8 | 13 | N | N | 643 |
| (3300014501\|Ga0182024_10047267) | | | | | |
| terrestrial-soil-palsa | 3300014501\|Ga0182024_10150440_2 | 3 | N | N | 640 |
| (3300014501\|Ga0182024_10150440) | | | | | |
| terrestrial-soil-peatlands soil | 3300001356\|JG112269J14319_100019 | 5 | N | N | 552 |
| (3300001356\|JG112269J14319_1000196 | 68_12 | | | | |
| 8) | | | | | |
| terrestrial-soil-pond soil | 3300007533\|Ga0102944_1012316_2 | 13 | N | N | 622 |
| (3300007533\|Ga0102944_1012316) | | | | | |
| terrestrial-soil-rice paddy soil | 3300005903\|Ga0075279_10000001_3 | 5 | N | N | 701 |
| (3300005903\|Ga0075279_10000001) | 0 | | | | |
| terrestrial-soil-surface soil | 3300005524\|Ga0070737_10002282_1 | 8 | N | N | 739 |
| (3300005524\|Ga0070737_10002282) | 0 | | | | |
| terrestrial-soil-surface soil | 3300005524\|Ga0070737_10031205_1 | 5 | N | N | 615 |
| (3300005524\|Ga0070737_10031205) | | | | | |
| terrestrial-soil-surface soil | 3300005524\|Ga0070737_10031205_1 | 5 | N | N | 628 |
| (3300005524\|Ga0070737_10031205) | | | | | |
| terrestrial-soil-surface soil | 3300005534\|Ga0070735_10023967_5 | 2 | N | N | 607 |
| (3300005534\|Ga0070735_10023967) | | | | | |
| terrestrial-soil-surface soil | 3300005542\|Ga0070732_10013271_3 | 2 | N | N | 520 |
| (3300005542\|Ga0070732_10013271) | | | | | |
| terrestrial-soil-terrestrial soil | 3300010373\|Ga0134128_10000310_1 | 4 | N | N | 670 |
| (3300010373\|Ga0134128_10000310) | 09 | | | | |
| terrestrial-soil-terrestrial soil | 3300010373\|Ga0134128_10011458_1 | 4 | N | N | 675 |
| (3300010373\|Ga0134128_10011458) | | | | | |
| terrestrial-soil-terrestrial soil | 3300010373\|Ga0134128_10096594_3 | 4 | N | N | 674 |
| (3300010373\|Ga0134128_10096594) | | | | | |
| terrestrial-soil-terrestrial soil | 3300010400\|Ga0134122_10000107_5 | 2 | N | N | 631 |
| (3300010400\|Ga0134122_10000107) | 7 | | | | |
| terrestrial-soil-terrestrial soil | 3300010401\|Ga0134121_10002041_1 | 2 | N | N | 564 |
| (3300010401\|Ga0134121_10002041) | 7 | | | | |
| terrestrial-soil-tropical forest soil | 3300004633\|Ga0066395_10000027_3 | 9 | N | N | 586 |
| (3300004633\|Ga0066395_10000027) | 2 | | | | |
| terrestrial-soil-tropical forest soil | 3300005332\|Ga0066388_100004304_ | 7 | N | N | 644 |
| (3300005332\|Ga0066388_100004304) | 4 | | | | |
| terrestrial-soil-tropical forest soil | 3300005332\|Ga0066388_100004304_ | 7 | N | N | 619 |
| (3300005332\|Ga0066388_100004304) | 2 | | | | |
| terrestrial-soil-tropical forest soil | 3300005764\|Ga0066903_100000051_ | 9 | N | N | 586 |
| (3300005764\|Ga0066903_100000051) | 27 | | | | |
| terrestrial-soil-tropical forest soil | 3300010047\|Ga0126382_10001209_1 | 5 | N | N | 651 |
| (3300010047\|Ga0126382_10001209) | 4 | | | | |
| terrestrial-soil-tropical forest soil | 3300010047\|Ga0126382_10001209_1 | 5 | N | N | 619 |
| (3300010047\|Ga0126382_10001209) | 2 | | | | |
| terrestrial-soil-tropical forest soil | 3300010048\|Ga0126373_10000093_1 | 4 | N | N | 598 |
| (3300010048\|Ga0126373_10000093) | 02 | | | | |
| terrestrial-soil-tropical forest soil | 3300010366\|Ga0126379_10001683_1 | 6 | N | N | 619 |
| (3300010366\|Ga0126379_10001683) | 0 | | | | |
| terrestrial-soil-tropical forest soil | 3300010376\|Ga0126381_100020658_ | 3 | N | N | 592 |
| (3300010376\|Ga0126381_100020658) | 4 | | | | |
| terrestrial-soil-tropical forest soil | 3300010398\|Ga0126383_10032213_5 | 2 | N | N | 570 |
| (3300010398\|Ga0126383_10032213) | | | | | |
| terrestrial-soil-tropical peatland | 3300017961\|Ga0187778_10004454_1 | 4 | N | N | 612 |
| (3300017961\|Ga0187778_10004454) | | | | | |
| terrestrial-soil-tropical peatland | 3300017970\|Ga0187783_10000008_2 | 16 | N | N | 565 |
| (3300017970\|Ga0187783_10000008) | 3 | | | | |
| terrestrial-soil-tropical peatland | 3300017972\|Ga0187781_10019688_5 | 13 | N | N | 705 |
| (3300017972\|Ga0187781_10019688) | | | | | |
| terrestrial-soil-tropical peatland | 3300018064\|Ga0187773_10011230_2 | 2 | N | N | 640 |
| (3300018064\|Ga0187773_10011230) | | | | | |
| terrestrial-soil-vadose zone soil | 3300012204\|Ga0137374_10001132_4 | 22 | N | N | 666 |
| (3300012204\|Ga0137374_10001132) | | | | | |
| terrestrial-soil-vadose zone soil | 3300012210\|Ga0137378_10000107_4 | 3 | N | N | 670 |
| (3300012210\|Ga0137378_10000107) | 7 | | | | |
| terrestrial-soil-vadose zone soil | 3300012532\|Ga0137373_10000316_4 | 22 | N | N | 666 |
| (3300012532\|Ga0137373_10000316) | | | | | |
| terrestrial-soil-vadose zone soil | 3300012532\|Ga0137373_10000407_4 | 26 | N | N | 479 |
| (3300012532\|Ga0137373_10000407) | 3 | | | | |
| terrestrial-soil-vadose zone soil | 3300012930\|Ga0137407_10020190_4 | 5 | N | N | 545 |
| (3300012930\|Ga0137407_10020190) | | | | | |
| wastewater-nutrient removal- | 3300005987\|1071089\|scaffold14955_ | 13 | N | N | 632 |
| wastewater effluent | 2 | | | | |
| (3300005987110710891scaffold14955) | | | | | |
| wastewater-nutrient removal- | 3300005988\|1071091\|scaffold06014_ | 13 | N | N | 632 |
| wastewater effluent | 8 | | | | |
| (3300005988110710911scaffold06014) | | | | | |
| wastewater-nutrient removal- | 3300006056\|1071094\|scaffold118627_ | 5 | N | N | 632 |
| wastewater effluent | 2 | | | | |
| (3300006056110710941scaffold118627) | | | | | |

TABLE 2
Amino Acid Sequences of Representative CLUST.018837 Effector Proteins*

>WP_081130164.1
[Metallibacterium scheffleri]
MKLSPALPPTGDVLIYEYGARVDGDCLPAVGDQIAKARRLYNDLVAVIRGIVDEMRGFVLKHAGSEALALQAR
IDGLSEAFDAARAANDEDRMKQIAGERRALWAELGEQVKAVRKAHRAEIQELFLSRIGKKSTCDTYQMRCKAV
GDGLGWATANQVLDAALQAFKTSFQRGQAPRFARGEEKIQDTLTLQFTAAGGVPVAALLSGDHSELSMVSSCG
RRKYGSFSFRLGSASADTYANGTWQYHRPLPDGATVGLARLVRRSVGKDFKWALQLMVKRPATEPAMMEGRKP
LVAVHFGWAGDASGRRVAGITDGADPGVARVLQLPVEVEDGIRRAAEFQSARDEARDVIMTTIKNIAWGDAVA
CLGESSQFMHGSEPWLRARLSEELSTIRRLPAQHVAPRRLHRLCGLLRATNQMHDELEAWRKQDRLAWQASAH
MARRARNLRKDFYRRVAIDLARRYSAIVLEPLDLAAAALKVNEITGEKTEFAKKARSGRVVAAIYELESSIRW
AAAKSGTALLDLSGAETAARCGICGGASQSDESNSQVLHCVECGAELDRKKNGAAIAWQFAHENLDEAVTDFW
AAVIAQRCEHAEKTREKKAKMAEGRRLARTLSAGVSAVGSRNV (SEQ ID NO: 1)
>WP_018079340.1
[Thiobacillus denitfificans DSM 12475]
MSEIKPSLLPQGNVLIYEYGARLDKDCIQAVGDQIIKSRRLYNDLVATIRGIVTEMKAFVLEKSGPDAQRCQE
EIDALNAAFDAARAENNEDAMKCIAESRREKWRELAVFVKEARKNHRSDIQSMYLSRIGKNSACETYRIRSKA
VADGLGWATANQVLDAALTAFKKSFARGNAPRFAVGEDKDQDTLTLQFTAAGGVPVDTILAGKHGEVALSPTN
GCGPRKYGELRFRLGAAKAATNATGTWQYHRPLPDGATAGLCRLIRRRVGKDYKWAIQMQVKRPPIEQEALAG
RKPLVAVHFGWAANDEGRCVAGITDGADPGQAYVLKLPAEVEQSLVRSSAIQSERDSARDAIVPRLKEIEVPD
MDIESVESLPPDSPEVRLARAADELKAIHRLPANHVAIRRLHRLCGMLRDVDFLPEWLEDWRKEDRLQWQSAA
HIARRARNTRKGFYRQTAIDLARQYSSIVLEPLDLAKAAVKIDEITGERTEFAKKARAGRVVAALYELESAIR
WAAAKAGSAMFELTGETASRCSICGGDVLPDETNGQLLHCTECGADLDRKQNGAAMAWQLANDDLESLVEAFW
TETFAARRSAENEQAEKKQKMAEGRRKARTPIGGENTEVSRDSGNGANA (SEQ ID NO: 2)
>WP_064217851.1
[Acidithiobacillus ferrooxidans]
MSTITYEYGVRLEPDCIQHVDHQIILARGTYNEMIAAMRSVHDAAQSFQMEKAGPEGRAIAARIEALNTAFKE
ARAQQQEESLLQAIAVERRQCWRDLGVILKGVRQEHKKTLQEVFYNRIGINKGTDTYAIRCKAVADGLGWATA
QDVLNRAIIAWKMSMKLGRAPQFARGDEKTQDALTVQFTEKGGMPKDKMLEGESAVIGVEQPENTGKRAYGHF
WFRLGSASEGHYARGTIQWHRDLPEDASMASARLVRKRTGCKMKYYMQYVINTAQIRQVSDHARKALLAVHMG
WSADISGRRVCGITDAADPELAQIIQLPPEIERNIQRAANIQGKRDQARDEIAPKIRAFDGSLPPEWDESTQD
YWSHWKVLPANHMAASRIHAWRKRLGDFAPEWMAEWCKADRMLWIAATHTAQRARNRRKDFYRNLAKTWASQY
EAIVIEKPDIKKAAKILDEATGERTEFAKKARAGRVLASLYTLDSAIRWACQKNGTAILDMNGEKTAATCAMC
ASEAIRADTEDGQVLHCADCGAVLDRKKNGAAVAWQLVNEQRENLVEEYWAEQLNKEREAAEAKASRLEKMQA
ARRAKREPALAD (SEQ ID NO: 3)
>JMEB01000165_11
[Acidithiobacillus thiooxidans]
MNLKVCGDIDDQIRRARAMYNNIIAVMRGIYDEMQTFTMEHAGPEGQALHEKIVAANVAFDAAKADNDEPRMK
QIAMERRELWKALSIILKEVRKEHKNTLKERFYSRIGNNSSTETYQCRAEAIVGGLGYATATKVLDNALKAWQ
MSMVKGKAPRFARGEEKDQDTLTLQFSQAGGVPVEDIFTGKRKDIGIEYPKKGFGPRSYSAFRFRLGAASEES
YAEGTVQLHRAIPENARIAMAHLTRKKAGRKYQYELQLLATLAEPINLLPDHRRKPLVAIHFGWSGDEEGRRL
AGIADNADPLEARLLTLPPDIEDDIREASALQAKRDTYRDEVFLRLKEENTLPTKGETPLSEHWNKIRKLPAQ
HVSANRMHHLAWLVKSELIEIPEWFETWRKADQRMWVQATSLARRARNRRKKYYEKVAIDLASRYEAILIEMP
DLKKSAEKVNEKTGEKTEFAKKARSGRVIAALYVLESAIQWAACKHGSAVLKIKGEKTASVCAFCEGDHLEEK
EEHDSQTLYCPDCGSTVDRKLNGAANAWKRAASDLESLVTEYWEETREKQMGKAETKRLKSEKMAEARRLKRQ
AASQASAGA (SEQ ID NO: 4)
>WP_051690567.1
[Acidithiobacillus thiooxidans]
MSQIKIVPQINGSQLVYKYGVRMNLKVCGDIDDQIRRARAMYNNIIAVMRGIYDEMQTFTMEHAGPEGQALHE
KIVAANVAFDAAKADNDEPRMKQIAMERRELWKALSIILKEVRKEHKNTLKERFYSRIGNNSSTETYQCRAEA
IVGGLGYATATKVLDNALKAWQMSMVKGKAPRFARGEEKDQDTLTLQFSQAGGVPVEDIFTGKRKDIGIEYPK
KGFGPRSYSAFRFRLGAASEESYAEGTVQLHRAIPENARIAMAHLTRKKAGRKYQYELQLLATLAEPINLLPD
HRRKPLVAIHFGWSGDEEGRRLAGIADNADPLEARLLTLPPDIEDDIREASALQAKRDTYRDEVFLRLKEENT
LPTKGETPLSEHWNKIRKLPAQHVSANRMHHLAWLVKSELIEIPEWFETWRKADQRMWVQATSLARRARNRRK
KYYEKVAIDLASRYEAILIEMPDLKKSAEKVNEKTGEKTEFAKKARSGRVIAALYVLESAIQWAACKHGSAVL
KIKGEKTASVCAFCEGDHLEEKEEHDSQTLYCPDCGSTVDRKLNGAANAWKRAASDLESLVTEYWEETREKQM
GKAETKRLKSEKMAEARRLKRQAASQASAGA (SEQ ID NO: 5)
>OJW42488.1
[Rhodanobacter sp. 67-28]
MKITPASLPQGDVRIYEFGARLDKDCLEAANDQFFKAHQLYNELVACMQGTLRDMQAYLLENAGQEAQSAQAR
VEALNEALSAAKAANDEDTMKAVASERREVWRTLAALLRDTRKVHKATLQERFLCRIGRKSTCATYQLRCDAV
AAGLGWATANATLDAALLAFKSSFVQGRAPRFAKAGESTQDSLTLQFTAAGGVSVSTLLEGRHTEFRVKASGG
CGPRRYGTLEFRLGPASSETYAAGTWQYHRAMPDDGAVGLVRLVRRRLGPKFQWAIQFQVRSPLPVNDSVGER
KPLVALHAGWAADLTGRRVAGIADGADPGLARVLQLPPEIEAGLQHSGEVESARSVARDNVVATLKAHAWPQD
LLDAAEQPTEDATPEATRRSQAAADLLVIRRLPATHVAIRRLHRLAQRLRDTADLPDWFEAWRKEDKLAWQKA
AHAAKRARNRRKGFYREVALGLATGYQAIVLQPLDLESAAKKVDDASGERTEFGRKARSGRVVAAIYELEGAI
RWAAAKCGTAVLELTGETAGHCAYCGGAVKPVEDDSQRLACTQCGADIDRKRNGAALAWQATEESLPTLVEDF
WRETLAARDGAAAKRKEKREKVAEARRASRVVE (SEQ ID NO: 6)
>LNFM01018448_6
[activated carbon metagenome]
MTMEQAMVGAVYESASAAGEEVMASRNETTQEETDAFSVSFSTVGPAEVMVYEFGCRIAKGDLDHLRDQLWRS
RRLFNEVAAQINQTVDEAKCFLSDRAGPVAGEIAVRLGVLDTEWKSAKALDDREALVKIAGERKSLRTRWYGL
LHKARREHGTELRERYLSRIGNRVGAATYALRCAAVDDGLDWAMGNEALAAALGAFGKQWPRFKPISFRRFDD
PTEVATLQFTAAGGVAVADILADKHSQIGMQLGREQAGRRMYVPFRMKLGSGAQKKAITGTVLYHRPLPAGAS
VPIARLVGRRIGKDVKHYLQFMVKLKQAEQPGANSKRAPMGVAHLGWYYQPTGRRLAEVASSEDPGLSEQLTL
PIEVAELLDRARELDGQRSKLRDGIVGSVVRELPVEGAPEQIAEEVAALRKMRIEHVAPRRLGKLVFIWSRNC
ADWQRDRLKAMQAWRLEDRMLWQSSAHTARRARNRRRKHYEQLALSLAGKFTNILIDVPDLAQVAKVKDEDTG
EHNGLGARARGGRFDAALYELTSAIEKAGARLGCNVGKIKGPTASTCAHCGGTTKMGKTVRDVVCEACGAVED
RAASAAAVAFGWASQNKDAVDEAVAAALDADRAKATRAAERKEKMAIARATSRAARTESDEDSADGSRELK
(SEQ ID NO: 7)
>3300004774|Ga0007794_10001723_8
[aquatic-freshwater]
MTIKVYKFGLLDPVSGWDQTAIDVLFLRNKLWNNLVAMEHDKRQAYRNLLLDSDTELAALQARLDAIEVEKAS
LITSKKALRAKARSRQVDTAEIDLEIKKLLEERKALGGQTKDLRERVKIEVKPLAAELDQQRYEKTKQLNKES
GLWWCNSMTVIAAYEVGRLRAMREKNELRFHGFDGTGKYSVCRTGGFSLDHVMTGKLSFVSIRTLPIANLDDL
SERGQRSRARHHLTMIVLRATTEEGTKIRHEVTWPIILHRPLPDDCLIKQIQVLRKRVGDRFEWTCSITVDTP
EELKARLDSPSISVCGIDLGFRQVNNDLRVATLADSSGGLRYYTIGKDWLDSMDYVEAIQSDLSGTANSVWAQ
LRLILKELDEYPEALRERITDMLKAGAKTPIRAMRAMQKTLSNEPDLMPDALALLDDWKKRIRRRTKEMHDLR
DKLINRRKDIYRNIACEIARDYSLVRIANLKLKDMVKLKRNDGTDTKLTDNARKNCNRAALSELTLYIQQACA
KNGVALEKIDTTYMTRTCYQCGYLNPANTINLLLSCEGCGAEYDQDDNAAKNYLNATKPGTG (SEQ ID
NO: 8)
>3300004776|Ga0007800_10001775_2
[aquatic-freshwater]
MTIKVYKFGLLDPVSGWDQTAIDVLFLRNKLWNNLVAMEHDKRQAYRNLLLDSDTELAALQARLDAIEVEKAS
LITSKKALRAKARSRQVDTAEIDLEIKKLLEERKALGGQTKDLRERVKIEVKPLAAELDQQRYEKTKQLNKES
GLWWCNSMTVIAAYEVGRLRAMREKNELRFHGFDGTGKYSVCRTGGFSLDHVMTGKLSFVSIRTLPIANLDDL
SERGQRSRARHHLTMIVLRATTEEGTKIRHEVTWPIILHRPLPDDCLIKQIQVLRKRVGDRFEWTCSITVDTP
EELKARLDSPSISVCGIDLGFRQVNNDLRVATLADSSGGLRYYTIGKDWLDSMDYVEAIQSDLSGTANSVWAQ
LRLILKELDEYPEALRERITDMLKAGAKTPIRAMRAMQKTLSNEPDLMPDALALLDDWKKRIRRRTKEMHDLR
DKLINRRKDIYRNIACEIARDYSLVRIANLKLKDMVKLKRNDGTDTKLTDNARKNCNRAALSELTLYIQQACA
KNGVALEKIDTTYMTRTCYQCGYLNPANTINLLLSCEGCGAEYDQDDNAAKNYLNATKPGTG (SEQ ID
NO: 8)
>3300009004|Ga0100377_1000348_44
[aquatic-freshwater-aquifer]
MTNQENFSIKAAKTPSGDVLIYEFGARLDKECAAEVDKQIKQARGLYNNIVALMRDTMDEMRADLVENAGPVA
RETQAAIDALNLKFAEAKARDDEGAMLLIAQQRRELWAQLSALLKEVRASLKSEHKSRFFSRIGINSSCATYQ
LRSVAVKEGLGWGTANEILDNVLGAWKKSLAMGKAPRFVSAAEKMQDTLTLQFTAAGGISVVDLLSRSKGDMI
LTPPSEAGKRKYGSFQFRMGAASSNSYATGTWQYHRPLPEGSSVGVARLIRRRVGKDTKYAIQLQVKIKEGIE
QAVRNRKPLATVHFGWAGDVEGRRVAGIADSAEPSSAQVIALPTEIEEMLARSTTIQGERDTERDNIVPVVKQ
LDPTKFDETLAEEVTALNKLPAQHIAIRRLHRLCRHLGDVDMLPEALAEWRKADRMRWQSETHLARRARNQRK
DFYRNIAINLARNYEVIAIEPLDLAKAAIKLDKMTGEKTELSKKARSGRVVAAIYELESAIRWAAVKTGAAVL
ELTAAKTASVCSICGGHVSDDTENSQILHCDDCGADLDRKQNGAAIAWQMVEPLREDLAVDYHQAKIDAARAT
KQKMVEKLGKLAEGRLKGREAKAGSAANPE (SEQ ID NO: 9)
>3300004236|Ga0066449_1000007_83
[aquatic-freshwater-freshwater sediment]
MINCYKFGCLQPTAGFDQSAIEHLFLRNKLWNTLVALDHEFRQRYRDLMLNSDEKLKSVQDSIDSINQEIEDL
VENKMKLRQKERTKNIDSKLLDERINVLKAKRKTLSADSKTERERVKVEIKPQIDLLNTERYEAKKLAYKESG
LWWGNYETVVAAYDTASQKAMKSNTELRFKSFDGSGKFAVRFEDGGLTIDELKAGASNLCRIETLNTSAFQNL
SQRSIKSKARHSLTMTIYTFNDEKGKKQRKEITVPIIFHREMEEGKIKTIHLQRKRLGNQFTWSASFTLKNDI
EPANVADHPATASCGIDLGYRLVKDGLRVATVADSQNNVEYLVLPKSWIDRMDYTETLQSGLSEAMTLMWAKL
KAEIAKIPEYPDAVAEIIKNMQKMGDRLPYKGIKRLYRVLKEQDATGSPVAGFNAVLDILKAWDKATYRQELE
MVNLKDKLLKQREHIYRNFAAGLTKKYAHIVVEDMGLAELAKTEKSETETNDMPNAVKANRQRASLYSLVEAI
RLSAAKVGSYFEKSKAAYSSMTCNVCGHLNPKTQNIHQSCESCNTMYDVDENAARNFLKGEYINEKVLKQG
(SEQ ID NO: 10)
>3300009432|Ga0115005_10004282_5
[aquatic-marine]
MATRVYKYGLIPIGYPPQAAIDELFRANSLKNTLVALHRESRENWDDARRSASILYSEKMDELDKKNEDITEA
FNGLNKARMDEGTKDETGNKRLLAERAIINRLKKEKGDIYAELKPLRKEADKSIDKKALNDAYRQKCNDAVSA
KVSGVYRRTAEQIYANFKTAKDKASKDNATLQFHRFDGTGYFQFRCNPKGVSTDGISVDAFMSANFDGYMRCA
VQSVDNSKKKPRIRINAVLAGGRTKASKVFQEFDWIYHRPLPADAQIQNGKILRTRVGDKFRYDLVLTIRVPD
VEMVQPAKLSGTIGIDVGFRKVGNTLLIGTVMSSDRSQKAVALEVPQMMVSALEHVVALQGELDDAASDLGKA
ITPLLKANPIDDEHSKYRLWRSLALRPLHVTLSFEQAYKLSLWLKHEPSLFPSEINLKVHTWWRSYSRKYREI
HNRRKKQLTHRKHFYRETAAKLVAENKLIVLEDINLTDFAETKSKNTKLSNKARAQRFMASLGEFRDAIKNAA
GREGVPVIDVNPAYTSKTCSDCGHLNKELRSEKEWTCPACGVVHDRDENAANNLQKMGQKYLLDVQKAASMVV
Q (SEQ ID NO: 11)
>3300009436|Ga0115008_10017733_3
[aquatic-marine]
MTTRVYKYGLIPIGYPPQVAIDELFRANNLWNTLVALHRESRENWDDARRSASILYSEKMDELDKKNKDIREA
FNGLNQARMDEGTKDETGNKRLQAERAIINRLTKEQKEIYAELNPLRKEADKTVDKKALNDEYRKKCNTAVSA
KVSGVYSRTAGELYAYFRTARDKAFKDKTTLRFHRFDGTGYFAFRCRSKAVGVNVDGISVEDFMSQGFMDYMR
CAVMSIDESKKKPRILISAVLTGGATKASKVVQEFDWIYHRPLPPEGQIQNGKILRTRVGDKFKYDLVLTVKL
PDVEMIQPAALNGTIGIDVGFRKVGNSLLIGTVMFSDSAQKAVALEVPTMVVSALEHVDALRSELDDVASDLG
KAITPLLKANPIDEEHDKYRLWRSLALRPLHVTLSFEQAYKLALWLKREPNLFPSEINEKVHTWWRSYSRKYR
EIHNRRKKQLTHRKHFYRETAAKLIAQNKLIVLEKIDLTDFAETKNKNTKLSNKARSQRFMAALGEFRDAIKN
AADREGVPVIDVNAAYTSKTCSECGYLNKELKSEKEWNCPECGVVHDRDENAANNLQKMGQKYLLDAAKTAVV
VVK (SEQ ID NO: 12)
>3300009436|Ga0115008_10017733_4
[aquatic-marine]
MAIDELFRANNLWNTLVALHRESRENWDDARRSASILYSEKMDELDKKNKDIREAFNGLNQARMDEGTKDETG
NKRLQAERAIINRLTKEQKEIYAELNPLRKEADKTVDKKALNDEYRKKCNTAVSAKVSGVYSRTAGELYAYFR
TARDKAFKDKTTLRFHRFDGTGYFAFRCRSKAVGVNVDGISVEDFMSQGFMDYMRCAVMSIDESKKKPRILIS
AVLTGGATKASKVVQEFDWIYHRPLPPEGQIQNGKILRTRVGDKFKYDLVLTVKLPDVEMIQPAALNGTIGID
VGFRKVGNSLLIGTVMFSDSAQKAVALEVPTMVVSALEHVDALRSELDDVASDLGKAITPLLKANPIDEEHDK
YRLWRSLALRPLHVTLSFEQAYKLALWLKREPNLFPSEINEKVHTWWRSYSRKYREIHNRRKKQLTHRKHFYR
ETAAKLIAQNKLIVLEKIDLTDFAETKNKNTKLSNKARSQRFMAALGEFRDAIKNAADREGVPVIDVNAAYTS
KTCSECGYLNKELKSEKEWNCPECGVVHDRDENAANNLQKMGQKYLLDAAKTAVVVVK (SEQ ID NO:
13)
>3300001351|JGI20153J14318_10007490_6
[aquatic-marine-pelagic marine]
MATRVYKYGLIPIGYPPKETIDELFKANVLWNNLVALHRKNREDWDDARRAASILYSDKIDELEKKEEDLDAA
WKAFQQARMDEGTRDETNNKRLKSERASINRLKAERAEIYKELKPLRKEADKEIDKKQLNDSFRAQVNEALSV
NNSGVYRAIADQIYENFKTAKDKSIKENATLRFHRFDGTGYYHFRCRRKGTNVDGISIDDFMSRNFEAYPRCA
VQNIDNSKKKPRIRINAVLAGGKSKASKIHQEFDLIYHRPLPIDAQIQNGKILRTRVGDKFKYDLVLTLKIPD
KEPISYNNLKGTIGIDIGFRRSVNSLLIGTVMSSDVTEEAYEIIVPPKIVEAFEHVIDLQSELDDAATDLGRI
ITPLLKAHPLDEDHSKYKMWRSLALRPAHVTLSFEQAYKLAIWLKHEPDTFPEEITKKVHTWWRSYSRKYREL
HNRRRNQLTHRKHFYREEAAKIVALNKLIVLEEINLTDFAETKEKNTKLSKKARAQRFMASLSEFRDAIKNAA
QRDGIGIIDVNPAYTSKTCSECGNLNKDLRSEKQWSCPACGVVHDRDENAANNLQKMGQSYLENIKKETSEII
E (SEQ ID NO: 14)
>3300009447|Ga0115560_1022222_2
[aquatic-marine-pelagic marine]
MATRVYKYGLIPIGYPPKETIDELFKANVLWNNLVALHRKNREDWDDARRAASILYSDKIDELEKKEEDLDAA
WKAFQQARMDEGTRDETNNKRLKSERASINRLKAERAEIYKELKPLRKEADKEIDKKQLNDSYRAQVNEAISV
RNSGIYNATAGQVLDNFKAARDRSFKENATLKFHRFDGTGYYHFRCRRRGAKVDGINVEDFMSRNFIANPRCA
VQSIDNSKKKPRIRINAVLAGGQSKASKVHQEFDLIYHRPLPIDAQIQNGKILRTRVGDKFKYDLVLTLKIPD
KEPISYNNLKGTIGIDIGFRRSVNSLLIGTVMSSNVSEKAYEIKVPPKIVEAFEHVIDLKSELDDAATDLGRI
ITPLMKAHPLDEDHSKYKMWRSLALRPAHVTLSFEQAYKLAIWLKHEPDTFPEEITKKVHTWWRSYSRKYREL
HNRRRNQLTHRKHFYREEAAKIVALNKLIVLEEINLTDFAETKEKNTKLSKKARAQRFMASLSEFRDAIKNAA
QRDGIGIIDVNPAYTSKTCSECGNLNKDLRSEKQWSCPACGVVHDRDENAANNLQKMGQTYLESLKKETSEVI
E (SEQ ID NO: 15)
>3300009505|Ga0115564_10016546_3
[aquatic-marine-pelagic marine]
MATRVYKYGLIPIGYPAKETIDELFKANVLWNNLVALHRKNREDWDDARRAASVLYSDKIDDLEKKEEDLDAA
WKAFQQARMDEGTRDETNNKRLKSERASINRLDTEKAEIYKELKPLRKEADKEIDKKQLNDAYRTKVNEAVSV
RNSGIYSATAGQILENFKTARDRSFKESATTLRFHRFDGTGYYQFRCRRKGTNVDGISIDDFMSRNFEANPRC
AVQSIDNRKKKPRIRIDAVLVGGQSKASKIHQEFDLIYHRPLPIDAQIQNGKILRTRVGDKFKYDLVLTLKIP
DKEPISYNNLKGTVGIDIGFRRSVNSLLIGTVMSSDVTEKAYEIKVPPKIVEAFEHVIDLQSELDDAATDLGR
IITPLLKAHPLDEDHNKYKMWRSLALRPAHVTLSFEQAYKLAIWLKHETDTFPEEITKKVHTWWRSYSRKYRE
LHNRRRNQLTHRKHFYREEAAKIVALNKLIVLEEINLTDFAETKEKNTKLSKKARAQRFMASLSEFRDAIRNA
AQRDGIGIIDVNPAYTSKTCSECGNLNKDLKSEKQWSCPACGVVHDRDENAANNLQKMGQTYLESLKKETSEV
IE (SEQ ID NO: 16)
>3300020165|Ga0206125_10004811_3
[aquatic-marine-seawater]
MWCEINMATRVYKYGLIPIGYPPKETIDELFKANVLWNNLVALHRKNREDWDDARRAASILYSDKIDELEKKE
EDLDAAWKAFQQARMDEGTRDETNNKRLKSGRASINRLDAEKAEIYKELKPLRKEADKEIDKKQLNDAYRTKV
NEAVSVRNSGIYSATAGQILENFKTARDRSFKESATTLRFHRFDGTGYYQFRCRRKGTNVDGISIDDFMSRNF
EANPRCAVQSIDNSKKKPRIRIDAVLVGGQSKASKIHQEFDLIYHRPLPIDAQIQNGKILRTRVGDKFKYDLV
LTLKIPDKEPISYNNLKGTIGIDIGFRRSVNSLLIGTVMSSDVTEKAYEIKVPPKIVEAFVHVIDLQSELDDA
ATDLGRIITPLLKAHPLDENHSKYKMWRSLALRPAHVTLSFEQAYKLAIWLKHEPDTFPEEITKQVHTWWRSY
SRKYRELHNRRRNQLTHRKHFYREEAAKIVALNKLIVLEEINLTDFAETKEKNTKLSKKARAQRFMASLSEFR
DAIRNAAQRDGIGIIDVNPAYTSKTCSECGNLNKGLRSEKQWSCPACGVVHDRDENAANNLQKMGQSYLESVK
KETSEVIE (SEQ ID NO: 17)
>3300010313|Ga0116211_1004493_2
[aquatic-thermal springs-hot spring]
MIKAFKYGMLEPVAGFDKAAIDVLYLRNKLWNSLVELEKAHRERYRTLITGSDDELSKIQARLDQIEAERAEL
VKRKRQARAMVRSKKVDTSEHDDRIDMLMAERNDLRTKAKDIRLQVKEKVKPAIADLEKERYEAVKHLIHEAG
LWWCNSETVIAAYDLARVKAMKENAELRFRSFDGSGKFAVRKTGGFALSDLVSGKLSFARLEALPDANFAHLS
ERGKRSRARHHLTMTILTYKDESGKLCRHEVTWPIILHRPLPPEGMIKFIHVQRKRIGKDFQWTCSITMEVDE
IQKTPIDHPSRAACGIDIGYRLVKDGLRVAVIADTSGKIDHLTLPQDWIEKMDHVESIQGHLDNSNDLAWGEL
KALLKSMHDYPESIAESIGRLLKAGDRTPVRGMRALHWRLRNEPETMPEVLSILDTWEAETCRREREMHRLRR
KLINRRKDLYRNFAYKVANRYVLIRIRGLSLKKLAAVNLEDGSDNQMPQAVRNNRTRASLSELTLCLQQAAVK
AGADFEKVFDVNSTTTCSTCGNQNLKMDREDIYFRCEKCDTLHDQDENAAKNLLRKEFYLAEQAVM (SEQ
ID NO: 18)
>3300009784|Ga0123357_10002363_9
[arthropoda-digestive system-termite gut]
MENHKFTIPDQANQGIIVYEYGIRLDKESKPLVWQQIQLSRKLYNNIVASMRQTFDAMNTFILERAGDEGKQL
NQAIEEGIERFKTAKAEQNEDDIKETVLFLREKRAKLSEQLKGVRTQYKEETKRNFFNRIGMRTSCETYQIRS
QAVKDGLGWATANEVLNSALKAFQARIKTGQPPKFAVGEEKQQDSLRTQFTQAGGCPVATLFESEHSGLSLRA
AAGFGRRKYGTFRFRLGEAKSDVWATGTCQFHREIPSGATVASAALVQRRIGRDLKHALQLVVKLPQQAEAQA
TQSKKFCTVHFGWASEEGIQYVMALADQENPTKAQLFQLPTDIETDFNRVENLASQRSKLLNDLVLQIKSGSI
VIPSQIKEVADEFDAIKRLPATHISLTRLHRICRLMIESDIFRPEALERWRRQDRLLLQDIAHIRRRALYRRR
DFYRVTASVIAKSYGAIVIETLDLKKANTKINMVTGEKSDKNKKSRSRQRMAALHELQRQLRQAAGKAGCVII
ELTGEKTTATCAFCNREGTTTTSESSQVLHCPHCGSQMNRKQNGAAVAWQLASPIIDDLVHEARSLAAVQSSE
RAASKILKAEKVATARKANRAAREPAATDK (SEQ ID NO: 19)
>ADIG01000806_20
[groundwater metagenome]
MIVQITPAPLPQGDVRIYEFGARLDHDCVRTVDEQIFKAHQLYNQLVACMQTTVRDMQAYLLDHAGPDAHAAK
ARVDGLNEAFNAARAANDENRMTTVATERREAWRALAAVLRIARKEHRTAMQETFLSRIGKKSACETYQLRCK
AVADGLGWATANATLDAALIAFKKSFALGRAPRFARIADSIQDTLTLQFTAAGGINIERLLDGKHTELALKPP
AVCGKRGYGTFAFRLGAASAETQATGTWQYHRPLPPGGTVGLARLVRRRIGPKTTWSLQLQVRSPLPEREHED
RRPLVTVHPGWAADLSGRRIAGIADAADPGLATVLQLPPDIEHGLQRAAELESTRSQARDALTPMLKVHPWPQ
ELLNAATPEEDASASGDSGPMAPERIMCRKVADEILALRRLPAQHIAIRRLHRLARWLRLAEVDVPDWLETWR
KEDKLRWQASAAAAKRARNRRRGFYRETALRLASQYQAIVIEPLNLADAAKKIDEATGERSDFAKKARAGRVV
AAIFELDSAIRWAATKCGTAVLDLTGETAQHCAICGGHSLKADDEDSQCLRCSDCGADIDRKRNGAALAWQAA
AAHLETHLEDFWRLTLENRASAAAKRDEKKTKLQEGRRAAMRETLET (SEQ ID NO: 20)
>CXWL01128655_18
[groundwater metagenome]
MIKAYKFGLLNPISGFDQAAMDVLYLRNKLWNQLVELEKNSRAAYRALMLDSSEELSVIQTRIDAIEVERADL
VSQKKKLRASVRSKKVDTAGIDAAVERLIAERTNLRAKAKQLREVVKVEIKPKAVELDKVRYAAVLALIKGSG
LWWGNSETVIAAYDVARVRAMKESAELRFRSFDGTGKFAYRESGGIDFDKFMSGKVNFARLNTLPDSDFAHLS
ERGRRSKARHHLTMTVLTSVDDAGKKVRHEVTWPIVMHRDMPAGAIKTIHVHRKRVGDQFNWTCSITIDVPEE
PKQLIDHPAKAACGIDLGFRLVKDGLRIATIADSDNRIEHVVLPLDWIEKMDYVEHLQSTLSETANLTWVRLR
KHLSELPDYPESIKERIHNILKAGERVPTRGMRSLLGALKAEPELLPEALQILAAWSDDIYRPAREMHNLRDK
LMKRRQDLYRNVSHCLSNKYAMVRVEDMDLRQIARVKKDDGSDNPLPDTVRDNRKRAALFEFVLSIKQSCVKT
GSVFEKMNPAYSSMTCSSCGHLNQPGMDIHYSCENCGTLHDQDENAAKNFLRGEYFSSPKQDVA (SEQ ID
NO: 21)
>OGCL01001770_13
[hot springs metagenome]
MIKAFKYGMLEPVAGFDKAAIDVLYLRNKLWNSLVELEKAHRERYRTLITGSDDELSKIQARLDQIEAERAEL
VKRKRQARAMVRSKKVDTSEHDDRIDMLMAERNDLRTKAKDIRLQVKEKVKPAIADLEKERYEAVKHLIHEAG
LWWCNSETVIAAYDLARVKAMKENAELRFRSFDGSGKFAVRKTGGFALSDLVSGKLSFARLEALPDANFAHLS
ERGKRSRARHHLTMTILTYKDESGKLCRHEVTWPIILHRPLPPEGMIKFIHVQRKRIGKDFQWTCSITMEVDE
IQKTPIDHPSRAACGIDIGYRLVKDGLRVAVIADTSGKIDHLTLPQDWIEKMDHVESIQGHLDNSNDLAWGEL
KALLKSMHDYPESIAESIGRLLKAGDRTPVRGMRALHWRLRNEPETMPEVLSILDTWEAETCRREREMHRLRR
KLINRRKDLYRNFAYKVANRYVLIRIRGLSLKKLAAVNLEDGSDNQMPQAVRNNRTRASLSELTLCLQQAAVK
AGADFEKVFDVNSTTTCSTCGNQNLKMDREDIYFRCEKCDTLHDQDENAAKNLLRKEFYLAEQAVM (SEQ
ID NO: 18)
>LNAP01002847_16
[soil metagenome]
MKKITIRKYGARLLGDSEPIIVKSMRDQNTLWNKLVEIERANTTEYRDIVAQSDDVLAALTQEYAAAEQRLKD
VQEMRNRVRAAKRSKQIEGAENYAAEIKAISSSLKDLRARMKECRARAKEAAKPRLEGLEDRRRAAVKQATNE
AAIWWAHSELVTNSFDVARVKALKSNAELRFHRFEGEGRIGVRIQDGILLGNQKGTSMLQVREATPEELGHLQ
AQRARKRLVAVDIRVGKRGEDGHIPKATFLVTIHEGMELLPNTPLKTVTVKREMHAGQPKWFMVFMFVESDAE
PEDKPLPPKAVGVDFGWRVVKDREWGERTGLRVATIANKDGTKQHITLPPELLARFERSTRLRSELDVAANEF
WVRTASLFTDDILATLSEDEWLRVLVGKAKRAHRPYPSLMEAITRAHAANPVLGPEADEQMQAWARRARRLNV
AAFGARRKAADHRKHLYRNVAARLVRECGLIAIKDTDFHKLAKLVDDDGKETELNKHARANRFMASPSELRQA
IKMAALREQRELVNVAPAHTTTTCSACGHVHGERPKDLIFVCDSCGKWHDQDENSAAICLKIALESKL (SEQ
ID NO: 22)
>3300007533|Ga0102944_1000048_72
[terrestrial-soil-pond soil]
MNDVTLSFRGLEPRESTSWSYGARVAGSEALEEQYTLAQRTYNNMVEVTRQALAAFNEWFAEKDPEIARLGTE
IERLGAQWAEAKARDDRDELARIAAERRPLRQQWYERCFAVRKDNRGEVNALLKQWVGSAKESRLYLARVEAV
KAGLYWATATAVMTAVQRAWDKQFPRLRPVAFSRRSEKRRETLVVQFTESGGVAMETLHTKHGGLWIEPPGEG
LLSAWANGRRPGRPDRYLRFRMRIGGRGREGVYVEGSVQMVRPVPEGARVMMARLVRERVATRYRYQLQLVLR
LAEPLSIPAEDKAPRVALDIGWYYEAGRGRRVIAYTAGANEDAVEQTYLDPSIDEAFDRVDDMNSRRSLARDD
VTIALRCCQWDGAPEALAEALSAINRLPVAHVSPARLAQMVWLWREHHGDYRPDVLEELWAWRRWDKKLYETS
AHLRRRTAGRRKKFYEGWARHFASRYATIVVVRPDLREAAMVKNAISGEHTALTARARQGRVRAALYEFLNVV
ATKAAEAGSVVIELTGRTTTECSACGEIMVVPEDNPATRLLVCHACGVSHDREANSAVLAFRVLDDEASVTKG
LAHAQEKADKARERRYKRRTAMRDARWKDEQTTTSGQ (SEQ ID NO: 23)
>3300007533|Ga0102944_1003721_10
[terrestrial-soil-pond soil]
MNQSPPANGECMENVTLSFRGLEPRESTSWNYGARVEASEALEEQFTLAHRTYNQMVEVTRHALAALTDWFCE
KDPEIARLGAAIERLSAQWSEAKARDARDELEQIAAERRFLRKDWYERCFAVRKDNRSEVNALIRQWVGLTKE
SRLYAVRTEAVKAGLYWASATAVMTAVQQAWDKQFPRLRPVAFSKRAEKTRETLVVQFTEAGGVPMSTLHSKH
GGLWIEPPGDGLLTAWANGRRPARPDRYLRFRMRIGGRGREGVYVEGSVQMVRPVPEGARVMMARLVRERVAT
KYRHQLQLVLRLAEPLSIPTETKEPRVALDLGWYYEAGLGRRVIAYTGGDNEDAVEQIYLPPGIDEAFDRVDD
MNSRRSLARDDVAITLRCCQWDDAPAPLAETLAAINKAPVAHVAQARLARLVWQWRNEHSDYRPDVLAELWSW
RRWDKKLYEASAHLRRRTAGQRKKFYEHWARYFASRYTTIVVVRPALREAAVIKNEASGEHTALTARARQGRV
RAALYDFLNAVATKAAETGSVVIEVSGRTTTECSACGAIMAVPEENPATRTLVCHACGVSHDREANSAVLAYR
VPDDDGAVTQSLEHAQEQADRARERRERRRQAMREGRWKGKQSAGGGD (SEQ ID NO: 24)
>3300007533|Ga0102944_1003721_8
[terrestrial-soil-pond soil]
MENVTLSFRGLEPRESTSWNYGARVEASEALEEQFTLAHRTYNQMVEVTRHALAALTDWFCEKDPEIARLGAA
IERLSAQWSEAKARDARDELEQIAAERRFLRKDWYERCFAVRKDNRSEVNALIRQWVGLTKESRLYAVRTEAV
KAGLYWASATAVMTAVQQAWDKQFPRLRPVAFSKRAEKTRETLVVQFTEAGGVPMSTLHSKHGGLWIEPPGDG
LLTAWANGRRPARPDRYLRFRMRIGGRGREGVYVEGSVQMVRPVPEGARVMMARLVRERVATKYRHQLQLVLR
LAEPLSIPTETKEPRVALDLGWYYEAGLGRRVIAYTGGDNEDAVEQTYLPPGIDEAFDRVDDMNSRRSLARDD
VAITLRCCQWDDAPAPLAETLAAINKAPVAHVAQARLARLVWQWRNEHSDYRPDVLAELWSWRRWDKKLYEAS
AHLRRRTAGQRKKFYEHWARYFASRYTTIVVVRPALREAAVIKNEASGEHTALTARARQGRVRAALYDFLNAV
ATKAAETGSVVIEVSGRTTTECSACGAIMAVPEENPATRTLVCHACGVSHDREANSAVLAYRVPDDDGAVTQS
LEHAQEQADRARERRERRRQAMREGRWKGKQSAGGGD (SEQ ID NO: 25)
>APMI01033782_24
[wastewater metagenome]
MKSTPDTISITPGATANGDMLTYEYGLRLDKESIAHVGAQIAMSRRLYNDLVAQIRTTVDALQAFVIDKAGDE
AVQIKVRIEELTTNFKAAKAEDNEPEMKRIAEDRRNQWKLLSALIKAASKANRAEINERFLSKIGKNSSCPTY
QLRGKAVAEGLGWGTANAVLDAALQAFKTSFALGRAPRFASGAEIDQDCLFLQFTAAGGVASASLLAGKQADL
QLLPTNGCGKRKYGEFKFRLGAAKADTYATGTWQYHRPLPDGSNIALARLVRRRIGMHDKWAIQLLVKPKTPI
RESVEERKPLVAVHFGWAADIAGRRVAAIADAADPGAATILALPPSIEEALDRAREIQGVRDKSRDEIAPQVR
SIEIPGSANETLIDLLGRVRKTRPQDISANRIHYLCRLLREADHLPDWLEAWRKEDKNRWQDQAHIAKRARNA
RKSFYREVAINLGRQYDAIAIEPLDLASAAMKVNEATGEKTDFAKKARAGRVVAALYEFESAIRWAATKTAAA
LIEVSGATASVCSVCGGHVEATKDDHQSIVCHDCGAVLDRKQNGAAIAWQSANDKREDVVTEFWSEYFADSEA
KKEKKAEKLAKMAEGRRNARTESAAEIA (SEQ ID NO: 26)
>NZ_JQKL01000024_23
[Clostridiales bacterium DRI-13]
METAATKNYLALSFGCLSPTRGEEYLLDQIKKKHDLWNKLVEKDREHREKVRQVMVFESETTKKIKELEEELN
SLREEIKNQRKTKRTGKVDLTDQKARIEEIKPQLKQLKEKFKEERSFIFEARKQELAQLEKERWAVVKELGKG
SGLYWCNLEDVVNSYDIGRKKAKAAGGEMRFHRWDGTGKVTVRFQKGLPVNEMFSCTNNLLQIDPVDKDAWYN
PVRAIRRKKSRTRVRLRACSENKKPLFIELPVVLHREIPEDALIRTASVIREKVGMRYRYKLNLVLEILGENT
NRILPALEGTAAIDLGWRTVKDGLRVACLVDDKGHSEELILDNDVLHEFNKIKDLQSIRDNLFNETKAKLMEL
LKTLELPDEAKERTSHMANWRSQQKMLRLHQYWRENRLPGDDEVWEVLEYWRKREIHLYEWQENLRDQVLRRR
KEIYRIFAAKITRKYKTIVLEEFTLNKTVQKPNPEEGPAGTLPANRNRFIAAISEFRNELANACRKNHVEFTY
VPAENTTITCHKCGHKEKFDAAAQIIHTCSTCGELWDQDYNAAKNLLAFSQKGGVK (SEQ ID NO: 48)
>WP_081908191.1
[Clostridiales bacterium DRI-13]
MSRLEARTRYLQAGQKRLGKIRKRGFFMETAATKNYLALSFGCLSPTRGEEYLLDQIKKKHDLWNKLVEKDRE
HREKVRQVMVFESETTKKIKELEEELNSLREEIKNQRKTKRTGKVDLTDQKARIEEIKPQLKQLKEKFKEERS
FIFEARKQELAQLEKERWAVVKELGKGSGLYWCNLEDVVNSYDIGRKKAKAAGGEMRFHRWDGTGKVTVRFQK
GLPVNEMFSCTNNLLQIDPVDKDAWYNPVRAIRRKKSRTRVRLRACSENKKPLFIELPVVLHREIPEDALIRT
ASVIREKVGMRYRYKLNLVLEILGENTNRILPALEGTAAIDLGWRTVKDGLRVACLVDDKGHSEELILDNDVL
HEFNKIKDLQSIRDNLFNETKAKLMELLKTLELPDEAKERTSHMANWRSQQKMLRLHQYWRENRLPGDDEVWE
VLEYWRKREIHLYEWQENLRDQVLRRRKEIYRIFAAKITRKYKTIVLEEFTLNKTVQKPNPEEGPAGTLPANR
NRFIAAISEFRNELANACRKNHVEFTYVPAENTTITCHKCGHKEKFDAAAQIIHTCSTCGELWDQDYNAAKNL
LAFSQKGGVK (SEQ ID NO: 49)
>GAB36148.1
[Gordornia otitidis NBRC 100426]
MTRVTVQTAGVHYKWQMPDQLTQQLRLAHDLREDLVTLEYEYEDAVKAVWSSYPAVAALEAQVAELDERASEL
ASTVKEEKSRQRTKRPSHPAVAQLAETRAQLKAAKASRREAIASVRDEATERLRTISDERYAAQKQLYRDYCT
DGLLYWATFNAVLDHHKTAVKRIAAHRKQGRAAQLRHHRWDGTGTISVQLQRQATDPARTPAIIADADTGKWR
SSLIVPWVNPDVWDTMDRASRRKAGRVVIRMRCGSSRNPDGTKTSEWIDVPVQQHRMLPADADITAAQLTVRR
EGADLRATIGITAKIPDQGEVDEGPTIAVHLGWRSSDHGTVVATWRSTEPLDIPETLRGVITTQSAERTVGSI
VVPHRIEQRVHHHATVASHRDLAVDSIRDTLVAWLTEHGPQPHPYDGDPITAASVQRWKAPRRFAWLALQWRD
TPPPEGADIAETLEAWRRADKKLWLESEHGRGRALRHRTDLHRQVAAYFAGVAGRIVVDDSDIAQIAGTAKHS
ELLTDVDRQIARRRAIAAPGMLRAAIVAAATRDEVPTTTVSHTGLSRVHAACGHENPADDRYLMQPVLCDGCG
RTYDTDLSATILMLQRASAATSN (SEQ ID NO: 50)
>BAFB01000202_4
[Gordornia otitidis NBRC 100426]
MPDQLTQQLRLAHDLREDLVTLEYEYEDAVKAVWSSYPAVAALEAQVAELDERASELASTVKEEKSRQRTKRP
SHPAVAQLAETRAQLKAAKASRREAIASVRDEATERLRTISDERYAAQKQLYRDYCTDGLLYWATFNAVLDHH
KTAVKRIAAHRKQGRAAQLRHHRWDGTGTISVQLQRQATDPARTPAIIADADTGKWRSSLIVPWVNPDVWDTM
DRASRRKAGRVVIRMRCGSSRNPDGTKTSEWIDVPVQQHRMLPADADITAAQLTVRREGADLRATIGITAKIP
DQGEVDEGPTIAVHLGWRSSDHGTVVATWRSTEPLDIPETLRGVITTQSAERTVGSIVVPHRIEQRVHHHATV
ASHRDLAVDSIRDTLVAWLTEHGPQPHPYDGDPITAASVQRWKAPRRFAWLALQWRDTPPPEGADIAETLEAW
RRADKKLWLESEHGRGRALRHRTDLHRQVAAYFAGVAGRIVVDDSDIAQIAGTAKHSELLTDVDRQIARRRAI
AAPGMLRAAIVAAATRDEVPTTTVSHTGLSRVHAACGHENPADDRYLMQPVLCDGCGRTYDTDLSATILMLQR
ASAATSN (SEQ ID NO: 51)
>WP_039994403.1
[Gordornia otitidis NBRC100426]
MHYKWQMPDQLTQQLRLAHDLREDLVTLEYEYEDAVKAVWSSYPAVAALEAQVAELDERASELASTVKEEKSR
QRTKRPSHPAVAQLAETRAQLKAAKASRREAIASVRDEATERLRTISDERYAAQKQLYRDYCTDGLLYWATFN
AVLDHHKTAVKRIAAHRKQGRAAQLRHHRWDGTGTISVQLQRQATDPARTPAIIADADTGKWRSSLIVPWVNP
DVWDTMDRASRRKAGRVVIRMRCGSSRNPDGTKTSEWIDVPVQQHRMLPADADITAAQLTVRREGADLRATIG
ITAKIPDQGEVDEGPTIAVHLGWRSSDHGTVVATWRSTEPLDIPETLRGVITTQSAERTVGSIVVPHRIEQRV
HHHATVASHRDLAVDSIRDTLVAWLTEHGPQPHPYDGDPITAASVQRWKAPRRFAWLALQWRDTPPPEGADIA
ETLEAWRRADKKLWLESEHGRGRALRHRTDLHRQVAAYFAGVAGRIVVDDSDIAQIAGTAKHSELLTDVDRQI
ARRRAIAAPGMLRAAIVAAATRDEVPTTTVSHTGLSRVHAACGHENPADDRYLMQPVLCDGCGRTYDTDLSAT
ILMLQRASAATSN (SEQ ID NO: 52)
>WP_013159911.1
[Meiothermus silvanus DSM 9946]
MPFGKKARHVKAYQFGADAPQEGMEAVLEQHRLRTDYYNALVEMELRQREERTALLANLAAESGLESPNQVYE
RLKAAGEKGIRKHPEYVAARERQKALYGHPRLLELQSRQREERNALRRSFGAKGLYSSNYLDVERAFDKARQS
PELRFRRYSPHEGRLAVLYTEGLPMREIGSDTRVQLPLPDPIIYRDRATRRKHQRVLMKFRVRSVERQPLWIT
VPVYLHRELPDGVCREVSLHWHRVADRLRWTVSVVVEVEGPPVASPTGRGAVAVDLGWRRVEGGLRAGFWVGE
DGAGGEIALSEGDLKQFSKVEDLRSIRDQHLNALKEALAAWLEAPPAPLPDWLAEETKTLPQWRSPARFAALF
RRWQSERVHADEAAYGLLEGWHKRDRHLWQYEANLREQMILRRREQYRVLAATLARQYDALIVEDFNLRAAAE
LDQGGSDLPDAARRYRTIASPSTLRDALVNAFAQRGKPVRKLNPAHTTTDCHACGGALVGDPAKELRLYCPTC
ERFYDQDENAARNLLRRAQEVQAQV (SEQ ID NO: 53)
>WP_096876841.1
[Methylomonas koyamae]
MIRTYKYSLKAPENFAEDCEDELRRMNDLWNRLIEIDRQRERSFKDLCRSTSAEYAAAQDEIEALREPIDNLY
DAIRAERIATRSKEPSDELRARRDELLGRRKALWEICKAIQKAIPKESQAPINEVYKTNVKLARQQSGCFWGN
YNAVIESFETAKSKAIKDGGRLHFKSFDGSGRFVNQIQGGMTVTELLAGSHSQAQLTNLVTTNKTKGRFAFTA
FTGKDDAGKRFRRQLFSEINYHRPIPADGVIKAVEVVKVPHDGKQKYKWHACFTVALPEVDIKHPKRNIAGVN
LGWRQFGGRLRVAVVVDDAGKKTEYFVPAELVSKFEAAETIQKAADDARNEMLSWLRTFYQDNRDEAPQEWRE
SIQGLLRNRPSVDAANHLMTIWRECVFAQEESRRYAAWLKSDAALRRSYTGCRQNAVKWREEIYRHIAKELAE
RYAVLAVTDTPLSTMSRTKAKDDLAVDNALPESARRNRVIAAIYSLKEWIGKQAAKTGSTVETITGKMTATCH
KCGYVAEKRLRGSQYATCKSCGSELELDENAAINCRNHASGAVLISDKPEKTGRFQRAKMAENDFARKIGDNA
SPLVT (SEQ ID NO: 54)
>WP_048895525.1
[Mycobacterium conceptionense]
MAITVHTAGVHYRWTDNPPEQLMRQLRLAHDLREDLVTLQLDYETAKAGIWSSYPAVAAAETELADAESAAEQ
AAAAVSEERTKLRTKRITGPLAQKLTAARKRVREARSTRRAAISEVHEEAKGRLVDASDALKAQQKALYKTYC
QDGDLFWATFNDVLDHHKAAVKRIGQMRAAGQPAQLRHHRFDGTGSIAVQLQRQAGQPQRTPELIADVDGKYG
RVLSVPWVQPDRWERIPRRERRMIGRVTVRMRAGQLSGEPQWLDIPVQQHRMLPLDADITGARLTVTRTAGTL
RAQISVTAKIPDPEPVTDGPDVAVHLGWRNTDTGVRVARWRSTEPIEVPFDFRDTLTVDPGGRSGEIFVPEAV
PRRVERAHLIASHRADRMNELRARLVDYLAETGPRPHPSREGEELGAGNVRMWKSPNRFAWLARVWADDESVS
TDIREALAQWRHQDWISWHHQEGGRRRSAAQRLDVYRQVAAVLVSQAGRLVLDDTSYADIAQRSATTKTEELP
NETAARINRRRAHAAPGELRQTLVAAADRDAVPVDTVSHTGVSVVHAKCGHENPSDGRFMSVVVACDGCGEKY
DQDESALTHMLTRAVQSAA (SEQ ID NO: 55)
>WP_061006603.1
[Mycobacterium mucogenicum]
MTTMTVHTMGVHYKWQIPEVLRQQLWLAHNLREDLVSLQLAYDDDLKAIWSSYPDVAQAEDTMAAAEADAVAL
SERVKQARIEARSKKISTELTQQLRDAKKRLKDARQARRDAIAVVKDDAAERRKARSDQLAADQKALYGQYCR
DGDLYWASFNTVLDHHKTAVKRIAAQRASGKPATLRHHRFDGSGTIAVQLQRQAGAPPRTPMVLADEAGKYRN
VLHIPGWTDPDVWEQMTRSQCRQSGRVTVRMRCGSTDGQPQWIDLPVQVHRWLPADADITGAELVVTRVAGIY
RAKLCVTARIGDTEPVTSGPTVALHLGWRSTEEGTAVATWRSDAPLDIPFGLRTVMRVDAAGTSGIIVVPATI
ERRLTRTENIASSRSLALDALRDKVVGWLSDNDAPTYRDAPLEAATVKQWKSPQRFASLAHAWKDNGTEISDI
LWAWFSLDRKQWAQQENGRRKALGHRDDLYRQIAAVISDQAGHVLVDDTSVAELSARAMERTELPTEVQQKID
RRRDHAAPGGLRASVVAAMTRDGVPVTIVAAADFTRTHSRCGHVNPADDRYLSNPVRCDGCGAMYDQDRSFVT
LMLRAATAPSNP (SEQ ID NO: 56)
>WP_011733919.1
[Pelobacter propionicus DSM 23791]
MKRVTITIDGEQTKGIVIGTIAANHTAAEWLLTASVSAKSAKVRFDPEEAVAETSSLVMIAPTRTEKYLYLVP
DEQVQPVTTIVRKYGLLSPLDWDCPDYPAGDAFEHLFLQNKLWNDLVTIEREHRAKYRELIGSDEETAQMDTE
IASIKDRLSVLDEGRKKLRVEHRKKKCPEIDCLDENIKKLKSELKAVASKAKETRAAAKDRIRAAGNDIENLE
KDRQAAVIKAYNNSGLWWGNYNAVLESYKKARIKALKDGAELKYHRFDGSGRFTNQIQGGMSVQDLLEGNRNV
ASLRLVSSGELGDISGKKPPSLDLQSVGSRRDSREYGILAITLYTGTDEQSKKFRRTLSFPVILHRPLPEGAT
LKSLSVHRKRVGTDFVWSVVFTFTTDCPTYDQRSSTGNRCGLNLGWKKQAGGGLRVATIYDGSDARHITLPQA
IIDGLDYVNGDLQGRIDSAANENHAWLLEQWGGDELPESLQELRSMLRRSKRPHPAKFAKAVIAWRNYPEYLG
DARDEAEQRRKATKRLTIEMAHKREKLLRRRMDFYRNTAKQLTSVYDVICLDKMDLRRLALLEKGDGTPNELT
KIARKQRQQAAISELRECLSKAAAKNGTQIEQVSTASSATCSACKGKMEQVDGIMWRCRECRALVDQDINAAA
NLFREVL (SEQ ID NO: 57)
>WP_018234394.1
[Thioalkalivibrio thiocyanodenitrificans ARhD1]
MKRQQEDTEALVYAYGARIPLDDPHLQEELRKQRAFWDALVEATLAAERELDDRMKADSPQYAAAVQALIDAS
QAVREAIERRNAERAKTRSRTTSVDGEVKERITEKNAARKEVWRLAGEWRKANKEAVSEHQARMKEEAKRLRQ
GCGLYWGNYNRVLDSFQRARQQTLKKGRRVRPSDPARDDGILAVQIQRTKSGLGASPEELFSGNVSQLQIDRP
PPGVEFLPANRRRREARVTARMRVDAAGHMIEFPVVLHRPVPPGARIKAAQLVWKREGERWRGQLCLTVSSPK
QEREHPGVEACGIDLGWRLQKDGALRVATVADSKSRLYTYTLPADWMRGMDQVERLSSHLDENAMEVAAWVHA
HRDELPEKLTQPAANWSPGKGSKWLRDKELHDAVRALNWEVPAEIRHWYERYRHLKTWRDNLRAKLLRSRREV
YRLLAADLAGRYAVIGIEDMDLSKIAKTKKRKDASDPELHATARAQRQRAAVHALRHEIEHQANKHGAQLVHV
SGKTTTTCRACGAATGQKDRASLIWTCEHCGAVWDQDLNAAGNILDSAMGASAPAATTLAKAKSRRYDLTQPN
FRERSKTGSRASARA (SEQ ID NO: 58)
>3300000944|BBAY81_10000005_89
[algae-green algae-macroalgl1 surface-ecklonta radiata 2]
MPVINWVYRSEEPTNVAAVKNQILLNHRYRNQLIELEHNRRATYKTLAASLCPAYADAVTIYDRAVAELDEAY
KDLRLSRQRARRRHEPTDSQKARINQAKAVRKTAIAQLDAAYKVAKKLIRDAHKVYQDQAAQEITQLADETES
QLKRQRKVRYFELVEEAGLDDGQIAHAREAKIARQQSGVYWGTSRIMEQIAEKTYKKGPPPKFRRWEGSGAIG
VRFQGGKPVAAVMENNSEILHIHIPPGSERLVIGQDRGEVAKGTIRFLVCRDDDGNPVFATFPYVHHRDFPAS
AKIIDGFAHLKRVGKKEYWEIRLTIKVDDVVSTVDKSNTCVLHLGHRMIDEQLRCATVMDATQQVSQLFLSSD
KLRRFSRPDSLQGIRADRFNIIRGEFLDWLASIDVPEWLVERTQTLASHQSPESLYRTVELWRDNRFVMDTET
SAQFFTNSLPWAESKLNSPAVRRKRHPSDIQTVFGIMEFWRSWDRHTLQESASINKKAIRNRKHVYRDWLRRL
SGRYTHLIVDSTNWATLGKKEKDDEKVVLVANQRRLARIASPGLLRQCAVEIFGQSNVSVVTSVNMTRTCSTC
GQVTEDWDSAKLEYHCSHCTYTVDQDINAANIMLSRIPDAVPYTEFAEAKRR (SEQ ID NO: 59)
>LSQX01035253_23
[anaerobic digester metagenome]
MITVFKYGVHYRWQVPEVLREQLWLGHQLREDLVTLQLEYEAGLKAIWSSYPDVAAAEESLATAAAEALEAAE
EVSRQRQVQRTKRITGPAADALAAARKRAKEARVVRRSAIAAVKDEAAERISALAAGLRASQKAKYAEYAQGK
GLYWATFGDVLDHHKTAVKMVAAKRAAGRPAALRHHRFDGTGAVAVQLQRPAGKPQRTPALISDPVASNWRNV
LHLPWVDPEQWEQMTRAEQRAQGRVIVRMRCGADIIEVPVQVHRMLPADADITGARLVVAREGSDYRISLTVT
ARIGDPEPVTAGPTVALHFGWRGSDAGPVVVRWQSDAPVDIPNDCSAFMVGDRWGGKIVMPSVIVDRLESAAA
IQAGRDEQLNTVRAAVVEWLTVNGPVPHPIRDGEEISSADASRWRRPARFAALAGWWRDAPPAGGEQIAEVLE
AWRASDKRLWNTQVHTAGRALRRRDDLYRQVAAIFADQAGLVVVDDTDMGAVAASRSDAPTAVTDPAARRRTY
AAPGVLRASIVAAAAREGVPVRSVSHKGMSVIHAECGTVNECDDRFLSALIKCEGCGKVYNQDVNALEVVMRE
GRRHTSVA (SEQ ID NO: 60)
>3300013131|Ga0172373_10056063_2
[aquatic-freshwater]
MGSRVFQFGCPFGPSAGLDETIEQMRLGRAYYNARQEVSRQVRNQTRSIYASCGSVSDLERAVEEAKERKDAL
ELEIKTARAETRTRWTKKSSAQDLKEARTVLKDARAELGAFRSRLREDPIVAAKLATITGGRPKRKDGEASRR
HDNTVKNNGTKALAMRALRAEYGPRGKGLGSGTYLLVEAAQGVSEADTPLYDREGQPQDPGFRCWSMGSKHVA
VHVQGFELTGATIFQPNDWAWIKPVDPRAWLKETPRGERKRLSRTMLHLRLKTGEDREPVWAVVPIIVHREIP
LTAKVTWIVLSMCQQGPRAVWTCEITVNEEAPQSVPEGRGTAAVVFGWRNVSGGILAATWLNTDGVAGQLVLG
DGDLDTTDADGGKGGIISGLTRVDSLKETRDKNLNAALASLVSWLRDHDMPEWMRLRTVKRQYDENHQEVQRV
LPSKAQALAYLAGWKAQGKLAALCLAWRENRFSGDDDAFRALESWRYHDNHLWRWQAAQNESAHLRRRERYRI
VGIELAKHARVLLDGTDFARIAFRPKTEDDKGYVQGPATNRTLVAPSELRDTVKQAAAKMLRDAVKVESANTA
ITCPRCGNVSKVQRFDDGDFKHSFCCVECGLTGDQDSIRCMNMLVADGHKDAVLEILRRQEEALRYQRRLSNG
GDYGEDASNRSENAGVQGAAREVQCDRRRGVLLPHDVGG (SEQ ID NO: 61)
>3300013136|Ga0172370_10027535_4
[aquatic-freshwater]
MVTKRLAYGLLEPTENLETVEDQMSKAHKYYNKLVEIENSIRPLRRESYNNGVKRLSPEYEGLEQQLLALQAS
KDTIENEIKQQRVLNRSKKLDSSNHKQTLAGIKLQIKLVYEKQKEERAKFKGKLKRPSKEKVEPEPHKRPSKE
KVEPEPHKRPSKEKVEPEPQWLAEQEEIDEKRNKLISEARKASGLYWGTYLSTEDAFKNACKATPPHKNLHFQ
RWTGEGKIRVCRNSEPTVSNSLFFIDPLPGDSWEKKKDKGVPSPRGEGKRSRMKTQLHLLVNSNRQKKIAPVW
ATFPMMLSRPLPDNGRIDSVEVIRKRCGPNWKWAAQVTCTFEETNKPPKGKAIVALDLGWRKIDGNIRVAAFG
AIDDAPISLPSESCPDLQQAVVKVGNGYELQLTPEVISGIRKSEELRSIRDSEFNDIRSILTRWLQENDVPEE
IRTLKKRGKLNVMSQLQFIHWINSLRSQAQLASLIYRWKDNRFEGYEEILEALENWRYADQHLWEWESEQRRG
AIARRNNLFKNFASWLRSIATMVVIEGDFKITDVAERKGLIEDTNRNEIAQSNRQLAGTSILRICIKNKLGND
CIGVPAKNTSKECHVCGEVVEFADPAALEQECHNHHQWDRDHNAWKVLLKRYASGDVIVKTLGTARKGKKKRN
SKKLATGEQKIVG (SEQ ID NO: 62)
>3300013137|Ga0172375_10012175_6
[aquatic-freshwater]
MGSRVFQFGCPFGPSAGLDETIEQMRLGRAYYNARQEVSRQVRNQTRSIYASCGSVSDLERAVEEAKERKDAL
ELEIKTARAETRTRWTKKSSAQDLKEARTVLKDARAELGAFRSRLREDPIVAAKLATITGGRPKRKDGEASRR
HDNTVKNNGTKALAMRALRAEYGPRGKGLGSGTYLLVEAAQGVSEADTPLYDREGQPQDPGFRCWSMGSKHVA
VHVQGFELTGATIFQPNDWAWIKPVDPRAWLKETPRGERKRLSRTMLHLRLKTGEDREPVWAVVPIIVHREIP
LTAKVTWIVLSMCQQGPRAVWTCEITVNEEAPQSVPEGRGTAAVVFGWRNVSGGILAATWLNTDGVAGQLVLG
DGDLDTTDADGGKGGIISGLTRVDSLKETRDKNLNAALASLVSWLRDHDMPEWMRLRTVKRQYDENHQEVQRV
LPSKAQALAYLAGWKAQGKLAALCLAWRENRFSGDDDAFRALESWRYHDNHLWRWQAAQNESAHLRRRERYRI
VGIELAKHARVLLDGTDFARIAFRPKTEDDKGYVQGPATNRTLVAPSELRDTVKQAAAKMLRDAVKVESANTA
ITCPRCGNVSKVQRFDDGDFKHSFCCVECGLTGDQDSIRCMNMLVADGHKDAVLEILRRQEEALRYQPAAE
(SEQ ID NO: 63)
>3300010293|Ga0116204_1010874_1
[aquatic-freshwater-anoxic lake water]
MLDQLRLASVYRNKLVEIELARRAATDDTLRELCPGLLECEAELADVNAKIAEATAEHKAKNAKARCLTDDKE
IKAALTQLKCIRKELATTRKRLRDDGFSPLTEADLSLVPGLAEATKIHAAAENPHAKAKAAAVMHECRSSWLN
DPSRPTPIRRLAIQIQLAEIGYAANEAQKTARKTSGLAPGSYLLVDQAADAFRKGAPPVFRGYQGEGRVGVQI
VGGMNSEEANSGRDTRLRIVHTPQAEQRVAKNGRVLPAPGAKRQAQQYTLWLRIGSDGRTPTWATWPLILHRP
IPETTRIMWAIVQRRIVGGHERWQLTLNLRDDTNAAFARRDVTASGVCGVDIGYRYIDDRAQRVAYWHGSDGA
SGELQLPSGKVAQWKKVDDLQSIRDGLHNEARAALRDWLATNAHPEWLDEATEHMHAWRRLSRLDRLVAQWRG
QRFDGDAEIMATLESWRTRERHLWQYQEQMRDQLLAWRKDFYRNFAAMLRRRYRTIAVEDMDLRSAIHDVLRP
EEERETVTAQRRAARFAALSVLVAAIKDSGADVVAVEQAGTTSTCSWCGASNEVGTGVIHTCVGCGREWDRDD
NAARNICARGEVAVKTR (SEQ ID NO: 64)
>3300010293|Ga0116204_1010874_2
[aquatic-freshwater-anoxic lake water]
MSSKNYTYGLQTPVGNRDRVLDQLRLASVYRNKLVEIELARRAATDDTLRELCPGLLECEAELADVNAKIAEA
IAEHKAKNAKARCLTDDKEIKAALTQLKCIRKELATTRKRLRDDGFSPLTEADLSLVPGLAEATKIHAAAENP
HAKAKAAAVMHECRSSWLNDPSRPTPIRRLAIQIQLAEIGYAANEAQKTARKTSGLAPGSYLLVDQAADAFRK
GAPPVFRGYQGEGRVGVQIVGGMNSEEANSGRDTRLRIVHTPQAEQRVAKNGRVLPAPGAKRQAQQYTLWLRI
GSDGRTPTWATWPLILHRPIPETTRIMWAIVQRRIVGGHERWQLTLNLRDDTNAAFARRDVTASGVCGVDIGY
RYIDDRAQRVAYWHGSDGASGELQLPSGKVAQWKKVDDLQSIRDGLHNEARAALRDWLATNAHPEWLDEATEH
MHAWRRLSRLDRLVAQWRGQRFDGDAEIMATLESWRTRERHLWQYQEQMRDQLLAWRKDFYRNFAAMLRRRYR
TIAVEDMDLRSAIHDVLRPEEERETVTAQRRAARFAALSVLVAAIKDSGADVVAVEQAGTTSTCSWCGASNEV
GTGVIHTCVGCGREWDRDDNAARNICARGEVAVKTR (SEQ ID NO: 65)
>3300008255|Ga0100403_1011992_3
[aquatic-freshwater-aquifer]
MTRVFEYGLPFDPFDGAELVDEQILLAHRYYNKLIELEHTRRSSILAVQRADPKVGPLLAAYDAANAEVEDLL
ARKREAKSRDRRVAAPELSEIEAAKEARRHLSVQLRKVKKVATDRLKPEYDLAEQATRDAKKAARAASGVFWG
TYSLIEQAADAAAKAKPVLRPGTHPRPWDQQPSFRRWTGEGMVAVQININRPLNDVTVFGDDLRLRITPVDPA
AWSDATSRGDRKCLARTNVTMRVGRNTGETATWPMVMHRPLPAGSRVTWAKVLRWRLDDRPHWFKYVLQLTVE
TADAPRHPGLVSLPPAIVAINCGWRALPNGSLRVVTWVGSDGAEGVLDLGCREYRDRIERAESIRSVRDQLRN
ELTSKLVGIGIDVTRWRSFDRFHRLFRELTAEGCERNEAVELLEAWHHRDRHLRQYQDGARGGALRFRREQYR
LLAVELARRYPVVCVESWDLRPVVTDEDRLPGPAAARVEGASSTARLALASAATREGCVVLTQIAAHVRLQTQ
TCHVCGYGAKKGEEWDAAAELVHTCEGCGETWNQDVNFCRNILAASRAAVTEIPELLVPKIMKRSARFAARHK
KVAT (SEQ ID NO: 66)
>3300014155|Ga0181524_10003409_23
[aquatic-freshwater-bog]
MLVYKWGIGPLPLEARQIIDREVRAAHRYRNRLVEIERSRRAAYRDLRNSLSPELAHLHAAYAAADRAVVEGR
RLLSGVPRAERARHPVAAEVRRLSAARSGAWKAYAEARDRTTADVFGAADQKYREAKQSVTAAVGFACAVARF
ARGADPGPIGPHVLAAITAAVRERALADPAVGEPWKAKTRSQMEHEALAKRARAECGCAVGTYLAVEAAAEKS
FAECAGDPPFSRLECERVGLQVRGGGLSADDVVGAAVGQVRVEFPADMTARANGTRYAVVHLHLSGRGDQATW
LHLPVVWHRDMAPEARVRWAYVVARRVGLVWHYELQLTCDSVDRARAQSAGARGTIALNLGWRALKNGDLRVA
TPWSGSASRAEDRLVLPRSFREGSDLADRLLSYADEHFLAVRDALAGWFKGGERSLFSPEQVAAWSLDTVHAW
RSHGRLARVALDLRRDWLEARGVDVPALWKAWRLERSPKLDLFGPLDEIKAWLAGRGVVSADQVLAVYLDWWR
AKDRHLVNWARNNDLRLRRSRRDRYRCYARDLAARYERVVIEQWNKSETAETPDPEADTRTEQEVRGNSNRVL
ACVSELVDALEAAFGEANVHRAPSERITVEHHGCGGESSDPLPQIPVTCFACGQVYDQDLNAAKHLYDRHSGE
PSGGVNVGGGARGAKKSRRVEGFGRAAE (SEQ ID NO: 67)
>3300014156|Ga0181518_10000096_28
[aquatic-freshwater-bog]
MLVYKWGIGPLPLEARQIIDREVRAAHRYRNRLVEIERSRRAAYRDLRNSLSPELAHLHAAYAAADRAVVEGR
RLLSGVPRAERARHPVAAEVRRLSAARSGAWKAYAEARDRTTADVFGAADQKYREAKQSVTAAVGFACAVARF
ARGADPGPIGPHVLAAITAAVRERALADPAVGEPWKAKTRSQMEHEALAKRARAECGCAVGTYLAVEAAAEKS
FAECAGDPPFSRLECERVGLQVRGGGLSADDVVGAAVGQVRVEFPADMTARANGTRYAVVHLHLSGRGDQATW
LHLPVVWHRDMAPEARVRWAYVVARRVGLVWHYELQLTCDSVDRARAQSAGARGTIALNLGWRALKNGDLRVA
TPWSGSASRAEDRLVLPRSFREGSDLADRLLSYADEHFLAVRDALAGWFKGGERSLFSPEQVAAWSLDTVHAW
RSHGRLARVALDLRRDWLEARGVDVPALWKAWRLERSPKLDLFGPLDEIKAWLAGRGVVSADQVLAVYLDWWR
AKDRHLVNWARNNDLRLRRSRRDRYRCYARDLAARYERVVIEQWNKSETAETPDPEADTRTEQEVRGNSNRVL
ACVSELVDALEAAFGEANVHRAPSERITVEHHGCGGESSDPLPQIPVTCFACGQVYDQDLNAAKHLYDRHSGE
PSGGVNVGGGARGAKKSRRVEGFGRAAE (SEQ ID NO: 67)
>3300014158|Ga0181521_10000063_92
[aquatic-freshwater-bog]
MLVYKWGIGPLPLEARQIIDREVRAAHRYRNRLVEIERSRRAAYRDLRNSLSPELAHLHAAYAAADRAVVEGR
RLLSGVPRAERARHPVAAEVRRLSAARSGAWKAYAEARDRTTADVFGAADQKYREAKQSVTAAVGFACAVARF
ARGADPGPIGPHVLAAITAAVRERALADPAVGEPWKAKTRSQMEHEALAKRARAECGCAVGTYLAVEAAAEKS
FAECAGDPPFSRLECERVGLQVRGGGLSADDVVGAAVGQVRVEFPADMTARANGTRYAVVHLHLSGRGDQATW
LHLPVVWHRDMAPEARVRWAYVVARRVGLVWHYELQLTCDSVDRARAQSAGARGTIALNLGWRALKNGDLRVA
TPWSGSASRAEDRLVLPRSFREGSDLADRLLSYADEHFLAVRDALAGWFKGGERSLFSPEQVAAWSLDTVHAW
RSHGRLARVALDLRRDWLEARGVDVPALWKAWRLERSPKLDLFGPLDEIKAWLAGRGVVSADQVLAVYLDWWR
AKDRHLVNWARNNDLRLRRSRRDRYRCYARDLAARYERVVIEQWNKSETAETPDPEADTRTEQEVRGNSNRVL
ACVSELVDALEAAFGEANVHRAPSERITVEHHGCGGESSDPLPQIPVTCFACGQVYDQDLNAAKHLYDRHSGE
PSGGVNVGGGARGAKKSRRVEGFGRAAE (SEQ ID NO: 67)
>3300014159|Ga0181530_10000119_98
[aquatic-freshwater-bog]
MLVYKWGIGPLPLEARQIIDREVRAAHRYRNRLVEIERSRRAAYRDLRNSLSPELAHLHAAYAAADRAVVEGR
RLLSGVPRAERARHPVAAEVRRLSAARSGAWKAYAEARDRTTADVFGAADQKYREAKQSVTAAVGFACAVARF
ARGADPGPIGPHVLAAITAAVRERALADPAVGEPWKAKTRSQMEHEALAKRARAECGCAVGTYLAVEAAAEKS
FAECAGDPPFSRLECERVGLQVRGGGLSADDVVGAAVGQVRVEFPADMTARANGTRYAVVHLHLSGRGDQATW
LHLPVVWHRDMAPEARVRWAYVVARRVGLVWHYELQLTCDSVDRARAQSAGARGTIALNLGWRALKNGDLRVA
TPWSGSASRAEDRLVLPRSFREGSDLADRLLSYADEHFLAVRDALAGWFKGGERSLFSPEQVAAWSLDTVHAW
RSHGRLARVALDLRRDWLEARGVDVPALWKAWRLERSPKLDLFGPLDEIKAWLAGRGVVSADQVLAVYLDWWR
AKDRHLVNWARNNDLRLRRSRRDRYRCYARDLAARYERVVIEQWNKSETAETPDPEADTRTEQEVRGNSNRVL
ACVSELVDALEAAFGEANVHRAPSERITVEHHGCGGESSDPLPQIPVTCFACGQVYDQDLNAAKHLYDRHSGE
PSGGVNVGGGARGAKKSRRVEGFGRAAE (SEQ ID NO: 67)
>3300014201|Ga0181537_10003972_13
[aquatic-freshwater-bog]
MSENMPTLVYRYGIAAPHDNADLVYEQLRLAHEYRCSLVRIERTRRAEERAARLAVSAEVAAAEAAVAAADAE
CERLATEIRKARSDARKRVETQQMRDALAKAREVRKERKTALFELRDRYQPQCRDCRATKSEDKPCPHVGQEA
QSFCLVLDAIAERAKESIRKARAESGLYWGSYLLVDRAMAASRKAPLYGDDGITPNDPKMPRFDGGGAVAIQF
QSSSVRPSNVRLADLGPDNARLQIVLPPWPEQCMPAPESHQGPFDPSRPPAGMRPDGTLAPATRADGSPARWL
RRRANRQALVRMCVKTEGRGKPVWAAWRLDYDRPLPAQATISWATIHRRMRGPHAEWSLCLTVEVAAEPAAEI
RSGQVAIDVGWRQMPCPGGAACHGQRTDCHELRVAAWRDHGGGSGELRLSARDIRALRQPAELRSKRDTQFDA
IKAAVAGWIRSASDAPEWMREAAKVMHAWRWQGRMVALVRQWAQERPNRAAPEEAVYQAALAWQTADWALWES
ERARDAWAHRRRREIYRVWAARMAETYGTLILERFDLRDVTERAPVGQDDSENETARSNRHLAAVSELRGALC
NAVRTRGSEVVGVTAVNSTRTCPSCGLVSDRNQAQAVQLACECGHVWDQDVEGAAPWLLAEYRERPGDAKLQA
GARAEAIAAARKGKKGNDWARAKRMGAAKKGRLQAARESAATEAQ (SEQ ID NO: 68)
>3300014201|Ga0181537_10021284_1
[aquatic-freshwater-bog]
MKLVYKYGLATPHDNRELVEEQMRAAHRYRNTLTEIERGRRAAVRQAEAEAGDMPKALQALRASEAELEAALT
AIRRHRARTSKRDEPVALKASAKAAREAKRAASKAFRDLRRRIAEDPMVVAAKDAIGERANELGRSARAHSGV
YWGSYLLVEAAASASFEDTPMYASDGRPTDPAFVRWTGEGEVGVQLQGGLGADEATACTDTQLQITQPDERAW
ERRGRTHRECEQMARQAQLRMRVQSDAKGKPVWATWRMDMHRPLPEGAIIKLATVHRVRVGPHSKWYVTITLD
VPARARVSPSSGTVAVDVGWRVVGDELRVAGWQDTTGARGELRLSPRDIAMLRAPEAMRSERDRRFDAARANL
LGWLRSHQELVPEWLAKATTTLHAWRSEARLVALYSRWSGSRFEGDEQPYYALASWRARARHEWAVESCARDQ
ALRRRRERYRVWAAQLASKYNTIVIEKFDKREVAVIPAPDVQVEQNAEQAARDKAARSNRFLAATSELCDCLV
TAARSRGCTVIAVPCEDTTRTCPVCGLVESRDAAAAIELTCECGASWDQDVDGAPAVLLARARERPGDTKILV
GAREDEKKNENGQKPESQWQRVRRMRAEKEARMGTAREAAPEGAE (SEQ ID NO: 69)
>3300014201|Ga0181537_10040512_3
[aquatic-freshwater-bog]
MTTRVYQFALLPPSGRDAALVDAQMRLAWEARQDMAMIERGRRSAMRALLDTPDVRAAEEALKAATRSTRKDV
IRVVSRARRDALERAVASERYDDEACQETGYCPLYEPERIEQLAKLATKGAYHYFGDRGLAWGTRLDVSGAAD
AARKAPLYDDDGLTPSDPHVERWYDAKRPPDSQLAVQLQGGLSTPDGLTGQDTRVRLVDGVLWLRVGSDGRAP
VWAKFAIARPHRTGKRGVRTTHRAIPDDAKWKWVRVSRRRDGPWMRWSVEITLDVEREDWRVRDPQVQGVLAV
EVCWDRPDDAIVVARWRDDSGRSGTIELPDRIATGLPKVHGIRAVRDTIRADMAKRLQRALTEDRDPKPVWLA
DAAGSMHLWKSSSRFHRLIQQWQDERCDAARPAYELLDAWRLRDNHLYEYETGARGNVLRWRKNWYQTLAAEW
ARRYRIVVLDDRNLSREARWGEASEIRFMASPFELRQAIRNAFGRDVAEHTVKQTEKEKDEDDRDWCERALDA
RNAGVARTERETSEIKDKRGGAWAKRKQAKTTRHAEREAARKAVVKAAE (SEQ ID NO: 70)
>3300014654|Ga0181525_10000532_4
[aquatic-freshwater-bog]
MAVYVYQFGLSAPFGENADLVYDQLFATHRYRNTLIEIERGRRAAVRAVIDASNAQTVALTAEVARWNAETEA
LAKRIKSQRASTRTRSESESDREALRQAREARKAAVTKLREARLAQRTDAAMTAAIDAINERANGLVRGAREL
TETYWGTYLLTEKAMQDSKALPLYGDDGISPNDPKFLRWEGDGALGVQIQGGAKAATILAEASTLLRMRPDAR
AYLERACDQRRIKDKTGLLTMRVGSDAKGGPIWATWHMHMHRLIPENAMIKGATVHLRKVGTKAEWSLEVTVE
HSRAALPPNDKTIAIDIGWRMIGDELRVAGWMDSDGKTGELRLSAKDIRLLRRPEEIRGERDRHFTLAKTALG
SFLASAAQVPDRLRAETAHLDRWGSADRLAAVVARWERFEGDAAIHNVMTAWFWRDRNLCDQEAGMRLQALRR
RKNKYREWAAWVTETYGTVVVEKFDLRAVALRGAVEDPAANETARSNRQLAALSEARTAIVNAASSRGRLIAA
MPAHDTTRACPSCGVVEAREAEASIVLVCPDCGATWDQDVTGAPVVLLGRWRERPGDAKILVSARDGANDNES
ETMRPNRWQKVKEARTAKVLRRESARAEASNGAE (SEQ ID NO: 71)
>3300014657|Ga0181522_10000394_52
[aquatic-freshwater-bog]
MKELRGNHELRNRLVEIERERRKAVRALCADLPELAAAQVCRTALDEALQTIKKARSETKKRSESAEDVKRAK
EARKAYQEALRALALARRARLSACEAEIKVVNDESAKREKEAYGESPVEAWGSKLDVFAAHAAVRAMPYWDEL
ADNDPHFVRWEGEGQIAVQLQGGLRVGAALSGGDRRFQLTDLVPEAFEATDAAKNPRDRRRLRGAVGRLRIGS
DARNPIWTEVRVQVHRPLPPSGIIKWARLSRRRVALAYAWSLEVTVDVPLSAVARPGVVGLDLGWRKKPDGSL
RVGYLAFRETAKDAIATRTRELVLPASLVQRFARLREAESERTHAFEMERMWLSRLLSTFVELPDWLKKESET
LSQWRSPARLARLARIWSENRFERDELPYERLRGWAAGDALRYQENEEARQSALRAREWYYGNWAAACANAYG
ALAVENMNISRLIRHRDPDADEPAHEERARSRAVAAPGRLRQVFAHAFEGRGGIVMLRPTKNTTITCPTCGDV
RKFDAAEILAPTCANGHTIDQDERAARNLCEGVSGEEAAEAARAREVRESTPKESRWAKVKRMKREKEEGALA
RGVGSAG (SEQ ID NO: 72)
>3300014657|Ga0181522_10000394_53
[aquatic-freshwater-bog]
MNRVYRYSCSPPKTEAERVMKELRGNHELRNRLVEIERERRKAVRALCADLPELAAAQVCRTALDEALQTIKK
ARSETKKRSESAEDVKRAKEARKAYQEALRALALARRARLSACEAEIKVVNDESAKREKEAYGESPVEAWGSK
LDVFAAHAAVRAMPYWDELADNDPHFVRWEGEGQIAVQLQGGLRVGAALSGGDRRFQLTDLVPEAFEATDAAK
NPRDRRRLRGAVGRLRIGSDARNPIWTEVRVQVHRPLPPSGIIKWARLSRRRVALAYAWSLEVTVDVPLSAVA
RPGVVGLDLGWRKKPDGSLRVGYLAFRETAKDAIATRTRELVLPASLVQRFARLREAESERTHAFEMERMWLS
RLLSTFVELPDWLKKESETLSQWRSPARLARLARIWSENRFERDELPYERLRGWAAGDALRYQENEEARQSAL
RAREWYYGNWAAACANAYGALAVENMNISRLIRHRDPDADEPAHEERARSRAVAAPGRLRQVFAHAFEGRGGI
VMLRPTKNTTITCPTCGDVRKFDAAEILAPTCANGHTIDQDERAARNLCEGVSGEEAAEAARAREVRESTPKE
SRWAKVKRMKREKEEGALARGVGSAG (SEQ ID NO: 73)
>3300009175|Ga0073936_10014029_2
[aquatic-freshwater-freshwater lake hypolimnion]
MNPASMAPKGMIVGLLMTYNIYLPLLFCKFEACRIYLGVLAIGQARYTSEKLEAKKMLDKGAKVTFTYDGNET
SGKILHTLAAGKKPAAYFDILTAADKALEADRCFDETRSCEDESYLVVTKRSKNTVAKIYWLSKDDLSGVQVV
VRQYGLLQPSNWQDDCFNHLYLQNRYWNCLVEIEQDNRNKYRALVGEDEDVAPIQDAIDGLKSRIADMAEQRT
QLKIEHCKKIGIHTEPLDNAIKAAKAEMKKLSNKAKEARAVAKERIRAAGPAFKLLEDERRQSVKEAYNNSQL
WWGNYNAITNSYNTARTRAMKEGADLRFHRFDGSGRFTCQIMGGMSTDDLLSGRNSVAQLRKVSNSEFTKIIK
SNPPALQLQLVGSRRDEREYGVLSITIYTAEDDQGKKTRRTLDFPIILHRPLPENATLKIISVNRKKIGTDYR
WAVTFTFSEETKESIVHTSKQTCGINLGWKQVAGGLRVATVSDGTSTRHVVLPQVIIDKLAYTESLQSRIDTA
TNENFIWLLGKMADPPEILKGDVTSLKRSKRPHPAKFAKFVIKWRNECSEFEPQALIEAEVMRKNVKRLSLEH
HHLRDKVLRRRIDFYRNEAKKIADKYSMIVMDKMDLRQMSALEKSDGTPNELADLARYHRKVAAISEFREWIG
KQAIKAGGAVEMIAIESTRTCNACDGVMAPSDGLMFRCKSCGTFVDQDENASANLLRAVT (SEQ ID NO:
74)
>3300015360|Ga0163144_10020017_5
[aquatic-freshwater-freshwater microbial mat]
MSVRVYKYGLRRPHEQGERVRAQMRAAHRYRNTLVEIERARRTAVRSAMSAYGNIGELEAAARSADVVVSDAV
RLAKAAKAEARSHSGVSSDQKAALLAARERKRDAVRLLRETRVLLRQDVVLSTEVDRVNELAAELRRNARKHC
GVYWGTYLLIEAADEAARKVPLYDGAEPSDPRFMRWAGEGRVGVSIAKGADIAVLDDTKDTRIRIEPGTMPKG
ADPASKRSAKRRHAVLAMRVGSGDQREPVFARWEMVMHRSLPAGARIKNAAVSLRLVGPREEWSVAITLDTTA
CAETATRGRGVVGVDLGWRMLNGDIRSAAWDGGDVSGFLALPAELIGQVEKVADLRSIRSKNFDASRAALVAA
MPADAPAWLRGATASLGQWKSIDRLTKLALRWRVARFDGDAAAYDALEAWRYNDHHLWCWESEQRTRTLRHRR
EIYRIFAAKLAREYETLAIENFDLRVFSVRAPVETDASIDTVTRAARVVVSPSELRLSLVNAFGPHRVVKVDA
ANTTRECSECHHINTWDAAAELSHTCAQCGARWDQDANAARVIRARGTAASPAPGAARNGDSANDSAAPIESR
WAKAKRMRAEKRSGEGGARKPVDAAAE (SEQ ID NO: 75)
>3300015360|Ga0163144_10020017_4
[aquatic-freshwater-freshwater microbial mat]
MRAAHRYRNTLVEIERARRTAVRSAMSAYGNIGELEAAARSADVVVSDAVRLAKAAKAEARSHSGVSSDQKAA
LLAARERKRDAVRLLRETRVLLRQDVVLSTEVDRVNELAAELRRNARKHCGVYWGTYLLIEAADEAARKVPLY
DGAEPSDPRFMRWAGEGRVGVSIAKGADIAVLDDTKDTRIRIEPGTMPKGADPASKRSAKRRHAVLAMRVGSG
DQREPVFARWEMVMHRSLPAGARIKNAAVSLRLVGPREEWSVAITLDTTACAETATRGRGVVGVDLGWRMLNG
DIRSAAWDGGDVSGFLALPAELIGQVEKVADLRSIRSKNFDASRAALVAAMPADAPAWLRGATASLGQWKSID
RLTKLALRWRVARFDGDAAAYDALEAWRYNDHHLWCWESEQRTRTLRHRREIYRIFAAKLAREYETLAIENFD
LRVFSVRAPVETDASIDTVTRAARVVVSPSELRLSLVNAFGPHRVVKVDAANTTRECSECHHINTWDAAAELS
HTCAQCGARWDQDANAARVIRARGTAASPAPGAARNGDSANDSAAPIESRWAKAKRMRAEKRSGEGGARKPVD
AAAE (SEQ ID NO: 76)
>3300015360|Ga0163144_10033243_8
[aquatic-freshwater-freshwater microbial mat]
MSIRVYKYGLRRPHEQSERVRAQMLAAHRYRNTLVEIERARRAAVRSAMSAYGNIGELEAAAHAADTVVLGVV
RLAKAAKAEARSHSGISSDQKAALSAAREHRRDAVRLLRETRVLLRQDVVLSTEVDRVSELACELRKSARKHC
GVYWGTYLLIEAADEAARKAPLYDGAEPSDPRFARWIGEGRVGVSIMKGADISVLDMEDTRIRIEPGTMPKGA
DPTSKRSAKRRHTVLAMRVGSDDQRGPIFARWEMVMHRPLPAGARIKNAAVSLRLVGPREEWSVAITLDTTAC
AETATSGRGVVGVDLGWRMLDGDIRSAAWDGGDLSGYLALPAELIGQVEKVADLRSIRSKSFDASRDALIAVM
PTNAPAWLRAATSSLRQWKSINRLTKLALRWRVARFDGDAAAYDALEAWRYNDHHLWCWESEQRTRTLRHRRE
IYRIFAAKLARKYETLAIENFDLRVFSVRAPVETDASIDTITRAVRVVVSPSELRRSLINAFGPHRVVKVDAA
NTTRECAECHHINTWDAAAELSHTCAQCSARWDQDANAARIIRARGAAASPQGRNSDSATTTESRWAKAKRMR
AEKRSGEGGDRKSVDTAAE (SEQ ID NO: 77)
>3300015360|Ga0163144_10033243_7
[aquatic-freshwater-freshwater microbial mat]
MSAYGNIGELEAAAHAADTVVLGVVRLAKAAKAEARSHSGISSDQKAALSAAREHRRDAVRLLRETRVLLRQD
VVLSTEVDRVSELACELRKSARKHCGVYWGTYLLIEAADEAARKAPLYDGAEPSDPRFARWIGEGRVGVSIMK
GADISVLDMEDTRIRIEPGTMPKGADPTSKRSAKRRHTVLAMRVGSDDQRGPIFARWEMVMHRPLPAGARIKN
AAVSLRLVGPREEWSVAITLDTTACAETATSGRGVVGVDLGWRMLDGDIRSAAWDGGDLSGYLALPAELIGQV
EKVADLRSIRSKSFDASRDALIAVMPTNAPAWLRAATSSLRQWKSINRLTKLALRWRVARFDGDAAAYDALEA
WRYNDHHLWCWESEQRTRTLRHRREIYRIFAAKLARKYETLAIENFDLRVFSVRAPVETDASIDTITRAVRVV
VSPSELRRSLINAFGPHRVVKVDAANTTRECAECHHINTWDAAAELSHTCAQCSARWDQDANAARIIRARGAA
ASPQGRNSDSATTTESRWAKAKRMRAEKRSGEGGDRKSVDTAAE (SEQ ID NO: 78)
>3300015360|Ga0163144_10062707_6
[aquatic-freshwater-freshwater microbial mat]
MSSRSKENRMFGHESKPTRNYVYGILAPTEGADLVDEQLRAAHQYRNNLVRLELDRREAVQQCLLAMRPAVAR
LTGEVADAVTAYDAAAAALKVRNARERNKRASADERQASKDAAALLKGLRGQLKAVRTEAFAADDVRAALDAI
ETVASERRREARGACGVYWGTYLTVEQAAGSFRSGAPPIFHRWTGEGRLAIQLQNGVEPAVLTLGQDKRLRIE
LTGECGRGKRPLAVAWLRVGSDGRAPVWAKFPMVYHRPIPVDAKIKWAFVHRRRCGTFWRWQLMLSVARDAWE
SPTTSGGSVGIDLGWRVVPEGLRVASWAGDDGRRGELILPADDLRRWSEPATRRAERDVRFGEFLPRVADWFA
ANAGRFGEEMRERVKTIRQWRSPARLAGLLRAWSAERVTGDEEIYGELVRWMREDSREWNSESGQRARASRWR
DDYYRCFVKRLASEYRVVHVEDMDLREIKRKPKAEEAESENQTARGNAFIASPGRLRELIREGFAETMSIDAA
WTTQRCHACGEIDGFDAAAELVRTCRHCGVAEDQDYRAAMNLLHGEQPDADEMAVVARGV (SEQ ID NO:
79)
>3300015360|Ga0163144_10062707_6
[aquatic-freshwater-freshwater microbial mat]
MFGHESKPTRNYVYGILAPTEGADLVDEQLRAAHQYRNNLVRLELDRREAVQQCLLAMRPAVARLTGEVADAV
TAYDAAAAALKVRNARERNKRASADERQASKDAAALLKGLRGQLKAVRTEAFAADDVRAALDAIETVASERRR
EARGACGVYWGTYLTVEQAAGSFRSGAPPIFHRWTGEGRLAIQLQNGVEPAVLTLGQDKRLRIELTGECGRGK
RPLAVAWLRVGSDGRAPVWAKFPMVYHRPIPVDAKIKWAFVHRRRCGTFWRWQLMLSVARDAWESPTTSGGSV
GIDLGWRVVPEGLRVASWAGDDGRRGELILPADDLRRWSEPATRRAERDVRFGEFLPRVADWFAANAGRFGEE
MRERVKTIRQWRSPARLAGLLRAWSAERVTGDEEIYGELVRWMREDSREWNSESGQRARASRWRDDYYRCFVK
RLASEYRVVHVEDMDLREIKRKPKAEEAESENQTARGNAFIASPGRLRELIREGFAETMSIDAAWTTQRCHAC
GEIDGFDAAAELVRTCRHCGVAEDQDYRAAMNLLHGEQPDADEMAVVARGV (SEQ ID NO: 80)
>3300020057|Ga0163151_10006104_16
[aquatic-freshwater-freshwater microbial mat]
MSVRVYKYGLRRPHEQGERVRAQMRAAHRYRNTLVEIERARRTAVRSAMSAYGNIGELEAAARSADVVVSDAV
RLAKAAKAEARSHSGVSSDQKAALLAARERKRDAVRLLRETRVLLRQDVVLSTEVDRVNELAAELRRNARKHC
GVYWGTYLLIEAADEAARKVPLYDGAEPSDPRFMRWAGEGRVGVSIAKGADIAVLDDTKDTRIRIEPGTMPKG
ADPASKRSAKRRHAVLAMRVGSGDQREPVFARWEMVMHRSLPAGARIKNAAVSLRLVGPREEWSVAITLDTTA
CAETATRGRGVVGVDLGWRMLNGDIRSAAWDGGDVSGFLALPAELIGQVEKVADLRSIRSKNFDASRAALVAA
MPADAPAWLRGATASLGQWKSIDRLTKLALRWRVARFDGDAAAYDALEAWRYNDHHLWCWESEQRTRTLRHRR
EIYRIFAAKLAREYETLAIENFDLRVFSVRAPVETDASIDTVTRAARVVVSPSELRLSLVNAFGPHRVVKVDA
ANTTRECSECHHINTWDAAAELSHTCAQCGARWDQDANAARVIRARGTAASPAPGAARNGDSANDSAAPIESR
WAKAKRMRAEKRSGEGGARKPVDAAAE (SEQ ID NO: 75)
>3300020186|Ga0163153_10017638_7
[aquatic-freshwater-freshwater microbial mat]
MTSVYRYGLLPPTLGADVVDDQMRAGHRYQNALVELERARRDAVAGVLSNNAIDEIDLEIKALDEELSARRAA
IQAERGATKRKRVPHTTATRDIARRRELRGTRRTLIAARRADPAVMAALAGIEERAKVLHKQFRADSGVYWCT
YLKVEQAMDAARKGAKFGPPSFRRWNGGGAVSMQLQRRGPERLLMTADNAIECDDPRLHLDLIPTPVPNRRGK
PLPRVRLRVGSDGARRPIWAEWPMIYHRPLPDGAVITWATVIRELVASSPRWALLLTIEHGGVAPTAARGAAV
AVDLGWRRAIVDGDITTRACGHTATDDSDESELHVHRDVFGALGKADNLRSIRDKRMNEMQAILVAWLRGCGS
EEHRERTRFVAQWRACARFAGLAIWWRDHRIEGDELIFVLLEAWRKRDKHLWLWEAHARRTARARRLDGYRVF
AADLARRYETLIVEKINLAKVAEKPKPESTREHNATASSQRTATAPSELRGALVNAFRGRGGTVVEVGAHPSA
TAMLGEWRERPVAEEKPGVARMSKFGRLRAERGGSWAQRSRPLEGGSASD (SEQ ID NO: 81)
>3300020195|Ga0163150_10003396_14
[aquatic-freshwater-freshwater microbial mat]
MTSVYRYGLLPPTLGADVVDDQMRAGHRYQNALVELERARRDAVAWVLSDGVIDEIDRGIEALSEELSAQRAG
IQAERGATKRKRVPHTAETRGLDDRRRELRGTRRTLIAARRADPAVMAALAGIEERAKVLSKLLSKSSGVHWG
TKGVVHQAMDAARKGAKFGPPSFRRWNGGGAVSMQLQRTGREKRPLTADDAIECDDTRLHLDLTPTPVPNRRG
KPRRGKPLPRVRLRVGSDGARRPIWAEWPMIYHRPLPDGAVITWATVIRELVASSPRWALLLTIEHGGVAPTA
ARGAAVAVDLGWRRAIVDGDITTRACGHTATDDSDESELHVHRDVFGALGKADNLRSIRDKRMNEMQAILVAW
LRGCGSEEHRERTRFVAQWRACARFAGLAIWWRDHRMEGDELIFVLLEAWRKRDKHLWLWEAHARRTARARRL
DGYRVFAADLARRYETLIVEKINLAKVAEKPKPESTREHNATASWQRTATAPSELRGALVNAFRGRGGTVVEV
GAHPSATAMLGEWRERPVAEEKPGVARMSKFGRLRAERGGSWAQRSRPPEPLEGGSASD (SEQ ID NO:
82)
>3300020203|Ga0163148_10001247_2
[aquatic-freshwater-freshwater microbial mat]
MFGHESKPTRNYVYGILAPTEGADLVDEQLRAAHQYRNNLVRLELDRREAVQQCLLAMRPAVARLTGEVADAV
TAYDAAAAALKVRNARERNKRASADERQASKDAADLLKGLRGQLKAVRTEAFAADDVRAALDAIETVFRERRR
EARGASDVYWGTYLTVEQAAWSFRSGAPPIFHRWTGEGRLAIQLQNGVEPAVLTLGQDKRLRIELTGEYGRGK
RGKRPLAVAWFRVGSDGHTPVWAKFPMVYHRPIPVDAKIKWAFVHRRRCGTFWRWQLMLSVARDAWESPITSG
GSVGIDLGWRVVPEGLRVASWAGDDGRRGELILPADDLRRWSEPATRRAERDVRFGEFLPRVADWFAANSGRF
SEAMRERVKSIRQWKSPARLAGLLRAWGDERVVGDEEIHAELVTWMREDSREWNSEAGQRARASRWRDDYYRC
FVKRLAIEYRVVHVEDMDLREIKRKPKAEEAESENQTARGNAFIASPGRLRELIREGFAETMSIDAAWTTQRC
HACGEIDGFDAAAELVRTCRHCGVTEDQDYRAAMNLLAGEQPDADEMAGVARGV (SEQ ID NO: 83)
>3300020203|Ga0163148_10001247_2
[aquatic-freshwater-freshwater microbial mat]
MSSRSKENRMFGHESKPTRNYVYGILAPTEGADLVDEQLRAAHQYRNNLVRLELDRREAVQQCLLAMRPAVAR
LTGEVADAVTAYDAAAAALKVRNARERNKRASADERQASKDAADLLKGLRGQLKAVRTEAFAADDVRAALDAI
ETVFRERRREARGASDVYWGTYLTVEQAAWSFRSGAPPIFHRWTGEGRLAIQLQNGVEPAVLTLGQDKRLRIE
LTGEYGRGKRGKRPLAVAWFRVGSDGHTPVWAKFPMVYHRPIPVDAKIKWAFVHRRRCGTFWRWQLMLSVARD
AWESPITSGGSVGIDLGWRVVPEGLRVASWAGDDGRRGELILPADDLRRWSEPATRRAERDVRFGEFLPRVAD
WFAANSGRFSEAMRERVKSIRQWKSPARLAGLLRAWGDERVVGDEEIHAELVTWMREDSREWNSEAGQRARAS
RWRDDYYRCFVKRLAIEYRVVHVEDMDLREIKRKPKAEEAESENQTARGNAFIASPGRLRELIREGFAETMSI
DAAWTTQRCHACGEIDGFDAAAELVRTCRHCGVTEDQDYRAAMNLLAGEQPDADEMAGVARGV (SEQ ID
NO: 84)
>3300020213|Ga0163152_10009495_14
[aquatic-freshwater-freshwater microbial mat]
MSSRSKENRMFGHESKPTRNYVYGILAPTEGADLVDEQLRAAHQYRNNLVRLELDRREAVQQCLLAMRPAVAR
LTGEVADAVTAYDAAAAALKVRNARERNKRASADERQASKDAAALLKGLRGQLKTVRTEAFAADDVRAALDAI
ETVASERRREARGACGVYWGTYLTVEQAAGSFRSGAPPIFHRWTGEGRLAIQLQNGVEPAVLTLGQDKRLRIE
LTGECGRGKRPLAVAWLRVGSDGRAPVWAKFPMVYHRPIPVDAKIKWAFVHRRRCGTFWRWQLMLSVARDAWE
SPTTSGGSVGIDLGWRVVPEGLRVASWAGDDGRRGELILPADDLRRWSEPATRRAERDVRFGEFLPRVADWFA
ANAGRFGEEMRERVKTIRQWRSPARLAGLLRAWSAERVTGDEEIYGELVRWMREDSREWNSESGQRARASRWR
DDYYRCFVKRLASEYRVVHVEDMDLREIKRKPKAEEAESENQTARGNAFIASPGRLRELIREGFAETMSIDAA
WTTQRCHACGEIDGFDAAAELVRTCRHCGVTEDQDYRAAMNLLHGEQPDADEMAGVARGV (SEQ ID NO:
85)
>3300020213|Ga0163152_10009495_14
[aquatic-freshwater-freshwater microbial mat]
MFGHESKPTRNYVYGILAPTEGADLVDEQLRAAHQYRNNLVRLELDRREAVQQCLLAMRPAVARLTGEVADAV
TAYDAAAAALKVRNARERNKRASADERQASKDAAALLKGLRGQLKTVRTEAFAADDVRAALDAIETVASERRR
EARGACGVYWGTYLTVEQAAGSFRSGAPPIFHRWTGEGRLAIQLQNGVEPAVLTLGQDKRLRIELTGECGRGK
RPLAVAWLRVGSDGRAPVWAKFPMVYHRPIPVDAKIKWAFVHRRRCGTFWRWQLMLSVARDAWESPTTSGGSV
GIDLGWRVVPEGLRVASWAGDDGRRGELILPADDLRRWSEPATRRAERDVRFGEFLPRVADWFAANAGRFGEE
MRERVKTIRQWRSPARLAGLLRAWSAERVTGDEEIYGELVRWMREDSREWNSESGQRARASRWRDDYYRCFVK
RLASEYRVVHVEDMDLREIKRKPKAEEAESENQTARGNAFIASPGRLRELIREGFAETMSIDAAWTTQRCHAC
GEIDGFDAAAELVRTCRHCGVTEDQDYRAAMNLLHGEQPDADEMAGVARGV (SEQ ID NO: 86)
>3300020219}Ga0163146_10006198_18
[aquatic-freshwater-freshwater microbial mat]
MSVRVYKYGLRRPHEQGERVRAQMRAAHRYRNTLVEIERARRTAVRSAMSAYGNIGELEAAARSADVVVSDAV
RLAKAAKAEARSHSGVSSDQKAALLAARERKRDAVRLLRETRVLLRQDVVLSTEVDRVNELAAELRRNARKHC
GVYWGTYLLIEAADEAARKVPLYDGAEPSDPRFMRWAGEGRVGVSIAKGADIAVLDDTKDTRIRIEPGTMPKG
ADPASKRSAKRRHAVLAMRVGSGDQREPVFARWEMVMHRSLPAGARIKNAAVSLRLVGPREEWSVAITLDTTA
CAETATRGRGVVGVDLGWRMLNGDIRSAAWDGGDVSGFLALPAELIGQVEKVADLRSIRSKNFDASRAALVAA
MPADAPAWLRGATASLGQWKSIDRLTKLALRWRVARFDGDAAAYDALEAWRYNDHHLWCWESEQRTRTLRHRR
EIYRIFAAKLAREYETLAIENFDLRVFSVRAPVETDASIDTVTRAARVVVSPSELRLSLVNAFGPHRVVKVDA
ANTTRECSECHHINTWDAAAELSHTCAQCGARWDQDANAARVIRARGTAASPAPGAARNGDSANDSAAPIESR
WAKAKRMRAEKRSGEGGARKPVDAAAE (SEQ ID NO: 75)
>3300020596|Ga0163149_10010333_13
[aquatic-freshwater-freshwater microbial mat]
MSVRVYRYGLRRPHEQGERVRAQMRAAHRYRNTLVEIERARRTAVRSAMSAYGNIGELEAAARSADVVVSDAV
RLAKAAKAEARSHSGVSSDQKAALSAARERKRDAVRLLRETRVLLRQDVVLSTEVDRVNELAAELRRNARKHC
GVYWGTYLLIEAADEAARKVPLYDGAEPSDPRFMRWAGEGRVGVSIAKGADIAVLDDTKDTRIRIEPGTMPKG
ADPASKRSAKRRHAVLAMRVGSGDQREPVFARWEMVMHRSLPAGARIKNAAVSLRLVGPREEWSVAITLDTTA
CAETATRGRGVVGVDLGWRMLNGDIRSAAWDGGDVSGFLALPAELIGQVEKVADLRSIRSKNFDASRAALVAA
MPADAPAWLRGATASLGQWKSIDRLTKLALRWRVARFDGDAAAYDALEAWRYNDHHLWCWESEQRTRTLRHRR
EIYRIFAAKLAREYETLAIENFDLRVFSVRAPVETDASIDTVTRAARVVVSPSELRLSLVNAFGPHRVVKVDA
ANTTRECSECHHINTWDAAAELSHTCAQCGARWDQDANAARVIRARGTAASPAPGAARNGDSANDSAAPIESR
WAKAKRMRAEKRSGEGGARKPVDAAAE (SEQ ID NO: 87)
>3300020596|Ga0163149_10010333_12
[aquatic-freshwater-freshwater microbial mat]
MRAAHRYRNTLVEIERARRTAVRSAMSAYGNIGELEAAARSADVVVSDAVRLAKAAKAEARSHSGVSSDQKAA
LSAARERKRDAVRLLRETRVLLRQDVVLSTEVDRVNELAAELRRNARKHCGVYWGTYLLIEAADEAARKVPLY
DGAEPSDPRFMRWAGEGRVGVSIAKGADIAVLDDTKDTRIRIEPGTMPKGADPASKRSAKRRHAVLAMRVGSG
DQREPVFARWEMVMHRSLPAGARIKNAAVSLRLVGPREEWSVAITLDTTACAETATRGRGVVGVDLGWRMLNG
DIRSAAWDGGDVSGFLALPAELIGQVEKVADLRSIRSKNFDASRAALVAAMPADAPAWLRGATASLGQWKSID
RLTKLALRWRVARFDGDAAAYDALEAWRYNDHHLWCWESEQRTRTLRHRREIYRIFAAKLAREYETLAIENFD
LRVFSVRAPVETDASIDTVTRAARVVVSPSELRLSLVNAFGPHRVVKVDAANTTRECSECHHINTWDAAAELS
HTCAQCGARWDQDANAARVIRARGTAASPAPGAARNGDSANDSAAPIESRWAKAKRMRAEKRSGEGGARKPVD
AAAE (SEQ ID NO: 88)
>3300004174|Ga0066406_1000030_21
[aquatic-freshwater-freshwater sediment]
MIRVYKYGIKPPFDFGEDCVDELRRMNNYWNRLVEIDREREVAFRNLCRSWSPEYATAMDRIDALKQPIDAIY
EEIRATRVKNRNKELPDEIKVRKDRLLGERKVLWETCKAIQKKLPKDLQEPLIQKYKTDCKLARQQSGLYWGN
YNAVSESFETAKSRTIKEGGRLHFRLFDGAGRFVNQIQGGMTAVDLFTGAKSQAKCSASVIRKSRGGTPHHSF
TFTAFTGRDVDGKRFRRELTADLAYHRPIPAEGTIKSIEIVRDIIDGHEKWHVCFTVSLPDIEIEHPKRNIAG
VNMGWRQIGSALRIAVIVDDKNQKREYFLPATVAHKFEHAESIQAKADEAENEMLVWLREIYQAANQAPQEWK
EAVQGVLRNRPARDAYAKLLSEWEGAQDLINGFDEYKAWHKENKKLHRYYAGTRRRAIAWREEIYRSIAKEIA
ENYAVIAITDTPLSQMSRTKSSGGLSIDNALPPAARRNRVIAGIYTLKEWIDKQAAKTGAVVEKITDKVTQTC
HKCSAIADKRVGSERYITCTNCESELEVDENAAINSRKLASGEVTPKKELRKAAKYQRVREAMNAKNDSARKM
ADNSSDGVA (SEQ ID NO: 89)
>3300004200|Ga0066422_1000628_7
[aquatic-freshwater-freshwater sediment]
MIRVYKYGIKPPFDFGEDCVDELRRMNNYWNRLVEIDREREVAFRNLCRSWSPEYATAMDRIDALKQPIDAIY
EEIRATRVKNRNKELPDEIKVRKDRLLGERKVLWETCKAIQKKLPKDLQEPLIQKYKTDCKLARQQSGLYWGN
YNAVSESFETAKSRTIKEGGRLHFRLFDGAGRFVNQIQGGMTAVDLFTGAKSQAKCSASVIRKSRGGTPHHSF
TFTAFTGRDVDGKRFRRELTADLAYHRPIPAEGTIKSIEIVRDIIDGHEKWHVCFTVSLPDIEIEHPKRNIAG
VNMGWRQIGSALRIAVIVDDKNQKREYFLPATVAHKFEHAESIQAKADEAENEMLVWLREIYQAANQAPQEWK
EAVQGVLRNRPARDAYAKLLSEWEGAQDLINGFDEYKAWHKENKKLHRYYAGTRRRAIAWREEIYRSIAKEIA
ENYAVIAITDTPLSQMSRTKSSGGLSIDNALPPAARRNRVIAGIYTLKEWIDKQAAKTGAVVEKITDKVTQTC
HKCSAIADKRVGSERYITCTNCESELEVDENAAINSRKLASGEVTPKKELRKAAKYQRVREAMNAKNDSARKM
ADNSSDGVA (SEQ ID NO: 89)
>3300004205|Ga0066415_1000057_23
[aquatic-freshwater-freshwater sediment]
MIRVYKYGIKPPFDFGEDCVDELRRMNNYWNRLVEIDREREVAFRNLCRSWSPEYATAMDRIDALKQPIDAIY
EEIRATRVKNRNKELPDEIKVRKDRLLGERKVLWETCKAIQKKLPKDLQEPLIQKYKTDCKLARQQSGLYWGN
YNAVSESFETAKSRTIKEGGRLHFRLFDGAGRFVNQIQGGMTAVDLFTGAKSQAKCSASVIRKSRGGTPHHSF
TFTAFTGRDVDGKRFRRELTADLAYHRPIPAEGTIKSIEIVRDIIDGHEKWHVCFTVSLPDIEIEHPKRNIAG
VNMGWRQIGSALRIAVIVDDKNQKREYFLPATVAHKFEHAESIQAKADEAENEMLVWLREIYQAANQAPQEWK
EAVQGVLRNRPARDAYAKLLSEWEGAQDLINGFDEYKAWHKENKKLHRYYAGTRRRAIAWREEIYRSIAKEIA
ENYAVIAITDTPLSQMSRTKSSGGLSIDNALPPAARRNRVIAGIYTLKEWIDKQAAKTGAVVEKITDKVTQTC
HKCSAIADKRVGSERYITCTNCESELEVDENAAINSRKLASGEVTPKKELRKAAKYQRVREAMNAKNDSARKM
ADNSSDGVA (SEQ ID NO: 89)
>3300004565|Ga0066503_104695_4
[aquatic-freshwater-freshwater sediment]
MIRVYKYGIKPPFDFGEDCVDELRRMNNYWNRLVEIDREREVAFRNLCRSWSPEYATAMDRIDALKQPIDAIY
EEIRATRVKNRNKELPDEIKVRKDRLLGERKVLWETCKAIQKKLPKDLQEPLIQKYKTDCKLARQQSGLYWGN
YNAVSESFETAKSRTIKEGGRLHFRLFDGAGRFVNQIQGGMTAVDLFTGAKSQAKCSASVIRKSRGGTPHHSF
TFTAFTGRDVDGKRFRRELTADLAYHRPIPAEGTIKSIEIVRDIIDGHEKWHVCFTVSLPDIEIEHPKRNIAG
VNMGWRQIGSALRIAVIVDDKNQKREYFLPATVAHKFEHAESIQAKADEAENEMLVWLREIYQAANQAPQEWK
EAVQGVLRNRPARDAYAKLLSEWEGAQDLINGFDEYKAWHKENKKLHRYYAGTRRRAIAWREEIYRSIAKEIA
ENYAVIAITDTPLSQMSRTKSSGGLSIDNALPPAARRNRVIAGIYTLKEWIDKQAAKTGAVVEKITDKVTQTC
HKCSAIADKRVGSERYITCTNCESELEVDENAAINSRKLASGEVTPKKELRKAAKYQRVREAMNAKNDSARKM
ADNSSDGVA (SEQ ID NO: 89)
>3300009686|Ga0123338_10029047_2
[aquatic-freshwater-glacier valley]
MNNSTPTNPSERSEPLAALERVRVVQFGACAPTAGWEAGFLQHRLRTRFWNAICEIERNHRDTVQATVGPLKE
AGMPSKDAYASEDVQALELARKGKRLVARQEAAQGGLFWTGYLEVERAMDTARRGLEPPRFKRFEAHEGKLNF
LFTNGLASSELCGDDLRVQFEPLELPEGCSARTRKANPYHVKLRVCSQDKKPVWLEFVAYLHRPIPEGSVRDV
ALIWRREGAKQRYSLSVTVREGGVIHAPAPFERAAINIGWKRLTHGLRVAYWRGTDGKHGEIVLSNAWLERYH
HALGIESVRAKPLNSIKEQMLAYFRTTPDVPEEMATRVTHLAQWRSAARFAVLYKFWVGQRWHGDDAGFNALE
AWHKRDVHLWQYGEGTSARLMRSRREQYRTFAAKMLERYGEIVMDKLDLRIFAELEARGDDLAPIARAQRVQA
APSTLRLALQNAYGREGRVVSWVGARTSSTCHVCRAPVVLGRDLIHTCQGCQSHWDVDDNACSNLLREPEAGI
KIPKASRSPRARKNLALGTAVGAD (SEQ ID NO: 90)
>3300001242|C687J13896_1000006_134
[aquatic-freshwater-groundwater]
MKRSKSDKSARVYKFGSPLKTAVESEVAMEQLRLQNRFWNALVEADKVFTEKYWAIRDGADARLPVLRKQIED
IKVRTEEIRTEIKKGRQDGIKGTPSDLKAEIVELKAQKKPLIAEKKEIWAFVKDTVKPQLHELDGERYDKNVA
IRQEYAQNGLYWGNYLAVMDSFETARMAIMKTQVEDGQKRPELQFHRFERVGRWTCQIQGGMNITQAFLGSNN
YFQIDRLPADAWTHPSRGERGRLKRTKARIRIGSGEKKTVPIWLEIPIVMHRPIPESAEIKSVSIHVSKLADK
FVWSLTVTVREDCSVPLERTGHCVAINIGWRAKGLATRIAYMLDSRGVEEEILLGSEYTVSNEKAASLQGIRK
KNFNETVAWFNEWKKANADIVPPWLSERTKMMMSWKSEAQLASVAIQWSGRGFRREGDPPEFRFHGDEEAFNK
IEAWRKQDKHLWQWHANLSDRIRGRRLCEYRKIALKLSKEYDVVIQEDFDLRKTKGKKKAEEGADNDDHIRRM
SDLASVSTFRTETIRAMRSAGKEHVKLDSKNITKTCPFCGGTIKPGRKTNIMVQCSKCGKVYDQDWAASKNLL
TAYLDSSGDVPPETP (SEQ ID NO: 91)
>3300005236|Ga0066636_10020712_3
[aquatic-freshwater-groundwater]
MTRVFEYGLPFDPFDGAELVDEQILLAHRYYNKLIELEHTRRSSILAVQRADPKVGPLLAAYDAANAEVEDLL
ARKREAKSRDRRVAAPELSEIEAAKEARRHLSVQLRKVKKVATDRLKPEYDLAEQATRDAKKAARAASGVFWG
TYSLIEQAADAAAKAKPVLRPGTHPRPWDQQPSFRRWTGEGMVAVQININRPLNDVTVFGDDLRLRITPVDPA
AWSDATSRGDRKCLARTNVTMRVGRNTGETATWPMVMHRPLPAGSRVTWAKVLRRRLDDRPHWFKYVLQLTVE
TADAPRHPGLVSLPPAIVAINCGWRALPNGSLRVVTWVGSDGAEGVLDLGCREYRDRIERAESIRSVRDQLRN
ELTSKLVGIGIDVTRWRSFDRFHRLFRELTAEGCERNEAVELLEAWHHRDRHLRQYQDGARGGALRFRREQYR
LLAVELARRYPVVCVESWDLRPVVTDEDRLPGPAAARVEGASSTARLALASAATREGCVVLTQIAAHVRLQTQ
TCHVCGYGAKKGEEWDAAAELVHTCEGCGETWNQDVNFCRNILAASRAAVTEIPELLVPKIMKRSARFAARHK
KVAT (SEQ ID NO: 92)
>3300014208|Ga0172379_10007070_15
[aquatic-freshwater-groundwater]
MRVYRYGLLRPTDEQELGLVREQMRLANKYRNWLVWLERGYRMALSELVDAHPSVAPFLSETEASEVKVDAKE
VKIRRKRKATRSRSESTEDRQEVATERVSLTERRNALSAARWAALKGPLKAEAKRMNDLWEEMQKETRRQSVC
GVFWGTSQIQDLAMKESRKALLWYRGKLALPDFVRWSDNQSVGVQVQDNIPPEDLFRQGSLVRIAPVSPQAWS
EAVPRGDRKRLQRTVLSLRVQSDAKRQPVWAHWPMIMHRAIPAGCVVTRVAVRCRMIGPREEWYATITVDDSK
AETAQPCGNGTVAMDLGWRAMKGGGIRVARWRDSDGGSGEFQLDEHIVSSLRKAEGLHATRDDNFNEARAKLQ
KWLAGAPDVPGWLRLDTETLGSWRSLERLQALAGKWKKNRFAGDEEGYAALEQWHYHDYHLWQWESDQRAKSL
RHRRELYRIFAAKMACRYSTLVLEDFEIPGVAKKPTVEEDSEYNKNAAHNRQLASPHEFRECMKAAFVARDGM
VQLLPCADTTRHCSVCGSLELFDQAKHLWHTCLACEGEGRATTWDQDDNAAQNLLDLWQNGADPVKTVSKALA
LANKREPAWIKAKRLARAKREADASGAVVQQPTEALEAE (SEQ ID NO: 93)
>3300014208|Ga0172379_10014650_2
[aquatic-freshwater-groundwater]
MGLVSGIKVYRYGLLAPTENAHLVGEQMWLAHRYQNTLIEIERARRAALRAVYVAHGDVAAMTAVCQAATAEV
ARLYRDAKAARSQSRKRQIPSEIGDALKVAKEASREAQARLRAARLAIKTDPSVVASREQIEERAAWLRRNAR
AYCGVYWGTYLGIESAVSQTAKMPLYDGSEPNDPRFSRWEHEGTVGVQLQGGLAGAGAMRCDDTRLRIEVGTA
PKGVDPTSRRSATRRYMVLAMRVDSDGRDPVWARWPMKMHRPLPDDAVIKWAHVHRRRRGPHDEWSVTLTIET
SAARPAAPTGAVGIDLGWRSLDTDGIRVAAWHGSDGRSGTLVLSEWDLSRLEKANDLRSIRDKKFDAARAALS
TWLENACVPAWFHEATAHLVQWKSIERLMGLVRRWKGSRFGGDDAAYEALEAWRYNDHHLWAWEAHQRVRALR
NRREIYRVFAARMAREYHTVVLEDWNISKIAKRPAVDEETVADGNKNSRTARQSVAVSELRLALTHAFGARVE
KVPCAFTTRDCHACGSVESWDQAAELVHTCSSCGVVWDQDANAAQNLLARFAARGGGDLDNAGTARGNETMNA
SETLKESRWARAKRVKAERIASDQVARE (SEQ ID NO: 94)
>3300014613|Ga0180008_1000021_8
[aquatic-freshwater-groundwater]
MIPDGKRKGEKRMILIYEYGIPFDPMEGHDFVEDQILMAHRYYNKLIEIERAKRARIRAIQQAHPILGPLVTE
SDETHELFNDIIDRQKKAKSKDNRYPEVDPEEWEAAKEISAEVRLRLTAAKAAVKAELTPAYEAASQEAKDRK
RLARANSKVYWGTYLITEAAAEAAVNAKPKSRPGKVPPPWHMCPAFRRWNGEGSLAVQIQKPKALTEVTVFGH
DNQFRITPVDPYAWDKSTPRGLRCRLGRTTFTMRVGMKRGETASFRMVMHRPLPPGSRITWAKIIRRRVDDRL
YRFRYFLQLTVETTLCVRHPGLDNADPVSIPVVAINCGWRALADGSLRVATWLGSDNRTGTLELGREEFRDRI
ERAESIRSRRDIDLDELKKAIEGFGEIFKSMEVECVEKWKSFSRFHGLYCDVLTEYAENPTEEKKELLELLTS
WHHRDRYLMQYENGCRGGALRFRREKYRLFALELAKAYPVVCIESWDLRRIVEDEHRLKEPSAARVEGASSIA
RQITRNTSLREGCVVLKQGDKEVELATQRCHLCGYGAKKRERWDAAKELVHVCGGCGAEWNQDVNFCENILTT
SRGDLVGAPQLLEPKIVIQLGRFQKRAAAKREREAAQADEQEE (SEQ ID NO: 95)
>3300014613|Ga0180008_1000021_9
[aquatic-freshwater-groundwater]
MIPDETTTSLFAGKLSDPGRNSHRHCSPGNLVIPDGKRKGEKRMILIYEYGIPFDPMEGHDFVEDQILMAHRY
YNKLIEIERAKRARIRAIQQAHPILGPLVTESDETHELFNDIIDRQKKAKSKDNRYPEVDPEEWEAAKEISAE
VRLRLTAAKAAVKAELTPAYEAASQEAKDRKRLARANSKVYWGTYLITEAAAEAAVNAKPKSRPGKVPPPWHM
CPAFRRWNGEGSLAVQIQKPKALTEVTVFGHDNQFRITPVDPYAWDKSTPRGLRCRLGRTTFTMRVGMKRGET
ASFRMVMHRPLPPGSRITWAKIIRRRVDDRLYRFRYFLQLTVETTLCVRHPGLDNADPVSIPVVAINCGWRAL
ADGSLRVATWLGSDNRTGTLELGREEFRDRIERAESIRSRRDIDLDELKKAIEGFGEIFKSMEVECVEKWKSF
SRFHGLYCDVLTEYAENPTEEKKELLELLTSWHHRDRYLMQYENGCRGGALRFRREKYRLFALELAKAYPVVC
IESWDLRRIVEDEHRLKEPSAARVEGASSIARQITRNTSLREGCVVLKQGDKEVELATQRCHLCGYGAKKRER
WDAAKELVHVCGGCGAEWNQDVNFCENILTTSRGDLVGAPQLLEPKIVIQLGRFQKRAAAKREREAAQADEQE
E (SEQ ID NO: 96)
>3300014656|Ga0180007_10000195_44
[aquatic-freshwater-groundwater]
MIPDGKRKGEKRMILIYEYGIPFDPMEGHDFVEDQILMAHRYYNKLIEIERAKRARIRAIQQAHPILGPLVTE
SDETHELFNDIIDRQKKAKSKDNRYPEVDPEEWEAAKEISAEVRLRLTAAKAAVKAELTPAYEAASQEAKDRK
RLARANSKVYWGTYLITEAAAEAAVNAKPKSRPGKVPPPWHMCPAFRRWNGEGSLAVQIQKPKALTEVTVFGH
DNQFRITPVDPYAWDKSTPRGLRCRLGRTTFTMRVGMKRGETASFRMVMHRPLPPGSRITWAKIIRRRVDDRL
YRFRYFLQLTVETTLCVRHPGLDNADPVSIPVVAINCGWRALADGSLRVATWLGSDNRTGTLELGREEFRDRI
ERAESIRSRRDIDLDELKKAIEGFGEIFKSMEVECVEKWKSFSRFHGLYCDVLTEYAENPTEEKKELLELLTS
WHHRDRYLMQYENGCRGGALRFRREKYRLFALELAKAYPVVCIESWDLRRIVEDEHRLKEPSAARVEGASSIA
RQITRNTSLREGCVVLKQGDKEVELATQRCHLCGYGAKKRERWDAAKELVHVCGGCGAEWNQDVNFCENILTT
SRGDLVGAPQLLEPKIVIQLGRFQKRAAAKREREAAQADEQEE (SEQ ID NO: 95)
>3300014656|Ga0180007_10000195_48
[aquatic-freshwater-groundwater]
MIPDETTTSLFAGKLSDPGRNSHRHCSPGNLVIPDGKRKGEKRMILIYEYGIPFDPMEGHDFVEDQILMAHRY
YNKLIEIERAKRARIRAIQQAHPILGPLVTESDETHELFNDIIDRQKKAKSKDNRYPEVDPEEWEAAKEISAE
VRLRLTAAKAAVKAELTPAYEAASQEAKDRKRLARANSKVYWGTYLITEAAAEAAVNAKPKSRPGKVPPPWHM
CPAFRRWNGEGSLAVQIQKPKALTEVTVFGHDNQFRITPVDPYAWDKSTPRGLRCRLGRTTFTMRVGMKRGET
ASFRMVMHRPLPPGSRITWAKIIRRRVDDRLYRFRYFLQLTVETTLCVRHPGLDNADPVSIPVVAINCGWRAL
ADGSLRVATWLGSDNRTGTLELGREEFRDRIERAESIRSRRDIDLDELKKAIEGFGEIFKSMEVECVEKWKSF
SRFHGLYCDVLTEYAENPTEEKKELLELLTSWHHRDRYLMQYENGCRGGALRFRREKYRLFALELAKAYPVVC
IESWDLRRIVEDEHRLKEPSAARVEGASSIARQITRNTSLREGCVVLKQGDKEVELATQRCHLCGYGAKKRER
WDAAKELVHVCGGCGAEWNQDVNFCENILTTSRGDLVGAPQLLEPKIVIQLGRFQKRAAAKREREAAQADEQE
E (SEQ ID NO: 96)
>3300014656|Ga0180007_10004731_7
[aquatic-freshwater-groundwater]
MFGHESQPSRIYAYGAKAPVVNGERVGEQIWLGHRYRNTLAEIELRRREQTDKMVVTLSPELPGVEAKLLEAD
QAIESAAAEIKLANKQARRQKATPEQKTKLAALRKERAALRKKRKALRDVVFSDSGTHDALTGIDQRAAAEQR
EARAESGLYWGTYLTVEQGCQSFRKGRPPRFLRWTGEGRIAVQVQGGLAPEDAFGGEDKRLIVEPLPEDAWSK
RSRGLKRTKAWLRIGSDDDRQPVWAVVPFVMHRSLPADCRIKWVYLHRRRVGTKDQWMLSFVIARQVWPQTDV
AGSGEIGIDLGWRLLDHGLRVAAWAGSDGESGELVLPIQDVGRWQKAQDLRGIRDTRLDAVIARFGEWLSGND
APDWLTERTRTLRQWRSAARLASVVLAWRDQRFAGDESIYADLEAWRKKDKHLYEWEANQRRKAVAWLKDLYR
NFAAAMARRYRVAVLEAVNWRDMGRRAGVGESDKAGAARRQRVIASPGRLAECIRERFADCVSAPAEYTTQRC
HACGEIDGFDARVEIVHTCGKCGKTWDQDYNAARNLLAFASGPVAKKTR (SEQ ID NO: 97)
>3300014656|Ga0180007_10004731_5
[aquatic-freshwater-groundwater]
MMFGHESQPSRIYAYGAKAPVVNGERVGEQIWLGHRYRNTLAEIELRRREQTDKMVVTLSPELPGVEAKLLEA
DQAIESAAAEIKLANKQARRQKATPEQKTKLAALRKERAALRKKRKALRDVVFSDSGTHDALTGIDQRAAAEQ
REARAESGLYWGTYLTVEQGCQSFRKGRPPRFLRWTGEGRIAVQVQGGLAPEDAFGGEDKRLIVEPLPEDAWS
KRSRGLKRTKAWLRIGSDDDRQPVWAVVPFVMHRSLPADCRIKWVYLHRRRVGTKDQWMLSFVIARQVWPQTD
VAGSGEIGIDLGWRLLDHGLRVAAWAGSDGESGELVLPIQDVGRWQKAQDLRGIRDTRLDAVIARFGEWLSGN
DAPDWLTERTRTLRQWRSAARLASVVLAWRDQRFAGDESIYADLEAWRKKDKHLYEWEANQRRKAVAWLKDLY
RNFAAAMARRYRVAVLEAVNWRDMGRRAGVGESDKAGAARRQRVIASPGRLAECIRERFADCVSAPAEYTTQR
CHACGEIDGFDARVEIVHTCGKCGKTWDQDYNAARNLLAFASGPVAKKTR (SEQ ID NO: 98)
>3300015370|Ga0180009_10002661_7
[aquatic-freshwater-groundwater]
MQAKVYVYGLRPPTHEAERVAEQLHLAHRYRNDLVAIERKRRERVAALLSASGLSAHEERLEAAEQVLEAALS
SLRAVRQAACKRAETSEQREAVKAARADVKAFREQLKEERKQLRPTLSAETETINDGAADERRAARAVCGVYW
GTYLLIEQADEQARKSPTPPQFQRWTGEGAVGVQLQGGLDTDTVFGADTRLQIDPVPPTAWDRRRSPERRTRV
RLRVGSDGRAPIWAEWPVTLHRPLPTGEIVWAKVLRQRVEAKSEWGLHLTIRVEDPTPTARSGAVGVDLGWRL
REDGLRSGYWVGSDGEHGEILVDQRTLDRLQKVKSLCSIRDRNLDELRPWLAEWLRARRAGLPEWLRERTQYL
HTWKAPRKFNALSVAWRAQRFPGDGEAVERLEAWRKQDKHLWTWETHQRERTLRCRREGYRLLAATLAERYGV
LVLEDLDLRVFQQRRPAEAEQGECQPARSQQPVAATSILRSCLINAFEAVGGRVVKLDPAGTTKECWLCGGTA
WSVQAEESVDRTCRECAALVDQDENAGRVLLARFERSGGIAGSADPDTSKSGQLRVSGGRWQRRKERCSKSGT
QDCTA (SEQ ID NO: 99)
>3300009760|Ga0116131_1003961_2
[aquatic-freshwater-peatland]
MKRKTSLVPTKVYRYGLLSPTSNGRLVDETIYRGHQFYNRLIEIERARRAEYRAERTRRFPELATVESLVEDL
TKQIETMRTAIVATKIATQSRAVATDSAAELKRLRDERKIAHDRLVEMRAACKSDLDFSAWVKIANEKAYGLV
KAARNSCGVAWGTYNLIAASAQQASATSTMDPEFRRYDGEGRIGVQIIGGMSVADLATDTQLQIAMPEFHDGM
TRGEWRRASRTVVKMRVGSDENRRPIWAEFPAVIHRPLPEDARIMSAVITRRRLGVFRRWEYSLCISCESNKF
DRTLPGLKQEGTATINFGWRQFSDGFRVATVNNDVTGIEEIRLPKTITDRFSKCEDLRSIIDMRFNIVRAELQ
EWLASHKADCPEWLTTSLEFLHLWKQPERLDRVVGNWAGLRFAADADIYSILADWRTKYRHLQDWQMMNRRQG
LNMRKEFYRLVASRLAQHNAKLVVEAFDVRQVAVLPRPEEVASGGTAARHNRFLVAVGNLRSSILLAAQKYHC
AVDVVKATNNTRRCNVCGKLLDWDPAKTVNRECPECSTWDQDVNATDNAVDRVASGEVVTMIAPAELAENGSI
RPATKRSWGAARNELDKMPSLL (SEQ ID NO: 100)
>3300018019|Ga0187874_10017489_1
[aquatic-freshwater-peatland]
MSAILVYKFGLLRPVDNATMVHQQVRAAHDYRNDLTMIERGRRAAIRSVLESEPDVAAALTGARAARALLDAA
LAVVASARASARTRAAGAPATGDVKSVRAVLHAAEGTFRQALQAVRTRSHVVSETDRINERAGELGRSARAHC
GVYWGTYLLIEADMQASRKMPLYDGVEPNDPRYQRWTGKGRLGVQIQKGMSASAVFGADTRIRIDPVNERAWP
ATSTLGWSERRRLQHTTLHLRVSSDGAAPIWAAWPMSMHRPFPEGARIKGAVVNLRRVAGREEWTVCITLDVT
DTQRAQCCGEGAVAVDLGWRLLCQPQAHNETGLRVGTWRGEDGAAGTMTLSHHWSGGELKARELRSIRDKAFE
AARDALAVWLASPGDRPAWLAAKTRALGQWRSAHRLAAVAQWWAAHRFDGDAQAFAALETWRYHDHHLWQWET
HQRETTLRDRREQYRIFAAGLARRYRTLVLEAFDLRKLARLPAPEQVDGEAQAPRSQRQLVAPSELRDALVKA
FVARGGEVVEVSAVDSTRICHACGVVELWDQAAELRHTCSACGVEWDQDDNAGANLLTRYRERPSGDETPGPA
RKAEKTGKEGSKWARAKALRAERDTRTGAARKALAKCAE (SEQ ID NO: 101)
>3300018025|Ga0187885_10005575_2
[aquatic-freshwater-peatland]
MEDVDLQYRMRYSCHNDLVAVELERRYTFRAYRSTLPEYAVVEQPYLELKKQRDAVREEIKLIRQKSRTRVET
PEQNARVAALNAELKKQDVFLKIAAKKVSGDLGLVAVGKEADEVAKAATKAILDDYAARGLTWGTRALVVQEL
QAAKNAERDPKIHPWDNSGRIGLQLQGKNLSEEMVASGKHIASEQKRLRAMTKELGKKSKVVESFAERLLKMK
QDRYAQKTPENRGLPISGLADDTRLQIVVPPEIAYQAASTRRGDRRRAARTTMKMRIGSTPKNAPIWMECKVT
MHRQLPADGIIKWAWIRKKMLGTHEIYHLQLIIEAPSFEQKIAVADRMEAIAVDVGWRVREKNVLRIAYLVDT
AGNRKEILLPTSIVEKLKHADSLRGKEDDAFNAIQDRLMEWIGLNKPILPAWFQDTFQFLAQSRSSKNLAWNV
REWGRRRFAGDTLIYEEMTAWRRQFLHLYEWETNERAKAMGERKNFFRHVGLDLARSAHNVLLEDFKLAKIVE
NAQPEEDDDNPQTQRHNRVMSAISEFRQAIASACSAWRSTLWKLPAAYTTQDCHACHQEKDKHSKWDAAPAIV
HTCQEKCGKTWDQDYNASMNLLGAWLRSRRTNRAA (SEQ ID NO: 102)
>3300018025|Ga0187885_10005575_1
[aquatic-freshwater-peatland]
MVRKSTEDPTRIWSFRITEITSPMEDVDLQYRMRYSCHNDLVAVELERRYTFRAYRSTLPEYAVVEQPYLELK
KQRDAVREEIKLIRQKSRTRVETPEQNARVAALNAELKKQDVFLKIAAKKVSGDLGLVAVGKEADEVAKAATK
AILDDYAARGLTWGTRALVVQELQAAKNAERDPKIHPWDNSGRIGLQLQGKNLSEEMVASGKHIASEQKRLRA
MTKELGKKSKVVESFAERLLKMKQDRYAQKTPENRGLPISGLADDTRLQIVVPPEIAYQAASTRRGDRRRAAR
TTMKMRIGSTPKNAPIWMECKVTMHRQLPADGIIKWAWIRKKMLGTHEIYHLQLITEAPSFEQKIAVADRMEA
IAVDVGWRVREKNVLRIAYLVDTAGNRKEILLPTSIVEKLKHADSLRGKEDDAFNAIQDRLMEWIGLNKPILP
AWFQDTFQFLAQSRSSKNLAWNVREWGRRRFAGDTLIYEEMTAWRRQFLHLYEWETNERAKAMGERKNFFRHV
GLDLARSAHNVLLEDFKLAKIVENAQPEEDDDNPQTQRHNRVMSAISEFRQATASACSAWRSTLWKLPAAYTT
QDCHACHQEKDKHSKWDAAPAIVHTCQEKCGKTWDQDYNASMNLLGAWLRSRRTNRAA (SEQ ID NO:
103)
>3300018057|Ga0187858_10035455_2
[aquatic-freshwater-peatland]
MSAILVYKFGLLRPVDNATMVHQQVRAAHDYRNDLTMIERGRRAAIRSVLESEPDVAAALTGARAARALLDAA
LAVVASARASARTRAAGAPATGDVKSVRAVLHAAEGTFRQALQAVRTRSHVVSETDRINERAGELGRSARAHC
GVYWGTYLLIEADMQASRKMPLYDGVEPNDPRYQRWTGKGRLGVQIQKGMSASAVFGADTRIRIDPVNERAWP
ATSTLGWSERRRLQHTTLHLRVSSDGAAPIWAAWPMSMHRPFPEGARIKGAVVNLRRVAGREEWTVCITLDVT
DTQRAQCCGEGAVAVDLGWRLLCQPQAHNETELRVGTWRGEDGAAGTMTLSHHWSGGELKARELRSIRDKAFE
AARDALAVWLASPGDRPAWLAAKTRALGQWRSAHRLAAVAQWWAAHRFDGDAQAFAALETWRYHDHHLWQWET
HQRETTLRDRREQYRIFAAGLARRYRTLVLEAFDLRKLARLPAPEQVDGEAQAPRSQRQLVAPSELRDALVKA
FVARGGEVVEVSAVDSTRICHACGVVELWDQAAELRHTCSACGVEWDQDDNAGANLLTRYRERLGGDETPGPA
RKAEKTGKEGSKWARAKALRAERDTRTGAARKALAKCAE (SEQ ID NO: 104)
>3300012183|Ga0136624_1011435_1
[aquatic-freshwater-polar desert sand]
MSTLVYAYGCAPNTPICEEVDEQLHLAHEFYNKLVELEIRHENALDAMWREYPDIASMMDQIDTTDLIITELK
KRGKAERVENVSTVTSEPLALELKRAKRGQKETRAALRTAKNRIKEDVALPKKHLLAEHQARAKAARIDFAHR
GLYWGTYNRVWADMKVAVEGVIRKRTGGEPARLHFRRWDGTGTLAVQLQRQDGDPPRDPQGLAEGTTKWRNVF
SVAPWMPPAEFDSMTRPAQLRIAEQGRVRMNVGASRVVKIPVLVHRMLPPDADVLGASLTVTRVAGRRRASVS
VIVNLPDAAPVGDDGPRVSVTLGWSSVPHGIQVAKLSADRPLRIPADIADLVHRGPDPHTTEITVPAAWCNRL
DSAMGLQSRRDTALDAIRSELVEYLRAHPDTSDRPITTTEVARWKAPARFAAVALRWRNNPPLPHGKMIAATL
EAWRRTDRRRWEAETHTRRRALGCRRDGYRRVAAWLARECSEVTMSSTDLSKLAHRTEVGASASNAVPEEVAQ
LARQQRVLVAPSELRESIVAACRREGVSVASGAVRAPVSENARSA (SEQ ID NO: 105)
>3300012682|Ga0136611_10000100_4
[aquatic-freshwater-polar desert sand]
MKSTLNWCYGAKTPDIEQAVSDAIFAAHTYRNQLCALELEKRARHYQVLVELSPDYVAACDAVTLVEVAAQAV
EDLITAEKVTQRTQTPKNIKHLRDRATALAAELQVKRAVRKVAQCSAYAMPAVIAALDRSTAQHKAARKQAKQ
ASGLYWGTEATVTESCRDFHKGPPPTFKRYDGTGQLSVQLQGGLDCADAERYNTLCYLGDSLGGKRRECFIRI
GSDNRAPVFACVPIVFHRALPAGEIKRAYLERRKIASHVRWTIRFTIDIERDIPDRPMPGEVAIHTGWRMEEG
SLRVATWLASDGSTGTLRLSQEHCADYLRLDSLEANRAAGLNEVIAELRTWAKSRELPEFLTEVKPHLHLWKS
QARLAKLVWHWAEARFDGDSAMFERLDSWRKTDKHLWQHHRRLTVRISRRRRDAYRVFAKSLSERYGVAILAP
IQVQKLTKKPTETRPPEDWELDQTQSRRHAAWAAVSDLTSCIRERFPLRCITVSSVNMTKECVNCGEINKADG
RKIQCRGCGQTYDCDDNAVANTLARGDAALLDGALLALVTEQELKEAAKQAKLVKLQEANNAARTTRQTDL
(SEQ ID NO: 106)
>3300013127|Ga0172365_10004082_5
[aquatic-freshwater-sediment]
MFGHTSDPSLIFRYGALPPVEDGPVLEQMRAAHRYRNKLVEIERDRREKAAAIVSAASPDLAGLERQYAELGE
QVESAAAEIKATNQRARKQRATLEQRAKLRTLRAERAEVYARLKEAKHTAYHSLAARAALDQLDAVTLDATKA
ARATCSVYWGTYLQIEAGLGSIRKGPPPRFLRWTGDGKLAVQIQGGMSRQEAEVGDSRLKIATLERRGKATNV
YLRIGTDEMRNPIWAIVPVIFHRPIPDDAQIKWVYLLARRVGTHTRWAVCFVLSRATGWGKPDLATDGAVGLD
LGWRILDHGLRVAYWCGSDGAGEEIVLPLRDVSRWQKADDLRAIRGKNFDAARDELALWLAGRDLPDWLIEQT
RALRQWRNATRLAALAIHWREDRFTGDEEAFAPLEAWRTQDKHLLEWEANQRRKAVAWRDDFYRRVAADLSRR
YKTLVIEDCNWREMGRLPEVGESNESGRAGSYRVIAAVGSLARVLRERFAETVSADPAYTTQRCHVCGQLAQA
ETRTSVWVKCNHCGEAWDQDRNAALNLLSAASGAVT (SEQ ID NO: 107)
>3300013127|Ga0172365_10004082_3
[aquatic-freshwater-sediment]
MIFRYGALPPVEDGPVLEQMRAAHRYRNKLVEIERDRREKAAAIVSAASPDLAGLERQYAELGEQVESAAAEI
KATNQRARKQRATLEQRAKLRTLRAERAEVYARLKEAKHTAYHSLAARAALDQLDAVTLDATKAARATCSVYW
GTYLQIEAGLGSIRKGPPPRFLRWTGDGKLAVQIQGGMSRQEAEVGDSRLKIATLERRGKATNVYLRIGTDEM
RNPIWAIVPVIFHRPIPDDAQIKWVYLLARRVGTHTRWAVCFVLSRATGWGKPDLATDGAVGLDLGWRILDHG
LRVAYWCGSDGAGEEIVLPLRDVSRWQKADDLRAIRGKNFDAARDELALWLAGRDLPDWLIEQTRALRQWRNA
TRLAALAIHWREDRFTGDEEAFAPLEAWRTQDKHLLEWEANQRRKAVAWRDDFYRRVAADLSRRYKTLVIEDC
NWREMGRLPEVGESNESGRAGSYRVIAAVGSLARVLRERFAETVSADPAYTTQRCHVCGQLAQAETRTSVWVK
CNHCGEAWDQDRNAALNLLSAASGAVT (SEQ ID NO: 108)
>3300013127|Ga0172365_10033732_1
[aquatic-freshwater-sediment]
MAICKVYRYGLLPPTENRDLVLKTLRLAHEYRNKLVEIDRQERAEIRAVQTSHGSIPALAAAAKSAIQAKETA
YQAIKAHKAQDRTRKVPEPLKATYEAAKAAASAASQALWQARAALRGDPTVAIRRDEISLRYNEKRKAARAAS
GIYHGTYMRVEAADQQARKMTPLWDGVEPSDVKFARWRGDGGVGLQMKEKPGPADLPTSRWCRIEPRGAPKGA
DPSSKRSAKRRHCTLALRVGSEEREPVWARWPMVMHRPLPEDGEILWVTVTLRHVGPRQEWVALFTVRHEDKR
QVPPAEPVDRVGVDIGWRKLEGGGVRVAAWRTDSGAEGELVLDEHMLGQLRKADDLRSIRDKNLDAARASLVA
AMPGMSLPDWFPKNVWQWRAPARFSNLAKRWKQNRFPGDDLPYAQLEAWRYHDHHLWAWETSQRTKALRHRLD
VYRVFAARMARTYTGLVIEDWDMRDTAEKPDAHEQEGDNEQARSNRVKSAVSELRRALVQAFVNVAKVPAAYT
TQTCSACGAIEKWDQAAELEHTCSACGAQWDQDYNAARNLLAYVEQPGGPDNGGVARDEKKPNDGAEVQESKW
AKAKRMGKEKRDRVDTARNTVPSAAE (SEQ ID NO: 109)
>3300013128|Ga0172366_10016188_4
[aquatic-freshwater-sediment]
MFGHTSDPSLIFRYGALPPVEDGPVLEQMRAAHRYRNKLVEIERDRREKAAAIVSAASPDLAGLERQYAELGE
QVESAAAEIKATNQRARKQRATLEQRAKLRTLRAERAEVYARLKEAKHTAYHSLAARAALDQLDAVTLDATKA
ARATCSVYWGTYLQIEAGLGSIRKGPPPRFLRWTGDGKLAVQIQGGMSRQEAEVGDSRLKIATLERRGKATNV
YLRIGTDEMRNPIWAIVPVIFHRPIPDDAQIKWVYLLARRVGTHTRWAVCFVLSRATGWGKPDLATDGAVGLD
LGWRILDHGLRVAYWCGSDGAGEEIVLPLRDVSRWQKADDLRAIRGKNFDAARDELALWLAGRDLPDWLIEQT
RALRQWRNATRLAALAIHWREDRFTGDEEAFAPLEAWRTQDKHLLEWEANQRRKAVAWRDDFYRRVAADLSRR
YKTLVIEDCNWREMGRLPEVGESNESGRAGSYRVIAAVGSLARVLRERFAETVSADPAYTTQRCHVCGQLAQA
ETRTSVWVKCNHCGEAWDQDRNAALNLLSAASGAVT (SEQ ID NO: 107)
>3300013128|Ga0172366_10018111_5
[aquatic-freshwater-sediment]
MSGEEFLLDQLRARVDYWNRLVEIERDFQAEKEQLLSAASAEMEQLATYIALTDGKLTEALSAGARARSQART
RRTPEPLAAEIAELRDRLRELRKQYREVRRTTFGNEKVKAALRTLGSERTAVIRKARREHVLKGLWWGNYLDV
ELAYKTARQKAGSRLRFQRTGPEGRVSVWFQHGLPTSDVWGKDSRLTIARVPEEAWTSDVRSVRRRLARTRVW
LRAGSNPDRSPRLIEAEMVMHRPLPHGLIRHASIIRERIASHYRHRLVITVAVQDIPTRDGREVGIDIGWRLF
EDRLRVAVAVDEENQLEELSLPQEMLGGFAQVRDLQAVRDTHFNGAKAMLAAFLHTAQMPDWLRDATSTLTQW
RSQGRLTALALQWRDRRFKDDAVYAMLEAWRKRDKHLWEWQANLRDKLLARRREMYRLWAISIARRYGTVVIE
EFDLRRIVSEDNIDVADRMRFIAALSQLRSILEHTCAREGVRIVKVPASYTTQDCAFCANREQFDARKEVRHR
CSKCGAEWDQDENAARNLLKRAKGSQVSRKEV (SEQ ID NO: 110)
>3300013129|Ga0172364_10001281_26
[aquatic-freshwater-sediment]
MAVEAQFRAAQWYRNRLIEITNKSREKYQQLMLRIPEIARLQETIDADKALKESLREEIKVASAKARKNVPLR
PGLREQIASLTKAIKENALTLRAAKDKAKAQIAEETNALYAETAAEQKALYNEAGQPGEIVHRKEGHPDIREP
RVPLAWGTRLLMNKAHEQACSTGMPLKVRHDPVGRIGVQLQKGRTISQIFSGKDGFLRIEPVPDDTWDPRPQN
APKKGERLTREQHKARKGTGGKTKSRTRVHLNIGEGRGEDRPFATFPITLYNRKLPVDGKVLWAWILRERIGT
RMEYKLQLSVESNTFKCESDGHGAIAFDIGWRVRSKNNLRIAYWFDDYGQSGEILLPEIIPSGLAKADSLQAI
RKRKFNRMRALLSKAKADAIKTGMAIPPALLTETETLSAWRSEDRLRRLVKHIWPNHRFAGDERWFNIAKNWL
HKELHLYQWECDERQQATARRTNFYRHTALEFARKYQTCVFENFKLTRIAVKEPVESEKADTPSNIQHNRVVS
ALSDFRDAFKNKMIFAKVPMEFTTIVCHNCRHPEKFNAAKELIRTCPKCNTTWDQDLNAAKNILSRFHCEGTS
GTDMGVQAA (SEQ ID NO: 111)
>3300013129|Ga0172364_10017363_4
[aquatic-freshwater-sediment]
MFGHTSDPSLIFRYGALPPVEDGPVLEQMRAAHRYRNKLVEIERDRREKAAAIVSAASPDLAGLERQYAELGE
QVESAAAEIKATNQRARKQRATLEQRAKLRTLRAERAEVYARLKEAKHTAYHSLAARAALDQLDAVTLDATKA
ARATCSVYWGTYLQIEAGLGSIRKGPPPRFLRWTGDGKLAVQIQGGMSRQEAEVGDSRLKIATLERRGKATNV
YLRIGTDEMRNPIWAIVPVIFHRPIPDDAQIKWVYLLARRVGTHTRWAVCFVLSRATGWGKPDLATDGAVGLD
LGWRILDHGLRVAYWCGSDGAGEEIVLPLRDVSRWQKADDLRAIRGKNFDAARDELALWLAGRDLPDWLIEQT
RALRQWRNATRLAALAIHWREDRFTGDEEAFAPLEAWRTQDKHLLEWEANQRRKAVAWRDDFYRRVAADLSRR
YKTLVIEDCNWREMGRLPEVGESNESGRAGSYRVIAAVGSLARVLRERFAETVSADPAYTTQRCHVCGQLAQA
ETRTSVWVKCNHCGEAWDQDRNAALNLLSAASGAVT (SEQ ID NO: 107)
>3300013129|Ga0172364_10018773_2
[aquatic-freshwater-sediment]
MSGEEFLLDQLRARVDYWNRLVEIERDFQAEKEQLLSAASAEMEQLATYIALTDGKLTEALSAGARARSQART
RRTPEPLAAEIAELRDRLRELRKQYREVRRTTFGNEKVKAALRTLGSERTAVIRKARREHVLKGLWWGNYLDV
ELAYKTARQKAGSRLRFQRTGPEGRVSVWFQHGLPTSDVWGKDSRLTIARVPEEAWTSDVRSVRRRLARTRVW
LRAGSNPDRSPRLIEAEMVMHRPLPHGLIRHASIIRERIASHYRHRLVITVAVQDIPTRDGREVGIDIGWRLF
EDRLRVAVAVDEENRLEELSLPQEMLGGFAQVRDLQAVRDTHFNGAKAMLAAFLHTAQMPDWLRDATSTLTQW
RSQGRLTALALQWRDRRFKDDAVYAMLEAWRKRDKHLWEWQANLRDKLLARRREMYRLWAISIARRYGTVVIE
EFDLRRIVSEDNIDVADRMRFIAALSQLRSILEHTCAREGVRIVKVPASYTTQDCAFCANREQFDARKEVRHR
CSKCGAEWDQDENAARNLLKRAKGSQVSRKEV (SEQ ID NO: 112)
>3300013129|Ga0172364_10045136_2
[aquatic-freshwater-sediment]
MAICKVYRYGLLPPTENRDLVLKTLRLAHEYRNKLVEIDRQERAEIRAVQTSHGSIPALAAAAKSAIQAKETA
YQAIKAHKAQDRTRKVPEPLKATYEAAKAAASAASQALWQARAALRGDPTVAIRRDEISLRYNEKRKAARAAS
GIYHGTYMRVEAADQQARKMTPLWDGVEPSDVKFARWRGDGGVGLQMKEKPGPADLPTSRWCRIEPRGAPKGA
DPSSKRSAKRRHCTLALRVGSEEREPVWARWPMVMHRPLPEDGEILWVTVTLRHVGPRQEWVALFTVRHEDKR
QVPPAEPVDRVGVDIGWRKLEGGGVRVAAWRTDSGAEGELVLDEHTLGQLRKADDLRSIRDKNLEAARAALVA
AMPGMSLPNWFPKNVWQWRAQARFSNLAKRWKQNRFPGDDLPYAQLEAWRYHDHHLWAWETSQRTKALRHRLD
VYRVFAARMARTYTGLVIEDWDMRDTAEKPDAHEQEGDNEQARSNRVKSAVSELRRALVQAFVNVAKVPAAYT
TQTCSACGAIEKWDQAAELEHTCSACGAQWDQDYNAARNLLAYVEQPGGPDNGGVARDEKKPNDGAEVQESKW
AKAKRMGKEKRDRVDTARNTVPSAAE (SEQ ID NO: 113)
>3300013130|Ga0172363_10000480_22
[aquatic-freshwater-sediment]
MAVEAQFRAAQWYRNRLIEITNKSREKYQQLMLRIPEIARLQETIDADKALKESLREEIKVASAKARKNVPLR
PGLREQIASLTKAIKENALTLRAAKDKAKAQIAEETNALYAETAAEQKALYNEAGQPGEIVHRKEGHPDIREP
RVPLAWGTRLLMNKAHEQACSTGMPLKVRHDPVGRIGVQLQKGRTISQIFSGKDGFLRIEPVPDDTWDPRPQN
APKKGERLTREQHKARKGTGGKTKSRTRVHLNIGEGRGEDRPFATFPITLYNRKLPVDGKVLWAWILRERIGT
RMEYKLQLSVESNTFKCESDGHGAIAFDIGWRVRSKNNLRIAYWFDDYGQSGEILLPEIIPSGLAKADSLQAI
RKRKFNRMRALLSKAKADAIKTGMAIPPALLTETETLSAWRSEDRLRRLVKHIWPNHRFAGDERWFNIAKNWL
HKELHLYQWECDERQQAIARRTNFYRHTALEFARKYQTCVFENFKLTRIAVKEPVESEKADTPSNIQHNRVVS
ALSDFRDAFKNKMIFAKVPMEFTTIVCHNCRHPEKFNAAKELIRTCPKCNTTWDQDLNAAKNILSRFHCEGTS
GTDMGVQAA (SEQ ID NO: 111)
>3300013130|Ga0172363_10009486_8
[aquatic-freshwater-sediment]
MFGHTSDPSLIFRYGALPPVEDGPVLEQMRAAHRYRNKLVEIERDRREKAAAIVSAASPDLAGLERQYAELGE
QVESAAAEIKATNQRARKQRATLEQRAKLRTLRAERAEVYARLKEAKHTVYHSLAARAALDQLDAVTLDATKA
ARATCSVYWGTYLQIEAGLGSIRKGPPPRFLRWTGDGKLAVQIQGGMSRQEAEVGDSRLKIATLERRGKATNV
YLRIGTDEMRNPIWAIVPVIFHRPIPDDAQIKWVYLLARRVGTHTRWAVCFVLSRATGWGKPDLATDGAVGLD
LGWRILDHGLRVAYWCGSDGAGEEIVLPLRDVSRWQKADDLRAIRGKNFDAARDELALWLAGRDLPDWLIEQT
RALRQWRNATRLAALAIHWREDRFTGDEEAFAPLEAWRTQDKHLLEWEANQRRKAVAWRDDFYRRVAADLSRR
YKTLVIEDCNWREMGRLPEVGESNESGRAGSYRVIAAVGSLARVLRERFAETVSADPAYTTQRCHVCGQLAQA
ETRTSVWVKCNHCGEAWDQDRNAALNLLSAASGAVT (SEQ ID NO: 114)
>3300013130|Ga0172363_10014785_2
[aquatic-freshwater-sediment]
MPRTRSKALPTKVYKYGCSAPLENQELVKEQWRLANRYRNALLENSLQWRSACQAVVSAEDTELRDIDTCIDI
LNVNIDSLISEKKKRNSAARKRLKHPDLEAKITDCKTERKRLYARRKLVKDIAYRSPGNKQALDAVHQAFKAA
NREARKIASKSGLGWGTYLQIEDSAKNFAKGKPAKFKRFDRDAGGSIAIQIQTPQGAPHLTVDRLLEGKDNRL
QLIPQPDGIHALVRLCVGGVDMSQRRSANNPPCYVTVRMNMHRPLPPDSHITWVKLIARRVGLKLKWDVHFTV
ARGSGFAPTIGSGVVGIDIGYRHLDDGSLRVAAWAGSDGRHGELILPATLVRALTRKQELQALRDEKFNVVRA
SLVEWCKHVSIPDWLKEAASTLALWRSQKRLHTLAQQWSQNRFTGDSSMYIVLDAWRKEDRHHLAWLANESEQ
GICRRKDIYGKFVAELRRHYGTVGLEDIDLREHAQADNLSKGVQNQRSIAAHSTLRSLLSTMQVIKVPAANTT
RRCHYCGHINNVGTDVGYYCDDCGWTGDRDYNASQNILREATYSLRAGGRSHVAT (SEQ ID NO: 115)
>3300013133|Ga0172362_10012573_3
[aquatic-freshwater-sediment]
MFGHTSDPSLIFRYGALPPVEDGPVLEQMRAAHRYRNKLVEIERDRREKAAAIVSAASPDLAGLERQYAELGE
QVESAAAEIKATNQRARKQRATLEQRAKLRTLRAERAEVYARLKEAKHTAYHSLAARAALDQLDAVTLDATKA
ARATCSVYWGTYLQIEAGLGSIRKGPPPRFLRWTGDGKLAVQIQGGMSRQEAEVGDSRLKIATLERRGKATNV
YLRIGTDEMRNPIWAIVPVIFHRPIPDDAQIKWVYLLARRVGTHTRWAVCFVLSRATGWGKPDLATDGAVGLD
LGWRILDHGLRVAYWCGSDGAGEEIVLPLRDVSRWQKADDLRAIRGKNFDAARDELALWLAGRDLPDWLIEQT
RALRQWRNATRLAALAIHWREDRFTGDEEAFAPLEAWRTQDKHLLEWEANQRRKAVAWRDDFYRRVAADLSRR
YKTLVIEDCNWREMGRLPEVGESNESGRAGSYRVIAAVGSLARVLRERFAETVSADPAYTTQRCHVCGQLAQA
ETRTSVWVKCNHCGEAWDQDRNAALNLLSAASGAVT (SEQ ID NO: 107)
>3300013133|Ga0172362_10022806_8
[aquatic-freshwater-sediment]
MPRTRSKALPTKVYKYGCSAPLENQELVKEQWRLANRYRNALLENSLQWRSACQAVVSAEDTELRDIDTCIDI
LNVNIDSLISEKKKRNSAARKRLKHPDLEAKITDCKTERKRLYARRKLVKDIAYRSPGNKQALDAVHQAFKAA
NREARKIASKSGLGWGTYLQIEDSAKNFAKGKPAKFKRFDRDAGGSIAIQIQTPQGAPHLTVDRLLEGKDNRL
QLIPQPDGIHALVRLCVGGVDMSQRRSANNPPCYVTVRMNMHRPLPPDSHITWVKLIARRVGLKLKWDVHFTV
ARGSGFAPTIGSGVVGIDIGYRHLDDGSLRVAAWAGSDGRHGELILPATLVRALTRKQELQALRDEKFNVVRA
SLVEWCKHVSIPDWLKEAASTLALWRSQKRLHTLAQQWSQNRFTGDSSMYIVLDAWRKEDRHHLAWLANESEQ
GICRRKDIYGKFVAELRRHYGTVGLEDIDLREHAQADNLSKGVQNQRSIAAHSTLRSLLSTMQVIKVPAANTT
RRCHYCGHINNVGTDVGYYCDDCGWTGDRDYNASQNILREATYSLRAGGRSHVAT (SEQ ID NO: 115)
>3300013133|Ga0172362_10025871_2
[aquatic-freshwater-sediment]
MAICKVYRYGLLPPTENRDLVLKTLRLAHEYRNKLVEIDRQERAEIRAVQTSHGSIPALAAAAKSAIQAKETA
YQAIKAHKAQDRTRKVPEPLKATYEAAKAAASAASQALWQARAALRGDPTVAIRRDEISLRYNEKRKAARAAS
GIYHGTYMRVEAADQQARKMTPLWDGVEPSDVKFARWRGDGGVGLQMKEKPGPADLPTSRWCRIEPRGAPKGA
DPSSKRSAKRRHCTLALRVGSEEREPVWARWPMVMHRPLPEDGEILWVTVTLRHVGPRQEWVALFTVRHEDKR
QVPPAEPVDRVGVDIGWRKLEGGGVRVAAWRTDSGAEGELVLDEHTLGQLRKADDLRSIRDKNLEAARAALVA
AMPGMSLPNWFPKNVWQWRAQARFSNLAKRWKQNRFPGDDLPYAQLEAWRYHDHHLWAWETSQRTKALRHRLD
VYRVFAARMARTYTGLVIEDWDMRDTAEKPDAHEQEGDNEQARSNRVKSAVSELRRALVQAFVNVAKVPAAYT
TQTCSACGAIEKWDQAAELEHTCSACGAQWDQDYNAARNLLAYVEQPGGPDNGGVARDEKKPNDGAEVQESKW
AKAKRMGKEKRDRVDTARNTVPSAAE (SEQ ID NO: 113)
>3300010155|Ga0098047_10009758_2
[aquatic-marine]
MPVKSKMKGDGRIYAYRASLPTKNLEIVQEQLYLVHKYRNRLVELELNRRSQVDQALRDLVPDLEPTELALKQ
LDDQIAAAKDAQKKANIKQRGRKVAKSDRDALKDLKAQRKVLYQKRKQLRKDTFSSTAWKSRQTQIENNAKVE
SKAARASCGLYWGSYAPVEEAARAFRRGAPPRFHRWTGEGKLAVQMQAQAGKPDFTPDTLTSCSSNLLRLELR
PEGIWVDGKRRPKKLGNALLWFRVGSTTVKPKRQPIWAEVPIKLHRPLPSDCKIKWCYLQRRKRGTKTIWEVC
FVLQGEHGAFDPGDQASEGHVGIDVGWRKYEDRLRIAVYSGSDGQEGELCLPDWWLGESRRVERIRGHRDKLL
DAAKTELKAWIKGRESLPDWLTEAGKHMHQWRSASRLAGLCLRWRGELIKPTTDGAAALASLEAWRERDKHLY
EYEAHLRAQLQGSRKDLYRKFAAMLSRKYATAYIEDLDLRKFHQLRAIEEGGDKGTDSIRAYVRDACLSELFD
AIKSRFRHHVKVDPANTTKQCHACSVVDASWVDHAKVDHECSSCSVTWDQDTNAARNLLNSGDEPVTQFGGPA
LAPVLVHTYTHKGPNRARRRARRRRALEKKRLNDAA (SEQ ID NO: 116)
>3300006805|Ga0075464_10026824_2
[aquatic-marine-aqueous]
MRVYKYRAYAPIVGAGIFDAQSRARHRYQNQLIEIERAWCGLDRATKKDPEAQARRKALVKAARQDAARRGLA
WGSYNGASDDVRRAVSALRGAARDEGPRFRRFDGGGRIKVQQQPGARVVVIDGDRVTFRLGHQGAVTVPVVMH
RPIPPDATIKEAQLHRERVADKYKWWVTITVAVPAPPPAPPRGVVGIDLGWARRGGKSERDGRRVAVASFADG
RELQVRCPESILAKIDHARGLRSLRDVKFNVAIAWLREHVCEHGAPEWLRAALRWSHAWRSQAKLAAVVLRWR
DARYDGDDGIYQTLEIWRRRDKHLWTWEVHETRKALAQRREIYRVAAAYIAEHAGEVRVEDIDLAEMAESDDL
PRAARRGRVDTAPSTFLAAVKNACSSRGVTYAVVSAKNTTRKCSGCGVVGRSVVGDTFACGGCGLVADRDANA
ARNIAASAPEAPREPKPKSADLRRAGKARHDAARAAAVKAA (SEQ ID NO: 117)
>3300006805|Ga0075464_10026824_2
[aquatic-marine-aqueous]
MDVRVYKYRAYAPIVGAGIFDAQSRARHRYQNQLIEIERAWCGLDRATKKDPEAQARRKALVKAARQDAARRG
LAWGSYNGASDDVRRAVSALRGAARDEGPRFRRFDGGGRIKVQQQPGARVVVIDGDRVTFRLGHQGAVTVPVV
MHRPIPPDATIKEAQLHRERVADKYKWWVTITVAVPAPPPAPPRGVVGIDLGWARRGGKSERDGRRVAVASFA
DGRELQVRCPESILAKIDHARGLRSLRDVKFNVAIAWLREHVCEHGAPEWLRAALRWSHAWRSQAKLAAVVLR
WRDARYDGDDGIYQTLEIWRRRDKHLWTWEVHETRKALAQRREIYRVAAAYIAEHAGEVRVEDIDLAEMAESD
DLPRAARRGRVDTAPSTFLAAVKNACSSRGVTYAVVSAKNTTRKCSGCGVVGRSVVGDTFACGGCGLVADRDA
NAARNIAASAPEAPREPKPKSADLRRAGKARHDAARAAAVKAA (SEQ ID NO: 118)
>3300009149|Ga0114918_10020022_2
[aquatic-marine-deep subsurface]
MGGKAGTVKTAKKHKHRWVEDLEKDLIENCSCGKSRKSQNSSVIVYGLGKPMFDEEGSECPSCNEESNGEPCG
AHRFIDQMRLGHSYGNKLTELYRASSERYREIIGSASKKMEVIVMKLDDLDDQIKGLNALLKADKDNKEAKKL
KKELTADRKIIRADRKELVEKLKQNKIIQARLKKNNIKLNSEIIKARGEFSKLGLMWGTYNLHEASAKQAQYA
PGRRGDPEFKRWEGHGRIGVQLQGGLPESKVWGDSRSFQIDKVDHETWSKLREDGSPDRAFRRKQCRTKVRVR
IGSAKAKPIWVEFPMTMHRPIPEGADIRDVTILQKKSGTIYRYSLHVQINENKTNQPERSGVVGVNLGWRKHQ
DNTLRVAYWYGDDGRYGEYLLDSEYLEKVKVMDGKQSKRSMALDVIKETFAVWLDGQDNLPEWIQEWRGIKFI
RDWRSSSRLASLVLRWRKNRFDGDALIFENLEEWRRADKHTCNQEGGIRNKNQLRRQDEYRNFAAFLARTYGK
VVVDDTNYANLARKPGPEDDDNKVARKQANLASPGKLRVNIKNACHKHGAIYVAASSKHITATCHKCGTINDW
DKSLSLTHWCSGCNAFWDQDMNAAINLCRSGGGKPPNFEHPGDARIELNDEVNKYDWLIQESAGMAGSKKQPI
ENLAVTL (SEQ ID NO: 119)
>3300006083|Ga0081762_1007854_6
[aquatic-marine-diffuse hydrothermal flow volcanic vent]
MRERARNWPVMVFSYGILPSFLKEEAAINILKEEAYRMNELWNKLVEIGRKYLETYSSNIEEDPAIAPLISQR
KEIENTLEETDKQIKQLRIKLKTKKHPALAELEEKKRELRRQLREIKASIRETKKQVKEKYREVFAQMEEEVK
EAVKKAPLYWCNKEVVRDKFWAAWRGVKNGNIPKFHRFDDRWCLTWRFTGGGMPVKDAFRKVLSGIVPPEVYK
LPTKKRNKMANLTCLFRQGEYRILVPIILHRPLPEGGYIKRVTFVRRPYGRDRVRLFLNFTVEVPPDKYYLPV
REERKGKIAALELGFRKVDGRIRVGVLYDPFTEEKFREIFIPQNIPERLEKVRKGQSKADEELEDIKNDLSKW
LVEPQVLPKLPEEIKKLITNRVAWVKTRDRGVWKVINLLKESGADPAAARNVERRMLKREKFLNDLQRTRIKA
LGARKRFYENLAKEIFDRYEMLIIKDISLKKLALKEMAEQLPDEARWVRFVAALGELVGCLERRAERTKGVLV
KLDPAYLTRTCHICNHINNPNRPEKLFWTCEKCGTKWDQDKNAAVNLYEQGIERLKLAQTG (SEQ ID NO:
120)
>3300010354|Ga0129333_10000304_8
[aquatic-marine-freshwater to marine saline gradient]
MRLGHRYQNDLIAIERGRRLAFAAVMSSDTRIAEAEAKITEIDAKISEAVERARQARVARRTKADTEQTKSEI
RSLKASKAAAVLDLRAIKPLVQSELRPRIAEVDARAHELQISARAHCGVYWGTYLLAEAAAEQAAKTTKGELR
FQRWDGSGQVSVQIQGGADVDDVVGDSDTRLRWPEYVEGIRKAKRTELAMRVSSEKGVPVWARWPMVYHRPLP
TNARIKRAIVSLRMRGPREEWSVEVTIDASTCRLRDRPDGGKVAVHLGWRKEPSGNVRVATWLGDDGDAGTIE
CPERVLTGFAKCESLRSIRDRNLDELRARLVLAREGWPVWLRDATSSLYQWRSPGRFVALAQRWKAAGVAPEH
ASDYGAIEAWRYNDHHLWRWEHDQRLNSTRYRREVYRIAVAELSRRYRRAILMAADWAEMAKLPGIGEGAPDL
PDEARAQRVETAPYVLTEALHSAMTEVVWVDPSYLSQACRHCDHKDTGDTWVRECTSCGKARDIDEAAVRTML
DLEEAGAWSWKKGGAKDESGKVREIRAPKWAKKHATEAAE (SEQ ID NO: 121)
>3300010354|Ga0129333_10000304_10
[aquatic-marine-freshwater to marine saline gradient]
MITRVYRYGLLAPTENSELVRQQMRLGHRYQNDLIAIERGRRLAFAAVMSSDTRIAEAEAKITEIDAKISEAV
ERARQARVARRTKADTEQTKSEIRSLKASKAAAVLDLRAIKPLVQSELRPRIAEVDARAHELQISARAHCGVY
WGTYLLAEAAAEQAAKTTKGELRFQRWDGSGQVSVQIQGGADVDDVVGDSDTRLRWPEYVEGTRKAKRTELAM
RVSSEKGVPVWARWPMVYHRPLPTNARIKRAIVSLRMRGPREEWSVEVTIDASTCRLRDRPDGGKVAVHLGWR
KEPSGNVRVATWLGDDGDAGTIECPERVLTGFAKCESLRSIRDRNLDELRARLVLAREGWPVWLRDATSSLYQ
WRSPGRFVALAQRWKAAGVAPEHASDYGAIEAWRYNDHHLWRWEHDQRLNSTRYRREVYRIAVAELSRRYRRA
ILMAADWAEMAKLPGIGEGAPDLPDEARAQRVETAPYVLTEALHSAMTEVVWVDPSYLSQACRHCDHKDTGDT
WVRECTSCGKARDIDEAAVRTMLDLEEAGAWSWKKGGAKDESGKVREIRAPKWAKKHATEAAE (SEQ ID
NO: 122)
>3300009507|Ga0115572_10029017_2
[aquatic-marine-pelagic marine]
MISRVYKYGAVPLKKFPEVKFPREQFPEEGVEELRRANKLRNSLVWLHRKNNEKFEAARVAADAEYGEIAEKL
DALEKTISQALTAKRQARAKAGTRDAKHPLVKAASETINELTKQRSDLWKALKPARIRADKRVDRKALTKQFD
DAVKVVQHVKETGGLSSHCANEIVRYFKESRSRALNERATLRYRRFDGTGFWFYRFREPGVNKNGVDFDGLLT
GNKTEARDNRNFVLTEKSRRGKRVIYKLRAKIAGGAKKDSKVYGHFDLILHRPIPENARIQSAKILRHRTGDK
FTYTVSFTLKLPDVEQQTVEGSVLGLDIGFREMERNNSYRIATLATNDQSRRVETIDIARENRRGFLARMNHI
DDLRSTMDENATELGKKLLPLLKTAKPLPDSHQQFIFTERLRKTRANVTLDFERSYKMARWFIRAPDEADFYG
PEIVGMVLRWWEENSFKYREMHNLRRKALAERKEVYRMEAARLVGFGIPIAVEKLDMSKWAERKDSDNELSNR
ALSSRFLVAPSELIAAIENAAKREGVPFIKVNAANTSKACHACGTINKALKGELIWTCEECETKHDRDINAAI
NIAKRGILQAKKEKKQ (SEQ ID NO: 123)
>3300017963|Ga0180437_10000100_151
[aquatic-non marine saline and alkaline-hypersaline lake sediment]
MTKTYVYGLPLGPTVNADLVEEQMRLAHKYRNALIEIERERREKVREVYDERDLALEGLVEEDKVAKSELKRA
TEDLKRQRAKTRSRSDTAEQRARVKEARKAAQEVAKRLSEARKELKLDEELQKRLSEANLTASEKSQAAQQGF
SREGLFWGTYLQVDNAMEDSRRDLKMWDEHGQPLDPKFLQWRGDGTVAVQLQGDKHPVEKIFSGEDTFLQVDM
EPPPEGVVSKTRRKKRRGVMRLRVGSTKSRGPVWAEFPIIMHRPLPQGVRIKWAVVKRRMISDRPRWTVHFSL
GLPAEYQHEEFGSGRGAVAVDIGWRKRGEDQIRVAYLVDGDEYAAYLRDRQDPLGRGDELLMEPEVVRGFDKV
ESLQSIRALNQNEMQKSLKGWIKSNKKNLPEWFREDVRYLHSWKSPKRYAGLLRKWGEKRWDGDGEGFQILKD
WLSGTYEESLGRRDGGDRHLWQWKESQEQKSLRRRKDHYRRVAAKLARKYKVLVIEDFKLTETQKHEPPESEK
VEIQAARNQQKEAACYELRMMFVQAFLARGGTVVWVDARMTTQRCFECGCLEPWDAIPEVDHVCVECGAKWDQ
DANAARNIMRLYRNDETLKMIDGSVPVEPKMSRRQKGRKKGKKIVQQRKSQEAAQPSV (SEQ ID NO:
124)
>3300017963|Ga0180437_10000153_25
[aquatic-non marine saline and alkaline-hypersaline lake sediment]
MTRKTSKTKRKKKPGKPRVRGPQLAYVYGLPFGPTKNAELVEKQIVLSQRYNNQCVEAERRLRATLREIYQQH
TLDLMGASDEMREAFTEVKRLEKLLREMQEDLRTKRKRSRSRSDTPQERMRLREVRDLKNEAWAKLRELKNGS
ESDDEPGKEEPRKKVELSDELKARRAEAQQREKQELHEAYVQFKDGTYEKVTETDEELGKLYWGTYLLVNRAR
EASRMSLRDSLWKWNEEKGIWVERDPKFKSLDDEVIFGVELQKGDSVERVLNCQNTMFQLDMEPEMGEEVLRH
RRIRRRGIARIRVGSGGKSGRDPIWAEFPVIMHRPLPPKARIKWAVVKREKITTRLRWTLHLHLEVDSGDCHK
DYGTGRGVVAVDIGWRKRGTETVEMGRKRKKRGLRKQEVEVPRIRIAYLIDDREYAAYLKNPDEGEVGHEQCM
SSKVVAGFQRVETLQQTRQLKQNEMLAELRAWIKARRSALPKWFRESTRGIAKWEAPKRFAWLLRLWRESRWK
GDERGFEILDRWQRGVYDEEARRLEGGDRHLWQWQESQRRKSLLQREDHYRCVDSALAREFKVLVLENIDLSK
MQKHELPGSDKVEIRRARRQQKEAALSEFRETLIQAFLSRGGTVVWVNPAMTTQRCFDCGHDAPWDPIPKVEH
TCEKCGRTWDQDANAARNMMRLYRENKIVKIADGSVLVREMSDAQKNRNKGKKVVRKRKKEEEERNGEGPAPL
ES (SEQ ID NO: 125)
>3300017963|Ga0180437_10000488_78
[aquatic-non marine saline and alkaline-hypersaline lake sediment]
MRVYKYGLLRPTTNADLVHEQIKIGHKYRNKLIELEIKRRDLIRAEVAKSSVVEDDFTDAKLAVEKFKHLDKL
LKQKNAQHRSKRHNNPDLKKDHTKARKEKTKAIKKLEETRRKVLKKCKETIKVFNDQYIEEEKKVRSECAPFW
GTYQVIEDAMKRSRKSLPLWDGLESNNPKFRRFNGIGRVSIQLQKDVIDKNNGMNVDLVFGTTDTRLQVAPVP
EEAWYSPIRSVRRKKSRTVLKMRIGSEGRAPIWAEWPMIMHRPLPDNGRIKRVTVNFRKIGPREEWTADFFIN
DSATLHEQYEVSGAIGLDVGWRLMDDGSLRVAFWEDDEGEKGEFRLSPTLMGAFKKADDLRSIRDKNRDEIKE
FLIQHFSKNPMPSWMLDFVKGKEDSKRPTNKQACVYLSKWKSIAKLTKLVQTWKEKGITKRHQKAYNRFEDWR
YHDFHLWQWETSQRKKAERRRKDNYRVLASKLSKQYHTLVLENFDLRKVARKKAADDDSLDIKAANHNRFVAN
ISELRLVLRNAFEKCGEIELVKAVNTTKICFWCGFINNFDQAKNLIHQCYSCGVVWDQDDNASTNIRRRRKQG
(SEQ ID NO: 126)
>3300017963|Ga0180437_10000692_13
[aquatic-non marine saline and alkaline-hypersaline lake sediment]
MGAARRRNPKVAAARKGKPPPKATGNCRNYRYGAHEPIANLDKVLDEMRGAHDLRNVLTCINRARSEMITAAL
GEHQSYKKATADLAALHQRRDKLEAQIRQQNSASRKRLGRHSPLSSELDTVRKRIDEGRTALKKLRRKLLKKD
PALKAVVEAADDMAKRETTRAEDACGLYWCTRNEQTGKRAKLRRFKKWRDSEATISVQIPGGLTVEQLLGGEN
NQARLELRPEGVWVQGARKRKVEPAEAARNKLRLDEDGYPMRKLGTAILHLRCMSDEDGKPIWAEVPITYHRE
IPADAKIKRCYLHRFRVGNRYHWSVRFSLERGKKGDDSWLHPRVATTGTAAIDIGWRWFPDRLRVAVWAGSDG
AEGELCLPKWWLDEMYSVRLDQRERDVLFNEIVSLVLPWFRSRRGELSDYVVQAIKTMHSWRDKGRLAALSMR
WRDDLAADPGANPAHVAMSIRLEEWRKRDKHIWCEEVNLRSQLQGSRKDLYRRFAAMLTSRYGRIVVEEFDLS
AVQKLPPASIDDGTYSRVKRHKGDAACSHLVGALKDAARQLDKKNPKWTTKRCHVCGKTERKWENPGELEHTC
KHCGVLWDRDVNAARNILAASGVAVDWTRPPLAPAARMTYPQVENREMRRSRRRKEALETTRASGDRQTA
(SEQ ID NO: 127)
>3300017963|Ga0180437_10006965_20
[aquatic-non marine saline and alkaline-hypersaline lake sediment]
MLKRGERGERGERGERGERGERGASMPRNPKKKMDGVGRNYKYGAYAPLTNEDEVRWQMVLGHRYRNRLVEVE
LDIRAKRDAIIQEVAPGLLALEDEINKMGEIIALHEKAQKEQNKKQRGRDVHPGVANLLRDLKAEKKGLVGKR
KALKAELFASDRWKQDGGDHLNQQRKEGRSNAYSEYKDEGLWWGVRSKILRESGSFISGAPPKFRGWHKSVRS
TRFVVQTQGGLTEEELLSGRNTTARLTLFPDGVWAEGKRRPKRMGDAILDLRIGSDEHRKPIWTSIPISYDRH
LPAEAKIKWIYLFKRLLVDKEKWEVVFALECPAAADYDAIRRRGGDKKRTNRNRKGIRLRKYAQSGVVAIDVG
WRKFEDYLLVGTCAASDGREWELRLDGNWLGQLRRVEGMQSYRDVLLNEQVKWLHPWLKSRKGSLPELLLPPS
RNLEKWGQRSVARLVKQWMRERPIGTLDEQRALARLDEWLSRENHVWHFQANLQHQLLLYRREEYRVWARRIG
EVYRCVVLEKLNYGDWHKKPPVERGGSVKADMAKKYLRDAGLSHLKNALKGGVLQVADVPHEGTTVNCHACGH
ADVWEDPAAKDHVCETCGLRWDRDVNAARNILAASGVTVAWEREPLAPTEAWTACSKSGLNRAQRRAISSSLA
IDSEIALAVGGSE (SEQ ID NO: 128)
>3300017963|Ga0180437_10006965_20
[aquatic-non marine saline and alkaline-hypersaline lake sediment]
MPRNPKKKMDGVGRNYKYGAYAPLTNEDEVRWQMVLGHRYRNRLVEVELDIRAKRDAIIQEVAPGLLALEDEI
NKMGEIIALHEKAQKEQNKKQRGRDVHPGVANLLRDLKAEKKGLVGKRKALKAELFASDRWKQDGGDHLNQQR
KEGRSNAYSEYKDEGLWWGVRSKILRESGSFISGAPPKFRGWHKSVRSTRFVVQTQGGLTEEELLSGRNTTAR
LTLFPDGVWAEGKRRPKRMGDAILDLRIGSDEHRKPIWTSIPISYDRHLPAEAKIKWIYLFKRLLVDKEKWEV
VFALECPAAADYDAIRRRGGDKKRTNRNRKGIRLRKYAQSGVVAIDVGWRKFEDYLLVGTCAASDGREWELRL
DGNWLGQLRRVEGMQSYRDVLLNEQVKWLHPWLKSRKGSLPELLLPPSRNLEKWGQRSVARLVKQWMRERPIG
TLDEQRALARLDEWLSRENHVWHFQANLQHQLLLYRREEYRVWARRIGEVYRCVVLEKLNYGDWHKKPPVERG
GSVKADMAKKYLRDAGLSHLKNALKGGVLQVADVPHEGTTVNCHACGHADVWEDPAAKDHVCETCGLRWDRDV
NAARNILAASGVTVAWEREPLAPTEAWTACSKSGLNRAQRRAISSSLAIDSEIALAVGGSE (SEQ ID NO:
129)
>3300017963|Ga0180437_10073069_2
[aquatic-non marine saline and alkaline-hypersaline lake sediment]
MGNVPLLEQQTKEAGERVSEASKSVKQYRSKNRTRKVPEWMRTELDAARLAKKDVAAKLREVRKQLRTPEIQA
EMDRINGLAGELRRSARAHCGLYWGSYLLVEDEMASSSKSPLYDKENPNEPNDPGFVRWHGEGHLGVQIQGGM
PTGLVQFHSTLLQIKKVDPVEGKLGKSHYLLRMRVGSNGRKPIWGEWPMVMHRPLDPGQIKGAAVSCRRIGLR
WQWTVEITVDKESGCRPRPCGYGQVAVNFGWRKVDGGIRVAYAVDYEGNEQELVLPDGEAEGIVRPSRVRERL
TDEQRAIQKRDGIIYGKACRLSDDGKSYEAEKVLSGRPDLLSRLSSRVRPARKPPILPALRKSDELRSIRDQR
FGHTLQSLIKWLKTIEVPCWLKDRTSHIHKWKSQNRLRKLIGYWRSNRFDGDETMFQSLEVWNHRDEHLLSWE
DSQRKKSQRRRRDLYRVWAAKLADRYYTIVLNSHDMAETARKPKVEATDDIPLSRSNRQLVSPSELKEALINA
KRSREGQTVENPAQKVTHTCHNCETEQDFDAASSIEHTCLACGETWDQDRNAAINSLRWFVERPSDAKILGTA
RKIKNLDENGVEKETRRQRISRLKREKDARMKALANDAASS (SEQ ID NO: 130)
>3300017971|Ga0180438_10000090_91
[aquatic-non marine saline and alkaline-hypersaline lake sediment]
MTRKTSKTKRKKKPGKPRVRGPQLAYVYGLPFGPTKNAELVEKQIVLSQRYNNQCVEAERRLRATLREIYQQH
TLDLMGASDEMREAFTEVKRLEKLLREMQEDLRTKRKRSRSRSDTPQERMRLREVRDLKNEAWAKLRELKNGS
ESDDEPGKEEPRKKVELSDELKARRAEAQQREKQELHEAYVQFKDGTYEKVTETDEELGKLYWGTYLLVNRAR
EASRMSLRDSLWKWNEEKGIWVERDPKFKSLDDEVIFGVELQKGDSVERVLNCQNTMFQLDMEPEMGEEVLRH
RRIRRRGIARIRVGSGGKSGRDPIWAEFPVIMHRPLPPKARIKWAVVKREKITTRLRWTLHLHLEVDSGDCHK
DYGTGRGVVAVDIGWRKRGTETVEMGRKRKKRGLRKQEVEVPRIRIAYLIDDREYAAYLKNPDEGEVGHEQCM
SSKVVAGFQRVETLQQTRQLKQNEMLAELRAWIKARRSALPKWFRESTRGIAKWEAPKRFAWLLRLWRESRWK
GDERGFEILDRWQRGVYDEEARRLEGGDRHLWQWQESQRRKSLLQREDHYRCVDSALAREFKVLVLENIDLSK
MQKHELPGSDKVEIRRARRQQKEAALSEFRETLIQAFLSRGGTVVWVNPAMTTQRCFDCGHDAPWDPIPKVEH
TCEKCGRTWDQDANAARNMMRLYRENKIVKIADGSVLVREMSDAQKNRNKGKKVVRKRKKEEEERNGEGPAPL
ES (SEQ ID NO: 125)
>3300017971|Ga0180438_10000124_114
[aquatic-non marine saline and alkaline-hypersaline lake sediment]
MGAARRRNPKVAAARKGKPPPKATGNCRNYRYGAHEPIANLDKVLDEMRGAHDLRNVLTCINRARSEMITAAL
GEHQSYKKATADLAALHQRRDKLEAQIRQQNSASRKRLGRHSPLSSELDTVRKRIDEGRTALKKLRRKLLKKD
PALKAVVEAADDMAKRETTRAEDACGLYWCTRNEQTGKRAKLRRFKKWRDSEATISVQIPGGLTVEQLLGGEN
NQARLELRPEGVWVQGARKRKVEPAEAARNKLRLDEDGYPMRKLGTAILHLRCMSDEDGKPIWAEVPITYHRE
IPADAKIKRCYLHRFRVGNRYHWSVRFSLERGKKGDDSWLHPRVATTGTAAIDIGWRWFPDRLRVAVWAGSDG
AEGELCLPKWWLDEMYSVRLDQRERDVLFNEIVSLVLPWFRSRRGELSDYVVQAIKTMHSWRDKGRLAALSMR
WRDDLAADPGANPAHVAMSIRLEEWRKRDKHIWCEEVNLRSQLQGSRKDLYRRFAAMLTSRYGRIVVEEFDLS
AVQKLPPASIDDGTYSRVKRHKGDAACSHLVGALKDAARQLDKKNPKWTTKRCHVCGKTERKWENPGELEHTC
KHCGVLWDRDVNAARNILAASGVAVDWTRPPLAPAARMTYPQVENREMRRSRRRKEALETTRASGDRQTA
(SEQ ID NO: 127)
>3300017971|Ga0180438_10000195_144
[aquatic-non marine saline and alkaline-hypersaline lake sediment]
MTKTYVYGLPLGPTVNADLVEEQMRLAHKYRNALIEIERERREKVREVYDERDLALEGLVEEDKVAKSELKRA
TEDLKRQRAKTRSRSDTAEQRARVKEARKAAQEVAKRLSEARKELKLDEELQKRLSEANLTASEKSQAAQQGF
SREGLFWGTYLQVDNAMEDSRRDLKMWDEHGQPLDPKFLQWRGDGTVAVQLQGDKHPVEKIFSGEDTFLQVDM
EPPPEGVVSKTRRKKRRGVMRLRVGSTKSRGPVWAEFPIIMHRPLPQGVRIKWAVVKRRMISDRPRWTVHFSL
GLPAEYQHEEFGSGRGAVAVDIGWRKRGEDQIRVAYLVDGDEYAAYLRDRQDPLGRGDELLMEPEVVRGFDKV
ESLQSIRALNQNEMQKSLKGWIKSNKKNLPEWFREDVRYLHSWKSPKRYAGLLRKWGEKRWDGDGEGFQILKD
WLSGTYEESLGRRDGGDRHLWQWKESQEQKSLRRRKDHYRRVAAKLARKYKVLVIEDFKLTETQKHEPPESEK
VEIQAARNQQKEAACYELRMMFVQAFLARGGTVVWVDARMTTQRCFECGCLEPWDAIPEVDHVCVECGAKWDQ
DANAARNIMRLYRNDETLKMIDGSVPVEPKMSRRQKGRKKGKKIVQQRKSQEAAQPSV (SEQ ID NO:
124)
>3300017971|Ga0180438_10013386_7
[aquatic-non marine saline and alkaline-hypersaline lake sediment]
MRVYKYGLLRPTTNADLVHEQIKIGHKYRNKLIELEIKRRDLIRAEVAKSSVVEDDFTDAKLAVEKFKHLDKL
LKQKNAQHRSKRHNNPDLKKDHTKARKEKTKAIKKLEETRRKVLKKCKETIKVFNDQYIEEEKKVRSECAPFW
GTYQVIEDAMKRSRKSLPLWDGLESNNPKFRRFNGIGRVSIQLQKDVIDKNNGMNVDLVFGTTDIRLQVAPVP
EEAWYSPIRSVRRKKSRTVLKMRIGSEGRAPIWAEWPMIMHRPLPDNGRIKRVTVNFRKIGPREEWTADFFIN
DSATLHEQYEVSGAIGLDVGWRLMDDGSLRVAFWEDDEGEKGEFRLSPTLMGAFKKADDLRSIRDKNRDEIKE
FLIQHFSKNPMPSWMLDFVKGKEDSKRPTNKQACVYLSKWKSIAKLTKLVQTWKEKGITKRHQKAYNRFEDWR
YHDFHLWQWETSQRKKAERRRKDNYRVLASKLSKQYHTLVLENFDLRKVARKKAADDDSLDIKAANHNRFVAN
ISELRLVLRNAFEKCGEIELVKAVNTTKICFWCGFINNFDQAKNLIHQCYSCGVVWDQDDNASTNIRRRRKQG
(SEQ ID NO: 126)
>3300017971|Ga0180438_10021273_1
[aquatic-non marine saline and alkaline-hypersaline lake sediment]
MPRNPKKKMDGVGRNYKYGAYAPLTNEDEVRWQMVLGHRYRNRLVEVELDIRAKRDAIIQEVAPGLLALEDEI
NKMGEIIALHEKAQKEQNKKQRGRDVHPGVANLLRDLKAEKKGLVGKRKALKAELFASDRWKQDGGDHLNQQR
KEGRSNAYSEYKDEGLWWGVRSKILRESGSFISGAPPKFRGWHKSVRSTRFVVQTQGGLTEEELLSGRNTTAR
LTLFPDGVWAEGKRRPKRMGDAILDLRIGSDEHRKPIWTSIPISYDRHLPAEAKIKWIYLFKRLLVDKEKWEV
VFALECPAAADYDAIRRRGGDKKRTNRNRKGIRLRKYAQSGVVAIDVGWRKFEDYLLVGTCAASDGREWELRL
DGNWLGQLRRVEGMQSYRDVLLNEQVKWLHPWLKSRKGSLPELLLPPSRNLEKWGQRSVARLVKQWMRERPIG
TLDEQRALARLDEWLSRENHVWHFQANLQHQLLLYRREEYRVWARRIGEVYRCVVLEKLNYGDWHKKPPVERG
GSVKADMAKKYLRDAGLSHLKNALKRGVLQVADVPHEGTTVNCHACGHADVWEDPAAKDHVCETCGLRWDRDV
NAARNILAASGVTVAWEREPLAPTEAWTACSKSGLNRAQRRAISSSLAIDSEIALAVGGSE (SEQ ID NO:
131)
>3300017971|Ga0180438_10044179_5
[aquatic-non marine saline and alkaline-hypersaline lake sediment]
MIVYQYGLRAPTSQIELIHDQLWLSHRYRNTLVEIERGRRAAVRRLNSTVGNVPLLEQQTKEAGERVSEASKA
VKQYRSKNRTRKVPEWMRTELDAARLEKKDVATKLREVRKQLRTPEIQAEMDRINGLAGELRRSARAHCGLYW
GSYLLVEDEMASSSKSPLYDKENPNEPNDPGFVRWHGEGHLGVQIQGGMPTGLVQFHSTLLQIKKVDPVEGKL
GKSHYLLRMRVGSNGRKPIWGEWPMVMHRPLDPGQIKGAAVSCRRIGLRWQWTVEITVDKESGCRPRPCGYGQ
VAVNFGWRKVDGGIRVAYAVDYEGNEQELVLPDGEAEGIVRPSRVRERLTDEQRAIQKRDGLIYGKACRLSDD
GKSYEAEKVLSGRPDLLSRLSSRVRPARKPPILPALRKSDELRSIRDQRFGHTLQSLIKWLKTIEVPCWLKDR
TSHIHKWKSQNRLRKLIGYWRSNRFDGDETMFQSLEVWNHRDEHLLSWEDSQRKKSQRRRRDLYRVWAAKLAD
RYYTIVLNSHDMAETARKPKVEATDDIPLSRSNRQLVSPSELKEALINAKRSREGQTVENPAQKVTHTCHNCE
TEQDFDAASSIEHTCLACGETWDQDRNAAINSLRWFVERPSDAKILGTARKIKNLDENGVEKETRRQRISRLK
REKDARMKALANDAASS (SEQ ID NO: 132)
>3300017971|Ga0180438_10056790_2
[aquatic-non marine saline and alkaline-hypersaline lake sediment]
MSRFHKDRLKVDAKIFSFNASEPMEGLEVIRSEMKLAHDYYNKLVELERARRSEIEEEQLRRFPELLRIEEEI
AVAEDSLVDLVRETKRRNSSRRSAKLPKEDRERIKIARGVLRELCKRRSEMKKGLRENADYQEAEKGITKKAK
GAAKEARHESGCFWPNYLQVEVAVESAKKPRKRRKGQRPVRWTYRPRFKRWEGRGRVSMQLQKGLSPERLESG
ADTRLRLVRGRVTKPGPRRERKQGTAMLWIRVGSTKEPGKRAQPVWAKVPFYYGGKRDRELPPDCSIKWCWLL
VDKIGLKERWRVQFSIDAPLGTLKHVDRASDGTVAIDIGWRLMGDRLRCAIWSGSDGEEGEIALTGSWVRAYS
RERAMRSYRERLFNCVLKELCSWAKEQEVLPEPLAEARALHAWKKHGKLASLSLKWRGKRDFRERSEKAASYL
REGGVVDLSGASEDDVLALLEGWRKRDKHVLEYESHLRDKLQATRLDLYRVCVANLRRRYKTCVLEEDVEDDE
RTKLMDLVKWHLLPDVIEAGDPGEEEQRRASKRGLRPACLFKLRAILKENMEIVGVPSEFTTKRCWSCGSVEE
WDQASEVEHTCENCGETWDQDVNAARNLLVASGVEATFFRPALAPAEVWTCGLRGTFPEPV (SEQ ID NO:
133)
>3300017971|Ga0180438_10072596_2
[aquatic-non marine saline and alkaline-hypersaline lake sediment]
MFGHDSLPSRIYRYGAKAPTVGAENVDRQMSLGHRYYNTLVEIERRRREKAAALVARVSPALASLEQRREALT
AAIAERREAVKKSNQEVRKKQATKDERDAIAALTAERKEVNVLYRDAKDLAYNSPEAAAGLAAIDQQADMEAK
TARAESGLYWGTYLQVEQSLPRKGPPPKFHRWMGDGKIAVQIQGGMTLEEAFAGRDQRFRLEPIPDNAWDKGN
RKHRRTRAWIRVASDGRDPVWAVVPVVLHRPIPDDAQIKWVYLLRRRVGCNNNWSLCLVISRQAWQRHDLAGD
GAVGINLGWRKVEGGIRVATWVGDDDESDTLVISERDAGRWQKAKDLRSIRDGRFNAIQEALVDWLGSHAVPE
WFSERTATIKQWRSQARLAALVIAWRSQRFEGDEGIFPAMEAWRKKDKHLYEWEANQRRKAVAWRNDLYRCFA
AKLSQRYETAVLGKTDWKTIGRRPSPENPEHASGGENRTLASPGILQRMIVERVARVELADAKHITQRCHACG
KLASFDARTNIFTTCRHCAETWDQDENAARNLLLSASGPVAQKTP (SEQ ID NO: 134)
>3300017987|Ga0180431_10022214_3
[aquatic-non marine saline and alkaline-hypersaline lake sediment]
MRVIVYEYGLRQPTSGIDDIDDQIHRAHRYYNKLIEIERWRRAQVKKAQLQVPEVANTKKVVEALREDLEALR
TQHKRAKSHDGKTHPPRAGAIKDTTAALKAARQGYRQAKKDAADILKPLYKKVDEERNALVRQARGESGVYWG
TYLCIEQFASQAAQTAKRESPDFRRWTGDGMLAVQIQNGLDAGALFGDDTRVQVAPIDSKAWDKSISRGKRKR
MQYTTLRLRVGSTGPGNREPVWAEWPLFMHRELPADASIKWVRVIRRRWDQRWKYRWVVQFTVEVPEAPGWQG
EGTRKGMVAINLGWRKLATDALRVATWVDTEGNVGELQLPVSFRQRLEKANSIRSIRDRKLDELKAAIVPLLP
ECSRWKSPKRFEGLLRQDDLPDGVRDLVNKWAYRDRHLWWFERGCRQGALRYRREIYRLFALEMAKKYPLVIV
EDYDLRPIVTDENRIKLPSHQRVEGSPSEARHVLLASVSRLGGMVIDGKSKLATQECHLCGYGKEKDERWDAS
PKIEHTCVGCGENWDQDVNNARVLLARAQVMLESGELLAQPKPKRSARFAKKHKKQNEAVL (SEQ ID NO:
135)
>3300017987|Ga0180431_10041976_5
[aquatic-non marine saline and alkaline-hypersaline lake sediment]
MFGHDSLPSRIYRYGAKAPTVGAENVDRQMSLGHRYYNTLVEIERRRREKAAALVARVSPALASLEQRREALT
AAIAERREAVKKTNQEVRKKQATKDERDAIAALTAERKEVNVLYRDAKDLAYNSPEAAAGLAAIDQQADMEAK
TARAESGLYWGTYLQVEQSLPRKGPPPKFHRWMGDGKIAVQIQGGMTLEEAFAGRDQRFRLEPIPDNAWDKGN
RKHRRTRAWIRVASDGRDPVWAVVPVVLHRPIPDDAQIKWVYLLRRRVGCNNNWSLCLVISRQAWQRHDLAGD
GAVGINLGWRKVEGGIRVATWVGDDDESGTLVISERDAGRWQKAKDLRSIRDGRFNAIQEALVDWLGSHAVPE
WFSERTATIKQWRSQARLAALVIAWRSQRFEGDEGIFPAMEAWRKKDKHLYEWEANQRRKAVAWRNDLYRCFA
AKLSQRYETAVLGKTDWKTIGRRPSPENPEHASGGENRTLASPGILQRMIVERVARVELADAKHITQRCHACG
KLASFDARTNIFTTCRHCAETWDQDENAARNLLLSASGPAAQKTP (SEQ ID NO: 136)
>3300017989|Ga0180432_10002388_5
[aquatic-non marine saline and alkaline-hypersaline lake sediment]
MRVIVYEYGLRQPTSGIDDIDDQIHRAHRYYNKLIEIERWRRAQVKKAQLQVPEVANTKKVVEALREDLEALR
TQHKRAKSHDGKTHPPRAGAIKDTTAALKAARQGYRQAKKDAADILKPLYKKVDEERNALVRQARGESGVYWG
TYLCIEQFASQAAQTAKRESPDFRRWTGDGMLAVQIQNGLDAGALFGDDTRVQVAPIDSKAWDKSISRGKRKR
MQYTTLRLRVGSTGPGNREPVWAEWPLFMHRELPADASIKWVRVIRRRWDQRWKYRWVVQFTVEVPEAPGWQG
EGTRKGMVAINLGWRKLATDALRVATWVDTEGNVGELQLPVSFRQRLEKANSIRSIRDRKLDELKAAIVPLLP
ECSRWKSPKRFEGLLRQDDLPDGVRDLVNKWAYRDRHLWWFERGCRQGALRYRREIYRLFALEMAKKYPLVIV
EDYDLRPIVTDENRIKLPSHQRVEGSPSEARHVLLASVSRLGGMVIDGKSKLATQECHLCGYGKEKDERWDAS
PKIEHTCVGCGENWDQDVNNARVLLARAQVMLESGELLAQPKPKRSARFAKKHKKQNEAVL (SEQ ID NO:
135)
>3300017989|Ga0180432_10021155_3
[aquatic-non marine saline and alkaline-hypersaline lake sediment]
MYRYGLLPPSSGAELVDEQMWLVHRYSNELVEIERARRKGFHAALAVVPEVAEALRVEEEALARYHVLRDSDS
DDRAAEFRKKGKRRRKSSPAVADALAALKAATDAVEDVRAAAVKAIGRDSKKKKTAKKKKKKKTATELVKMTP
AEVASVTEAINENDAVHGDAAKRKRAEFIAQGLYWGNYQVAEASIPRKGPPPKFRRWEGRGHIAVQIQGGMTY
AELLSCNHTMARLEIRDDWGSNRRTSRHGLLWIRVGSKGKGGREPVWATFPVCWHRHLPEGARIKRIDVTRRI
QGVRAVWAVCVTVQTPGASLTKQALVKPVTKALPKAVGLDVGWRSTDDGGIRVAVLYDGDRHYEVALPHWFAE
GDRLVSDLQSIRRCRFNAVKDQLLAALREGKHKEQAETFATLASWDSQARLARAVREWEGCPAYLTEWRAKER
HLYQWERDAKRYLVEWRKNWYCHWVAWISQRYKNVVIEKFDIAKIKKKAEAGEDKEEATGPHSLAAPGELRRI
LLSTCSREGVQVHLAPAGNTTRKCSVCGKLRRKKKGEGVALMQECSGCGRVMDQDANASRNLYGFASAGVIPE
TPVAFAVPEAAWYGRFSLTPKKIQSRVARLQAALETSPPDSDGKGG (SEQ ID NO: 137)
>3300017989|Ga0180432_10021155_5
[aquatic-non marine saline and alkaline-hypersaline lake sediment]
MAFHHSTQPTTSRVYRYGLLPPSSGAELVDEQMWLVHRYSNELVEIERARRKGFHAALAVVPEVAEALRVEEE
ALARYHVLRDSDSDDRAAEFRKKGKRRRKSSPAVADALAALKAATDAVEDVRAAAVKAIGRDSKKKKTAKKKK
KKKTATELVKMTPAEVASVTEAINENDAVHGDAAKRKRAEFIAQGLYWGNYQVAEASIPRKGPPPKFRRWEGR
GHIAVQIQGGMTYAELLSCNHTMARLEIRDDWGSNRRTSRHGLLWIRVGSKGKGGREPVWATFPVCWHRHLPE
GARIKRIDVTRRIQGVRAVWAVCVTVQTPGASLTKQALVKPVTKALPKAVGLDVGWRSTDDGGIRVAVLYDGD
RHYEVALPHWFAEGDRLVSDLQSIRRCRFNAVKDQLLAALREGKHKEQAETFATLASWDSQARLARAVREWEG
CPAYLTEWRAKERHLYQWERDAKRYLVEWRKNWYCHWVAWISQRYKNVVIEKFDIAKIKKKAEAGEDKEEATG
PHSLAAPGELRRILLSTCSREGVQVHLAPAGNTTRKCSVCGKLRRKKKGEGVALMQECSGCGRVMDQDANASR
NLYGFASAGVIPETPVAFAVPEAAWYGRFSLTPKKIQSRVARLQAALETSPPDSDGKGG (SEQ ID NO:
138)
>3300017989|Ga0180432_10043261_1
[aquatic-non marine saline and alkaline-hypersaline lake sediment]
MSTKVYKFRLYAPILNGDLVEEQLKLANAYRNKLIELERDRRVVARELNAERRSVLGEYIDAAEELKTRLKRE
VNRLKAMKAMKARGARKSPELKDQEKLVTQIRQERKAAVEDLKAREANLKTTSELQAKYDKLWEDLTNKTKEE
RNLNGLYWGTGGFQEQAMQKSSETLHLGKDPRFKRWDGCGTVAVQVQKPLQMPLKDFFHGKSTLINFIMDDEG
ASGTKRHGVVQLRVGSDRKKPIWAEWPLVMHREMHERAVITGAQIHKTRTADKFKYHLCVTAKLPDDVRKERC
GDGVVALDIGWRKLLDGNLRVAYWKDREGNGGQLVLDPAVLSGLGKDASLQAICRGLLNKLYKAFYTWLSSVA
NLPENFQQIYEEMTAEKAYWKEFRALQKIVREARAGGLPELDALEEKLAEMKARQKEVRTWKVQGKFSGLLED
WRNNRWDGDNAGFTMLDDWWRGTYNPESGHREGGCKHLWQWRSNQREKSQRRRKHQYRNLGAEFSRKAGVLVL
ENFDLTDMQRDAEPEEKKKNPEAKLNQRYAACYELREAFIQAFQSRGGRVVKIDPQMTTQICARCGCDTRWDA
ALEIEHTCERCGATWDQDENAADNLLKLYEGGGSIQEVTVVKKDPRWKRLKAEKAAKLEDRGGARKD (SEQ
ID NO: 139)
>3300017989|Ga0180432_10045094_6
[aquatic-non marine saline and alkaline-hypersaline lake sediment]
MVTEYTTKVYTCGLRPPAENADLVSEQIRLGHRYYNRLIEIEHEKRQRDHEIVGAHGDADALQAAIDEQVVVV
EQVVARIRRWRIANGKKVASKDLRMELAAAKKSLKAARAELRELRRVIKQDPEIAASRVALWAEDSAARKRAR
AECGIPHGTYIQVEQAVEAACKAPMAPGCETPWWELPRFKRWKGEGCVGLQLQQRDGEYMDTDALFGRSDPRL
QIDPVPSTAWDRRRSREQRTVVRMRIGSECRRCGALCTSIHCPEGGDGGAAYRSPVWASWPMILHRPLPEGAL
IKWAKVKRERIVGKARWRWSLHLTIDEPEQEPRCGEGTVAVDVGWRKTETGMRVGYWQDDSGDHNSINIDHEI
LDRLRKVDELESIRKRNMNAAKSQLRAWLATWEEVPDWMREASRHMHAWRSQNRLAGLALHWRQNRWEGDNPG
YEDLEDWRKQDKHLWAWQDNLRGKVLRRRREVYRVAAARLAERYDTVVLTDFDLRDTQRHPSDTSTREEIDAV
KWQQKAAACSVLRGCIRNAFTSRGGRIVEVEAKLMSRTCHGCGHDGEWAKPEELEHTCQGCGETWDRDVNSTT
NMLRAARERSDDDDGRPKKRAAKWAKRHGRSKNENDDDGTSRNAGDKVA (SEQ ID NO: 140)
>3300017991|Ga0180434_10002646_1
[aquatic-non marine saline and alkaline-hypersaline lake sediment]
MRVIVYEYGLRQPTSGIDDIDDQIHRAHRYYNKLIEIERWRRAQVKKAQLQVPEVANTKKVVEALREDLEALR
TQHKRAKSHDGKTHPPRAGAIKDTTAALKAARQGYRQAKKDAADILKPLYKKVDEERNALVRQARGESGVYWG
TYLCIEQFASQAAQTAKRESPDFRRWTGDGMLAVQIQNGLDAGALFGDDTRVQVAPIDSKAWDKSISRGKRKR
MQYTTLRLRVGSTGPGNREPVWAEWPLFMHRELPADASIKWVRVIRRRWDQRWKYRWVVQFTVEVPEAPGWQG
EGTRKGMVAINLGWRKLATDALRVATWVDTEGNVGELQLPVSFRQRLEKANSIRSIRDRKLDELKAAIVPLLP
ECSRWKSPKRFEGLLRQDDLPDGVRDLVNKWAYRDRHLWWFERGCRQGALRYRREIYRLFALEMAKKYPLVIV
EDYDLRPIVTDENRIKLPSHQRVEGSPSEARHVLLASVSRLGGMVIDGKSKLATQECHLCGYGKEKDERWDAS
PKIEHTCVGCGENWDQDVNNARVLLARAQVMLESGELLAQPKPKRSARFAKKHKKQNEAVL (SEQ ID NO:
135)
>3300017991|Ga0180434_10013735_9
[aquatic-non marine saline and alkaline-hypersaline lake sediment]
MFGHASDPSVIYRYGALPPTHNLDAAFEQLRAAHRYRNKLVEIERDRRDKTAAVVSAASPDLAGLESQYAELG
ERTAAAAKQIKATNQRARAQRATPEQKAVLRKLRAECKDVYSRLKEAKALAYKSLEARTALDQADAAALNAAK
KARAECECYWGTYLQVEQGLSGIRKGAPPRFLRWTGNGKLAVQIQGGMSREEAEHGDGRLRIATTERRGKATN
VYLRIGTNEDRSPIWAVVPVIFHRPIPDDARIKWVYLTARRVACHTRWHVCFVLSRAEGWRKPDLATSGTVAV
DLGWRLLDHGLRVGYWRGSDGGSEEILLPTRDVARWQKADDLRAIRGERFNGVVDWLAKWLAGRDLPDWLIER
TRTLRQWRSAARLASVVIHWRENRFAGDKDGFAAVEAWRKKDKHLYEWEANQRRKAVAWRDDLYRRVAADLSR
RYKTAIVEDCNWRDVGRKPDVGENNDSGAAARQRTIAAPGRLKQLLVERFAETVKAEAAYTTQRCHACGELAH
VETRTSVWVTCQQCGAAWDQDDNACRNMLDMVAKGPVT (SEQ ID NO: 141)
>3300017992|Ga0180435_10018121_11
[aquatic-non marine saline and alkaline-hypersaline lake sediment]
MTKTYVYGLPLGPTVNADLVEEQMRLAHKYRNALIEIERERREKVREVYDERDLALEGLVEEDKVAKSELKRA
TEDLKRQRAKTRSRSDTAEQRERVKEVRKAAQEVAKRLSEARKELKLDEDLQERLSRANLTASEKSQAAQQGF
SREGLFWGTYLQVDNAMEDSRRDLKMWDEHGQPLDPKFLQWRGDGTVAVQLQGDKHPVEKIFSGEDTFLQVDM
EPPPEGVVSKTRRKKRRGVMKIRIGSTESRGPVWAEFPIIMHRPLPQGVRIKWAVVKRRMISERPRWTVHFSL
GLPAEYQHEEFGSGRGAVAVDIGWRKRGEDQIRVAYLVDGDEYAAYLRDRQDPLGRGDELLMEPEVVRGFDKV
ESLQSIRALNQNEMQKSLKGWIKSNKKNLPEWFREDVRYLHSWKSPKRYAGLLRKWGEKRWDGDGEGFQILKD
WLSGTYEESLGRRDGGDRHLWQWKESQEQKSLRRRKDHYRRVAAKLARKYKVLVIEDFKLTETQKHEPPESEK
VEIQAARNQQKEAACYELRMMFVQAFLARGGTVVWVDARMTTQRCFECGCLEPWDAIPEVDHVCVECGAKWDQ
DANAARNIMRLYRNDETLKMIDGSVPVEPKMSRRQKGRKKGKKIVQQRKSQEAAQPSV (SEQ ID NO:
142)
>3300018065|Ga0180430_10011859_2
[aquatic-non marine saline and alkaline-hypersaline lake sediment]
MFGHKADPSLIYRYGAKTPIEHCDVVDAQIRAAHRYYNQLVEIELRRREQATELVRSLSPELDTLTEWREELS
ETIDSVRAEIKAANQRARRKTTTKAQRDQVKALRKQRKAVTELWREAKAAAYDSPDAKAGLAAIDEAANESRR
QARAACGVYWGTYLAIEQSIPKTSAPPTFHRWTGDGRVVVQLQGGMSAAEAFACRDNRFRIEPVPEEAWDRGQ
PKRLQRTRAWVRVDSDGRDPVWAVVPITLHRPFPEDCRIKWVYLIRRKVASKDKWSLCLVLSRVEGWQKTDLG
ASGSVGIDLGWRLVAEGLRVAYWAGDDGESGSVVLPMRDVGRWQKARDLQSIRATNFDAIVLRLAGWLAGREL
PDWLTERTKTLRQWRSQGRLAAVVIQWRAERFDGDAEIFAEVEAWRKQDKHLWEWEHNQRRKAIAWRENVYRQ
FAAMLSRRYRVVCLEATDWRHFMRKVAAEEDGQGGAGAQRYLRIASPGQLSRLLAERFAEVVRVDPKHTTQRC
HVCGELAQFDAATSLHTKCRHCGAEWDQDYNAARNLLGAASGPVPQETP (SEQ ID NO: 143)
>3300018065|Ga0180430_10038979_3
[aquatic-non marine saline and alkaline-hypersaline lake sediment]
MFGNKALPSVIYKYGARRPVTNADEVDRQVRDAHRYRNKLVEIERDRRSCVNAKLMQLAPRLLSLETEIERLD
NLIAEKRSEIKRANATRRRRDVTPEQRAELRQWQADRKALRTELKERKADAFADPRIRTALAKVDAEALAASK
AARAASGVYWGTYCQVEQSLSGMRSGAPPRFLRFDGTGKLAVQLQGGLSVAKAFAGEDRRLIIEPVPPKAYLP
GEPKALQRTRVWLRIGSDGREPIWTIVPITLHRPLPDDASIKWVYLTRRRVATKDRWSVCFVLARESGWQKPG
LARNGSVGVNLGWRVMDDGVERGLRVARWVGDDGTEGELRLPMPDVERWKKTEDLQAIRDQRFNAAVSLLADW
LADPGCLLPDWLVERTATLRQWRSAARLAAIAIQWRGERFEGDDTAFATLEAWRKKDKHLYEWQANQLRKAIA
WREDLYRNLAATLSRRYHTVCLANTDWRDLARRPTAEQAETDAGARRYQRVASPGALGRLLRERFAETVTVDS
RHITQRCHACGEVNQFDAAAHVRATCRHCGAEWDQDINAARNILRAASGPVACETP (SEQ ID NO: 144)
>3300018080|Ga0180433_10006034_17
[aquatic-non marine saline and alkaline-hypersaline lake sediment]
MRLANRYYNCLVVIERERRQAVRDALQECDQRHGIDALQDVVDTLKTQRDEAREEIKRARSKTRSRSDTEDQR
KRVKDLTVQIKEACELVKRARRDIRDDEQAKEQMKAADNIARTKTREARAICGVFWGTYLTIEAAIDAARKAP
LWQYGKPNDPRFRRFGGRGSVSMQLQGGLAASDVFGDDRRLQIELSPQRKSNSNRSKIRRYGVIRLRVGSSKR
DPIWAEWPLLMHRQLPDLSTIKWARIVCDRVANEERWSLQLTIDIPEPVKVSDERKGTVAINPGWRLLDYGVR
VGYIVDDCNETDEIVIDPGVLSGLRKVEDLRSIRDRTQNTMMEEFLPWLRSHKNILPAWLTERTKTIGQWKAA
ARFAALAHVWSVSRFGGDVLGYELLEQWRKQDLHLWQWESFQRRKSIGRRRNQYRRLAKQLAHRYHTLVLDTT
NLAEIQRHKSTESEEIEIPAARLQQRDAATAELRSYLAEAFHATGGVVVKVNHKRATRRCHVCGHEGPWNQCD
EVVHKCESCGSSWDQDENNCRNLLERLGDGDKITTKRQAKWDRLGRHNKTARKLDDNDVEIQTN (SEQ ID
NO: 145)
>3300018080|Ga0180433_10006034_18
[aquatic-non marine saline and alkaline-hypersaline lake sediment]
MPTKVYKYGVRPPTKNADIVHEQMRLANRYYNCLVVIERERRQAVRDALQECDQRHGIDALQDVVDTLKTQRD
EAREEIKRARSKTRSRSDTEDQRKRVKDLTVQIKEACELVKRARRDIRDDEQAKEQMKAADNIARTKTREARA
ICGVFWGTYLTIEAAIDAARKAPLWQYGKPNDPRFRRFGGRGSVSMQLQGGLAASDVFGDDRRLQIELSPQRK
SNSNRSKIRRYGVIRLRVGSSKRDPIWAEWPLLMHRQLPDLSTIKWARIVCDRVANEERWSLQLTIDIPEPVK
VSDERKGTVAINPGWRLLDYGVRVGYIVDDCNETDEIVIDPGVLSGLRKVEDLRSIRDRTQNTMMEEFLPWLR
SHKNILPAWLTERTKTIGQWKAAARFAALAHVWSVSRFGGDVLGYELLEQWRKQDLHLWQWESFQRRKSIGRR
RNQYRRLAKQLAHRYHTLVLDTTNLAEIQRHKSTESEEIEIPAARLQQRDAATAELRSYLAEAFHATGGVVVK
VNHKRATRRCHVCGHEGPWNQCDEVVHKCESCGSSWDQDENNCRNLLERLGDGDKITTKRQAKWDRLGRHNKT
ARKLDDNDVEIQTN (SEQ ID NO: 146)
>3300018080|Ga0180433_10012134_6
[aquatic-non marine saline and alkaline-hypersaline lake sediment]
MWLVHRYSNELVEIERARRKGFHAALAVVPEVAEALRVEEEALARYHVLRDSDSDDRAAEFRKKGKRRRKSSP
AVADALAALKAATDAVEDVRAAAVKAIGRDSKKKTAKKKKKKKTATELVKMTPAEVASVTEAINENDAVHGDA
AKRKRAEFIAQGLYWGNYQVAEASIPRKGPPPKFRRWEGRGHIAVQIQGGMTYAELLGCNHTMARLEIRDDWG
SNRRTSRHGLLWIRVGSKGKGGREPVWATFPVCWHRHLPEGARIKRIDVTRRIQGVRAVWAVCVTVQTPGASL
TKQALVKPTTEALPKAVGLDVGWRSTDDGGIRVAVLYDGDRHYEVALPHWFAEGDRLVSDLQSIRRCRFNAVK
DQLLAALREGKHKEQAETFATLASWDSQARLARAVREWEGCPAYLTEWRAKERHLYQWERDAKRYLVEWRKNW
YCHWVAWISQRYKNVVIEKFDIAKIKKKAEAGEDKEEATGPHSLAAPGELRRILLSTCSREGVQVHLAPAGNT
TRKCSVCGKLRRKKKGEGVALMQECPGCGRVMDQDANAARNLYGFASVGVIPETPVAFAVPEAAWYGRFSLTP
KKIQSRVARLQAALETSPPDSDGKGG (SEQ ID NO: 147)
>3300018080|Ga0180433_10012134_6
[aquatic-non marine saline and alkaline-hypersaline lake sediment]
MAFHRSTQPTTSRVYRYGLLPPSSGANLVDEQMWLVHRYSNELVEIERARRKGFHAALAVVPEVAEALRVEEE
ALARYHVLRDSDSDDRAAEFRKKGKRRRKSSPAVADALAALKAATDAVEDVRAAAVKAIGRDSKKKTAKKKKK
KKTATELVKMTPAEVASVTEAINENDAVHGDAAKRKRAEFIAQGLYWGNYQVAEASIPRKGPPPKFRRWEGRG
HIAVQIQGGMTYAELLGCNHTMARLEIRDDWGSNRRTSRHGLLWIRVGSKGKGGREPVWATFPVCWHRHLPEG
ARIKRIDVTRRIQGVRAVWAVCVTVQTPGASLTKQALVKPTTEALPKAVGLDVGWRSTDDGGIRVAVLYDGDR
HYEVALPHWFAEGDRLVSDLQSIRRCRFNAVKDQLLAALREGKHKEQAETFATLASWDSQARLARAVREWEGC
PAYLTEWRAKERHLYQWERDAKRYLVEWRKNWYCHWVAWISQRYKNVVIEKFDIAKIKKKAEAGEDKEEATGP
HSLAAPGELRRILLSTCSREGVQVHLAPAGNTTRKCSVCGKLRRKKKGEGVALMQECPGCGRVMDQDANAARN
LYGFASVGVIPETPVAFAVPEAAWYGRFSLTPKKIQSRVARLQAALETSPPDSDGKGG (SEQ ID NO:
148)
>3300018080|Ga0180433_10020043_6
[aquatic-non marine saline and alkaline-hypersaline lake sediment]
MRVYKYGLPWLLDEEEAPDGAKGGRMAVERQLRAAHVYQNKLIELIRARRLVHRDAVMGYGRVAELHKQVDDI
NELYQDARDDLKKTRQKERRRAESDRQKAMVAKLRELYKESLAQLYKERRKAFRDSAVKELCKEADESFYEQQ
RQERTRDLTDEERQRGWERPFWGTKQIVEASVKQAHESMPLWDGVRPNDPRFRSWDGSGILGVQNQRPLFSTL
NDEQQMDPAVQDIFGKDTSLRVDPVDPEAWHNPKRCERKRKSRTVLWMRVGSDENRQPVWACWRMIMHRPLPD
GAQIKRASVSKRIVGERQKWTVQIYVDDQGCKRAPSCGDGSVTIDLGWRQQQNGVRIATWLGSDGRQGKFKLP
QKVIERMFSERGIRKTRDENLDRMRPCLEAWIRNQKCLPEWLEKRTKMIGRWRSHARFRALAQYWRGCRFPGD
EEGYDMLEAWRYRDHHLWNYERGRSMKSRGWRDQLYCQFGAWLARQYGTVVWENFNIAKMAKRPKLGDDYENE
RARAMRHAVAVATFRDKVENAFDTRGGRSRYVSAVNTTRRCHVCGLVDAFDAASSVKRVPPCPGCGASWDQDE
NACVNMMETYDRGDSSSKPRKTNGARNGKKTNGNAVEGESHWARMKRLKREKDAHK (SEQ ID NO: 149)
>3300018080|Ga0180433_10021337_5
[aquatic-non marine saline and alkaline-hypersaline lake sediment]
MFGHASDPSVIYRYGALPPTHNLDAAFEQLRAAHRYRNKLVEIERDRRDKTAAVVSAASPDLAGLESQYAELG
ERTAAAAKQIKATNQRARAQRATPEQKAVLRKLRAECKDVYSRLKEAKALAYKSLEARTALDQADAAALNAAK
KARAECECYWGTYLQVEQGLSGIRKGAPPRFLRWTGNGKLAVQIQGGMSREEAEHGDGRLRIATTERRGKATN
VYLRIGTNEDRSPIWAVVPVIFHRPIPDDARIKWVYLTARRVACHTRWHVCFVLSRAEGWRKPDLATIGTVAV
DLGWRLLDHGLRVGYWRGSDGGSEEILLPTRDVARWQKADDLRAIRGERFNGVVDWLAKWLAGRDLPDWLIER
TRTLRQWRSAARLASVVIHWRENRFAGDKDGFAAVEAWRKKDKHLYEWEANQRRKAVAWRDDLYRRVAADLSR
RYKTAIVEDCNWRDVGRKPDVGENNDSGAAARQRTIAAPGRLKQLLVERFAETVKAEAAYTTQRCHACGELAH
VETRTSVWVTCQQCGAAWDQDDNACRNMLDMVAKGPVT (SEQ ID NO: 150)
>3300018080|Ga0180433_10021840_7
[aquatic-non marine saline and alkaline-hypersaline lake sediment]
MVDDQMRLGNRYYNRLIEIECARRDAIREAMADNDRRHGLSDAIARHARLDEQYNAAKEALKAKRSRARCRVD
TAPERAAVRDLRAQRSKAAAELKEARKGHRGDSTMHAAFDAANEEAKLQRRASRAICGVYWGTYLQIEAAVDQ
AAKETSLFFKGKPRDPRFRRWGGSVMVATQLQGGLLALSALACDDSRLQIEMAELGPGPHSRRQLKLRKGTLR
LRVGSDGRSPIWAEWPLQMHRPLPENGVIKWAKVIRRMVSDRDKWELQLTVEISPEQPCHGEGTVAVDLGWRR
KEDGTIRVGYVVDDCGLEEEIILDPGVVSGLRKSEDLRSIQDKAQAEMAARVIGWLKAQAELPPWLILATGFV
DKWKSARRWRRLASIWAEHPDVGSEALSVLSAWAADSLHLWRWEAHQRRKSCLRRKDQYRCLAKRLAAEYRHL
VLESKFLAKLQRHVEAEDEDVEIKAVRLQQRDAAGYELKQCLIYAFRRANGTAVEVDPAMTPQRCNACGFVGR
WDAAVEIDHTCEACGATWDQDANACRNLLERERPGDDSGQEAKRQGKWARKKAAKRTARKTVPSGAESFEAGV
(SEQ ID NO: 151)
>3300018080|Ga0180433_10021840_7
[aquatic-non marine saline and alkaline-hypersaline lake sediment]
MIRVYRYGLRRPTTNADLVDDQMRLGNRYYNRLIEIECARRDAIREAMADNDRRHGLSDAIARHARLDEQYNA
AKEALKAKRSRARCRVDTAPERAAVRDLRAQRSKAAAELKEARKGHRGDSTMHAAFDAANEEAKLQRRASRAI
CGVYWGTYLQIEAAVDQAAKETSLFFKGKPRDPRFRRWGGSVMVATQLQGGLLALSALACDDSRLQIEMAELG
PGPHSRRQLKLRKGTLRLRVGSDGRSPIWAEWPLQMHRPLPENGVIKWAKVIRRMVSDRDKWELQLTVEISPE
QPCHGEGTVAVDLGWRRKEDGTIRVGYVVDDCGLEEEIILDPGVVSGLRKSEDLRSIQDKAQAEMAARVIGWL
KAQAELPPWLILATGFVDKWKSARRWRRLASIWAEHPDVGSEALSVLSAWAADSLHLWRWEAHQRRKSCLRRK
DQYRCLAKRLAAEYRHLVLESKFLAKLQRHVEAEDEDVEIKAVRLQQRDAAGYELKQCLIYAFRRANGTAVEV
DPAMTPQRCNACGFVGRWDAAVEIDHTCEACGATWDQDANACRNLLERERPGDDSGQEAKRQGKWARKKAAKR
TARKTVPSGAESFEAGV (SEQ ID NO: 152)
>3300001256|JGI12210J13797_10495608_9
[aquatic-non marine saline and alkaline-hypersaline mat]
MARKTSKTPTKIYSYGARLDEGDMATARHTLWMAQDYYDDRVRIEQARRLAYREARAQVCPWLRDAEVKIDLL
ELDLEKVREELKSKRKSEFRRATGTDLATMAKELLALLKPMRKEARAQRKAASADPGVQAEGQRLDLLAKTLL
KSCSKYYGAKGLDWRTRGRVDDETRQAFADTASRPWRLGQCKKGFCGRVGGQVLAARGVFLDTDRLFSDWSTV
VQIDPLPDHTWDTRSGRRKAITAGRISVGSLGPRRPVWLRFTAVIHRRPPRGIIKNAWLFFRERGGRVEAKFQ
FTLESEEFLRASPEPVHACAIAMTPSRNLSAAVAVGTDGTIQYLSLPEKVWDRFEFAESIRSAADLAFDEVRP
SLVEAGLIPHQSRSRRRARRAAMGYAREALDAKAVWSTWRDERLGDGVDLWDSPDVVTDWAGRKGHDPLAVLC
LVWSKKDGHLDRYEDNVRHKARGYRSETYRTWVSALASKYRLFVDPYDAKYLKHAPNPEDDPRIANIERARSR
MSLYSLMTTLREKGATEVEADAVEPGAAAHVMRAASVLAKAGEDTTKAVAKIEESRRMVEMARQLDAAE
(SEQ ID NO: 153)
>3300001256|JGI12210J13797_10495610_14
[aquatic-non marine saline and alkaline-hypersaline mat]
MARKTSKTPTKIYSYGARLDEGDMATARHTLWMAQDYYDDRVRIEQARRLAYREARAQVCPWLRDAEVKIDLL
ELDLEKVREELKSKRKSEFRRATGTDLATMAKELLALLKPMRKEARAQRKAASADPGVQAEGQRLDLLAKTLL
KSCSKYYGAKGLDWRTRGRVDDETRQAFADTASRPWRLGQCKKGFCGRVGGQVLAARGVFLDTDRLFSDWSTV
VQIDPLPDHTWDTRSGRRKAITAGRISVGSLGPRRPVWLRFTAVIHRRPPRGIIKNAWLFFRERGGRVEAKFQ
FTLESEEFLRASPEPVHACAIAMTPSRNLSAAVAVGTDGTIQYLSLPEKVWDRFEFAESIRSAADLAFDEVRP
SLVEAGLIPHQSRSRRRARRAAMGYAREALDAKAVWSTWRDERLGDGVDLWDSPDVVTDWAGRKGHDPLAVLC
LVWSKKDGHLDRYEDNVRHKARGYRSETYRTWVSALASKYRLFVDPYDAKYLKHAPNPEDDPRIANIERARSR
MSLYSLMTTLREKGATEVEADAVEPGAAAHVMRAASVLAKAGEDTTKAVAKIEESRRMVEMARQLDAAE
(SEQ ID NO: 153)
>3300005917|Ga0075115_10002831_4
[aquatic-non marine saline and alkaline-saline lake]
MAKVAK GEKMTFVYEYGLRPPSLNADIVDNQLILGNRYRNALVSIERKRRDAIRGWINKPVEKESIAYSEATE
SFSIAETAMKKQRASTRSRSDTADQRDEVKDLRKKKKDALSVLKAARVKAKKEELFKAEMDDVENQSKQEIKD
ARSECGLYWGTYLVIEAAMAASRKKMPLWDKHFEPANPRYQRWQGTGTVAVQVQKSQQTTADHTMECTGRLIQ
LDMEKISDEERSKMSKRRQKRCFGTLRMRVGSEGRDPIWAEWPIIMHRPLPSDSTITEVRVIKKKISDHGKWN
VHITIKTPDGYYKQHNGVDDKCGSGPLALDLGWRLLGTGELRVAYTTDEDGTEEEIRLDHNILTGLKKSDELQ
GLCDDLQNKMKSTLNEWKKTHHLPDWFAEESSHIHAWKKTHKFVRLLHSWSKNRWDGDSEGFDILNDWHFGAY
KEDLGRRDGGSRHLWQWREHQRKKSLLRRKDQYRVLAARLSRKYSVLILEDLNLSKLQEHNKSEDDAVEIKEA
RWQQRAAACYELRECLKQAFLSRGGRVLKVKAAMTTQRCFCCGCEKKWDPIPSINHTCDQCGKTWDQDANAAK
NIMLLYDKKEFSEQSSGVKKEDAESLSKWGKIGRHKKTSLKLTDNQPEQLN (SEQ ID NO: 154)
>3300005918|Ga0075116_10002890_7
[aquatic-non marine saline and alkaline-saline lake]
MAKVAKGEKMTFVYEYGLRPPSLNADIVDNQLILGNRYRNALVSIERKRRDAIRGWINKPVEKESIAYSEATE
SFSIAETAMKKQRASTRSRSDTADQRDEVKDLRKKKKDALSVLKAARVKAKKEELFKAEMDDVENQSKQEIKD
ARSECGLYWGTYLVIEAAMAASRKKMPLWDKHFEPANPRYQRWQGTGTVAVQVQKSQQTTADHTMECTGRLIQ
LDMEKISDEERSKMSKRRQKRCFGTLRMRVGSEGRDPIWAEWPIIMHRPLPSDSTITEVRVIKKKISDHDKWN
VHITIKTPDGYYKQHNGVDDKCGSGPLALDLGWRLLGTGELRVAYTTDEDGTEEEIRLDHNILTGLKKSDELQ
GLCDDLQNKMKSTLNEWKKTHHLPDWFAEESSHIHAWKKTHKFVRLLHSWSKNRWDGDSEGFDILNDWHFGAY
KEDLGRRDGGSRHLWQWREHQRKKSLLRRKDQYRVLAARLSRKYSVLILEDLNLSKLQEHNKSEDDAVEIKEA
RWQQRAAACYELRECLKQAFLSRGGRVLKVKAAMTTQRCFCCGCEKKWDPIPSINHTCDQCGKTWDQDANAAK
NIMLLYDKKEFSEQSSGVKKEDAESLSKWGKIGRHKKTSLKLTDNQPEQLN (SEQ ID NO: 155)
>3300011414|Ga0137442_1000121_10
[aquatic-sediment-groundwater sediment]
MKRKTSTTPTRIWSFGALEPTENQKALLDQLFFANRYYNTLIEIERKRRNRFREIRSEAVPELSMLEKRYQQL
DADYVQMVAALPKPEKGKRKTLTPEVLANKEERKTTSARMKVLRAAFLEDADAKIQTAKADEEAQLAVKAARA
ATDLYWGTYLLIERQVDEARKSKSDPDFRRFDGQGRVGVQLQGGLSTPELLSGEDSRLRLQPRTSTPRVKKPK
AQHEVRIRIGSLGRDPIWATLPVIVHRPLPEDAEVKWAWVRIVRCGRRRIYSLQLTLESATFDRSQSGVGTVA
INFGWRANEDGSRRVAYAVDDAGKEQVLSIPASIEKDTTQANSLRSLRDLHFEEAKRSLVAFAALHPKAMPEW
YAEEAKFLHQWRNPARLVRLAQRLAEEHPVDSNELLRWRQERLGGARFGRHWRSPGAPKQDLFAPFPEVMSWS
TTRGIGALNFYLELWARKDKHLWGWEASLRRSVDLRRNDLFRTWAKRMTAYAEVRVEEFDLRKMTAIPAVGEE
PRDSSFRSAQRAASPGKLRERIAEACGAKVMKGAAFHNTVTCFLCSHVNERSMEHRTVCAGCGEEFDQDANNC
RNQLRERPSGAPEAGGARNPQKDPVVSDGYDESTVDRDVPSGVVAAE (SEQ ID NO: 156)
>3300011431|Ga0137438_1001223_2
[aquatic-sediment-groundwater sediment]
MKRKTSTTPTRIWSFGALEPTENQKALLDQLFFANRYYNTLIEIERKRRNRFREIRSEAVPELSMLEKRYQQL
DADYVQMVAALPKPEKGKRKTLTPEVLANKEERKTTSARMKVLRAAFLEDADAKIQTAKADEEAQLAVKAARA
ATDLYWGTYLLIERQVDEARKSKSDPDFRRFDGQGRVGVQLQGGLSTPELLSGEDSRLRLQPRTSTPRVKKPK
AQHEVRIRIGSLGRDPIWATLPVIVHRPLPEDAEVKWAWVRIVRCGRRRIYSLQLTLESATFDRSQSGVGTVA
INFGWRANEDGSRRVAYAVDDAGKEQVLSIPASIEKDTTQANSLRSLRDLHFEEAKRSLVAFAALHPKAMPEW
YAEEAKFLHQWRNPARLVRLAQRLAEEHPVDSNELLRWRQERLGGARFGRHWRSPGAPKQDLFAPFPEVMSWS
TTRGIGALNFYLELWARKDKHLWGWEASLRRSVDLRRNDLFRTWAKRMTAYAEVRVEEFDLRKMTAIPAVGEE
PRDSSFRSAQRAASPGKLRERIAEACGAKVMKGAAFHNTVTCFLCSHVNERSMEHRTVCAGCGEEFDQDANNC
RNQLRERPSGAPEAGGARNPQKDPVVSDGYDESTVDRDVPSGVVAAE (SEQ ID NO: 156)
>3300011441|Ga0137452_1000071_9
[aquatic-sediment-groundwater sediment]
MKRASREAGQVVVYRYGCPSWADLPESGMVQLRLAHDLRNELVAVEYRYRELIDGIWSSQSAVSVAELALADA
TAAVERAAALMLAQRKIDRSTIPRAGAKQALAEARAARREAKLTVKVAKAIDKEAAGPLLADAKAARYAAITS
TRAEYVVAGLFWATANDVVQNHDTAAKLVALAWKQGRPARRRTRPWKGTGTITTQVMWQAGKPARTPGVLASA
TSPWRNVFRIEPGRSRGEWPGQPSSGGTVRDDHATVHLRIEKGAEAICLPIVLHRPLPTDGDVAGVQITRRRI
AGCYRLSIAITVRLPEPTPALGGVPVSVTFGWAAAGDGAVHVARLGAPFGLGPPPPWLVKHLVAIPASATDVD
VFAPAIWRLLLARDDSIRGHRDDLLDGLREQVITALDEGVEVRLWPDDEDLLRSPVVARWRAPRRFVTLARAW
PVEHPMAAMLEAWRLRDRHLWEYESHERDQVIARRRDAYRSVAAWICGQASEILLDYPPVAELRQVPDVNEED
EYVARAGRRQVQFAAPGDLRAAIEVAARRRGVKVIDVRVPPE (SEQ ID NO: 157)
>3300006855|Ga0079044_1002244_2
[aquatic-thermal springs-hot spring]
MRVYRYGAKLRGPLDPVAEEQVELANRFWNELVDMHRKYGELLQKAQEEASPALAALRAEMAALAEEKIRLRG
LIKKSRQKARGNVPADPAIKEQLRAVSQRIKELKPIVKMEKEKAKTASSDERHRLSEQQKLEKKRLRQKYAAL
GLYWSNYNAVLQGFDTAVKRELETQGRLRVRKHAPSGAAVWTVRIQHPTGAREYTWADATRGDPSKPFSIIMP
DSEREEFTTHDGRTLSRRRLPVARLRVRAERAKTPDGTWVEFGGHHIDVPFYMHRQPPPTARVVMARLVRKRI
ADCYEYHLCITVDEPPAPKRSGTAAGVDLGWRRLPDGAVRVAYVAGEDGAKGALAVPQSTLDRLAHAERLQGI
RDSALEGIRSDFVAWAKPLLGNPALPDFVAAALAGDREHGIPPLASWRSPRRFARLTGQLVRWAADHPNQAAA
LPDWPAWNRRIQSWNRQDKPLWRTLSFLRVKAIAHRNEQYRIFAKRLAERYAYIVLEDMDIQDMNRKPQAEQA
PETSQQKLRHLARAAAPAAVRSAIENASWRWGSTFVKVDPANTTRRHAPCGNLVEQNYAESVMVYCPECKVWY
DQDENAAVNLLLRIRENPPPAPTNPKPANGSRWQRAKAKAR (SEQ ID NO: 158)
>3300006855|Ga0079044_1002244_2
[aquatic-thermal springs-hot spring]
MGPPAEAPGPRRDTAHSTEEEIASMRVYRYGAKLRGPLDPVAEEQVELANRFWNELVDMHRKYGELLQKAQEE
ASPALAALRAEMAALAEEKIRLRGLIKKSRQKARGNVPADPAIKEQLRAVSQRIKELKPIVKMEKEKAKTASS
DERHRLSEQQKLEKKRLRQKYAALGLYWSNYNAVLQGFDTAVKRELETQGRLRVRKHAPSGAAVWTVRIQHPT
GAREYTWADATRGDPSKPFSIIMPDSEREEFTTHDGRTLSRRRLPVARLRVRAERAKTPDGTWVEFGGHHIDV
PFYMHRQPPPTARVVMARLVRKRIADCYEYHLCITVDEPPAPKRSGTAAGVDLGWRRLPDGAVRVAYVAGEDG
AKGALAVPQSTLDRLAHAERLQGIRDSALEGIRSDFVAWAKPLLGNPALPDFVAAALAGDREHGIPPLASWRS
PRRFARLTGQLVRWAADHPNQAAALPDWPAWNRRIQSWNRQDKPLWRTLSFLRVKAIAHRNEQYRIFAKRLAE
RYAYIVLEDMDIQDMNRKPQAEQAPETSQQKLRHLARAAAPAAVRSAIENASWRWGSTFVKVDPANTTRRHAP
CGNLVEQNYAESVMVYCPECKVWYDQDENAAVNLLLRIRENPPPAPTNPKPANGSRWQRAKAKAR (SEQ ID
NO: 159)
>3300009503|Ga0123519_10000481_19
[aquatic-thermal springs-hot spring]
MRQLRVAHEVYNTLVQYERERRKAVADATRETDAEVARLEAEVEGLLSRLADLRAAIQAARAGGGDNARLAEA
QAEARECRRLLGEAKGALRETKRVARQNPALRERLEAIKAEHHRRQLALYHEVVEVGKRLYWPSWNDTKAAVE
QAAKKTKNGDLRFRRWTGEGSLYTQVQGKQPVCETATSRWVRIDPVPPEAHDPATPRGERRRLCRTRFYLRIG
STGPREDPVFAVFPMVYHRPLPEGAVICGARIVRRKNADREYWQAVVTVDLPDEAAQKSGPRVCALDIGWRDR
RPGGSDEPPPLRVAAWYDGDRTGEVLVDPSVFERCAKADAIRSTRDRMLDDLRAWLCEARKDLPEHLAEALAG
CGLWRAAGKFARLRGLLSSGDVPAEVRDRFLAWYHRDRHLWQYEHGMRLNAIRDRDNAYRIAAKRFAQEYDVL
IVEATGTPQKERDPKAPAAMDLRPLIKEPDPEDAPPRDQQRERKENKAHHQRFIAAAGTFRRYLLEAAAKYGT
RVVMVPCEQTTLECWVCGAKYEFDRWPLMHECESCGTTWDQDQNAARNLFARGAVAAKGPGPLEVQGKPRLPR
WHKRHKAYREGGAG (SEQ ID NO: 160)
>3300009503|Ga0123519_10000481_22
[aquatic-thermal springs-hot spring]
MATRNCRYGLLAPVEGRDEVMRQLRVAHEVYNTLVQYERERRKAVADATRETDAEVARLEAEVEGLLSRLADL
RAAIQAARAGGGDNARLAEAQAEARECRRLLGEAKGALRETKRVARQNPALRERLEAIKAEHHRRQLALYHEV
VEVGKRLYWPSWNDTKAAVEQAAKKTKNGDLRFRRWTGEGSLYTQVQGKQPVCETATSRWVRIDPVPPEAHDP
ATPRGERRRLCRTRFYLRIGSTGPREDPVFAVFPMVYHRPLPEGAVICGARIVRRKNADREYWQAVVTVDLPD
EAAQKSGPRVCALDIGWRDRRPGGSDEPPPLRVAAWYDGDRTGEVLVDPSVFERCAKADAIRSTRDRMLDDLR
AWLCEARKDLPEHLAEALAGCGLWRAAGKFARLRGLLSSGDVPAEVRDRFLAWYHRDRHLWQYEHGMRLNAIR
DRDNAYRIAAKRFAQEYDVLIVEATGTPQKERDPKAPAAMDLRPLIKEPDPEDAPPRDQQRERKENKAHHQRF
IAAAGTFRRYLLEAAAKYGTRVVMVPCEQTTLECWVCGAKYEFDRWPLMHECESCGTTWDQDQNAARNLFARG
AVAAKGPGPLEVQGKPRLPRWHKRHKAYREGGAG (SEQ ID NO: 161)
>3300006865|Ga0073934_10032691_1
[aquatic-thermal springs-hot spring sediment]
MFGHESLPSRIYSYGTMKLGDFPGRDKAEEQMRLAHRYRNRLVEIELARRRAVEEALRRLSPDLVGCELAIEA
QERALEVARSSIRRASAEARKKVASPEARDAAKTAIAHLKRERAKRMSLRKALFSSSDWEAEEKRISDEAGAA
IRKARAECGLYWGTYLHVEGTVKRTGAPPRFHRWDGSGHLAVQIQHGMTWAEALAGADNRLRVRHAPPTNSKH
SQLLHVVSVRVGSTEDGFPVWADVPRVVLHRPIPDGARIKWVHLIRRRIGCSQKWHVQFVVSAESWERTDRAT
SGTVGINVGWRMRPDGSLRVAAFCGDDGRRGELCLPSRWLAQWKKTEDIRSIRDRNFDDVRTAIANWVKGTIP
EHVRALTGEVMPELPPWWRQRAATLASWKSPARLAALTLHWRANRFAGDAVMFPLVEDWRRRDRHLYEYERHL
ADQLLAEREDLYRVFAADLRRRYKTAIVMELDLRDFHVLPPAEEPTPDGALREHTRDACLSLLHRCLDESMSE
VIRSDPRNVTRMCRECGGLNDWDRKVLHRVCSWCHAEWDQDENAARNLRDRTGGGASDKVA (SEQ ID NO:
162)
>3300001340|JGI20133J14441_1002607_2
[aquatic-thermal springs-hypersaline mat]
MPFGKKRSDKVAIVYEYGCLPPEGGLPAVAERQLVLADDFWNSLADIDRRHRAKMREILDDGELGKLNAHITS
CKARIEELRGQIKGVNQRERRNAGVDANTKAEIARLKAEVKATAARIKEIKPEHIAKQKPLLEENDALRQAAV
KRARQWFSDRGLYWGTYNAVLRSYETAHKVLLKSGEQMQAHRYTGEGRWVVQIITTAGEKPTTAEDLATGTMV
QIDPVDFSDWKHISRGERRRRARTKCRIRVGSEGRAPVWLELPCVMHRPLPEGAEIVGADVTRRLVGPARWEY
RLHLTLRVPAPVPADAAKPAIGVDIGWRALPNGGTRVAYAVGEDGSRKEVVCPDDILAGLAKSSDLRSLRDEK
MNRIKAFLRDVIPGLDSADLSEQTEHLAMWKSPKRLIRLYRWWKEHRVEGDTEAFGRLHEWYYHDYWHLYQYE
DDMRQQVLARRRDMYRIAAKEIAERASVVVIEEFDLRKFAQEDQPEDGEDNKIQRARRVAVAPSEFRIALRQA
CAARGVRVVEKPAQNTTRVHVVCGQVVAADYAADVTVRCPRCGVAYDQDANAALNLLGAGRGESTPAAS
(SEQ ID NO: 163)
>3300009784|Ga0123357_10000018_105
[arthropoda-digestive system-termite gut]
MITVVQYGVWHKWMRDVPRDVMDQLWLSHCVREDFVSTTLAYDARLKEIWSSFPVVGEAECRLLEASDALDVL
LEEQRVVRQSSRSKKVSADLRSRLADARGVVRAARVGRRDAIQVAKDAAMPLIVQAKDAQREARRGLYAKYCS
YGVPDRDGRVIRLYHATFNDVRVMHEAAEKRLASSRKQGGRGQMRHHRFDGTGTLCVSLLRTAGDPPRTPMVV
ANSESGAYRNVLGVPWVAPVVWEGLSRSQRRADGRVGVRMRVGYGDDLKSPTHVDIPVQAHRFLPAEADITGA
KLSIRRRGTKLIGSLSITAKDVPDPLPVKDGPSIVLHWGWRDVPTGGAEVARWVSTSPLDIPVDMRGVFTCHD
ESRMSGAVIAPAVMFTKLDHVEALQSELDTAFNEARGVLSEWLRAHPDVVVDDPTSREPVVLTGAVVGAWRSH
ERLARLAWAWHRECPAGVEDMESVLWEWRCGHRHVSNIAANTRARAINARRDVYRNVAAVISGQCGGVGVDDM
DLARLASRGASSELPDTVTAPGSRRRVYAAPGELRYCIVSACQKDGVTVVTLDTAENSHTCHACGYANPGDDR
WLNPMVLCDGCGKVFDQNTNALLNLVDKYTATLAV (SEQ ID NO: 164)
>3300009784|Ga0123357_10000074_42
[arthropoda-digestive system-termite gut]
MVVAPPPVCDLRGNIPWILSWIIDEPAMLGMLLALYAGTLSEMIRVYRYGLLAPTMNGKLVKEQMRAAHRYRN
ALIEIECARRDALRRLLTESGLRELEEETAAANEAVHAAAAAAREARMTVQSKSEPIDARQRMRDAREVSRRA
LDALRVRRREVRENHAVQRAMDEINERAARLRRGARALCGVYWGTYLLVEDADHRARAAALYDGAQPNNPRCS
RFVGAGRVGVQIQKGFPCETLFGSDARLRVAPVDSGAWHSMRRGERRQLSRTTLSLRIGSEGRDPIWAQWPML
MHRPLPEGSIVKRATVSVRRRGPRDEWAVEITVDVADEILAVQRTDSNESAVAIHIGWRAIGNELRVAAWAGS
DGRSGELRLPASLLGAFAKVEELRSIRDRNLAAARDALSGWLAAAASIPEWLREATVGIMEWRVPSRLAVLAK
QWRNVRFVGDEKAYEALEAWRYHDYHLWSWEDSQRIHALRARRELYRIFAAQLAREYVSIVIEDFDLRVVAKR
HLVEDASIEWRGLRRNRQAAAVSELRASLQNASKSRSARIELLDTRSFLQPCHACGSKERFDSVEPLDHSCSG
CGAIWDRDSNAALVLLQRWRREHACGGEVAHADVDTKPVPEGRWVRARRHRAEKDAHARFGASDNSEWFGNGN
ASVISIESPS (SEQ ID NO: 165)
>3300009784|Ga0123357_10000076_32
[arthropoda-digestive system-termite gut]
MITVIKYGVWHTWSRHIPDSVRDQLWLAHCAREDLVTTTLDYHDALKDIWSSFPEVAAAEQRIRDADDLLATL
LDEQSKARQASQSKKVPTDLRQRLTQARASVRSAKQERRDAITTAGVIATPLIAQAKDAQYARRKELYTTYCT
RGIPDRDGRIVRLYHATFNDVRTSHETAEKRISASRANGSPAQMRHHRFDETGTLAVSLLRQAGAPPRTPQVL
ADTETGKYRNVLAMPWFTPDAWAGKTRAQQRVDGRVTLRMRIGYADDLSSPTFVDLPVQAHRFFPPEADITGA
KLTIQRRGTTFHATVSITGKGLPDPTPVTSGPAVVLHWGWREVDTDIVEVARWAADAPLHIPDDMGDVFTTDG
SGTGGSILTPKTVFTRLNHVEKLQSEQGTAFSSAKNALVSWLSTHATPMGDPTSKQPQPLAPALVDAWRSPDR
LARLAWLWRDDRPDGADDLTADLLAWRGAYRHTATLIANTRAKAISHRNDVYRNVAAVISGQAATVGLDSMDL
ATIAATSARSELSGDVTQPGARRRTYAAPGTLREYIAAACAKDGVTVSSLDSSHASRTHYECGHTNPRDTKWL
NPIVRCDGCGESFDQNTNALHHLQARQRDLSLTA (SEQ ID NO: 166)
>BBPF01004549_6
[groundwater metagenome]
MPFGRKAKPCRVFEYGCLPPVSGKDELLKELRLRNNYWNKLVEIDRLIRQRSALILLLPGDIEAAHLDAQIDL
MRGEIKKGRQRTRSSITDADLKQRIKDSIAELRVLWEQNKKDRKPLIETTRADLAAIETEWRVARKAARADSG
LYWCNYDDVDTAYDVARKETAKKWAFPKFRRFDGTGKVTVRWQNGLNANNVFDGTGTLLQIAPVHQDAWNHPV
RSNRRKASRTTVRFRVRSENRSPVWVELPMVMHRPLPAGGEIRSASLVCGYVGGKPTYKLVITVAPPAHTLPE
EGMHRGIRPTVGINLGWRKKDNDIRIAYWADEEGRHGELTLTSNTLAQFSKLNDLKSIRDKYFNEAISALALY
ISEGTIPDWLKADTTHLNKWRSKPRLLALVGKWRETRFTGDEIIYEALFYWRGRELHLHQWEANLRDQVQRHR
RERYRIFAAQLAKDYSQIFIENHNLVVTKKKKATEDGTYLTTEVDTLRTIASPGILRGQIENACRREGVIFTK
LDAKHITSKCHICGWQEKWNAAATITRECPGCKTEWDQDYNAARLLLQRGLDGGYLAVPQTTLEDDPNFCIGS
(SEQ ID NO: 167)
>BBPG01001333_4
[groundwater metagenome]
MPFGRKAKPCRVFEYGCLPPVSGKDELLKELRLRNNYWNKLVEIDRLIRQRSALILLLPGDIEAAHLDAQIDL
MRGEIKKGRQRTRSSITDADLKQRIKDSIAELRVLWEQNKKDRKPLIETTRADLAAIETEWRVARKAARADSG
LYWCNYDDVDTAYDVARKETAKKWAFPKFRRFDGTGKVTVRWQNGLNANNVFDGTGTLLQIAPVHQDAWNHPV
RSNRRKASRTTVRFRVRSENRSPVWVELPMVMHRPLPAGGEIRSASLVCGYVGGKPTYKLVITVAPPAHTLPE
EGMHRGIRPTVGINLGWRKKDNDIRIAYWADEEGRHGELTLTSNTLAQFSKLNDLKSIRDKYFNEAISALALY
ISEGTIPDWLKADTTHLNKWRSKPRLLALVGKWRETRFTGDEIIYEALFYWRGRELHLHQWEANLRDQVQRHR
RERYRIFAAQLAKDYSQIFIENHNLVVTKKKKATEDGTYLTTEVDTLRTIASPGILRGQIENACRREGVIFTK
LDAKHITSKCHICGWQEKWNAAATITRECPGCKTEWDQDYNAARLLLQRGLDGGYLAVPQTTLEDDPNFCIGS
(SEQ ID NO: 167)
>OGZV01009429_1
[human gut metagenome]
MNKVSITKVFKYRCFEPVEGLALFDEALDNRHTLWNKLVEIDRDFREKSTQIITPIKSDYQVLDQEIKNLQDS
IKAIKRKTRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRVNTIFYIEPVNQELYNSPI
RGVRKKASVTRCHIRVGSADKGKPIFVTLPMVYHRPLPMDGKINAINVKRHYIDHKPIYECLITVTYAIEAPT
INPNSCVAVDLGWRMTKDGLRAAYAIDTDDKTIECIVTQRQLNEFDTICGLSSTRQKHLNDCIHVIKAWMANK
NLPDWLTDAVAYIDKWKSYTHIFDLHSAFLQHKKSGNQEIVSYLEAYIERENHLRTWQSNLQNQVIARRNYEY
QNFAAKLANMYDVLVLEKLSITNIVKHQKAIIGSQQSTAVDRNRTIVAPYVLKTILINAFRSRGKQFVEVNAS
YSTKVCHHCGALEEVHQSSIMHTCTQCHTVWDQDYNACINLLALYNHDNKGGVSCV (SEQ ID NO: 168)
>OKWZ01000119_10
[human gut metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHICTQCHTVWDQDYNACINLLALYDNKVGAY (SEQ ID NO: 169)
>ODGR01000476_16
[human metagenome]
MNKVSITKVFKYRCFEPVEGLALFDEALDNRHTLWNKLVEIDRDFREKSSQIITPIQSDYQVLDQEIKNLQDS
IKAIKRKTRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRVNTIFYIEPVNQELYNSPI
RGVRKKASVTRCHIRVGSADKGKPIFVTLPMVYHRPLPMDGKINAINVKRHYFDHKPIYECLITVTYAIEAPT
INPNSCVAVDLGWRMTKDGLRAAYAIDTDDKTIECIVTQRQLNEFDTICGLSSTRQKHLNDCIHVIKAWMANK
NLPDWLTDAVAYIDKWKSYTHIFDLHSAFLQHKKSGNQEIVSYLEAYIERENHLRTWQSNLQNQVIARRNYEY
QNFAAKLANMYDVLVLEKLSITDIVKHQKAMIGSQQSTALDRNRTIVAPYELKTILINAFTNRGKQFVEVNAS
YSTKVCHHCGALEEVHQSSIMHICTQCHTVWDQDYNACINLLALYNHNNKVGVSCV (SEQ ID NO: 170)
>ODIG01000268_14
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVIHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>ODIP01002140_2
[human metagenome]
MNKVSITKVFKYRCFEPVEGLALFDEALDNRHMLWNKLVEIDRNFREKSSQIITPIQSDYQVLDQKIKNLQDS
IKAIKRKTRSTAKEETRNIQDTIKELKVKRKEAYQEFKRLKHEAIEREKPLLDCLNNERKEQVKQLRSKSGLH
GFNFDDVIHNIYDVARVKAMKQGTLLRFKRYSKNGKIAVRPYSNSPLYGADIHKVNTIFYIEPVNQELYNSPI
RGVRKKASVTQCHIRVGSADKGKPIFVTLPMVYHRPLPMDGKINAINVKRHYIDHKPIYECLITVTYVIEAPA
VNPSSCVAVDLGWRMTKDGLRAAYATDKDNKTMECIVAQRQLNEFDTIRGLSSTRQKHFNDCIHVIKAWIANK
NLPDWLIDAVAYIDKWKSYTHIFELYNAFLQHEKSGNQEIVSYLEAYIERENHLRIWQSNLQDQVIAKRNYEY
QNFAAKLANMYDVLVIEKLSITDIVKHQKAIIGSQQSTALDRNRTIVAPYVLKTILINAFTSRGKQFVEVNAS
YSTKVCHHCGALEEVHQSSIMHTCTQCHTIWDQDYNACINLLTLYNHDNKVGVSCV (SEQ ID NO: 171)
>ODIW01000227_18
[human metagenome]
MNKVSITKVFKYRCFEPVEGLALFDEALDNRHMLWNKLVEIDRNFREKSSQIITPIQSDYQVLDQKIKNLQDS
IKAIKRKTRSTAKEETRNIQDTIKELKVKRKEAYQEFKRLKHEAIEREKPLLDCLNNERKEQVKQLRSKSGLH
GFNFDDVIHNIYDVARVKAMKQGTLLRFKRYSKNGKIAVRPYSNSPLYGADIHKVNTIFYIEPVNQELYNSPI
RGVRKKASVTQCHIRVGSADKGKPIFVTLPMVYHRPLPMDGKINAINVKRHYIDHKPIYECLITVTYVIEAPA
VNPSSCVAVDLGWRMTKDGLRAAYATDKDNKTMECIVAQRQLNEFDTIRGLSSTRQKHFNDCIHVIKAWIANK
NLPDWLIDAVAYIDKWKSYTHIFELYNAFLQHEKSGNQEIVSYLEAYIERENHLRIWQSNLQDQVIAKRNYEY
QNFAAKLANMYDVLVIEKLSITDIVKHQKAIIGSQQSTALDRNRTIVAPYVLKTILINAFTSRGKQFVEVNAS
YSTKVCHHCGALEEVHQSSIMHTCTQCHTIWDQDYNACINLLTLYNHDNKVGVSCV (SEQ ID NO: 171)
>ODJA01000260_38
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>ODJP01000229_55
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>ODKZ01007116_1
[human metagenome]
MNKVSITKVFKYRCFEPVEGLALFDEALDNRHTLWNKLVEIDRNFREKSSQIITPIQSDYQVLDQEIKNLQDS
IKAIKRKTRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRVNTIFYIEPVNQELYNSPI
RGVRKKASVTRCHIRVGSADKGKPIFVTLPMVYHRPLPMDGKINAINVKRHYIDHKPIYECLITVTYAIEAPT
INPNSCVAVDLGWRMTKDGLRAAYATDTDDKTIECIVTQRQLNEFDTICGLSSTRQKHLNDCIHVIKAWMANK
NLPDWLTDAVAYIDKWKSYTHIFDLHSAFLQHKKSGNQEIVSYLEAYIERENHLRTWQSNLQNQVIARRNYEY
QNFASKLANMYDVLVLEKLSITDIVKHQKAMIGSQQSTALDRNRTIVAPYELKTILINAFTNRGKQFVEVNAS
YSTKVCHHCGALEEVHQSSIMHTCTQCHTVWDQDYNACINLLALYNHNNKVGVSCV (SEQ ID NO: 172)
>ODM001000523_12
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>ODTN01000195_35
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>ODTP01000194_18
[human metagenome]
MNKVSITKVFKYRCFEPVEGLALFDEALDNRHTLWNKLVEIDRNFREKSSQIITPIQSDYQVLDQEIKNLQDS
IKAIKRKTRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRVNTIFYIEPVNQELYNSPI
RGVRKKASVTRCHIRVGSADKGKPIFVTLPMVYHRPLPMDGKINAINVKRHYIDHKPIYECLITVTYAIEAPT
INPNSCVAVDLGWRMTKDGLRAAYATDTDDKTIECIVTQRQLNEFDTICGLSSTRQKHLNDCIHVIKAWMANK
NLPDWLTDAVAYIDKWKSYTHIFDLHSAFLQHKKSGNQEIVSYLEAYIERENHLRTWQSNLQNQVIARRNYEY
QNFASKLANMYDVLVLEKLSITDIVKHQKAMIGSQQSTALDRNRTIVAPYELKTILINAFTNRGKQFVEVNAS
YSTKVCHHCGALEEVHQSSIMHTCTQCHTVWDQDYNACINLLALYNHNNKVGVSCV (SEQ ID NO: 172)
>ODWI01002981_3
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>ODZZ01005262_2
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>OEED01000500_25
[human metagenome]
MNKVSITKVFKYRCFEPVEGLALFDEALDNRHMLWNKLVEIDRNFREKSSQIITPIQSDYQVLDQKIKNLQDS
IKAIKRKTRSTAKEETRNIQDTIKELKVKRKEAYQEFKRLKHEAIEREKPLLDCLNNERKEQVKQLRSKSGLH
GFNFDDVIHNIYDVARVKAMKQGTLLRFKRYSKNGKIAVRPYSNSPLYGADIHKVNTIFYIEPVNQELYNSPI
RGVRKKASVTQCHIRVGSADKGKPIFVTLPMVYHRPLPMDGKINAINVKRHYIDHKPIYECLITVTYVIEAPA
VNPSSCVAVDLGWRMTKDGLRAAYATDKDNKTMECIVAQRQLNEFDTIRGLSSTRQKHFNDCIHVIKAWIANK
NLPDWLIDAVAYIDKWKSYTHIFELYNAFLQHEKSGNQEIVSYLEAYIERENHLRIWQSNLQDQVIAKRNYEY
QNFAAKLANMYDVLVIEKLSITDIVKHQKAIIGSQQSTALDRNRTIVAPYVLKTILINAFTSRGKQFVEVNAS
YSTKVCHHCGALEEVHQSSIMHICTQCHTIWDQDYNACINLLALYNHDNKVGVSCV (SEQ ID NO: 173)
>OEFT01000529_3
[human metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVIHNIYEVARVKAMKQGILLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHICTQCHTVWDQDYNACINLLALYDNKVGAY (SEQ ID NO: 169)
>LAZR01002400_15
[marine sediment metagenome]
MNLGRVYYNSLVEAENERRTTMWGGDRPPSPATHVCKKSCSTCDKAESKKRKPRKHECKKFCPVCRAHYKALR
KQYRSEPPLDVKPFRKKAAEGGLYWGTYLVIEQDFSAAWKETESFSLVKFRSWRQGDMCAVQIQRDKDPDRMF
LIKSAPDPRKKKQQRYTLRLRVGSKGQAPVWAEPLPFEMHRPLQGTATWVKIARKYVADRVIWSVQFTRRDIP
ERKDNAERGAVAIDVGWRKTDDGMRIAYARGDDGAEYELVLPPKWMKHADQADRIRSARDQNLVELQKQERFW
SVILAVCGFSNKKLFARLKSTLSVRRVAKPGEHTKWIKKERHLWQYEAGCRNRSVTRRRNDVRVWLRDLRRRY
AHAVIKDSCHKKMKENKTSLPKPARRQGHHAAPGEVIEEITRVFGRITGVSVVCAVDTTNHCPACSFVNSYGP
ERVVTCGGCGVVEDRDRVSTQNMMNMYAIGNVRNPTTRKSTPRFAKKHKDPEAP (SEQ ID NO: 174)
>LAZR01002400_19
[marine sediment metagenome]
MTKVYKYGALPGGDTLCAQMNLGRVYYNSLVEAENERRTTMWGGDRPPSPATHVCKKSCSTCDKAESKKRKPR
KHECKKFCPVCRAHYKALRKQYRSEPPLDVKPFRKKAAEGGLYWGTYLVIEQDFSAAWKETESFSLVKFRSWR
QGDMCAVQIQRDKDPDRMFLIKSAPDPRKKKQQRYTLRLRVGSKGQAPVWAEPLPFEMHRPLQGTATWVKIAR
KYVADRVIWSVQFTRRDIPERKDNAERGAVAIDVGWRKTDDGMRIAYARGDDGAEYELVLPPKWMKHADQADR
IRSARDQNLVELQKQERFWSVILAVCGFSNKKLFARLKSTLSVRRVAKPGEHTKWIKKERHLWQYEAGCRNRS
VTRRRNDVRVWLRDLRRRYAHAVIKDSCHKKMKENKTSLPKPARRQGHHAAPGEVIEEITRVFGRITGVSVVC
AVDTTNHCPACSFVNSYGPERVVTCGGCGVVEDRDRVSTQNMMNMYAIGNVRNPTTRKSTPRFAKKHKDPEAP
(SEQ ID NO: 175)
>FLSK01003024_2
[metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHICTQCHTVWDQDYNACINLLALYDNKVGAY (SEQ ID NO: 169)
>OFLM01000072_9
[metagenome]
MNKVSITKVFKYRCFEPVEGLALFDEALDNRHTLWNQLVEIDRNFREKSSQIITPIQSDYQVLDQEIKNLQDS
IKAIKRKTRSTAKEETRIIKDTIKELKVKRKEAYQEFKRLKHEAIEREKPLLDCLNNERKEQVKQLRSKSGLH
GFNFDDVIHNIYDVARVKAMKQGTLLRFKRYSKNGKIAVRPYSNSPLYGADIHKVNTIFYIEPVNQELYNSPI
RGVRKKASVTQCHIRVGSADKGKPIFVTLPMVYHRPLPMDGKINAINVKRHYIDHKPIYECLITVTYVIEAPA
VNPSSCVAVDLGWRMTKDGLRAAYATDKDNKTMECIVAQRQLNEFDTIRGLSSTRQKHFNDCIHVIKAWIANK
NLPDWLIDAVAYIDKWKSYTHIFELYNAFLQHEKSGNQEIVSYLEAYIERENHLRIWQSNLQDQVIAKRNYEY
QNFAAKLANMYDVLVIEKLSITDIVKHQKAIIGSQQSTALDRNRTIVAPYVLKTILINAFTSRGKQFVEVNAS
YSTKVCHHCGALEEVHQSSIMHICTQCHTIWDQDYNACINLLALYNHDNKVGVSCV (SEQ ID NO: 176)
>OFLO01000090_50
[metagenome]
MNKVSITKVFKYRCFEPVEGLALFDEALDNRHTLWNQLVEIDRNFREKSSQIITPIQSDYQVLDQEIKNLQDS
IKAIKRKTRSTAKEETRIIKDTIKELKVKRKEAYQEFKRLKHEAIEREKPLLDCLNNERKEQVKQLRSKSGLH
GFNFDDVIHNIYDVARVKAMKQGTLLRFKRYSKNGKIAVRPYSNSPLYGADIHKVNTIFYIEPVNQELYNSPI
RGVRKKASVTQCHIRVGSADKGKPIFVTLPMVYHRPLPMDGKINAINVKRHYIDHKPIYECLITVTYVIEAPA
VNPSSCVAVDLGWRMTKDGLRAAYATDKDNKTMECIVAQRQLNEFDTIRGLSSTRQKHFNDCIHVIKAWIANK
NLPDWLIDAVAYIDKWKSYTHIFELYNAFLQHEKSGNQEIVSYLEAYIERENHLRIWQSNLQDQVIAKRNYEY
QNFAAKLANMYDVLVIEKLSITDIVKHQKAIIGSQQSTALDRNRTIVAPYVLKTILINAFTSRGKQFVEVNAS
YSTKVCHHCGALEEVHQSSIMHICTQCHTIWDQDYNACINLLALYNHDNKVGVSCV (SEQ ID NO: 176)
>OFLU01000140_22
[metagenome]
MNKVSITKVFKYRCFEPVEGLALFDEALDNRHTLWNKLVEIDRDFREKSSQIITPIQSDYQVLDQEIKNLQDS
IKAIKRKTRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRANTIFYIEPVNQELYNSSI
RGVRKKASVTRCHIRVGSADKGKPIFVTLPMVYHRPLPMDGKINAINVKRHYIDHKPIYECLITVIYAIEAPT
INPNSCVAVDLGWRMTKDGLRAAYATDTDDKTIECIVTQRQLNEFDTIRGLSSTRQKHFNDCIHVIKAWMANK
NLPDWLTDAVTYIDKWKSYKHIFELHDAFLQHKKSGNQEIVSYLEAYIERENHLRTWQSNLQDQVIARRNYEY
QNFAAKLANMYDVLVLEKLSITDIVKHQKAIIGSQQSTALDRNRTIVAPYELKKILINAFTNRGKQFVEVNAS
YSTKVCHHCGALEEVHQSSIMHTCTQCHTVWDQDYNACINLLALYNHDNKVGVSYV (SEQ ID NO: 177)
>OFLV01000230_3
[metagenome]
MNKVSITKVFKYRCFEPVEGLALFDEALDNRHTLWNKLVEIDRDFREKSSQIITPIQSDYQVLDQEIKNLQDS
IKAIKRKTRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRANTIFYIEPVNQELYNSSI
RGVRKKASVTRCHIRVGSADKGKPIFVTLPMVYHRPLPMDGKINAINVKRHYIDHKPIYECLITVIYAIEAPT
INPNSCVAVDLGWRMTKDGLRAAYATDTDDKTIECIVTQRQLNEFDTIRGLSSTRQKHFNDCIHVIKAWMANK
NLPDWLTDAVTYIDKWKSYKHIFELHDAFLQHKKSGNQEIVSYLEAYIERENHLRTWQSNLQDQVIARRNYEY
QNFAAKLANMYDVLVLEKLSITDIVKHQKAIIGSQQSTALDRNRTIVAPYELKKILINAFTNRGKQFVEVNAS
YSTKVCHHCGALEEVHQSSIMHTCTQCHTVWDQDYNACINLLALYNHDNKVGVSYV (SEQ ID NO: 177)
>OGCY01000078_30
[metagenome]
MNKVSITKVFKYRCFEPFEGLELFNEALDNRHTLWNKLVEIDRDFREKSSQIITPIQSEYQILDQEIKNLQDS
IKAIKRETRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRVNTIFYIEPVNQELYNSSI
RGVRKKASVTRCHIRVGSADKGKPIFVTLPMVYHRPLPMDGKINAINVKRHYIDHKPIYECLITVIYAIEAPT
INPNSCVAVDLGWRMTKDGLRAAYATDTDDKTIECIVTQRQLNEFDTIRGLSSTRQKHFNDCIHVIKAWMANK
NLPDWLTDAVAYIDKWKSYKHIFDLYDAFLQHKKSGNQEIVSYLEAYIERENHLRTWQSNLQDQVIARRNYEY
QNFAAKLANMYDVLVLEKLSITDIVKHQKAIIGSQQSTALDRNRTIVAPYELKTILINAFTNRGKQFVEVNAS
YSTKVCHHCGALEEVHQSSIMHICTQCHTVWDQDYNACINLLALYNHDNKVGVSYV (SEQ ID NO: 178)
>OGJO01000473_2
[metagenome]
MNKVSITKVFKYRCFEPVEGLSLFDEALDNRHMLWNKLVEIDRDFREKSSKIITPIQSDYQVLDQEIKNLQDA
VKAIKCKTRSTAKEETRTIQDTIKELKVKRKEAYQEFKRLKHEAIEREKPLLDCLNNERKEKVKQLRSKSGLH
GFNFDDVTHNIYEVARVKAMKQGTLLRFKRYSKNGKIAVRPYSNSPLYGSDIHRVNTIFYIDPVNQELYNSPI
RGVRKKASVTQCHIRIGSAVKGKPIFVTLPMVYHRPLPMDGKINAINIKRHYIDQKPIYECFITVTYLIEAPA
VNPNSCVAVDLGWRMTKDGLRAAYATDKDHKAIECIVTQRQLNEFDTIRGLSSTRQKHFNDCIHVIKAWIANK
NLPDWLIDAVAYIDKWKSYTHIFKLYNAFLQHEKSGNQEIISYLEAYIERENHLRIWQSNLQDQAIAKRNYEY
QNFAARLANMYDVLVLEKLSITDIVKYQKAIIGSQQSTALDRNRTIVAPYELKTILINAFTSRGKQFVEVNAS
YSTKVCHHCGALEEVHQSSIMHTCTQCHTVWDQDYNACINLLALYDNKVGAY (SEQ ID NO: 169)
>OGJT01000109_37
[metagenome]
MNKVSITKVFKYRCFEPVEGLALFDEALDNRHTLWNKLVEIDRDFREKSTQIITPIQSDYQVLDQEIKNLQDS
IKAIKRKTRSTAKEETRNIQDTIKELKVKRKEAYQEFKRLKHEAIEREKPLLDCLNNERKETVKQLRSESGLH
GFNFDDVIHNIYDVARIKAMKQGTLLQFKRYSKNGKIAVRPYSSSPLYGSDIHRINTIFYIEPVNQELYNSSI
RGIRKKASVTRCHIRVGSADKGKPIFVTLPMVYHRPLPMDGKINAINVKRHYIDHKPIYECLITVIYAIEAPT
INPNSCVAVDLGWRMTKDGLRAAYAIDTDDKTIECIVTQRQLNEFDTIRGLSSTRQKHFNDCIHVIKAWMANK
NLPDWLTDAVAYIDKWKSYKHIFDLYDAFLQHKKSGNQEIVSYLESYIERENHLRTWQSNLQVQVIARRNYEY
QNFAAKLANMYDVLVLEKLSITDIVKHQKAIIGSQQSTALDRNRTIVAPYELKKILINAFTNRGKQFVEVNAS
YSTKVCHHCGALEEVHQSSIMHTCTQCHTVWDQDYNACINLLALYNHDNKVGVSNV (SEQ ID NO: 179)
>OGJZ01005194_5
[metagenome]
MNKVSITKVFKYRCFEPVEGLALFDEALDNRHTLWNKLVEIDRDFREKSSQIITPIQSDYQVLDQEIKSLQDS
IKAIKRKTRSTAKEETRNIQDTIKELKVKRKEAYQEFKRLKHEAIEREKPLLDCLNHERKEKVKQLRSESGLH
GFNFDDVIHNIYDVARIKAMKQGTLLRFKRYSKNGKIAVRPYSSSPLYGSDIHRVNTIFYIEPVNQELYNSSI
RGVRKKASVTRCHIRVGSADKGKPIFVTLPMVYHRPLPMDGKINAINVKRHYIDHKPIYECLITVTYAIKSPA
VNPDSCVAVDLGWRMTKDGLRAAYATDTDNKTMECIVAQRQLNEFDTIRGLSSTRQKHFNDCIHVIKAWMANK
NLPDWLTDAVAYIDKWKSYKHIFDLYDAFLQHKKSGNQEIVSYLEAYIERENHLRIWQSNLQDQVIARRNYGY
QNFAAKLANMYDVLVLEKLSITDIVKHQKAIIGSQQSTALDRNRTIVAPYELKTILINAFTNRGKQFVEVNAS
YSTKVCHHCGALEEVHQSSIMHTCTQCHTVWDQDYNACINLLALYNHDNKVGVSYV (SEQ ID NO: 180)
>OGKO01001669_8
[metagenome]
MNKVSITKVFKYRCFEPVEGLALFDEALDNRHTLWNKLVEIDRDFREKSSQIITPIQSDYQVLDQEIKNLQDS
IKAIKRKTRSTAKEETRNIQDTIKELKVKRKEAYQEFKRLKHEAIEREKPLLDCLNNERKEKVKQLRSESGLH
GFNFDDVIHNIYDVARIKAMKQGTLLQFKRYSKNGKIAVRPYSSSPLYGSDIHRVNTIFYIESVNQELYNSPI
RGVRKKASVTRCHIRVGSADKGKPIFVTLPMVYHRPLPMDGKINAINVKRHYIDHKPIYECLITVTYAIEAPT
INPNSCVAVDLGWRMTKDGLRAAYATDTDDKTIECIVTQRQLNEFDTIRGLSSTRQKHFNDCIHVIKAWMANK
NLPDWLTDAVAYIDKWKSYKHIFDLYDAFLQHKKSGNQEIVSYLEAYIERENHLRTWQSNLQDQVIARRNYEY
QNFAAKLANMYDVLVLEKLSITDIVKHQKAIIGSQQSTALDRNRTIVAPYELKKILINAFTNRGKQFVEVNAS
YSTKVCHHCGALEEVHQSSIMHTCTQCHTVWDQDYNACINLLALYNHDNKVGVSYV (SEQ ID NO: 181)
>OFCI01000292_37
[metagenomes unclassified sequences.]
MWYAHRYGNVLVEIERARRAAVRLAYGPLATFEHAAKEAGTVVRDAREAIKRARAEHRARVETDAMKLALSSA
REAEREAKRALFEARRQLRESGAVDAALADIEARAGELRRGARALSGVHWGTYSLIEAAHDASRKMPLYDGVE
PNDPRFARWEHEGQVGIQVHQKGSTGTGMVAEELHAGIGGEWLRVERVHDTRQGRRAGTRARLTMRIGTSDVD
GTPIVAAWPMVMHRAVPPGAIVRRATVSLRRRGPREEWSVELTIQVPSTIAPEERQGHVAINVGWRSMGDTIR
VASWVGSDGQKGELHLSKRTIEGLRKPEGLRSTRDKNFNDARNALLLALAGLNVPEWYTLRAKALSQWRSPQR
LAALATEWKTKRFAGDESAYLALETWRYHDHHLWAWEASQSVGSHRHRREVYRVFGSQLAKRYRTLVLADFDK
RVVAIRPAVGDANDKTQNETARSNRQLASTSELEREAANAFTSRGGTAEYLSAVDITHTCADCGALSTFDAAE
RIHAACSSCGVVFDQDENAARVLCERWRDGEKVGVARVAEPQEKVGSKWQRAKARKEERQAARKAVASAAE
(SEQ ID NO: 182)
>3300006048|Ga0075363_100000001_25
[plants-endosphere-populus endosphere]
MAFGNKSKPTRKYNYGCRGFLSGPREINPDGTKALRQRFDGHDLAVTQMKLAHQFQNKLVEIERARREAIDPI
LVRHFPALTTLHNAAQEQQERLQELRDELSQSNSQNRQLMSDPELVEQINAQKRVVSQAWEFYSDQRDAAFAD
PKVKADLKDNDKSFEQRKADARNAAVEGGLYWATSLQVVGRVKRTGPPPKFKSWRGEEVISVQFQRKPDKTSP
KEPVLDSKGNPKIHPRSKKPTFAHVGGSSLRTCDVFTPNTNCWIERTYQPPLTAPHPKYVVIHFRVSSDEKGK
PVMASIPAVMSRPLPEDGEVKWVHLSRRKIGTHYRWDVQFDIARDAWTYHPAGQDRAQEGTVAVALGWRLIDG
EIRVIEWVGDDGVTGTVRIPKELVEGWSYLDTLQSIRDTLFEAERAELVDWFVNYPNPLPEEWTERAQTLIQW
RSADRFMWLIWWWKDHRIPGDEEIFQRMWGRIQLNPITGRNQYTGGRLQSKHLCDWRAHKRDKIKNWRKDFFR
KVAIDLSYQYKDVVIAEIDWNKLAENPEVENGNDIVNKRYRALSSCAQLRDEITRYMNEVTESARNIVTTCCQ
CGESCDHPKSGRWIRCESCGGEKRDRAVNAATNLLNRALGASGAKVPARV (SEQ ID NO: 183)
>3300006048|Ga0075363_100000001_20
[plants-endosphere-populus endosphere]
MVAAKISHLLEFPSMAFGNKSKPTRKYNYGCRGFLSGPREINPDGTKALRQRFDGHDLAVTQMKLAHQFQNKL
VEIERARREAIDPILVRHFPALTTLHNAAQEQQERLQELRDELSQSNSQNRQLMSDPELVEQINAQKRVVSQA
WEFYSDQRDAAFADPKVKADLKDNDKSFEQRKADARNAAVEGGLYWATSLQVVGRVKRTGPPPKFKSWRGEEV
ISVQFQRKPDKTSPKEPVLDSKGNPKIHPRSKKPTFAHVGGSSLRTCDVFTPNTNCWIERTYQPPLTAPHPKY
VVIHFRVSSDEKGKPVMASIPAVMSRPLPEDGEVKWVHLSRRKIGTHYRWDVQFDIARDAWTYHPAGQDRAQE
GTVAVALGWRLIDGEIRVTEWVGDDGVTGTVRIPKELVEGWSYLDTLQSIRDTLFEAERAELVDWFVNYPNPL
PEEWTERAQTLIQWRSADRFMWLIWWWKDHRIPGDEEIFQRMWGRIQLNPTTGRNQYTGGRLQSKHLCDWRAH
KRDKIKNWRKDFFRKVAIDLSYQYKDVVIAEIDWNKLAENPEVENGNDIVNKRYRALSSCAQLRDEITRYMNE
VTESARNIVTTCCQCGESCDHPKSGRWIRCESCGGEKRDRAVNAATNLLNRALGASGAKVPARV (SEQ ID
NO: 184)
>3300006048|Ga0075363_100000020_49
[plants-endosphere-populus endosphere]
MIVYKYGALKPKVIGGTFEDLLQYQRHSNAFYNALIEIERWRIAARDIVELAQSAPLSDEQKTEHRLAYNAAC
RAAGAATPIGWGQKQAVTEMVAAAMKTRRSDEFKARQRASKKGYDFLKRVMTCARPRHRRFDGEGILAATVQG
ATGLKASAVLTKPGPVQISGDGKHRTVTLRLREGLSLEVPIVYHRPLPERAEKEGVPYDVRVIFARLVIDRIG
DRWTYSVHLTIDAAPRVHVAQGGLGRCAVNFGWRRVPGGIRVAYAVDDDGNETSCVFPDALLGRQKHAESLRS
LADEIAAAYLGDAARRTKARCSALADPDAIHRELGREWFTMEQAAKRDGTDAEHWARRDRHLYQWERDEYASV
LRARREIYRLWARKLAASYDSVIIEAFDMRSVVKRTPSEDDIPAARHYRFLVGPHCLRLEIQSVFGARCEVLK
PAKRTLTCHACGALCKWDKARELRHDCESCGAAWDQDANNAKNQLLDAAE (SEQ ID NO: 185)
>3300006178|Ga0075367_10000108_6
[plants-endosphere-populus endosphere]
MAFGNKSKPTRKYNYGCRGFLSGPREINPDGTKALRQRFDGHDLAVTQMKLAHQFQNKLVEIERARREAIDPI
LVRHFPALTTLHNAAQEQQERLQELRDELSQSNSQNRQLMSDPELVEQINAQKRVVSQAWEFYSDQRDAAFAD
PKVKADLKDNDKSFEQRKADARNAAVEGGLYWATSLQVVGRVKRTGPPPKFKSWRGEEVISVQFQRKPDKTSP
KEPVLDSKGNPKIHPRSKKPTFAHVGGSSLRTCDVFTPNTNCWIERTYQPPLTAPHPKYVVIHFRVSSDEKGK
PVMASIPAVMSRPLPEDGEVKWVHLSRRKIGTHYRWDVQFDIARDAWTYHPAGQDRAQEGTVAVALGWRLIDG
EIRVTEWVGDDGVTGTVRIPKELVEGWSYLDTLQSIRDTLFEAERAELVDWFVNYPNPLPEEWTERAQTLIQW
RSADRFMWLIWWWKDHRIPGDEEIFQRMWGRIQLNPTTGRNQYTGGRLQSKHLCDWRAHKRDKIKNWRKDFFR
KVAIDLSYQYKDVVIAEIDWNKLAENPEVENGNDIVNKRYRALSSCAQLRDEITRYMNEVTESARNIVTTCCQ
CGESCDHPKSGRWIRCESCGGEKRDRAVNAATNLLNRALGASGAKVPARV (SEQ ID NO: 183)
>3300006178|Ga0075367_10000108_6
[plants-endosphere-populus endosphere]
MVAAKISHLLEFPSMAFGNKSKPTRKYNYGCRGFLSGPREINPDGTKALRQRFDGHDLAVTQMKLAHQFQNKL
VEIERARREAIDPILVRHFPALTTLHNAAQEQQERLQELRDELSQSNSQNRQLMSDPELVEQINAQKRVVSQA
WEFYSDQRDAAFADPKVKADLKDNDKSFEQRKADARNAAVEGGLYWATSLQVVGRVKRTGPPPKFKSWRGEEV
ISVQFQRKPDKTSPKEPVLDSKGNPKIHPRSKKPTFAHVGGSSLRTCDVFTPNTNCWIERTYQPPLTAPHPKY
VVIHFRVSSDEKGKPVMASIPAVMSRPLPEDGEVKWVHLSRRKIGTHYRWDVQFDIARDAWTYHPAGQDRAQE
GTVAVALGWRLIDGEIRVTEWVGDDGVTGTVRIPKELVEGWSYLDTLQSIRDTLFEAERAELVDWFVNYPNPL
PEEWTERAQTLIQWRSADRFMWLIWWWKDHRIPGDEEIFQRMWGRIQLNPTTGRNQYTGGRLQSKHLCDWRAH
KRDKIKNWRKDFFRKVAIDLSYQYKDVVIAEIDWNKLAENPEVENGNDIVNKRYRALSSCAQLRDEITRYMNE
VTESARNIVTTCCQCGESCDHPKSGRWIRCESCGGEKRDRAVNAATNLLNRALGASGAKVPARV (SEQ ID
NO: 184)
>3300006195|Ga0075366_10000160_13
[plants-endosphere-populus endosphere]
MAFGNKSKPTRKYNYGCRGFLSGPREINPDGTKALRQRFDGHDLAVTQMKLAHQFQNKLVEIERARREAIDPI
LVRHFPALTTLHNAAQEQQERLQELRDELSQSNSQNRQLMSDPELVEQINAQKRVVSQAWEFYSDQRDAAFAD
PKVKADLKDNDKSFEQRKADARNAAVEGGLYWATSLQVVGRVKRTGPPPKFKSWRGEEVISVQFQRKPDKTSP
KEPVLDSKGNPKIHPRSKKPTFAHVGGSSLRTCDVFTPNTNCWIERTYQPPLTAPHPKYVVIHFRVSSDEKGK
PVMASIPAVMSRPLPEDGEVKWVHLSRRKIGTHYRWDVQFDIARDAWTYHPAGQDRAQEGTVAVALGWRLIDG
EIRVTEWVGDDGVTGTVRIPKELVEGWSYLDTLQSIRDTLFEAERAELVDWFVNYPNPLPEEWTERAQTLIQW
RSADRFMWLIWWWKDHRIPGDEEIFQRMWGRIQLNPTTGRNQYTGGRLQSKHLCDWRAHKRDKIKNWRKDFFR
KVAIDLSYQYKDVVIAEIDWNKLAENPEVENGNDIVNKRYRALSSCAQLRDEITRYMNEVTESARNIVTTCCQ
CGESCDHPKSGRWIRCESCGGEKRDRAVNAATNLLNRALGASGAKVPARV (SEQ ID NO: 183)
>3300009500|Ga0116229_10010095_9
[plants-peat moss-host associated]
MTTLVYQFHLDPPVSGERAARQQMLAAHRYANDLIAIERGRRDALRAVHDTPAVREAEVLLKAATRSTRKAAV
KALWAARREAERIASEVDETLPEVAAAKAALDALPKDAPARVRSVARQTLRAARAEAGDALARIQIFDEALRR
GARALTTAHWGTYLSIEASADQARKAPLYADDALTPASPRFRFGARRGYLDESDARSVWWCARSQVGMHVQGR
VCRTSGVFAGRDAWVRLEDAEPISHDHNTRRAILALRLDVDTWVRWPIRMHREIPDAARWSWVRVSCRPQQGA
RGKELWSVEITVDDTAPRPRELAADTLRGAVAVELLWSPLDDGTMRVARWLDSEGKRGEIVLPRELVRGLGEI
PSGIRSVRDQLLNDLRPKLTRALRECTETMPTWLREAGATLHLWKSPSRFVDLARRWRASKCDAARAAYELLD
AWELRDTHLDDYENGTRARSLRRRREVYRVLAARWAQSYATVLVPDRDLSREARWGEESERRFLASPQELRDC
LRKAFGDGAVDVPWRGPHGVVDDGDEDADVPEWLEKAIEQWRDEEKSGSARKGGKEKKNGEVAMSAWARRQAA
ARDRDLGKETARKAANNDAE (SEQ ID NO: 186)
>3300009701|Ga0116228_10018148_5
[plants-peat moss-host associated]
MKIVYRYGLRAPIGHTPTNPTSKEVCKCPVCEQLFLAHSYANTLTEIERGRRAAVRALHAQVGDTGALELAVT
EANQACEKAASNIKRLRAQAFSVLAQRGHTEGAAKRGTRVTPPEMARELADARKRKQEATTRLVEHRRKIRED
PAMIVGEHEITERAKELQRSARKYAGVYWGTYLLVERAHGASIASLPLYDGAGPNDPRFDRYRGEGSLAVQIQ
QQTGDPAFTVEQLSGSDSRVQIQKEAGRLHTRLKRDEEGRVVREGPLVEMRRGNCIVARPQAVRETYSMVGDT
MLRLRIGSENRAPVWAEWPLKLSKPLPKGAIVSWVTVTKRMTGPREEWSVQFTLDTVDEVVQRDVDEEDARVV
AVNFGWRLMGEELRVAYWRSETGRNGDLRLPASMLAIRAEAEEVQSRRDKEFDETRARLCKWLASTEVPTWLR
DATKALAQWRSQARLVHLAKGWRVQRFAGDQEAFETLEAWRYHEHHLWQWESSVRATAVRSRDDLYKRWAKIL
ADNFDVLVIAGDFDVRKVAERPEVDEEIQGPAAAATSRQFAAPGRLREILCHAFTKAGSKIAKERGADITRTC
QVCGLVEEFDAARSVIRATPCSGCNATWDQDDNACIELLARYDRTCEKSKNTSSDDGARAEPKNETKKAAGSH
WDRARQKKVEKLKKVEAARQALENTG (SEQ ID NO: 187)
>3300005577|Ga0068857_100000008_197
[plants-rhizoplane-corn rhizosphere]
MEPVKKSNARKTSTSETKVYSYGAFLPKESEQIKLINDQLYFAHKYRNKLVEIERKRRNRFRNLRKLMSPELR
QLENDLLLTEEKIIELRKSFGGRAENSLDPKFPKKNRQLTPQAVEIKDLKLKKKDLSAKIKSIHTQLNLDYFK
EADSKFSLLKKERLEQKAKELNKDSLGPNDVNRHNVVNNLYKEMIDGGNRFWAIKAKISKSAEASNKRARSNC
KCSSGTYVSIEEAAKQSFSNSKFEPKFKSFDGSGKIGMQLTQNKGLSIKDALSGSSPVLKIDLHPEVYLRQNK
KKNKVLATARIKLFGTEKTGKFVDIPFIMHRQMPEDATIKWVFLVVSKIGYRSIYNIQFTIESNSFTQPSAIR
PDDVAINLGWKVNDQTDDITVATSFDGKNYNELILPSKMRANIVYKETLISHADKHFDSVKKDVSKWLKNSNL
DECITKYFTNLPQWRSHKKLLFVSKELAKVFLPDNTWYDLWKKWKTHCKENKPWKNCSDKDDLFTTLDNTIQW
CKDNSIDDPNVQMAFYLKTWAEKEIHLINWARGIESKLRKHRKEIYRCFAKKLSSTYGKVIVENWDKSKTAET
PDVENDNRTKQEENANAVRQFVGVSVLTDALKQKFGKDFCEENAKNISKEHFKCGGELINQKELSDVHCKKCN
KSVNVNYNAAAHLFDRHGERSGAVKLPGTARKTRKSPELLA (SEQ ID NO: 188)
>3300005338|Ga0068868_100030384_5
[plants-rhizoplane-miscanthus rhizosphere]
MRIYAYGARPPVENAELVFEQLRLAHAYQCALVAIERRRRVVVDRLYQSACPAEWNAYEAATARVQEIIPRMR
MTRTRPGDMLPPDMEMQVEEREIVKQIKADLAAAREAEQVARQAWYAAKKMATPRLRARLRMCDRGAYARAKR
AYNLASAVGLAWGTRLKIAESVERAGKAAAKHGTLPHFPRFDGGGTIAVQIQGGLDAEAVFGGVDTRFRLNVV
DAATWNALQGKSATQSVKKSGRVVDLPQPIEGSRRSTLRRGPIARLRIGSEGRQPIWAAWPVTLRRGLPLKAL
IKWVQIHARKIGSRTEWQLLVTVDDAKPAVHAEGPILAVNLGWRNLEDGGLRVGYAVGSDGREEEVRVPPRYT
SGVAHVDSIRSIRDKLFEAVKEFLSDWSDESPRPPDWLVDATRHIDQWRSPARLVTLLREWERKRFVGDRKTW
ERLSAWKTKNAHLRFWECDERRKLLRMRLDFYRCLAARWASQYARVVVTDMDLRDFAKLPEPEEAADTEGQTQ
RRSRVLAAPSELRGAIKNACSTRGTGYEEKKAAWKTQTCNACGVVFAFAAKQDLLHICECGARWDQDANHCRN
LLASGPVLHGAMGSLAPTGKQMESTDRPAVDGRWKKRRSRTTVEALKKTAKSA (SEQ ID NO: 189)
>3300005841|Ga0068863_100041042_2
[plants-rhizoplane-switchgrass rhizosphere]
MPRARKEKSPTKIYTYGLLPPEHGGEDLMRMLRAGHNYRNALLEVERDRLAETEDFWAKRGRYTELVARVREL
EAVRFPRKDDPRRQAHYDLIDDLREKVREKREATIQGSLPAEEGRRRLRSRELKAEAKKRGETLTKEQMTRLL
DREPGCVSVRRKAQLDYEAQSRARGVEPSPKGMVAHLRALGLNTITQEIDDRATQKAKKAREHFKVYYGTYLL
IEAQVERALEKTQFPRFKRWTGEGRVGAPVDTNFGLSVDSIHDCQFDSKHGWEENRGNTVLQIDPVGVSCAKG
FHTRARVCVDSRGRSGSKRLSVWVDFRINYHRELPRGAKICGAWINIYTLGTRVKYELQLQVQDDSFQVQPRH
GTGVAAINLGYRSSGRVAYVLSEDGKGRELLVSPRIEASIGRADTSRSDRENSANRMCDMLLGWSSELGFPEA
FLVGDGESKIDTWSGSVTRRSRSLSTRISALKDAREESLSNKLRGILGTWAKLRQDKQTRPSDEATYAAFVEW
FHQDKIYQNNEAFTRSGARNYRDQVVRDWAHELCDRYEMLLVDGTDYAKLKMRPKDKSVMPIENQTEIAHRRD
NFAPGNLRSIIEEVARARGVTVDRHDPSGLTQRHHACGWDEPWDAMPRIEHKCAGCGETFDQDANFCVGLFER
FRGILPPAPARSPRTSRKNRSSSGSERAGGGQEAAE (SEQ ID NO: 190)
>3300013306|Ga0163162_10000022_153
[plants-rhizoplane-switchgrass rhizosphere]
MNRVYEYGLLDPVVNAQIVEDQLRAAHRYRNLLVEIERERRTRVREILSSHADAAPLAEDVARLTVELEQAQA
KIKQVRAVSRRRSETTDDRMAVRDVTTRLKAAREQLKIVKAAVAQDPSCQEALAQAEQRCHDRRIEERARCGV
FWGTYLLIEEDVDRARKGKMDPKFVRFTGEGRVSVQLQGGLEWGGIAEDTRIQIRDAPDPRQGRRAGTRKWLR
LRVGSTGRDPIWAEWPLILHRPLPEGAVIKRATVTRWRRDCRRWEWRLQLILDVSRCVGTKPRGTEGACALNL
GWAKTERGLRVGFVVGSDGERTEIVLPGSILDRLDKANAIRAQRDQNLDVMKPLLAAWIAAHPLPEVLHAKIE
HLHAWRSADRFFGLARLWRQHRFDGDTEGYELLESWRYRDEHLQRYEAGMRRGALGHRREVYRLVGAALSRRY
RMLIVDDTDLRTFQRSPAPESDRVEFDAVKRSQHVAAPSDLRMQLANAFGEDGVAELSAVDVTRRCHACLTLN
DWDRASSGREHACVGCQQVWDQDVNACLNLLREWRTVAPGWEAARVAKASNRQASRAERLQQARRKKKPAEAI
AG(SEQ ID NO: 191)
>3300009148|Ga0105243_10000126_60
[plants-rhizosphere-miscanthus rhizosphere]
MPVIVYEYGLSPPKVNAALVEEQFRLAHKYRNMLTEIELERRTKIRAIMASHPDMVPFETELAEVQAEIEKLR
GEINAIRMAARKRASTPEQSRRIKTLAARARELRTEIKERRKRVAAELAPDLKAIQDAAVQRRKDERAKSGVY
WGTYLLQEAAADQARDQPMPPKFTRWNGDGRVSVQIQQGLAKEGLWGESRQVQIATRIDSLVYDHEVTRRGDR
RRLYRTTLRMRVGSTDRQPVWAEWGIAMHRPIPDGAVIKVVTVSRRRCNSTQWWWRVQFTLDTTDCKPRQRPE
YGVVACNLGFSQTDSGAIRAGYLVGDDGFEQEILVAKSDLYRGRDLTPEQKQKAMTYVRDCLAESSEIRGARD
KSLAEFKTRFLEWYQIAKATTFGEDAIPEWFRDRMEHFHMWRSPARVREMMLHWASNRWAELDDPESRWPDSR
GFEMMSTWVDEDTKAEVKESSLRNKALGDRREAYRIVAATLAKRYKTLLIDDTNLKHLQDGPEPEDAEGDIPA
VKYQQRLAAGSELRQVLINAFGGTNVVKMKPSNMTVTCSGCGARDVSWDRADGFRKHRCSACREIWDQDANFC
RNLLKEYARGEAPEAKVAKPSRSQRFHESRKKKAAADQQEQG (SEQ ID NO: 192)
>3300006846|Ga0075430_100000057_67
[plants-rhizosphere-populus rhizosphere]
MATIVYRYGVRTRTETGRYDLPAEVWQQIHLSHRLRNALVEVEHRHDEAMRDLWSAHPQVAEVEQRLAAAEQM
VAELIDQARLEHSQDRTTATRRGTATNLREARRAVRDARAARRAAIGEAYPVVKPGIEAVRAARKAAIKDLYR
EYCQDGDLYWATYNAVVADHRIAVQAVERKRRQGQAAQLRYQRWDGTGTISVQLQRQAGQPARSPELLASGDG
QWRNVLQVRPWMPPEQFDGLTRGERKRHGRGEAVWSVGGGRTVTLPIQVHRMMPADADVCEAQLVVTRTGAHW
SAALCVTVRLPDPDPVEGRSPLALHCGWRHRPDGSVRVGTWASPEPLVPPANLADVLAAHDSGRWGEIVIPAS
WLELAGRPAALRSRRDLALEPVQRKLAEWLDQNPQPDGDDGRPGLTGGDVRRWRSANRFAALAIRWRDTPPPG
EGAAEMTAVLEAWRRQDKHLWEWEAHSRARLRGRRDDAWRKVGAWLAEQAGVLVVDDVDLAALRQRGDVADDD
PVLPGTAAGQARARAALAAPGRLRQCATGAADRRGVAVRTVESGYLTRTCPHCGERGDAHPRYAQSAVVTCPS
CGRSYDQDRSAATLMLDRERSGDGPGKGERSQQ (SEQ ID NO: 193)
>3300006853|Ga0075420_100000070_3
[plants-rhizosphere-populus rhizosphere]
MATIVYRYGVRTRTETGRYDLPAEVWQQIHLSHRLRNALVEVEHRHDEAMRDLWSAHPQVAEVEQRLAAAEQM
VAELIDQARLEHSQDRTTATRRGTATNLREARRAVRDARAARRAAIGEAYPVVKPGIEAVRAARKAAIKDLYR
EYCQDGDLYWATYNAVVADHRIAVQAVERKRRQGQAAQLRYQRWDGTGTISVQLQRQAGQPARSPELLASGDG
QWRNVLQVRPWMPPEQFDGLTRGERKRHGRGEAVWSVGGGRTVTLPIQVHRMMPADADVCEAQLVVTRTGAHW
SAALCVTVRLPDPDPVEGRSPLALHCGWRHRPDGSVRVGTWASPEPLVPPANLADVLAAHDSGRWGEIVIPAS
WLELAGRPAALRSRRDLALEPVQRKLAEWLDQNPQPDGDDGRPGLTGGDVRRWRSANRFAALAIRWRDTPPPG
EGAAEMTAVLEAWRRQDKHLWEWEAHSRARLRGRRDDAWRKVGAWLAEQAGVLVVDDVDLAALRQRGDVADDD
PVLPGTAAGQARARAALAAPGRLRQCATGAADRRGVAVRTVESGYLTRTCPHCGERGDAHPRYAQSAVVTCPS
CGRSYDQDRSAATLMLDRERSGDGPGKGERSQQ (SEQ ID NO: 193)
>3300006854|Ga0075425_100000037_57
[plants-rhizosphere-populus rhizosphere]
MIVYKYGALKPKVIGGTFEDLLQYQRHSNAFYNALIEIERWRIAARDIVELAQSAPLSDEQKTEHRLAYNAAC
RAAGAATPIGWGQKQAVTEMVAAAMKTRRSDEFKARQRASKKGYDFLKRVMTCARPRHRRFDGEGILAATVQG
ATGLKASAVLTKPGPVQISGDGKHRTVTLRLREGLSLEVPIVYHRPLPERAEKEGVPYDVRVIFARLVIDRIG
DRWTYSVHLTIDAAPRVHVAQGGLGRCAVNFGWRRVPGGIRVAYAVDDDGNETSCVFPDALLGRQKHAESLRS
LADEIAAAYLGDAARRTKARCSALADPDAIHRELGREWFTMEQAAKRDGTDAEHWARRDRHLYQWERDEYASV
LRARREIYRLWARKLAASYDSVIIEAFDMRSVVKRTPSEDDIPAARHYRFLVGPHCLRLEIQSVFGARCEVLK
PAKRTLTCHACGALCKWDKARELRHDCESCGAAWDQDANNAKNQLLDAAE (SEQ ID NO: 185)
>3300006903|Ga0075426_10000611_28
[plants-rhizosphere-populus rhizosphere]
MKRRTSPLPTRIWSYGCLRPTTNTDAFFDQLRKAHVYYNTLIEIERDRRAEYRKDRAKLCPDIEKFEAEFLEL
DKAVDLFRATMKAEKKKKDDTGELKRLKDARKAIGEKLKALRLEMKNSPELKKLQEKEKEVVSGKVRAARKSS
GVYWGTYLLIEKAVETARRSKMDPRFAKWRGTGRIGIQLHHVKWSDIVDGKSQMFQVDPLPETQWDTRKGRRH
AYTKARVRVGTEKSATTGKQVPVFVEVPLYLHRRPPADAKLTWAWIFVTRKGPTLRYQLQLSVESNLFSAGLP
EQPKKSVCAVDVCWRKMDHGLRLGLAVDHHGNQFEMVLPKAVPELIEMGDNMKSAADRIFNGTKDFVSKWIKE
NGLPGAIEPARVSQWLSHRKLRGLTRQWLAETIGFERARELWRAWCFERVGSRKNPLTVPKKDLFAPAEEAFA
WAEKHGLTKPFEQMAFYLELWSRKDRHLEQWAADQFYRATMIRRDAFRNWSRFLVNNYETILLEDMTHTTFAK
DSVVEAEKSFDVLHRQRNEAAPGLFMQTLRSAVGAHVVPMDPADTTNDCAHCRHRNDWSQTERSKNVVLTCAG
CGKMFDQDANAARTMLIRYFEGDTGSGGSKDKPKPASPPPSKPPPRALTKRRKPGAEPRASV (SEQ ID
NO: 194)
>3300006914|Ga0075436_100000782_9
[plants-rhizosphere-populus rhizosphere]
MKRRTSPLPTRIWSYGCLRPTTNTDAFFDQLRKAHVYYNTLIEIERDRRAEYRKDRAKLCPDIEKFEAEFLEL
DKAVDLFRATMKAEKKKKDDTGELKRLKDARKAIGEKLKALRLEMKNSPELKKLQEKEKEVVSGKVRAARKSS
GVYWGTYLLIEKAVETARRSKMDPRFAKWRGTGRIGIQLHHVKWSDIVDGKSQMFQVDPLPETQWDTRKGRRH
AYTKARVRVGTEKSATTGKQVPVFVEVPLYLHRRPPADAKLTWAWIFVTRKGPTLRYQLQLSVESNLFSAGLP
EQPKKSVCAVDVCWRKMDHGLRLGLAVDHHGNQFEMVLPKAVPELIEMGDNMKSAADRIFNGTKDFVSKWIKE
NGLPGAIEPARVSQWLSHRKLRGLTRQWLAETIGFERARELWRAWCFERVGSRKNPLTVPKKDLFAPAEEAFA
WAEKHGLTKPFEQMAFYLELWSRKDRHLEQWAADQFYRATMIRRDAFRNWSRFLVNNYETILLEDMTHTTFAK
DSVVEAEKSFDVLHRQRNEAAPGLFMQTLRSAVGAHVVPMDPADTTNDCAHCRHRNDWSQTERSKNVVLTCAG
CGKMFDQDANAARTMLIRYFEGDTGSGGSKDKPKPASPPPSKPPPRALTKRRKPGAEPRASV (SEQ ID
NO: 194)
>3300007076|Ga0075435_100000061_47
[plants-rhizosphere-populus rhizosphere]
MKRRTSPLPTRIWSYGCLRPTTNTDAFFDQLRKAHVYYNTLIEIERDRRAEYRKDRAKLCPDIEKFEAEFLEL
DKAVDLFRATMKAEKKKKDDTGELKRLKDARKAIGEKLKALRLEMKNSPELKKLQEKEKEVVSGKVRAARKSS
GVYWGTYLLIEKAVETARRSKMDPRFAKWRGTGRIGIQLHHVKWSDIVDGKSQMFQVDPLPETQWDTRKGRRH
AYTKARVRVGTEKSATTGKQVPVFVEVPLYLHRRPPADAKLTWAWIFVTRKGPTLRYQLQLSVESNLFSAGLP
EQPKKSVCAVDVCWRKMDHGLRLGLAVDHHGNQFEMVLPKAVPELIEMGDNMKSAADRIFNGTKDFVSKWIKE
NGLPGAIEPARVSQWLSHRKLRGLTRQWLAETIGFERARELWRAWCFERVGSRKNPLTVPKKDLFAPAEEAFA
WAEKHGLTKPFEQMAFYLELWSRKDRHLEQWAADQFYRATMIRRDAFRNWSRFLVNNYETILLEDMTHTTFAK
DSVVEAEKSFDVLHRQRNEAAPGLFMQTLRSAVGAHVVPMDPADTTNDCAHCRHRNDWSQTERSKNVVLTCAG
CGKMFDQDANAARTMLIRYFEGDTGSGGSKDKPKPASPPPSKPPPRALTKRRKPGAEPRASV (SEQ ID
NO: 194)
>3300007076|Ga0075435_100000750_29
[plants-rhizosphere-populus rhizosphere]
MIVYKYGALKPKVIGGTFEDLLQYQRHSNAFYNALIEIERWRIAARDIVELAQSAPLSDEQKTEHRLAYNAAC
RAAGAATPIGWGQKQAVTEMVAAAMKTRRSDEFKARQRASKKGYDFLKRVMTCARPRHRRFDGEGILAATVQG
ATGLKASAVLTKPGPVQISGDGKHRTVTLRLREGLSLEVPIVYHRPLPERAEKEGVPYDVRVIFARLVIDRIG
DRWTYSVHLTIDAAPRVHVAQGGLGRCAVNFGWRRVPGGIRVAYAVDDDGNETSCVFPDALLGRQKHAESLRS
LADEIAAAYLGDAARRTKARCSALADPDAIHRELGREWFTMEQAAKRDGTDAEHWARRDRHLYQWERDEYASV
LRARREIYRLWARKLAASYDSVIIEAFDMRSVVKRTPSEDDIPAARHYRFLVGPHCLRLEIQSVFGARCEVLK
PAKRTLTCHACGALCKWDKARELRHDCESCGAAWDQDANNAKNQLLDAAE (SEQ ID NO: 185)
>3300009100|Ga0075418_10076301_2
[plants-rhizosphere-populus rhizosphere]
MTEKPPTKIYTYGLLQPTKNGHEFSKMCRAAHDYYNALLEIERTRQREEDDFWAKRGGYVDLLDEFRQLEAMR
PRRDDPKREEIFARRKELRKKLWELRDVTVDRSLPIEDANRRNRHRELKKAAKAEGRNITDAEISASLDKDPS
CVSPRRRAQLEYTEEAKARGVNVSGRGLNQYLRDRGLLKVTQPIDDRAAEDQKRARDHFELYYGTYLLVEPAA
EQAIERSEMFPAFKPWRGEVGRVGAPVNTNTGISVEAIHNCFNEDPKTGERTFSDGGNTVLQIIPIRKEVRES
RRVRVKGAPSAHQQGMKFLNQTVMRICVRSEGRVGAQRIPVWVEFPMQYHRDLPPNAKVTAAWVIASQLGTRT
VYKLQLQVQDEAFRNPVKPCGRSTMAVNLGYRSTGRVAYALTQDGRYEVMDVKDRVGKCIDEADELRSLRDRD
ANRMRNDLFEWRDVEAYPQSFLEGDGEKFVPHWSGDEKRRRSVKVRSMKTRIDGLVHARDDSLPTRLRAIYET
WERMREQGLLRSVDDRIFKVFREWYIEDKRSQDKEAHQRLNAHGTRELDIYAWAHRLCDEASLILVEDTNYAT
MKLKSNRRPKEELPVEISVSIARRRDMYAPGRMRKILEQVAVKRGVKIVRLSSVGLTQRHHKCGFDEPWDAMR
SIQHKCEGCGVTFDQDRNFCEGLFERYRGTLPAAPARKAGKGKKSRDLPAEAE (SEQ ID NO: 195)
>3300009100|Ga0075418_10076301_2
[plants-rhizosphere-populus rhizosphere]
MRSMTEKPPTKIYTYGLLQPTKNGHEFSKMCRAAHDYYNALLEIERTRQREEDDFWAKRGGYVDLLDEFRQLE
AMRPRRDDPKREEIFARRKELRKKLWELRDVTVDRSLPIEDANRRNRHRELKKAAKAEGRNITDAEISASLDK
DPSCVSPRRRAQLEYTEEAKARGVNVSGRGLNQYLRDRGLLKVTQPIDDRAAEDQKRARDHFELYYGTYLLVE
PAAEQAIERSEMFPAFKPWRGEVGRVGAPVNTNTGISVEAIHNCFNEDPKTGERTFSDGGNTVLQIIPIRKEV
RESRRVRVKGAPSAHQQGMKFLNQTVMRICVRSEGRVGAQRIPVWVEFPMQYHRDLPPNAKVTAAWVIASQLG
TRTVYKLQLQVQDEAFRNPVKPCGRSTMAVNLGYRSTGRVAYALTQDGRYEVMDVKDRVGKCIDEADELRSLR
DRDANRMRNDLFEWRDVEAYPQSFLEGDGEKFVPHWSGDEKRRRSVKVRSMKTRIDGLVHARDDSLPTRLRAI
YETWERMREQGLLRSVDDRIFKVFREWYIEDKRSQDKEAHQRLNAHGTRELDIYAWAHRLCDEASLILVEDTN
YATMKLKSNRRPKEELPVEISVSIARRRDMYAPGRMRKILEQVAVKRGVKIVRLSSVGLTQRHHKCGFDEPWD
AMRSIQHKCEGCGVTFDQDRNFCEGLFERYRGTLPAAPARKAGKGKKSRDLPAEAE (SEQ ID NO: 196)
>3300009156|Ga0111538_10081463_8
[plants-rhizosphere-populus rhizosphere]
MQRQKDDSITSRVYVYGCVPERVAPVHNEDRALEQMRLGQRLWNVLVAIDRARVARYRRIMADEAQERIDALR
DQAAALRDEIKTRRKQARKRSVDIGDLAERLAAVKSELSALIEEQKRTSTERHDARRAELTAMQERTNHRIKR
ARQAAASLGLFWGTYNDIVQRSDAGRKHGGELHFRGFRGEGTLTAQIMGGAIVTRCVEGAHTFFQVDPPQPGR
KWRYARMRIGSEERGGVKLAPVWLEIPIVYHRDLPPAGMIKSVSMTRRMLAGKPRWQLNVTLNLPAPKPTTRT
AAVAIDIGWRLLPEGVRVAYWMDDAGQHGQVLIPSRDISQFERVRSLRSNCDLSRDEILPGLAEWFGGLELPA
EWAQRVAYLSQWRSSDRLAGLYDWWRDHRLPGDAETFEAYTTWRKQYLHLAHWWRNLQDQMTLRVREQYRVFA
AQLAGRYGVVYIEDFDLSSVARKPKTEGDGEKSASSTYRQMVSPSMFRGALLNALQREGATVTELPAEYTTRI
CSTCGYGREWDQAESVMHRCGGCGEMFDQDENAAKNLLRLVAQGVAG (SEQ ID NO: 197)
>3300005548|Ga0070665_100000073_173
[plants-rhizosphere-switchgrass rhizosphere]
MTTLAFKYGLGDPLDWDTDIADQLYLQNKLWNRLVEIERDARTRYRAVVGEDDAIAPLVRDIEAAKAQKEALL
TERKGLRAKARKRVPTPEIDARIAECATVVRELAQRIKTERVAAKERLAPHVRAIEEWRFGAVKEARNASGLW
WGNYNAVCASYDTARSRAMKDGAELQFHRFTGEGRLTCQIQGGTTPEQIVDGKCSLVRVDPLSAGAHSHPSRG
ERRRLQRTKIAVTAYMKDGERRLLTLPMQMHRPLPDGAIVKQVVVTRRKIGTRYRWHAVFTCSVPDAQPVQHA
STSACGVNFGFRQVLGGLRVATVSTSPSKTPDYLVLPEEWLRAMDRCEALQSARDEHLLPMHAAARELTRGED
APESLRDKLDRIARAPKIGSALLASLVLAWRDTHADWQSDKLVGFEAWRRNDKRAAEEQANLRDKLHASRTER
YRLWARELVRDHALVGCGKIELRKLAELEKQDGTENDLHARARSNRQRVSLYSLQLELARAAQLAGARVVMAD
GPLTSTCHACGATTLIKPDIMQVCDHCSAVWDQDHNAALNALSYAQQSPPPRERSGDAQDTDQENQVFGEPAE
EKKDSARNVRLAA (SEQ ID NO: 198)
>OBLM01000011_1
[soil metagenome]
MHSRVYLYGLLPPTPACAPLVEQQMGRAHRYRNVLVEIERERRAKVREVMAAHPDMAPLEEQVNALVAERETA
LQALPRKAARNDPARANVRAMATRIRDLRGQIKAARKAVLADAEVARQLAEADEFSRERVRRARATCNVYWGT
YLLQEADADRARMERMPPKFHRWTGEGRVSVQLQGGLEQDKMWGGDTRMQIDPVAPEAHDPLSPRGVRRRAHR
TVLRLRVGSDAQRGPIWAEWPMLMHRPLPKGAIIKVATVSRRYRNCTTWDWQVLLTVSIPDESARPAPAAGVV
ALNLGFCERPDGSLRAGYLVGDDGWTQEIVVPASTSELLGKCDSIRSFRDKNLDAMRPLLSAWRQDQNLAFER
VCRDVIAACETTPPELDGAFYRLAMYIVNGGHSLPSWLHERIQSVHAWRSHDRFRKLALTWRDRRFPGDHAAY
ELLEVWRYRDQHLEHYESGMRRRTLLRRREGYRIIAAAAAARYRTLLVDDTDLRHFQRKPDPEDGATVPEQIG
LATMRVNQRLTACSGLREALASAFGSRVVKMSSQNVSRRCHACGDINLAMSSAREQTCTGCAATWDVDQNACL
NLLGEHRRDDPDRETARVAKLANAKPSRGKRLSAARASNGATVLAREASGN (SEQ ID NO: 199)
>OCTA010000646_37
[soil metagenome]
MKRRTSTAPVRIYAYGCRLPTQGGELVEQQLLFRHRYYNKLIEIELDCREKMRAARSASSEDVAHAESAFAIY
ETEIVGVLDAIKAKKGAARAAKVDAAEERAVLAVLRDMKRKTIDDLHAAKLAARTPELLAEFTAIQEAANAEV
RDARSKCGVYWGTYLLVEQEVEQAVKAARKNHEDPGFRRFMSVPNRQGQATIARGRVGVELIHGVPVATIMAG
TDTRLQIHMEKSDSKRGQTMARAKIRVGTAENGRSPIFAEFPFRMHRPLPADGVVKWAWISKSTKGRWVDWSL
QIVVEAASLHRPVRQPSDGGVVAFDIGWRVRLHEVVNELRVAYWHDDQGNHGELVLPSDDRVRVDSRGRKHRP
EGVRGRFDHVDSLRSIEDKNFDAIRSELVAWKTGRDLPEWFVSALEWLHAWKSHRKLGAVFDQWRSNRFSGDD
GMFAKVETWWKQHRHLYDWESCERDRALNARKNTFRQWAPQFTRKYAVVVLEEKFLAEVAKLQAPDSTKANMP
RPTRRNRTVAACGEFVLALKNAAPGNGCTVDAEPCEDTTATCARCGYVERFDHRPLAHACARCGDVSGPVDQD
RTAAENLLAAYAGKMSRSQSASEGGNIVGDPDGSLVIPAQEGVS (SEQ ID NO: 200)
>ODAK010001378_33
[soil metagenome]
MIRVYRYGVASPHDGADLVYAQMRGAHAYRNTLIEIERGRRGALRDLESAEVRGLTAEVAAADEACQAIGSTI
KVARAESRKRSERKVDLERLAEARSVKRAITGRLYEARRNHMLATRSAVDIVNELAKCLLKSAREHCGVYWGT
YLLAEEAMGASSSAPLFGKDGITQNDPKFIRWTGDGAVRVQIQKGASVAAVRADAEHSQLQIREPTGAWSHPT
RSERRRLAKRGEVRIRVGSEPNGKPVWAAWRLDMHRPLPEEARIKEATIHVRQRGAHSEWSLLVTVDVPPAAV
VPSESRGEAVGVDVGWRLIDGCIRVAVCMGAGGAVTELRLDAPTIRLLRSSEALRSKRDERFNAAKARIRKAA
AEPVAPEWLRESGKTMHAWRSPQRLAQLHARWAEERFSGDDMVFGRLEAWWWTDRHLWSTEAQASLQGHRRRK
DIYRVFAAKLAARYDVIVLEKFDLRKVARTEETGEETPAGDNDTSRSNRQLASVSEFRACLLDAARSRGRSVV
MVDASETTRTCHVCGLVEAFDAAAHLRRTCACGSEWDQDENAAEVILARWRERPGDAKILVPARSTEIYCETM
ELLETRWQRVARLRKEKLARMDTARKSASNAAE (SEQ ID NO: 201)
>ODAK010029943_5
[soil metagenome]
MMVFKYGTVPARIAPVIGAEQAATQLRLANRLWNLLVAIERARVARYRKVMFDAAQGRIELLKAKLSALRGKI
QVRRQAGRRRVDVSDLTAESQEIRAAIKAEIKAHKATSAERHDARRAELDALSETSKSRIKRARQAAASMGLF
WGTYNDIVQRADVGRRAGELHFRRDTGDGTLTAQIMGGADPEECMTAHSFFQIASKCPLSGLVAGDVAETDAQ
PVKWQYARMRIGSTGERQPIWLAIPIVLHRPLPDGARIKSVSMTKRKTTWSLNVTVAEPAPTPKLIGPRVAID
LGWRVVPSGVRVAYWADTLGGEGQVVVSDEDIGQFGRVRSLRSRCDTMRDEYLPVLAAWTSGRELPAEWQAET
IALVQWRSPDRLARLIRWWARLPGDAEMFSRASAWRKQYLHLANWWRNLEDQMRGRLREQYRIFAAGVAKKYS
TVYLEVFHLPDVIETPAAESEEVRTAESRYRQMVSLSVLRAAVRNACTREGCTVVDVAPEYTTLGCHLCGTIT
EWDTAASLMHQCKGCGAVWDQDQNAAINLLARGASGGAPPTANQPDRPRKWDRVRDRSRKSAQAAESAILAAA
AVEMPAQRLSC (SEQ ID NO: 202)
>ODAK010029943_6
[soil metagenome]
MTKPLSGLVAETGLLFRAFRAARPSSKCLLSGLVVETECDNVVMMVFKYGTVPARIAPVIGAEQAATQLRLAN
RLWNLLVAIERARVARYRKVMFDAAQGRIELLKAKLSALRGKIQVRRQAGRRRVDVSDLTAESQEIRAAIKAE
IKAHKATSAERHDARRAELDALSETSKSRIKRARQAAASMGLFWGTYNDIVQRADVGRRAGELHFRRDTGDGT
LTAQIMGGADPEECMTAHSFFQIASKCPLSGLVAGDVAETDAQPVKWQYARMRIGSTGERQPIWLAIPIVLHR
PLPDGARIKSVSMTKRKTTWSLNVTVAEPAPTPKLIGPRVAIDLGWRVVPSGVRVAYWADTLGGEGQVVVSDE
DIGQFGRVRSLRSRCDTMRDEYLPVLAAWTSGRELPAEWQAETIALVQWRSPDRLARLIRWWARLPGDAEMFS
RASAWRKQYLHLANWWRNLEDQMRGRLREQYRIFAAGVAKKYSTVYLEVFHLPDVIETPAAESEEVRTAESRY
RQMVSLSVLRAAVRNACTREGCTVVDVAPEYTTLGCHLCGTITEWDTAASLMHQCKGCGAVWDQDQNAAINLL
ARGASGGAPPTANQPDRPRKWDRVRDRSRKSAQAAESAILAAAAVEMPAQRLSC (SEQ ID NO: 203)
>3300005602|Ga0070762_10000001_34
[terrestrial-soil]
MKLVYKYALASPHENFDLIDLQMRAAHRYRNTLVEIERGRRAAVRLVEAEAGDMPAAQRALTMAIGARELADG
AIKRHRARSRKRDEPQEMRDTLRAARVAERDAAKAFRELRLKIKDSPAMIAARDAIGERAKELQRSARANCGV
YWGSYLLVEGAVSDSFSDTSLYNKDGHANDPAWARWTGEGSVGVQIQTATADKATKSLTVERAASGNDSRLRI
VLPDERAWDRSGRTHRECENMARQAQLSIRIGSNGRDPVWGSWRMDMHRPLPVGSTIQLATVHRKRVGPYDRW
HVTFTLDVPASTRASTAGTGTIAVDVGWRVMGDELRVAGWQDDTGDRGELRLSAKDLAVLRAPEAMRSARDLR
FDAARLALSVWLRDHREILPDWLRVISANVHAWKAEARMVALRNRWMDARFADDEAAYDALTNWAFRARHDWA
VESCARGQALRRRREKYRVWAAQLATKYDTIVIENFDKRRVAATSRDATTENETARANRVLASTSELVSCMET
AARSRRAALFAVPCADTTRTCPTCGLVESRDAAAAVRLECECGARWDQDVDGAPLVLLARWRERPGDAKIVVS
AREQEKTNENGEKKEGRWAKVARLRAEKVARMATAREADADGAE (SEQ ID NO: 204)
>3300005602|Ga0070762_10000001_32
[terrestrial-soil]
MWSIGASVATRCCRRRPSDRYGSDKRNKEIVTMKLVYKYALASPHENFDLIDLQMRAAHRYRNTLVEIERGRR
AAVRLVEAEAGDMPAAQRALTMAIGARELADGAIKRHRARSRKRDEPQEMRDTLRAARVAERDAAKAFRELRL
KIKDSPAMIAARDAIGERAKELQRSARANCGVYWGSYLLVEGAVSDSFSDTSLYNKDGHANDPAWARWTGEGS
VGVQIQTATADKATKSLTVERAASGNDSRLRIVLPDERAWDRSGRTHRECENMARQAQLSIRIGSNGRDPVWG
SWRMDMHRPLPVGSTIQLATVHRKRVGPYDRWHVTFTLDVPASTRASTAGTGTIAVDVGWRVMGDELRVAGWQ
DDTGDRGELRLSAKDLAVLRAPEAMRSARDLRFDAARLALSVWLRDHREILPDWLRVISANVHAWKAEARMVA
LRNRWMDARFADDEAAYDALTNWAFRARHDWAVESCARGQALRRRREKYRVWAAQLATKYDTIVIENFDKRRV
AATSRDATTENETARANRVLASTSELVSCMETAARSRRAALFAVPCADTTRTCPTCGLVESRDAAAAVRLECE
CGARWDQDVDGAPLVLLARWRERPGDAKIVVSAREQEKTNENGEKKEGRWAKVARLRAEKVARMATAREADAD
GAE (SEQ ID NO: 205)
>3300006796|Ga0066665_10000988_15
[terrestrial-soil]
MSEQLDDTPEQPNEVEETKKRKQRNKGKHPARIWSVFSRYLVSGREHFDKQVLLAHRFRNKLVELELQRRAAA
NVVIAQASSELQPLIDALAAAEQVLEVSLQELKAVRAKHRRRAESAAQRDAVTNARTARNQASKALSKARKDA
FASEAAQVGLWLAEEHHFQAVLAARHAFINDGLYWPTATDVQDRARAMRKGAPPVFRRFGGAEQAGRIAVQIQ
QRTDKSQSEGGITFEEAFSCSHGFFRLEKKPGRDPLPEIADQPDYKSKRQQLLTYARAWLRVGSEGKGARAKP
CWVVADVLLTRQAPKTARIVQVYLDHSVIGDRERWRLSLVLTNQEGWPKPNRASGCMVGIDLGWRLLDTGELR
VAYACGADGQHHELRLPASLVKVWRRPDRIQQERDNLFNDVKARLLEWLKGREDLPDWLKEQAEHLHLWKSST
RLSRLVDHWAGRDINWSSQRRIAGDEEILASLRGWVKRNLHLRDYQYHEREQLAAHRLDVYRKWADGLARLYQ
TAVLEDADWRDLARLPSPEDDAVNETARYNQRMASPGLLASVITNMFAITSRVECANTTRECWRCGHTEAFDA
EAQLIRVCPGCGDACDQDESAARVLLARGQALNQSQVAEAAPSS (SEQ ID NO: 206)
>3300018429|Ga0190272_10000030_113
[terrestrial-soil]
MAVVVHVYGVPPVLHGERVRLPAEVDEQLSLAHCLREDLVTLEHQRQDAVTAVWSSYPQIAAIETQLTAAETE
LTDRSAAAAAERSAARKKGPTESSEAVRQLKARIKDLRSQRRTAIADAHPTATPRLTAIADAHRAAIKALYAD
YSQGRGLYWATYNDVVAHHQVATKRVAAERKAGRPANIRHHRYDGSGSITVQLQRQTGAPPRLPATIADEQNG
PWRNVLYLTPWVDPDTWATLARAEQRRRRLGVVRLDLGNKRHLSIPVLVHRMLPADADITSARLVVRRVAGHR
KIELHVTARIKDPVTRSGGPAVALHLGWRREDGGAVRVATWRSTAPVHVPDDLNDLVHADTDHTGTISLPARW
WHRVSTQPEMAARRATSLNDIRDQLVAALTDNPLTITADDEDAQPVPTAAAVATWRSPARFAHLARTWATDCP
AGHQATAAALENWRRSDRRLWEQQAHGTANTLAARADAYRRTMSWLLTGASRLVLDNTAIADLARRADPATEP
TLPTAVTDRVAHQRIGASPGQLRSIATTTANSYGVAVAVMPHTGITRTHYRCGHLNPADDRYSAARIVTCDGC
GQHYDQESSATLMLLAASGDVAAPGSATARNPDTSAHA (SEQ ID NO: 207)
>3300018432|Ga0190275_10000082_154
[terrestrial-soil]
MTTLEARVAQYGCLAPIENADVVRQQMRLGARYYNELIALERCRRAVYRDLRRKYVDLESVEARVEELAAELM
SLREAIKGVRKEARRRVDTADLDQRAKDVQSALRVARVALKDARQAARDNAELRAAVEQLDERAKIWSKALRA
MRAPWWGTYLLEEASAEQARKATIDPSFRRARGRERISAAGEGSAEGRIGVQVQGGMTVAELYGCEDTRLRIE
PVSPDAWHASSRGVRRRCSRTRLWMRVESAGRSPVWAVFPLILHRPIPDDARIKGAVVRLRVLGFREQWTVSV
TYAREPAVMPERPGIVALDLGWRQRPDGSLRVAYCADDQGNHREVVMPESVRMRLRKARDLREIQDLHFNRAV
RWLARWLDAGKAPEWLARERPHLGQWRSHGRLRRLVLDWRRTRFVGDERIFAAMERWLHRSRHLYQWEVDAQR
KALLARRELYRCTAAQIARAYGRVVIEQFDLATAKRLKAPEQGEDAPLAQRAQLHASAPGEFRQCLTQAVQRE
GGLVISVDASGTTSHCHACGGVCSWEQGEELWHRCEYCGELWDQDHNAAINLLRRFTRDHSGDATNPAPARKP
SKRAERFRKRHAQPAATDVAE (SEQ ID NO: 208)
>3300018481|Ga0190271_10027355_3
[terrestrial-soil]
MELNAKPDDLELDDDIPAGEEEEEKPDLDARVAQYGCLWPIQGEDLIRQQMRAGHQYMNNLIFIERCRRTCYR
DLRREHANITEIEDLCQTLAHELDELRDQIKGARKAARSRVETKELNAKAAEVLKRLQPARKELKAARTAAAQ
NEVLKAAVKELDARVLLCQKFLRKQTDCFWGSYILVEASMKQVKKSKIDPYFRRWKGEGRIGVQIQHGMTVQR
ALDGVDRRLRIMPAPTSFLGENKSASHARHKARHLLYIRVDSEGRYPVWAVFPMIMHRDMPPDALIKGVTVHC
RKRGLRDKWSCDITFTKPAVKPAKKPGVVAIDLGWRKRPDASLRVAYWVGSDGQDGEIRMPERVSRRLRHSDG
LREVQDLSFNRMKSRLKLWLNAVDARDETLDKPMVPDFLTAIQPHMDKLRSHERIRKLVKQWEHERFEGDEHI
FWAVKQWFRSSIHLYPWEVSQRKSTLRYRREMYRLAALELSKRYGTLVLENFDLSKAKRKNAPEQGPDAPKAQ
RTQLHASAPGEFRQALVQVFLREGGEVFRVDAMGTTSSCHACGATCKWDQAEEISHRCEHCGTLWDQDYNAAK
NLLLRYALPQAS (SEQ ID NO: 209)
>3300019874|Ga0193744_1000265_21
[terrestrial-soil]
MIVYKYGALKPKVIGGSFDDLLAYQRDSNVFYNALIEVERWRIAARDIVEMDQAGPLSDEQKTEQRLAYNAAC
RAAGQASTIGWGQKQAVTEMVAAAMKTRRADEFKARQRATKKGYDFVKRVMTCARPRHRRFDGEGLLAATVQG
CSGLKSSAVLSKSGPVQISGSGKHRTVTLRLREGLSLEIPIAYHRPLPERAEKEGVPYDVRVIFARLMIDRIG
DRWIYSVHLTIDAAPRAHAAQGGLGRCAVNFGWRRVEGGIRVAYAVDDEGNETSCVIPDSLIGRQKHAESLRS
LADEIADAYLGAAARRTKSRRQALASPDATHPGLGKIRFTLGQAANHAPEDAEHWARRDRHLYQWERDEYASV
LRSRREIYRLWARKLAASYDSVIIEAFDMRSVVTRAPNKDNIPAARHYRFLVGPHYLRAEVQSVFGKRCEISK
PAKRTLTCHACGALCKWDKARELRHDCESCGAAWDQDANNAKNQLLDAAE (SEQ ID NO: 210)
>3300020021|Ga0193726_1013919_1
[terrestrial-soil]
MIKNYEYGLLDPTANAQLVDDQMRAAHRYYNQLVEIERERRAEIAAILVGHPDTEALAARVADLARQREEARL
AIKATRQATRDRSETSQMRDRVKDLATELRAARATLKTARDVIKTDAVIVAAISACDDRATTRVKARRAACEA
YWGSYKLSEEAVDAAKKAKAPPHFKRWTGDGRVSVQLQGGISDGELFGTDTQVQVAPVSPDAHDLRKPRGVRR
LASRTILRLRVQSTEKGRPIWAEWPMILHRPIPEGARVKIATVSRRRRDCRRWDWRVLLTLEIPDGASEHRRL
IPASGAIALNLGWCKRPEDAVRAGYVLSDDGVIDREVIVPPSTINRVEKSEAIRSQRDKDLDAMRVTLVAWLR
AHEAGLPAWVVERTILSREPRAVPQVDTPRAEAVRDASQRTRAWHVAQWRSAARFRALAFAWRSQRFDGDGEG
YQVLEDWRYRDEHLERYESGMRRGGLLDRRERYRMLAADLAARYRTLVVDDFDLRTFAEIPKPEDESANVKPH
RKQQRYAAGSELRAALLNAFGPTRVLRESSVDVTRACAAIVVDEATGAEHTCGQLDLWDHTVAREHTCSGCGA
TWDQDQNACKNLIGRWRERLGADGSVETARVATPRKESRSERLRRTRWKREPEAEAASTTEPHPSPSRVAPVA
PATAAPTCQKSPIVTDGAATITATAPPPSPLRAPSPVVPGQSAVRANRPIAAPG (SEQ ID NO: 211)
>3300020021|Ga0193726_1013919_1
[terrestrial-soil]
MEISRASGSHRVMPMCTQPVHACQCLQERATWDHVIKNYEYGLLDPTANAQLVDDQMRAAHRYYNQLVEIERE
RRAEIAAILVGHPDTEALAARVADLARQREEARLAIKATRQATRDRSETSQMRDRVKDLATELRAARATLKTA
RDVIKTDAVIVAAISACDDRATTRVKARRAACEAYWGSYKLSEEAVDAAKKAKAPPHFKRWTGDGRVSVQLQG
GISDGELFGTDTQVQVAPVSPDAHDLRKPRGVRRLASRTILRLRVQSTEKGRPIWAEWPMILHRPIPEGARVK
IATVSRRRRDCRRWDWRVLLTLEIPDGASEHRRLIPASGAIALNLGWCKRPEDAVRAGYVLSDDGVIDREVIV
PPSTINRVEKSEAIRSQRDKDLDAMRVTLVAWLRAHEAGLPAWVVERTILSREPRAVPQVDTPRAEAVRDASQ
RTRAWHVAQWRSAARFRALAFAWRSQRFDGDGEGYQVLEDWRYRDEHLERYESGMRRGGLLDRRERYRMLAAD
LAARYRTLVVDDFDLRTFAEIPKPEDESANVKPHRKQQRYAAGSELRAALLNAFGPTRVLRESSVDVTRACAA
IVVDEATGAEHTCGQLDLWDHTVAREHTCSGCGATWDQDQNACKNLIGRWRERLGADGSVETARVATPRKESR
SERLRRTRWKREPEAEAASTTEPHPSPSRVAPVAPATAAPTCQKSPIVTDGAATITATAPPPSPLRAPSPVVP
GQSAVRANRPIAAPG (SEQ ID NO: 212)
>3300020034|Ga0193753_10002988_10
[terrestrial-soil]
MKEIRVYKHWAEPASAVDHHRLQSQLKLAYQYRRMLAMIENAARVAQRALVQADPAIAMLINQLAVLHEADPP
ATIVITAAQEALRLARRDLHKTDAYKLEARAIGDRRQVLVRGARGLFSAQGLAWGTYQHVEEAHDQSCSENPY
WEDVKVRLTPGFGAIAVHIQNRVLPSGTLVGGRDTFVQIDAERYGLSTFRNGWRAIDPDGPSGRVQIPAGERR
PCGGGAPRLQRIRIRTGSDGRAPIWTEFHMLLHRPLPPGKILWVRAHQTRVGIRTMYNIQFVVDIDTAGRAPR
ARPAGGAVMDDRAPQARSHHVGDATDDRAPQARSMMSHAIVGVDIGWRKLENGDWRVAMAVIPDGTTDELVVP
HDVLRRADKSADLRSIRDQSRDAMRTRLLAFRETVVASLEDATPAPSADWLEATRTMHAWLKFGRFVRLRHWW
AQHRFAGDEEIYSALCAWLDNDRHLIDWQEFNIRRMKRQIDGLYQAWAMRLARSFDVIAIEDMNLTDLKASSP
GLVSDLAHERGMVVGLSHLIGWLKRATAGYNTRLVEVDPAYTTRNCRKCGFCRPASAELVIKCEACGFAEDQD
ITAGHNITARAVATLEEPTPEATPVKRRVRRTRRRPNEATTEPNNG (SEQ ID NO: 213)
>3300020034|Ga0193753_10002988_9
[terrestrial-soil]
MIDRSEIDPGNRDAQLYQRGTTQLIDRLEIDPGANQGAIMKEIRVYKHWAEPASAVDHHRLQSQLKLAYQYRR
MLAMIENAARVAQRALVQADPAIAMLINQLAVLHEADPPATIVITAAQEALRLARRDLHKTDAYKLEARAIGD
RRQVLVRGARGLFSAQGLAWGTYQHVEEAHDQSCSENPYWEDVKVRLTPGFGAIAVHIQNRVLPSGTLVGGRD
TFVQIDAERYGLSTFRNGWRAIDPDGPSGRVQIPAGERRPCGGGAPRLQRIRIRTGSDGRAPIWTEFHMLLHR
PLPPGKILWVRAHQTRVGIRTMYNIQFVVDIDTAGRAPRARPAGGAVMDDRAPQARSHHVGDATDDRAPQARS
MMSHAIVGVDIGWRKLENGDWRVAMAVIPDGTTDELVVPHDVLRRADKSADLRSIRDQSRDAMRTRLLAFRET
VVASLEDATPAPSADWLEATRTMHAWLKFGRFVRLRHWWAQHRFAGDEEIYSALCAWLDNDRHLIDWQEFNIR
RMKRQIDGLYQAWAMRLARSFDVIAIEDMNLTDLKASSPGLVSDLAHERGMVVGLSHLIGWLKRATAGYNTRL
VEVDPAYTTRNCRKCGFCRPASAELVIKCEACGFAEDQDITAGHNITARAVATLEEPTPEATPVKRRVRRTRR
RPNEATTEPNNG (SEQ ID NO: 214)
>3300020156|Ga0196970_1000866_40
[terrestrial-soil]
MAYGHTALPAINWVYGCKRPFEGEELIRSQLRQANRYRNVLVAIERRRRTNFEQLVLRLCPELQKLETQRNNL
TQEIIELRAAMKAENARQRKTVRNPESTRRIKELQAQRQLLRPRIKELRDATYTHPTVKVVDENAAAWVKRAR
AACGIYWGTYLVREATVKQAIKDARPGLPEFKRFTGQGAVAFQSQQGTSTALLEAGGGNNLVQMHWNEPRNRR
GRRRGELWFRIGSDANRRPIWAKASISQHRPFPPDTVIKFGHLHLTKCGTRESWSVRFQLVRESGFVRTGLAA
AGRVGVDIGWRRVPGGLRVAYWVGDDGREGQELLPEDFLASKQYVEELRSRRSLEFDAVRQRLATWLQMTSNV
PEWLLDRTHSLAQWKSVDRLCWLVKAWAEQRFSVDESIFPVLWRWRGQSLRLKEEESHGQRKLVVRRRQLYRE
IALRLAQEYRTICVEDFNLQKLLTKPQVEQDAVEAGVTYHSQLAAVGELRMFLAERAADVLRLPAQGTTQHCH
MCGAKSNASDKSQLVHTCQSCSAQYDQDRNAALWLLRGGVPEYAIDGA (SEQ ID NO: 215)
>3300020579|Ga0210407_10000200_14
[terrestrial-soil]
MTMIRVYKYGLLRPIQNEALVRAQLRAKHDYRNTLIEIERGRRTAMRNVEEQHSELATAMAASRAALVELEES
RQAIRLARSKSRSRSETNVMKERVKQARIVRRTTSQALYDCRARVRPEMISARDVINERAAELVRGARALTTS
YWGSYLLAEDEVKAAAKQPLYDDSTPNDPRFERWTGEGQIGMQIQNGMTPGEVLSSEDTRLRISEPNWNDGKH
VRTLRTLSLRVGSEGRKPVWASWPLIMHRPLPPAARIKRCNVSLRRHSSREIWSAELTIECPNVTSAIREEHG
WGRGGGVEGAVGVDIGWRVVTDDDAGLRVCAYASEDGQDIGELRLSPHEITRLRKADEIRSIRDKRFDAIRLI
VRDKLATLEVPAWLSMSTLHMHVWRSPARLVSLSKRWSKERFANDEEVFDLLESWRYWDSQHYQWECDQRTKA
LRRRREKYRVFGARLAEKYEVLVLEDRAEDDRTKPMDLRKFARRAQTEMEPENETARSNRHLAATSELRQALE
EAFISHGGRVELAPCEDTTRTCTACGVVDRGLDAETEIDVTCSSCGAKQDQDVRASNNLCERWRKAQNAGGAR
NAKAAKSEGRWKKARRLRTEKQQRMGTFRNASDNSAE (SEQ ID NO: 216)
>3300020580|Ga0210403_10000550_35
[terrestrial-soil]
MTMIRVYKYGLLRPIQNEALVRAQLRAKHDYRNTLIEIERGRRTAMRNVEEQHSELATAMAASRAALVELEES
RQAIRLARSKSRSRSETNVMKERVKQARIVRRTTSQALYDCRARVRPEMISARDVINERAAELVRGARALTTS
YWGSYLLAEDEVKAAAKQPLYDDSTPNDPRFERWTGEGQIGMQIQNGMTPGEVLSSEDTRLRISEPNWNDGKH
VRTLRTLSLRVGSEGRKPVWASWPLIMHRPLPPAARIKRCNVSLRRHSSREIWSAELTIECPNVTSAIREEHG
WGRGGGVEGAVGVDIGWRVVTDDDAGLRVCAYASEDGQDIGELRLSPHEITRLRKADEIRSIRDKRFDAIRLI
VRDKLATLEVPAWLSMSTLHMHVWRSPARLVSLSKRWSKERFANDEEVFDLLESWRYWDSQHYQWECDQRTKA
LRRRREKYRVFGARLAEKYEVLVLEDRAEDDRTKPMDLRKFARRAQTEMEPENETARSNRHLAATSELRQALE
EAFISHGGRVELAPCEDTTRTCTACGVVDRGLDAETEIDVTCSSCGAKQDQDVRASNNLCERWRKAQNAGGAR
NAKAAKSEGRWKKARRLRTEKQQRMGTFRNASDNSAE (SEQ ID NO: 216)
>3300020580|Ga0210403_10001296_17
[terrestrial-soil]
MIRVYKYGLLPPTQNINLVRDQFRAAHEYRNLHVEIERGRRAAVRELFDTDEIRAASELLSRTNGAERLPIYK
SLAALRSKRLKESSTRVDEIEELAAGLRRGARALTRCYWGSYLTIEAASDQVRKMPLYGRDGITPNDPRFIYW
SGESQIGVQLQGGLTIPVLHEARDTRLRLERVSLEPARGRHPASRCRMLWIRIGSEGRAPIWATFPLRYHREL
PSNATIKWARVSLRREGLREEWSCEITIDIPGAHPRTLDTSLTGAIAVSLEWTAAVNELLVARTLDCQTGEYD
ELRLPARMVTGLRKPDGIRSVRDKNLNELRPRLIAAFREPMAPWLAAMVARIPHWRSPDPFHALAMRWRREKC
DDAREAYDILQTWELRDAHLWDYEAGSRREALRERRELYRVWSAKLSRRYKTVVLSDADLSVEARTTKEVQTD
RQTAAVYELRQSLRNAFAGEESMGPGSNVQELCDRWNGEQTTGNIRNGEKSNTFEEVKGGAWAKRKSKKSSAK
SILDATR (SEQ ID NO: 217)
>3300020581|Ga0210399_10010852_9
[terrestrial-soil]
MSVLVYKYGLRPPIEQADRVMLEMRAAHRYRNTLVEIERGRRAAQRALLAEQPQLAPFELALTVAQAELTQAY
LEIRAARQTTRRRSETEPMRVRLRETRAAVRDARGSLYLARAWLRADPALATARDRIDGVAEGLRKNARAYRG
CEWGTGGLIEKADEQARQMPLYDGAEPNDPRFQRWTGEGRISVQLQGGLELAGLEADTQLRIGDGVRLPGQTK
PSKHAERYRTLWMRVGSDERRKPIWAVFPLKLDRPLPTNAIVKLAVVSRRLDGPRVSWTVELTLDTTTCARRE
SCGHGIVGIDLGWRVFGDEIRVCAWDGDDGETSELRLHGRLLSGLSRADDLRAVRDKNFNAALAAYLAWTDRQ
GPLPAWMRPRGIHQWRAPGRLAGLCLRWSRSRFAGDAVGFDALDVWRRRDLHLWWYESGQRRGSLAARKDLYR
RFAAWLARRHDTLVLEDFDLTRVSFKGQANAQANANRHRVATSELRLILIHAFKSRGGRVVMMNPYMSTHECP
VCHAVTAFDAAAYVTYSCLGCGASWDQDESAAKILRERGSDVGDPQSARSENGPDSGGLAESRWAKAKRMKRE
KEAARNETGKGA (SEQ ID NO: 218)
>3300020583|Ga0210401_10033176_5
[terrestrial-soil]
MIRVYKYGLLPPTQNINLVRDQFRAAHEYRNLHVEIERGRRAAVRELFDTDEIRAASELLSRTNGAERLPIYK
SLAALRSKRLKESSTRVDEIEELAAGLRRGARALTRCYWGSYLTIEAASDQVRKMPLYGRDGITPNDPRFIYW
SGESQIGVQLQGGLTIPVLHEARDTRLRLERVSLEPARGRHPASRCRMLWIRIGSEGRAPIWATFPLRYHREL
PSNATIKWARVSLRREGLREEWSCEITIDIPGAHPRTLDTSLTGAIAVSLEWTAAVNELLVARTLDCQTGEYD
ELRLPARMVTGLRKPDGIRSVRDKNLNELRPRLIAAFREPMAPWLAAMVARIPHWRSPDPFHALAMRWRREKC
DDAREAYDILQTWELRDAHLWDYEAGSRREALRERRELYRVWSAKLSRRYKTVVLSDADLSVEARTTKEVQTD
RQTAAVYELRQSLRNAFAGEESMGPGSNVQELCDRWNGEQTTGNIRNGEKSNTFEEVKGGAWAKRKSKKSSAK
SILDATR (SEQ ID NO: 217)
>3300005435|Ga0070714_100002341_12
[terrestrial-soil-agricultural soil]
MSLKVYRFGARMPLDRDLVVAQLRAAHDYRNELIQYERGRREAMRALYDTPEIRDAEALLKQATKSDRKAAKR
ALYTLRREVLEARRDEAQAINALHHELQLGARALTRCYWGSYLDVESAMQQARAAPLYDEDGLTPSNPRFLRW
REPMQGQIGMQLQASRPLTTADAMRGADTRVRVERRDGPYATLWIRVGSEGRAPVWARVPIKMHREIPNAATW
KWVRVSCEPRSLRDNPEYRETWSVEITVDDPAPRARDLDTSRDGAIALSWSWDVLANESIRVASYVDTFGRRG
DIVCPASIAKGIRKPDGIRAVRDMVWNEEQKEIIHRIKRNANAPRWLVEAANTMHLWRSIARVHELARRCRIE
GIAEGPAYEALHKFVERDLHLYDYEKNARDEALRERREWYRLHACWIARTYRHALVSDHDLSREARWGDESDV
RFTAAPDQLRGAIKNALGDDAIVAYWDHEPEWCERACAAYLVGGARGEMFAERKEKTSNAWAARKKKKTETMT
ARKEAANASE (SEQ ID NO: 219)
>3300009095|Ga0079224_100000262_28
[terrestrial-soil-agricultural soil]
MKRKKSQDESINWKYGCKSPRGEGAEILRQQMREAHNYRNKLVELELLKRQEFYDLERELFPEYADLQEQEKQ
QADKVEDLRKQWKKANARARTRTEQHLLKAQIAEEKQTLKDLRARMKEMKEQVRNSEELKERSKQIKKRHYQR
LKDLRAETPAFWGNVGFVDQAAQSMASGSPPKYYRWEGEGFIGVSFRTPITPEVLMSGRNTRAWLEPCKTANN
KAGRVKKTTLHIAVESENRKLKMASLPIYFHRDFPDGCEITSVRVFCKKVGQREEWSAVFQLRSASFAKPDAA
ADGMAAIHFGWRRVDDGLRVASVVDEDGTEEVLILPESIIDSYAYVKDIQAIRDYLWNETIAILSAFLKQHSD
SLPEPVKEASQNMHLWKGRGRLVHLINVWSDHRFPGDEEMFLKLTRKGTPANEYHDSGWLHRDKHLWDIEANV
RDNAALRRKALYREFAAKMRRKYRHLITAKLDLKKIVSVKNPEEEDDAAMKHHSRVAAIHSLQAALSDSMRDG
WIVVPAAKQASTCHECGAKFQDDSGDAYISCENCGSTFDREFNACKNLLFGPKQVNAAPALV (SEQ ID
NO: 220)
>3300009095|Ga0079224_100170797_3
[terrestrial-soil-agricultural soil]
MAKRQREGTEALVYAYGLLDDQPELYSDPNVAAEVQRQRDFWDLLVRLEQEHEERVYQYLDEHAPEYRAAFEA
LCEKRRELDRLIERKRRERAEAKQKVEDPELDSAIKNATRDWKLAQKEMWAALKKARREHKEALAALRAEFYA
RIPKCKDSPLFWANYNRVRQSFDATLKRVRKQGQTVRFSDPHRDDVCLTVQIQKVRGVVGCSFEDLLSGRVSQ
LKIAPIDEAAWYTTRANRRRLCRTEVTMQVDRAGNTVRALVAVDRPVPPEARIKSAQLVWRRVGERYVGKLCL
TISMPAVERTNGSTAACGIDVGWRRTDDGGLRVATIVDSSGNVDHLELPADWMSGMDQVKRLSQYLDDAALDI
ATLLLGRDDLHPAIAAAIKRWRPGLGAGHVNVAALRDAVRELGFTGLPAELCCGVRSWRDRKERCCWYHRHIH
LSTWRDNLRRKLLLRRREIYRLAALTLAERYAVIGIEKLDLAKMAMTKKREDGSDPTLHSAARAQRQRACLHE
FRTELEHQARKRGARLELVDASKTTITCHECGAETQPTRRDRMMIHMACDSCGAVWDQDVNAARNILLAAIGA
SGDMTPPDDDGGSGAYKRHSEEISDRSQLGAPLL (SEQ ID NO: 221)
>3300010343|Ga0074044_10013672_1
[terrestrial-soil-bog forest soil]
MNILVYRYGLRAPHENRDLALSELRSSHEYRNKLIEIECARRKRVRAAEDALLGKPRLKLAEAQSALDAAIKA
VSKHRAETRKRTTPAEMLATLKAAREAQHAASKAFRSARQLVQPRCSDCRKKDLPTPCEHATPEGVGLLAELD
AAQDEAKESIKKFRNESGPFWGSYLLVDKAAGQSFSELALYDIDGKPNDPSFLRWTGEGTLGVQLQGGLSVEA
ALAGQDTQLRISSPPVACWDPSTGSRKARSRQSRESEVWLRQGSVGRAPIWCKFGLHMQRPLPPGAQIMWAEA
HCRRVGPHFDWYLTLTLKVDDAVALKPRIIPTRDAVAIDVGWRVFGEGETHELRVAYWSDGSNDAPVVIREKD
IRVPGFVIPPRGELRLDTATLNQLTQPEGVRSERDVLFDGVRARLIEWLKTPHENEPEWVDSDGVVVTLREHC
KALHAWRSQAKMAALTSRWGEWLKEHPDGDKWAYDMLVAWRGQDRYLWAVESRWRDRARLRRRELYRLFGVAL
ARTYGTVVLEEFDKREIAKRPKTEDDGEAHPARSNRQLAAVSELCECVAEAGTSRGRNVVEVPCENSTRECPV
CGCVDERNAARKVTISCACGHVWDQDDGAADTLLGRWRKRPGDAKMAGAPRKPKILNGDGSVENRMQRAKRKG
AEKALRKMELSKTTT (SEQ ID NO: 222)
>3300010343|Ga0074044_10041345_4
[terrestrial-soil-bog forest soil]
MTTRVYQFGLRPPIEGIDLVRAQLRAAHHYRNELIAIERGRRSALRQMDDTEEVRKAAGAIGGTAKADRRKAI
ANLRDARRRARESKPEEFKRIAEREHEMLLSARANTSCFWGSYLDIESAHRQARSAPLYGDDALEPSDPRFIR
WTGVEPPSGTYPRLPQSGEGQIGMQIQKKGKSSENGKSSENGKPSEEKRRLVTSDVFACLDTRVRLKRGGAKD
GDPRYGFLSLRVGSEGRAPLWATWPIKISREIPDAAEWKWVRVSLRHEGRRERWSCEITVNDPAPAARSLDSR
LRGVIAVEWEWSKLEDDSIRVARWADSMGETGMVELPGSIAKGIRKPDGIRAVRDMISHELRPRLARLIREAK
GPKPPWLVAAANTLHLWKSPRRAYELAERWTDPTLLPTARVVLFEWRARDEHLWDYEAGARSEALRERREFYR
LLAARWARRYQSIILSDQDLSREARWGEESDLRFTASCCELRGALRNAFGPDAFDGKYARSEQEDWQWCEQAR
DAWMAGGARKDAMCAKRKEQTGNAWAKRKAAAAAKRAEKGSACEPPGKSV (SEQ ID NO: 223)
>3300005468|Ga0070707_100000083_12
[terrestrial-soil-corn, switchgrass and miscanthus rhizosphere]
MPVIVYEYGLSPPKVNAAIVEEQFRLAHKYRNMLTEIELERRTKIRAIMASHPDMVPFETELAEVQAEIEKLR
GEINAIRMAARKRSSTPEQSKRIKTLAARARELRTEIKERRKRVATELAPDLKAIQDAAVQRRKDERAKSGVY
WGTYLLQEAAADQARDQPMPPKFTRWNGDGRVSVQIQQGLAKEGLWGESRQVQIATRIDSLVYDHEVTRRGDR
RRLYRTTLRMRVGSTDRQPVWAEWGISMHRPIPDGAVIKVVTVSRRRCNSTQWWWRVQFTLDTTDCKPRQRPE
YGVVACNLGFSQTDSGAIRAGYLVGDDGFEQEILVAKSDLYRGRDLTPEQKQKAMTYVRDCLAESSEIRGARD
KSLADFKTRFLEWYQTAKATTFGEDAVPEWFRDRMEHFHLWRSPARVREMMLHWASNRWAELDDPESRWPDSR
GLEMMSTWVDEDTKAEVKESSLRNKALGDRREAYRIVAATLAKRYKTLLIDDTNLKHLQDGPEPEDAEGDIPA
VKYQQRLAAGSELRQVLINAFGGTNVVKMKPSNMTVTCSGCGARDVSWDRADGFRKHRCSACREIWDQDANFC
RNLLKEYASGGEAPAAKIAKPSRSQRFHESRKKKAAAADQQEQG (SEQ ID NO: 224)
>3300006163|Ga0070715_10000067_44
[terrestrial-soil-corn, switchgrass and miscanthus rhizosphere]
MPKKPNPNKRVSSDTRGARIWSYGVLFPREREVNDAIRSLLHQANRYQNCQVVIERVRRQRYRVIRSAASPEL
ARLEQEYKDLGLAIDAEVDTMRAQRASVRRRTTDPVIAAKIKALKAKRAAVNIELKIAREKANAILRPIQDAY
NRHRKPGGVKAAPRTAEKLNAAARQTTLEEDWPELAKQLLRLEDWATRRVKQAREASGLPPGTYLLVDQATAA
TKKEPTDPRPKRFDGTGRIGVQLFDFTPQTLFSRERKQLQIDPLPATQWDTRPGRRKARTELRIDFGGNAFEM
KAAFKMILHRPLPQDASIKWAWIHVTRIGSRLHYSLQLTMRSDTFQLKPGGQGVVAVNLGWRIKEDGAMRVAY
VMDEFGTERELAMPPELRGGFVLAENLRSYSDQHFNVAKKAIGEFVKTDAAPAWLKEQCTSMHAWQRHGRLLR
IARMLAIAEFPDTVLPSGERVRNGMLSELWKRWKEHRLAAVPKLDLFDTYQVITDWARARGATDLKAATLYLW
VWKKKNDHLYNWECGLRAHKQKCRKQLYRAWATELATTYSTILVEKFDLRDTREKSAPEAEQEENPTSLIRSQ
NFAAPSELRDAIVAAAGTGRVKEQKSHNNTVTCHECGHTSDRDRRFEALIQVCESCGVVKDQDKNNCENQLSR
YFSGESPGGGLDPESARNHENSSDLKTDRDAAE (SEQ ID NO: 225)
>3300014498|Ga0182019_10003703_1
[terrestrial-soil-fen]
MKRKTSPIPTKVYKYGLLAPVENVKLVDHAFYLGGKFYNKLIEIERTSRNEYRQERARRFPNHDKVEKLVATL
SDQKKKLSEIIKASKIATKSRNVPPELATEYKTLAAAYKSAKLRQDAEREQCKKDRDFSAWTLTHNEKKNALV
ADARKNSGLMWGTYNAIAASVQQAGASAILDPEFKSYRGEGRIVVQIQGGIYLAELGSDTQLQINLPNLNESL
TRGEWRQSSRTIVKMRIGSDKHKKPIWATFPAVFHRPLPSDARIMSATITRRRLGVFQSSGRYEYHLCISCES
TMFDNEAVRPRLQDPTQQREVREHRGTSTINFGWRQFDQGKDKRLRIAMTNNEVTGLEPLWLPREIILGLQKC
ENLRSIIDMKFNEVRALLTTWLTPHKQDCPAWLAESLQFLHTWKQPDKLDRVVANWGSGMRFPADADIYPVLA
EWRTKHRHLSEWMMRQRRKSYNHRDDYYLKTAARLAQSSSRMVIENFTISKVAVKPGPEVEKTGGNEARHNRT
LAAVSELRSALIHACSKHHCPMDITPAVNNTRRCNVCGKLLDWDPAIKVDRQCPECSNWDQDVNATDNTNDKV
ASGDVVTMVVPAKTSENGEFEAGTISTFGSARKRLHNLEKTLTIQE (SEQ ID NO: 226)
>3300001131|JGI12631J13338_1000296_13
[terrestrial-soil-forest soil]
MPVTALPDGTISTARYAARGPVPAPVTEELRLGNWLDNLLTEYELDYEAAKAAAWEEDPHVALLLAAVTAAEQ
AWQAARDESAACKQKLGYAKRTGTPARIAAAKAAAAQAQQAYRAAVKARQEAAAALRDIKGLRWHVAKAAINA
AAEERDRKIAATYGPYRDRGGYWANWAEHAKHHKTAAKRVRDMRKQGQPAQLRYHRFDGTGTVVVQIQRELGV
TPEIRAQVTALKAAGRTPGQIKAETGVRAMTAAKMKPEGAVKEGDPPCTAAALADETGKWRSSVRLTPELPAG
FEELPRGERRRIASQGMFAIRTGSAANLAVSVVPVTVHRRMRGDGDVKYAKLTVTRNGPDKDMSVSLTQRVPA
PQPRAGGRLVCVHAGWRALPDGSLRVAVISGAGPLTPGLAAPGGRDARAGELTGVVRDLGDGCHEVVIPARWR
DQDAATAKTRSVRDLARDTAIAAAADWLAASPRYETTDGEPLPAAHEVRRWQSPGRLAVLGQRAARGDYGDDA
AGLGELIAGWAVPDLEAWRREARGRRHLTRRRDDAWANVAAWLCTGTREVRVDEWDIRAVTRRPGPGETDDPQ
AAAARANRTLAAPGALRQRLTITAVLAGVTVTVLDPPDAGSVLQVHAGCGGVLDRDARRESIVVQCPGCGARV
DQDVNMVRLMAARQPSA (SEQ ID NO: 227)
>3300001593|JGI12635J15846_10002852_1
[terrestrial-soil-forest soil]
MPVTALPDGTISTARYAARGPVPAPVTEELRLGNWLDNLLTEYELDYEAAKAAAWEEDPHVALLLAAVTAAEQ
AWQAARDESAACKQKLGYAKRTGTPARIAAAKAAAAQAQQAYRAAVKARQEAAAALRDIKGLRWHVAKAAINA
AAEERDRKIAATYGPYRDRGGYWANWAEHAKHHKTAAKRVRDMRKQGQPAQLRYHRFDGTGTVVVQIQRELGV
TPEIRAQVTALKAAGRTPGQIKAETGVRAMTAAKMKPEGAVKEGDPPCTAAALADETGKWRSSVRLTPELPAG
FEELPRGERRRIASQGMFAIRTGSAANLAVSVVPVTVHRRMRGDGDVKYAKLTVTRNGPDKDMSVSLTQRVPA
PQPRAGGRLVCVHAGWRALPDGSLRVAVISGAGPLTPGLAAPGGRDARAGELTGVVRDLGDGCHEVVIPARWR
DQDAATAKTRSVRDLARDTAIAAAADWLAASPRYETTDGEPLPAAHEVRRWQSPGRLAVLGQRAARGDYGDDA
AGLGELIAGWAVPDLEAWRREARGRRHLTRRRDDAWANVAAWLCTGTREVRVDEWDIRAVTRRPGPGETDDPQ
AAAARANRTLAAPGALRQRLTITAVLAGVTVTVLDPPDAGSVLQVHAGCGGVLDRDARRESIVVQCPGCGARV
DQDVNMVRLMAARQPSA (SEQ ID NO: 227)
>3300009813|Ga0105057_1000075_5
[terrestrial-soil-groundwater sand]
MEDAMEADQTPATAEPAAGAVIVYRCGLRAPLDWGRDCDDQLYLMTRLWNTLVEIEHAHREAYFAATASDPVV
AATEAEITGLERLLEQLYAQRAELRKAARKRVRTPELDERIAELKAKLKARRAEAKEARKAARETIKPQLETL
EAERREAVKVARNASGLWWGNYNAVCADYDRARSAVIKRGGKLQFRRHDGSGRLVNQIQGGMSVADLLGRAHS
QVQVTGGAWAVNARGHLTATVYTRTAAAARAAGAGGTRRTVTWPLQLRRPRPGPYAQARIKEVVITRRRRGHK
FDWHVSFLCQLPATEPALPAGRACGIDVGWRRLNDGVRVGTIVYSSGEREFVVLPERLVAAARRAQDIASRRD
KIFNDLIVSWRAIDWTNAPEELAATAVRLQKSKLSPPQLHGLVYAWRRHPFFAPDAFTVADRWLAEDKKLWET
EASLARHVSNARRDLYRGAAKRLVATCGLIGIEDIDLAALARRKTPAGGDNEIAQATAWWRRIAAPGELLAAI
SHAARRDGALIHKHSGKSTWICAQCGTESMPSDRSQLVHTCPHCSHTWDQDVNAARNLLAAALASAPVTLDGP
AALAWEKPRDPNDLEE (SEQ ID NO: 228)
>3300009813|Ga0105057_1000075_5
[terrestrial-soil-groundwater sand]
MEDRMEDAMEADQTPATAEPAAGAVIVYRCGLRAPLDWGRDCDDQLYLMTRLWNTLVEIEHAHREAYFAATAS
DPVVAATEAEITGLERLLEQLYAQRAELRKAARKRVRTPELDERIAELKAKLKARRAEAKEARKAARETIKPQ
LETLEAERREAVKVARNASGLWWGNYNAVCADYDRARSAVIKRGGKLQFRRHDGSGRLVNQIQGGMSVADLLG
RAHSQVQVTGGAWAVNARGHLTATVYTRTAAAARAAGAGGTRRTVTWPLQLRRPRPGPYAQARIKEVVITRRR
RGHKFDWHVSFLCQLPATEPALPAGRACGIDVGWRRLNDGVRVGTIVYSSGEREFVVLPERLVAAARRAQDIA
SRRDKIFNDLIVSWRAIDWTNAPEELAATAVRLQKSKLSPPQLHGLVYAWRRHPFFAPDAFTVADRWLAEDKK
LWETEASLARHVSNARRDLYRGAAKRLVATCGLIGIEDIDLAALARRKTPAGGDNEIAQATAWWRRIAAPGEL
LAAISHAARRDGALIHKHSGKSTWICAQCGTESMPSDRSQLVHTCPHCSHTWDQDVNAARNLLAAALASAPVT
LDGPAALAWEKPRDPNDLEE (SEQ ID NO: 229)
>3300014489|Ga0182018_10031574_1
[terrestrial-soil-palsa]
MIRVYQFALRTPVDNEPLARAQLLAAHRYRNQHVAIERGRRWAVRLCEASEEVDEAVALVQSATKSTRKDALK
DLRAARKAARETHADELARIAELDAEIRRNARSHTSSYWGSYLTIEQSSDQVRRMPIYEPDGLTPSDPRFVRW
TGAGQIGVQLQGGALTPDVLAGRDTRIRLIDGVLWLRVGSEGRDPIWAKWPIVQHREIPSGADWKWARVSLRK
EGPWERWSCEITLEIPGEHPRNLDKDPQGAIAVEVTWDKPGDALVVARWRDDAGRTGTIELSEYDEQGIRKPD
GIRSVRDQLLNDLKKRLPRAYAECRGDLGPPWLGEAIDGAQYWRSQSRAHTLLTRWRAEKCDAARAAYEILDA
WWLRDMHLWEYEAGARGQALRRRREKYRVLAVTWSREYRHVILDDRVLSREARFGDASDLRFTAGPSELRQCL
EHAFGGRNGGNVTTHPVRDDAAKSETEERDWCERAIDAWIAGGARATKKVSESTGVKGGAWSLRKSKKSQKQA
ENGTAREPVAKGAV (SEQ ID NO: 230)
>3300014501|Ga0182024_10047267_8
[terrestrial-soil-palsa]
MSKFRVYQYGLKAPTENAELVREQMFLAHRYHNTLIEIERGRRGAIRTLTRGHNATIRQLEADLLEADALVGK
IVREIKTQHSETRSRLSTQTDKEELKVARQKKKEIKSQLIEARYLDKNNPSIINERTNINDLAKEAIKSARKH
CGVYWGTSQLIDDAVEASRKMPLYNGEKDNDPSFKPWKHQGSVGVQIQKRDDIQGMDVKNVFGADTCFRIDCV
NEGAFYAEKRGDRRKQRKTTMRMRINSDDKGKPIWSYFPMTMHRPLPDGGIIKKAKVRLKKIGSREEWSLSIT
VDMSNVLMTTNNNHEAVAIDIGWRDMKDDNGQTTGFRMCKSRGTDGKIEEIKLDPKIISAIKKANELRGLRDD
NFNKERASFVAWAKVNVLPDWLVKETKTIAQWRSISRLVKLFKQWKNNRFDGDEVIFGVSGKWNKGDKSVITG
TGLAGWAYHDFHLWNWEANQRTKAIRRRKEFYRVEASKLAKQYQTLVLEDFDLSDVSQTAEPEAEDDNQRGRS
NKTISSPSEFKLALINAFDARNGKIEKVNPKGTSYICHLCKSKEHLDSTFHIHTCSKCHQTWDREDNATANIL
TLWRERLSDEQNAVSARKDENGNENKGVEETRYQRRNRAKQEKKARLETARNAEANIAE (SEQ ID NO:
231)
>3300014501|Ga0182024_10150440_2
[terrestrial-soil-palsa]
MIKTEKYGLLSPTLNADLVLLEMRKAHDYQNNLIEIERWRRDEIRKIESIYGNIPQLTIDYDIASNDYETILK
TKKKNNSNARANVSTPELNQQLKDAKSKRKACENKLKQARLSSRKDDKIKKAKDNISILENKKQSLLRKSDAA
PWYGTYMLIDHAFGIPSAKGKTKGMPLYNGINPNNPIFRNYNGEGRIGIKQFQPYEPINKIININPTSKFLQI
VPIPPPKLKKDGSQRKIGNKNLKLLRIRIGTGEKNAPIWAEFPMVYHRPLPSNSVINMVQITKKIIASREKWA
VSISYEDNIQFSKNEIKKVVAFDLGWREFPDRIRIACWKDNDGKSGEISLPIGLGIKKDSNDKIIKNKDGNDK
LFHSTINKLRKVKELKSGRDLDFDRVKQLFGYFVNSGIIFPQWFQDWLNTTNKNGKKINDITYISKWRSQTKL
SKLILQWKNNRFTGDEDIYMWLEYWRYHDFHLRDYEYNLRNKSIGAIESLYKNTAAHYANNYDAAIFEDINLS
NIAKGKVGSTNRQLTAPSKFRNACKNAFNMRGKCYEEIIARNTSRECAVCHVLNDIGGKLEYFCSGCNVELDR
DENAAENILERGRKKLSDNNTYIDSHENCEHDSNAKNAVGARIDENCNENNNLQYA (SEQ ID NO: 232)
>3300001356|JGI12269J14319_10001968_12
[terrestrial-soil-peatlands soil]
MTRRVYQFPLRAPIEGAALVRAQLRAAHEYRNDLVSIERGRRSALRAVDDVPAVREAIAVVLAATKSTRRSAI
ALLRDARKEARAKAADELVRIGVLDAQARRDARAITPTWWGTYLDIEAAHNQARSAPLYEPDAVTPSDPAFAR
GPRLGREAFAPDDARAAWWLGDGQIGVQLQKGLPTPGALAGADTRVRLVLRPADHPRDRYGTLWLRVGSEGRD
PVWAQWPIKLHRAIPDSAIWKWVRVSVAREGTRERWSVEITVDDAAPRPRDLDRSLAGAIAVEWEWSLLDSGA
IRVARWADTRGGSGELLLPERIATGIRKPDGIRAVRDLELNALKDSLQQALREASDVATPRPPWLADAASTLH
LWRSPDRFRGLLYRWQRERYDGARSAYEMLEAACHRDDHLYDYEVGARRGALGARRDLYRCVAARWSQSYRTV
LMSDQDLSREARWGPESEVRFTAGCFELRSCLRNAFGDADAIDSRWRDAPGEQEDREWCERTRDAWSAGGARG
DGRFAIRKEKTTNAWAARKAKSKAKRGGDEASRDPDGKGAE (SEQ ID NO: 233)
>3300007533|Ga0102944_1012316_2
[terrestrial-soil-pond soil]
MAGRKKKDAPPSRVWIFGCGEPVDGAELVRDQLFETHRYRNDLARHVLNSRQVYREARSEICRVAALEQEHLE
AKEALDVLRQEQKAWSAAARRRVQSPELQQAIKDAKQKKRDVLERLKQAREEVEQDPELQQARAEINKRAAAE
KKRLYNESPAVWGSRLRVDESWLQMRWGRMDPKFRRFDGSGRVVVQIQKGMSVAQAFECKDTRFQLARPTRDW
DRRTGRRGGTRTMFRIRVGSEGKRRTPVWATFPVTLHRELPEDAQIKWVEVRAVRQGPDLRFQLHLTLEWHGF
DPRSRGAGAVSIVCGWRAMHDGTVCVGRWIDDSGRSDVLVLPADVGEAEKHASSLRSISDLHFDAARRVFKSR
RHLLPAWVTEESAYLDKWRSHARLAKIVGRLTAEILGDEAAHVWRQWRRYRQREGLDLHAPYEELSTWLSEQG
EVDPARQTAFYLEWWRRKNRHLHRVECNVRTKALRRRKAIYRNWAASFARRYETVLVDDFDLRQFARNAAPEE
DARQDYLHGVMRLAAPGELRLAFLHSLGGARAVKVPATEGLRRCYLCGSPMRRPDRSGTVEHDCGVITPWQTI
RGLDMLRGAGVDTVAAEQKLLEGHEAMKKLFRELARKG (SEQ ID NO: 234)
>3300005903|Ga0075279_10000001_30
[terrestrial-soil-rice paddy soil]
MSETDTMAFKFRILRIVSDEARIKREIREAHQLYNDLVAIERKRRDDTRLFWADRGGYADKLAGLRAAAEDAE
KAAALTAKGDAGKKERQEIWAPVDTLKREIWELQRKTEEELSDPAKVRRKQRARELQAEAKARAGKALKKEAL
AALLDAEPDCMSPRDRRRLELVREYEARGVAVSGKAVAQRLRDEGLVGPTEQIEEAARKAGYEAYLKRGVSPG
TRAIIADAFERSLEDLEPWATQRFSRWDGHGSFGVQVQGGSLTEEVYSGEHTQVRLRRLEDTGKHREGSRRSG
RRHELRVRIGSDGRAPVWAVFEAIVDRPLPPEASIKRVVVTCDRLGVLDIYHVVFTCSVPSSVYHKRSGEQRG
TVAVDFGWRSLGGEEMRVGYWVNDRGESGEIRLPGVGVARRGTTGKNSPYEPKIRGQVPIRQLDKHTRDLTEI
MAREFAEYDGGKICGGALRDVASWLSANVSIVPEWLTERTTGIHVWRSQHRLYWLAQDWKAQRFDGDAEIFER
LSAWASDWAHLAEWERRQHAAILAARNEHYRLVAVGLAKKYERIVINGADFAAAKRRKTKDETDRLVMIDDRS
RSQAHLAAAGELREEIVRSAKKWQAIVMKAKPNKATCHACGSTCVYDAAKDLAHECEHCGVRWDQDENCCRNM
LCEWSGDGQTAGGARVSPNAKKSGEVLRSKKRDEDGGPIGEAAE (SEQ ID NO: 235)
>3300005524|Ga0070737_10002282_10
[terrestrial-soil-surface soil]
MSTGTVPPAAGNTPPTGEVTVARRYGAKPGPIPAAIREELDRAHWLVNRLVEIDRECDEQVTAVWEADPQVGP
VLAAVRDAEDARAAASEELRRAKVKLGAVKSGRSRTGLDVARARVDAAREAVRQASAALKQARDRAGAVKQER
WPAAAPVIRAAQEARDAAIRATYPEFVARGGYWATWNDITGAHKRRRDRVQQLRRQRRPAQMRYKRRDGTGTL
TVQLQRQLGVSAAERAHVTGLRDAGLAPSQIAALITAGVPAAEVTPARVARLRAAGLSAPEVTQALVAGADPA
AAAAASRTPGKAARRAALGEVLAAAAAARPPRTPGRRWRPQSVARVRAGGKDTPGDPPFTPDVLAGPAGPAAL
QVRPVLPPGYPQLPRRQQRALARQGEVVFRTGSAANAAYTTIPVVLHRPLPAGGDVKMGRLTVTRCGPDLEQS
VSITARVPAPPPAAGHTAAVHIGWRALGDGAIRVAVITGPRIPPPRQLAEAGVVRPVGGCWEVVVPPRWQVAL
TRVDGMRSARDREWQQVRDRVAAAIPPGHDTLPPPAQVRAWRSPGRMVTLAAACEAGEHGGHGRAIADLVTPW
ARRDRAAWRNESRARRRILRRRDDAWAVIAAWLTAGAGTVIVDDWELPPLGRRPGLTEEDDPQWRAARANRVL
AAPGALRARVRVTAELAGVAVAEFPVPRPGQAHAGCGHPLDPDARREDVLVPCLGCGVKVDQDINMLTLMLDG
AREGAPQAG (SEQ ID NO: 236)
>3300005524|Ga0070737_10031205_1
[terrestrial-soil-surface soil]
MTAYSYGCPAWAVPLAPRETDPVTVIAALAGTAQALRQAQAALPGGGEREWKRWAQALQARDQAREAVWVTGE
EALAGQLRLACRLWNRLVEVTRGHERARAAVWASDPAVAAAQETLDAARAAVAACHERIRVSRQADRTTVPRD
ADKQALDEARAAARAAREARDAAREAAFPRLRARFAAAAQARLAGVKAAAAEATEAGLGWAACNDITWRRFPA
ALQKVDRERAAGRPAELRFRRWDGTGTVTVQVMGGAGIPPRTLPALNSGRHPRSAVMRLQPWRDPSAGRPKGA
DRHGTLTLTAGRSRRHGPLRLQIPVVLDRYLPADADIAEVKVTRFREGTRHRLRVSAACYVPAPPGPPPGGAT
VAVRLSWRAAGGGWVTAAQVGSSSPLPPLPRSLEHAVRGTAGGPRLPLVRVAPGSLSAEVLYYAGWRRLLERG
EATQAVRGQNTDILREKVTAALRDDPALAAAVKVTAGEVARWRAPRRFAALARRWPAGHPLRPLLEEWRRRDR
HLQDYQACETAQVLAARRDAWRCTAAWLCAGASAVVIDGTRLDAEKQAPGDDEEDPEGARGARRLLHRSAPGE
LRAAVEAAAARRGIPVTILKAAVEPAAGGGA (SEQ ID NO: 237)
>3300005524|Ga0070737_10031205_1
[terrestrial-soil-surface soil]
MTAPGGEGTPARVMTAYSYGCPAWAVPLAPRETDPVTVIAALAGTAQALRQAQAALPGGGEREWKRWAQALQA
RDQAREAVWVTGEEALAGQLRLACRLWNRLVEVTRGHERARAAVWASDPAVAAAQETLDAARAAVAACHERIR
VSRQADRTTVPRDADKQALDEARAAARAAREARDAAREAAFPRLRARFAAAAQARLAGVKAAAAEATEAGLGW
AACNDITWRRFPAALQKVDRERAAGRPAELRFRRWDGTGTVTVQVMGGAGIPPRTLPALNSGRHPRSAVMRLQ
PWRDPSAGRPKGADRHGTLTLTAGRSRRHGPLRLQIPVVLDRYLPADADIAEVKVTRFREGTRHRLRVSAACY
VPAPPGPPPGGATVAVRLSWRAAGGGWVTAAQVGSSSPLPPLPRSLEHAVRGTAGGPRLPLVRVAPGSLSAEV
LYYAGWRRLLERGEATQAVRGQNTDILREKVTAALRDDPALAAAVKVTAGEVARWRAPRRFAALARRWPAGHP
LRPLLEEWRRRDRHLQDYQACETAQVLAARRDAWRCTAAWLCAGASAVVIDGTRLDAEKQAPGDDEEDPEGAR
GARRLLHRSAPGELRAAVEAAAARRGIPVTILKAAVEPAAGGGA (SEQ ID NO: 238)
>3300005534|Ga0070735_10023967_5
[terrestrial-soil-surface soil]
MEKRESTVVTIAPVRMCGTTIRVYRYGLAPPLDWDDECDAEMARLDRFYNALVEIEEAAQAEYRRLSSSDETA
LLETRIAAAEEAKDWGAAKALRAALKEIRAALRKANAAAIDAAEEKRKADAKAARQNCGAYWSSYNAVIRSVE
LARQKAIKEGAAFGKRTHEPGKGDWRMTVQIQGGASVADVLGGKNSQLRIAAPAHFGALRDRPAGMSRKACRH
GRVTMVVHNTGGLRRVTWPLMMHRPIPPEAIIVGAEIVKRRRLGSRWDDWHLCVTVREPAPAPHESPDCAGVN
IGWRRLSVERGLVIDGAGLRIATIWDGATLNHVILPEEIISAAWRCDELTSAIDKRVDAATARIFKDSPDHPV
ARQLGDTFVESGRLRVRDLWTFANAMTPAPDWLLAMLRACSRDRRERAGLLRRMARRRRDIYRVAAKTIAENY
GRIAICAVDWAKLARLRESGKDNPLPPPARGYRKIAAPGEFEAELRRAIKARGGTIMDIKDSVSFLCHACGKE
HAPSERSAAHHTCPSCGATWDQDRNAAMNLFAALDSSGPAAIKPADSLENGKGEMQSTAYVGRFQRRAKQAQE
AKLANKEALENASELVDETIGCK (SEQ ID NO: 239)
>3300005542|Ga0070732_10013271_3
[terrestrial-soil-surface soil]
MIRVYKYGLLPPTQNINLVRDQFRAAHEYRNLHVEIERGRRAAVRELFDTEEIRIASELLSRSSGTERLPIYK
SLAALRSKRLKESSVRVDEIEELAAGLRRGARALTRCYWGSYLTIEAASDQVRKMPLYGRDGITPNDPRFIYW
SGESQIGVQLQGGLTIPVLHGARDTRLRLERVSLEPARGRHPASRCRMLWIRIGSEGRSPIWATFPLRYHREL
PTNATIKWARVSLRREGLREEWSCEITIDIPGAHPRTLDTSLTGAIAVSLEWTAAVNELLVARTLDCQTGEYD
ELRLPARMVTGLRKPDGIRSVRDKNLNELRPRLIAAFKEPMAPWLAAMVARISHWRSPDPFHALAMRWRREKC
DDAREAYDVLQTWELRDAHLWDYEAGSRREALRERRELYRVWSAKLSRRYKTVVLSDADLSVEARTTKEVQTD
RQTAAVYELRQSLRNAFAGEESMGPGSNVQELCDRWNGEQTAGNIRNGEKSNTFEEAKGGAWAKRKAKKSSAK
SILDATRQG (SEQ ID NO: 240)
>3300010373|Ga0134128_10000310_109
[terrestrial-soil-terrestrial soil]
MAFGNVAKPSRIYTHKSRPPHEGAQLASDQLYRAHQYANALIETERQRWNATQDLMRSMFPRIAELEARIDAI
NAAIEAKNAAVKHQNQAARSRTATAEDRAEVKQLQSERRDAAKELTAERHRQTGVPKPRKEAWERIHAVINAT
TRDEKERKALRKAAQAAIDAGESPAPLTAEEDREAFGDPEYRRRRAAIDETANADRRAKRAVCEVYWGTYLCV
EGAVDKSVEDCAKGQPVRNGKPPVPPGPPRFRRWSPEGKLGVQLQGGLSWLDALAGTDSRLRIELQPLAARPT
VSKSGKPLPLADPNSRRSRENSQVVVWARIGTEEDGRSPIWMKTVAHLHRHPPADAVIKWVYLQRNLKGVQTW
WEAQFVFEREEGWAKDRETLRGEAAIDLGWRKVADGLRVAVLIDDDGERMECVLPDSWLESWAKAQSIQGFRD
TEFDAIRPALVHWLKARREAGTLPEWLGEATGSLHQWKSCERLGKVVWQWKDQRFDGDEAMFERLVEWRNRDR
HLHNYARGMEETAVRRRDEHYHRWAALVRRRYALVKVEDTNWREMQHRPEATDDGKNATIGAFRCAAVGRLLE
TIQEHVWNVVAVEPAWTTKTCHACGHVDDFDSAKELVHTCSACGEIWDQDDNAARGLLKGRVLKWLTREGRKL
PKAERVRDVEAAV (SEQ ID NO: 241)
>3300010373|Ga0134128_10011458_1
[terrestrial-soil-terrestrial soil]
MFGHESKPSRIWSFKARPPHEGGGLVTEQLRLAHRYRNALVALARRKHEDTEAALLRYRPRLGTLFGLASSLS
EAFSAAEDSVKARSVAGRRRDVLRGDERKAHAELRRAKEGAWAAYRRERSAAFRVPAVRSELDAIDAAFYSGW
RAARGVAVNEWGLYWGTYLPAEAAFNQSLKPPGAGKDDASVSAKENIRKRWGPPEFRRRLRGEEFVGHEGAVT
VQLQGGLDWYAAKLGNDTKLRILPVPAGLTPTTVTRPAFTRAERDRIKRLRREASLVRKELGRLGTPADPGGG
DIDGRRSKLVAWGHSVRGEIARIRAGATLRTTPLPPPDPGSKRSRAGRRAEVWVRVGSRGPAGREPAWARAVA
YIDREPPPGTVIKWVHLRRTLAGRSARWSVQFVLSRDSWDAETAAAGAAGVDVNWWMTPVGLRVATAAGSDGS
VSHLFVPNDVVDAWRKHESLQSIRSVNADAARAHLLAFRSSGVRLPGWFREASAYAHAWKGGAKLAELVWAWK
GRRFPGDAVVYARLEAWRKQDRHLHDWHGAQHDKVRRIRNDLYRKWARGLARRFRLGALKDTNYAAVRRAAPA
GEEDKGFTRLYSGIASPGLLSRYLREAFAECAELPANNVTRECHGCGMVNAFDQARVRFHACQGCGASWDVDE
NAARNLLRRAAGARREAI (SEQ ID NO: 242)
>3300010373|Ga0134128_10096594_3
[terrestrial-soil-terrestrial soil]
MPTKVYTYGAKLPIHGLELLDDSIIHGHRYYNALIALNRARAEKIDALRHQLIPGLDDAEAAVVAAESGLEDL
RAAVRQRNAERRTKTATPEDRQAIKAAVASLKEAKAIRKEIRSRCRDCVEYKESLDKVWNDFSEASKAAYNES
PCWWGTKLHISQSVERAVESSAKIGGVPRFRRWEQVRFDPAAGDDGAFVGAGDGVAAVQLQGGMTVEELLAGA
DWRLRMEFVADGKRTSLRAARKVIFSLRLGSDGREPRWVRIPAYYHRGGELPVDAKIMWAMIVRRALAPRRQR
DGSWRPWYEYSVQLTVRTGEARPTADGGICGVNLGWRTKPDGSLRVAVAVGGNQETHECVLPAHMLAGWKLGE
TLQGHRSVNFDRIRAVLADWLRGSTEPPLGLEVSAVELDRLATLANVAEDADRFRRGARTLRAWAANWTRPAD
LPAWLTTESQYVGLWKSHARLAQLLDRWRSQRFNGDEAAFSALMVWREREAHLQQWETALRKRLDGQRKDIYR
KFAARLARRYQVVATEDTDYRALKLRKPAEDNADDAAVKEHMRHGAPGLMRQYLRGRAAVELRIKSKDISRIH
IDCGGINSEDRRPSILIVCPHCKVEYDQDVNAARNVLARAEVVNETPGAAREAQPKETGDDTLNDGGKRGRWS
KRKADRSRKQVESAAQQ (SEQ ID NO: 243)
>3300010400|Ga0134122_10000107_57
[terrestrial-soil-terrestrial soil]
MPIRVFEYGLKAPICNGPLVEQQMRSAHTYRNLLTQMERERRQEVRTIMAAHPDMAPLEARLAEIVTQRDEVR
KAITTSRGITRSRSESPELRALARELGKQIREIRAEIKANRQAVAAAVKTDLDRIEIASVRRIKEARAACGVY
WGTYLLQEADADRARQEAFPPQFRPWRGEGRVSVQLQKGLSIEELHGDDTQLHIDPVPPAAHDIGTRRGDRRR
ARRTMLRMRVQSDGRQPVWAVWPMRYHRPLPPGSVIKVATVSRRRHDCRSWDWMLHITVEIPDAAMKPSPASG
VVALNLGFCLRPGGTIRAGYIVGSDGVEQEILVPRSVIDAISKCDSIRSIRDKNMDAMKAQMGQWLATVRATH
EKIVQEIAARPPPADVDSAWYRFCAYWSMNGPAWPAWFAEATATLHAWRSADRFRRLAFRWRDNRFVGDATGF
RILEGYSDQRFHDERDAWRYRDEHLERYESGMRRRALLRRRESYRIAAARLSATYRTLLIDDTDLRDFQRSPA
PESESIEITRIKRNRGLAAGSELRLTMVNAFNGLSGRVVKISPATKPCHACGVVNTWDRTEDREHTCTACHQR
WDQDANACRNMLLAHERDQATDADAPSKKRLSRSERLRKNRDAPKAA (SEQ ID NO: 244)
>3300010401|Ga0134121_10002041_17
[terrestrial-soil-terrestrial soil]
MQRKTTTAPTRVYSYGCSFRGVTKNADLVEEQYRRAHAYQQKLVELELQRRAAVRAVLATDAGVVKLAEVVAQ
HEKALTDDLVAAAAIKQATRSKKLPPELRDRLAQTKQDLRAARDAWKAARRLFATDPNTKAALDKTNTDHVVA
VKAARAASGLAWGTYLILERAADQQRNEKMDPKHRGYTGEGRLAVQFQGGTTVAGVFGGEDTRMRIEPKPGGS
RKRHICKLRIGSDGRAPIWAEIEVFIHRQLPADSRITWARLKRTRRGRDYLYDLQITLESQTFAGVLQDMAHR
RVAVDLGWRVTDKGAGGLRVAYWRDSDGRHEELRLPAKLLSSLDYPDQLLGIETNWFERAKAKLLAWRADVVL
PEEHRSYTGTLAHWQSPLTLASYVWWWREHRFAGDELIFATMDEWRVRRFWHYRDWRMFQRDKALAARKDFYC
VFASKLVVDCKELVLEDFDLSAFATKDTGPSAFRYWRRTGAPSELRLCLIAAAKKVGAKITLVDPAMTTRRCQ
ACGSEEPWDQKTETVHTCKTCGTTWDQDDNATINMLASGSMVSGTSESLDPAE (SEQ ID NO: 245)
>3300004633|Ga0066395_10000027_32
[terrestrial-soil-tropical forest soil]
MPITRIYRYGLLEPFLGAELVAEQMSLAHRYQNSLIELERGRRERVRAIMLGAPSLEEAQAIVDRAVKDLLDA
RQKIKDVRKAAQRRAETEADRASVSEIVVRLREARRVLKETRAAVRADSAIALSIAGVNDEIAEEQKRRRAAC
GVYWGSYLLVEQAMDAARKAIVDPRFRRWDGSGRIAVQLQGGLSWADACAGDTRLRVDLAPRAVGKGKPRPTV
SLRVGSNGRDPVWASWPIILHRPVPEDATIMWAAVHRTILGGKARWHLLLTLRLPDDFVVEKGGKGTVAVDLG
WRQRENGLRVGYMRDDAGDAGEILLEPAIVDGFKKVDDLRSIRDKRIDVMRPRLAEWLRERELPDWLAAERAT
MHLWKSAARFSRLAEIWRGKRWDGDVEGFDLLWAWRAKDRHLWLWEANLRDKVLARRLDRYRVLGAELARKYH
TLVLEDFDLRNLQRHAKPESETVEIGPVRGRQRIAAPSLLRQKLVDAFVARGGRVVEVPSANTTRSCHACGLV
EAWDPVTNLMHACTGCGALWDQDDNACRNLLLRERLGADEASEAARPTETEPKTSKWGRLGRHKKRPLASGNA
NE (SEQ ID NO: 246)
>3300005332|Ga0066388_100004304_4
[terrestrial-soil-tropical forest soil]
MLVGWFRYNEARPAGGDVATCNNATMKRSEFNGRVLVYRFRCPVAASNIPEAAETQFRLAHELRNELVAVERA
HADAVAAIWADHPDVAATLQAQAVAEAAVAELVERAGKERIADRAKEPRAQTRAEIKAARATLAAARQDTKAA
RSAAYPLVRPAMADAQTRRRQAIGDAGRGAKTRGLFWHTHDAVLAGHDTAVKRVAALRAEGKPAELRSRRWDG
EGRIRVTLMRHEWSHGCGAQPCGQPSPECPRRQPGDPLRTPALLAGGQGPWRNVCRLPAHMDPAICAEHPPRR
HGERETILLRVGSEEREPIWWELPVFVHRPLPPGADVAFVEVRRERLAGQTRLSVCVTVRLPPVATLTEGAVA
AIHPGWRSVTGGIRVMVIAASRPLGPIPERFAPVVRPLTGNHVEIIAPEEWVRVLGHADSVRSIRDQALDVIR
RKIVDALAEDVPGVEVSAADVARWRSPGRFAALVRQWPNDHPLADVLWAWRRQDRHLWKGEAHERDQIAARRT
DTWRHVAVWLCDQVAVIGHAPTPIAELSRVPVIEDGDDRQATLARAHRALAAPAELVSLIEIAAGQRGVRIVE
IDGARLTATHHVCGEVTGDLARDSVMLWCSRCGIAFDQDANAATASLARTIGDLSSAKVQ (SEQ ID NO:
247)
>3300005332|Ga0066388_100004304_2
[terrestrial-soil-tropical forest soil]
MKRSEFNGRVLVYRFRCPVAASNIPEAAETQFRLAHELRNELVAVERAHADAVAAIWADHPDVAATLQAQAVA
EAAVAELVERAGKERIADRAKEPRAQTRAEIKAARATLAAARQDTKAARSAAYPLVRPAMADAQTRRRQAIGD
AGRGAKTRGLFWHTHDAVLAGHDTAVKRVAALRAEGKPAELRSRRWDGEGRIRVTLMRHEWSHGCGAQPCGQP
SPECPRRQPGDPLRTPALLAGGQGPWRNVCRLPAHMDPAICAEHPPRRHGERETILLRVGSEEREPIWWELPV
FVHRPLPPGADVAFVEVRRERLAGQTRLSVCVTVRLPPVATLTEGAVAAIHPGWRSVTGGIRVMVIAASRPLG
PIPERFAPVVRPLTGNHVEIIAPEEWVRVLGHADSVRSIRDQALDVIRRKIVDALAEDVPGVEVSAADVARWR
SPGRFAALVRQWPNDHPLADVLWAWRRQDRHLWKGEAHERDQIAARRTDTWRHVAVWLCDQVAVIGHAPTPIA
ELSRVPVIEDGDDRQATLARAHRALAAPAELVSLIEIAAGQRGVRIVEIDGARLTATHHVCGEVTGDLARDSV
MLWCSRCGIAFDQDANAATASLARTIGDLSSAKVQ (SEQ ID NO: 248)
>3300005764|Ga0066903_100000051_27
[terrestrial-soil-tropical forest soil]
MPITRIYRYGLLEPFLGAELVAEQMSLAHRYQNSLIELERGRRERVRAIMLGAPSLEEAQAIVDRAVKDLLDA
RQKIKDVRKAAQRRAETEADRASVSEIVVRLREARRVLKETRAAVRADSAIALSIAGVNDEIAEEQKRRRAAC
GVYWGSYLLVEQAMDAARKAIVDPRFRRWDGSGRIAVQLQGGLSWADACAGDTRLRVDLAPRAVGKGKPRPTV
SLRVGSNGRDPVWASWPIILHRPVPEDATIMWAAVHRTILGGKARWHLLLTLRLPDDFVVEKGGKGTVAVDLG
WRQRENGLRVGYMRDDAGDAGEILLEPAIVDGFKKVDDLRSIRDKRIDVMRPRLAEWLRERELPDWLAAERAT
MHLWKSAARFSRLAEIWRGKRWDGDVEGFDLLWAWRAKDRHLWLWEANLRDKVLARRLDRYRVLGAELARKYH
TLVLEDFDLRNLQRHAKPESETVEIGPVRGRQRIAAPSLLRQKLVDAFVARGGRVVEVPSANTTRSCHACGLV
EAWDPVTNLMHACTGCGALWDQDDNACRNLLLRERLGADEASEAARPTETEPKTSKWGRLGRHKKRPLASGNA
NE (SEQ ID NO: 246)
>3300010047|Ga0126382_10001209_14
[terrestrial-soil-tropical forest soil]
MDNAAYRVLVGWFRYNEARPAGGDVATCNNATMKRSEFNGRVLVYRFRCPVAASNIPEAAETQFRLAHELRNE
LVAVERAHADAVAAIWADHPDVAATLQAQAVAEAAVAELVERAGKERIADRAKEPRAQTRAEIKAARATLAAA
RQDTKAARSAAYPLVRPAMADAQTRRRQAIGDAGRGAKTRGLFWHTHDAVLAGHDTAVKRVAALRAEGKPAEL
RSRRWDGEGRIRVTLMRHEWSHGCGAQPCGQPSPECPRRQPGDPLRTPALLAGGQGPWRNVCRLPAHMDPAIC
AEHPPRRHGERETILLRVGSEEREPIWWELPVFVHRPLPPGADVAFVEVRRERLAGQTRLSVCVTVRLPPVAT
LTEGAVAAIHPGWRSVTGGIRVMVIAASRPLGPIPERFAPVVRPLTGNHVEIIAPEEWVRVLGHADSVRSIRD
QALDVIRRKIVDALAEDVPGVEVSAADVARWRSPGRFAALVRQWPNDHPLADVLWAWRRQDRHLWKGEAHERD
QIAARRTDTWRHVAVWLCDQVAVIGHAPTPIAELSRVPVIEDGDDRQATLARAHRALAAPAELVSLIEIAAGQ
RGVRIVEIDGARLTATHHVCGEVTGDLARDSVMLWCSRCGIAFDQDANAATASLARTIGDLSSAKVQ (SEQ
ID NO: 249)
>3300010047|Ga0126382_10001209_12
[terrestrial-soil-tropical forest soil]
MKRSEFNGRVLVYRFRCPVAASNIPEAAETQFRLAHELRNELVAVERAHADAVAAIWADHPDVAATLQAQAVA
EAAVAELVERAGKERIADRAKEPRAQTRAEIKAARATLAAARQDTKAARSAAYPLVRPAMADAQTRRRQAIGD
AGRGAKTRGLFWHTHDAVLAGHDTAVKRVAALRAEGKPAELRSRRWDGEGRIRVTLMRHEWSHGCGAQPCGQP
SPECPRRQPGDPLRTPALLAGGQGPWRNVCRLPAHMDPAICAEHPPRRHGERETILLRVGSEEREPIWWELPV
FVHRPLPPGADVAFVEVRRERLAGQTRLSVCVTVRLPPVATLTEGAVAAIHPGWRSVTGGIRVMVIAASRPLG
PIPERFAPVVRPLTGNHVEIIAPEEWVRVLGHADSVRSIRDQALDVIRRKIVDALAEDVPGVEVSAADVARWR
SPGRFAALVRQWPNDHPLADVLWAWRRQDRHLWKGEAHERDQIAARRTDTWRHVAVWLCDQVAVIGHAPTPIA
ELSRVPVIEDGDDRQATLARAHRALAAPAELVSLIEIAAGQRGVRIVEIDGARLTATHHVCGEVTGDLARDSV
MLWCSRCGIAFDQDANAATASLARTIGDLSSAKVQ (SEQ ID NO: 248)
>3300010048|Ga0126373_10000093_102
[terrestrial-soil-tropical forest soil]
MPIRVYQFGLLPPIDGEARVRVLMRQCHEYRNELVAIERGRRAALRALHDTQEVADAVALVKASKGKPLREAI
GKLYKARRAAEKAASHCPGVAEASVPEDASDAERSRLRRVNLEARAAAGDAVARITLLDESIRRDARALSPLS
PGAWANYQTIEAAATQVRAMPLYERDAVTPSDPRFVKGPRAGQAFPVSNPKSCWWLGDQQVSMHIQGRTVTTA
DVLAGKDAWVRLELEPARLHGGTNGGKQQYSQYGVLKLRVANDTRCAVWASWPIKLHRAIPNAAKWQWVRVSC
RRLARREVWTVEITLNDPQKIQPRPDVSGAVAVELLWTPLDDGSMRVASWRDSFGATGELLMSSRMVGAIRKA
DGIRSVRDTLLNALRPALAEKIQHSADKLPTWLREVGNVLHLWKSQDRFYELALRWRKDKVDAARDAYELLQE
WELRDAHLLDYEAGSRRNGIGWRNHYYSNWAAGLARRYKAVIVPDRDLSLEARFGDDSDRRTTVSPQKLRDML
LNAFGEDAVKAVWKGPHGVPEDSDDTWLEVVSEQWRNEENTGGACAAEKDNAVADVGGSAWAKRKARARERAA
GKDGARKDVGNTAE (SEQ ID NO: 250)
>3300010366|Ga0126379_10001683_10
[terrestrial-soil-tropical forest soil]
MFGRKRDTAGATIRAYEFGTRPVGDMALASQQLYRRHQLWNQFVEIERDTRAKYRALVATESTQRVAALHEQL
EELRQQIRLHKKSAAYEHVLHGKLRSLVEAMQSALADRSPETVAIVKPRLYFADIVSYDGLRERAKQLRQALG
VMRAEAKAESRAKAEQLKAEREQLERERTDRVKAAMAASNLYWCNSDDVRASYEVARKRAMREGTELHFHRFD
GGGKLSVRYQQGLPVAAAFGTDTRLQIDPIDFSIWESADRAVRRRCWTKARLRIGSNDQRQPVWLTLDCWLDG
RPGRHLPMEGTIRAAAVIRRRVGTGFTHRLVLTVEAPLERRDLAVERHGTVGLDVGWRLTADNGLRVAYWSDG
TAQGALTLPANMVGLFEKVHDLQSIRGQHFNAAVERLQGYLEEHPGAMWLQEQCRYIAQWRSPGRLIVLLRDW
QSDGTDGELLEHLQAWRKKEDHLYSWQANLLDQTAAHRRELYRIFAASLGRYARVAIEEFDLRKVLQKPRPED
GAETPDGHMRTIAAVSVLRSAIENYCRREGVEFVVVPSSGTTRRCHVCQSEQQFDQRSDLVHRCTTCGETWDQ
DYNAAENIRQFADRPAGDTILPSAKISPEAPEARV (SEQ ID NO: 251)
>3300010376|Ga0126381_100020658_4
[terrestrial-soil-tropical forest soil]
MIVHTLYIKTVDSETRKAVLAQMRAGHNYRNELVAIERGRRWALRQAHETPEVAEAIALLKAATRSNRFERLK
ALTRARRQAEEATEKPQVYLACEAARIALRDEAAGTPRKKAAQAVYDAAREALCQSDEFHYAVGERDNWIRKA
AYNDAKCNWGIRAVVNQAFEALRKTGLYERDGVTAWEPRFRRWAPNRPTGTIGVQLQGGLDVKDALSGDDTQV
KLLLNPPIYKGSRQQRVRQFGELWVRIGSDGRSPIWARFNVMAHRALPTDAKIKWVKVHLTRTGPFERWEAHL
TLDASAPPRPITNDRAVAVELLWSPQDDGSITAAHWRDGDGAEGFFALPAIIPTSIRKPDGIQSVRELLLNKM
RPDLVALIKHYGSDLPVWLKEATNTLHLWESPLRFYDLAQCWRDSGFMGAKGAYDRLHEWELRDDHLWRYEAC
ARKTAIRRRRDFYSCLAKNLSAQYRYVILPDRDLSRERRFGEERDIQFTVAPQELRAALTRCFGDETIEAPWR
GAHGVEEDEYGDIDWLLFALEYGRDEKEARAAREAAKSSGDVKLTGGKWARIKAKRKEKDDAERGTRKESGKD
AEGLGGER (SEQ ID NO: 252)
>3300010398|Ga0126383_10032213_5
[terrestrial-soil-tropical forest soil]
MKRQAENVRSMVYQYGTVPARVAPVEGEELALSQMRLAQRLWNVLVTIERARVAGYRSIMRDEVQEQIDALRE
RKDATWQEIKATRQKARAKVATPGLDAEMMRIKTALRLLVEHKQSTKQQRHDARREQLNALAERANQRIKRAR
QAAASMGLFWGTYNAVIQSADAGRKHAGELRYQGFRGEGTVTAQVMGGATPEQCVAGGHPFFQVAPATPGQKW
RYARVRIGSTSERQPLWVAIPVVYHREIPAEARIKSVSATRRILAGKVRWSLNVTVTLPPAEPRPAGQMVAID
IGWRLLPDGVRVAYWQDGTGNHSEVRIADSDIAQFRKISDLRSICDRAREEFLPSLVEWLKPYELDEEWTHRA
RALAQWRSNDRIAALIRWWADHRLSGDAEIYQTAVEWRRQYLHLANWWRNQQEQMTLRVREQYRRFAAGIASQ
FATVIVEDFDLRQVTETTEKAVGTYRQMVSPSLFRAAVINACKREGVEIRIVSGAYSTGACHNCQHIEVWDQA
ASILHRCGACGALWDQDHNAAINLLASGGVVLWRINLVAAIGPLSQDRSQTGGKEAVES (SEQ ID NO:
253)
>3300017961|Ga0187778_10004454_1
[terrestrial-soil-tropical peatland]
MTARVYEYGLLDPTYNAQLVDDQMRAGHRYRNVLVEIERDRRTEVRNLLARHPDVEPLEAELLAARNDLADRR
GQIRLQRKSTRTRSEGASMREAAHAAKLRVAEIRQRVNDAKAAIKEDAVVQAAIATADARAAERVRQARASCA
TYWGTYILHEADVARARASGGEVHFARWTGEGRVSAQIQGGIPSTDLAADTQVQIAPGQSIKDRRVPPNAKIL
RLRVQSDAKGKAIWAEWPMILHRPLPEEGRIKVVTVHKRRRDCRRWQWTVTFTVELLDGWTRGKCGEGAIALN
LGYCRSYDHLKGAIRAGYLIDDRGQEREVIVPTSIIDRINKSEAIRSQRDKDVDTMRALLVAWLRDHEAILPG
WIVDRTILAKAPKDANSPEAPRIWHITAWKSAARFRALAFAWRAARFQGDDVGYDLIERWRYRDEHLQRYEAG
LLRGALLHRRDLYRQLAAELSAKYRTIVLHDTDLSDLQRSPHPEEDRREIGGAKYNQRIAAGSILRGALDNAF
KRAGGEVVIVDDHRITKACWKCSEAEDWNQLDREHICGACGTRWDQDANACHNMLARERAGAEGRRQAARAAK
TADRKETRSERLRRGLATKRKTEAARAM (SEQ ID NO: 254)
>3300017970|Ga0187783_10000008_23
[terrestrial-soil-tropical peatland]
MFGNKSLPSRIYSYGANPAIENQKLVEDQMFLAHRYRNAMVEAEIERRKKVDEKLLSLSPSLARIEEKLAAAT
EELEKLRESIKEDHKLYFTKTAKDPVKTKAIAAQKKLVKGLYTERKALRTKLFASSKWKKEQEATEAEALAAH
KELREKSDLYWGTYLLVEQSMQGSRSGAPPRFMRWDGDGHIALQIQNGMTVEEALSGADTRLAIIPGRVEVDG
SRTKETGIKRKLGTALCKFRIGTDEKTHSPIFASIPFHMHRPLPEDAQIKWVHFIRRRVSTHCEWRLQFVLSQ
KKWVKEDQAQEGTVGIDLGWSLDQEGYLQVACFAGSDGESGRLFLPADWLGEMKRVEGIRSIRDQNFNDAKAL
LQAFLRTSKEKSWLKEEAKTLPQWRSPAKLAWVIQKWRKERIPGDQHIFQAMEAWRLRDKHLYEFEANLRDQL
LRRRESIYRNFAADLRRSYKTARILKLALKEVHELPQAEEAPENPQLREHSRDACLSFLVRCLRESMARTVEI
DPKNQARRHHGCGSLEDLGANEKLHTCSRCGEIYERHENTARNLLGMMPAGAGV (SEQ ID NO: 255)
>3300017972|Ga0187781_10019688_5
[terrestrial-soil-tropical peatland]
MERLPTETSPLDVVEMEREIDEARTATGWPTATVVHRYGIASPHEGADIVHEQIRLARAYRRELVTIERARRA
AARQAMTELAPEVGFAEATVVGADAACQWLAAEIRAARAATRKRAESRGMRDRLTRARASLRTHRAELFALRS
RYATQCADCRKAKSESVPCPHATAEARRLLERVDAVNDQAAAAQRRARGECGVYWGSYLLVERAMQASRAMPL
YADDGVSPNDPSVPHSLSDSLGCQIQSTRPLTVAGAAAGTDSRLRIQPPPWPEAWLHEARLDPSAQSHPSHRL
PGQRPDGTPAPATRADGTPARWVRDRACRQGEVRMRVGANGEWAAWRLDEHRAMPPNAAVKWATVVRRQRGPH
TEWSLCLTLEVPLPEALPQTGRTVAVDVGWRQIGDELRVAAWQDSDGQKGELRLTKADLHALNASAEVRSLRD
GKMESIKQRLAQWSAVASPQDCPEWIREALRTVRLWRNPVRLVRLLRQWREAGEPTRVVPRVAFDHLVAWADD
DRHRWAEQESRRVWGLRRRRERYRVFAAELAKRYDAVVLEQFDLRRVAARPQTGRELESENEVARSNRQRASV
SELRDALRNACRSRGRVVVAVDATDSTRTCPSCGLVADRGQDERVVLRCECGHEWDQDRDGAALVLLRRYREH
PGDAKTLVAARAGATLAEPPKKKNDRWARARRMSTQKKERAQGARDSG (SEQ ID NO: 256)
>3300018064|Ga0187773_10011230_2
[terrestrial-soil-tropical peatland]
MENQGEESAAAVPSEEEAPLDARVYQFGLLPPRVNRDLVEDQMYLGHRYRCQLVEFERDRRDAVREILSSQSG
VEETEARIADLAARRDAARAEIASKRSRSRSRSDSAEERATVREIGRQLKALRAEAKEARSIVASDEHVGAWI
SAENDRAAQRQKDARVACGVYWGTYLLHESDAQRARTGKSYPKFPRWNGDGRVAVQLQGGLSPQKLDHGQDTR
LQVVSSSHRTGRRLGRGSLLRMRVQSNGRDPVWAEWPMILHRPLPEGVRIKTATVSRRRRGSQVDWCATITVD
EPPRPIRATATEEAVAINLGYARRPNGGIRVGYWVGSDGAGGEILCQGSAAYRPRSSEEQTRAAVTHVEESLK
KADSIRSFRDRGMNEMRARLIAWIDDFVGGDPPDGVPWWIADARRHLHLWRSPNRFASLLRRWERGWWPDLDG
GYAILLAWSRRDLHLERYETGMRTTARRDRREGYRLLAARLAARYRTLVVDDADFRNFQRSPEPESDYVEVDA
QKWQQRVASPSELRFAFLSAFGVDRTAKEPCEDVTRRHAPCGHVVDVAGDSRELRCPHCSEVEKREVLFDQDA
NACDNLLRGWLRKAPEMRQARTKRPPSIRRQRMIAGAKKKREAKAAEERRREARGG (SEQ ID NO: 257)
>3300012204|Ga0137374_10001132_4
[terrestrial-soil-vadose zone soil]
MATLVYRYGVRAHGSARQQDAVVSDPAMLEQLRLGHELRNALVGVQHRYEDGKRAVWSGFASVAAADHRVTTG
ETAVAELEKQARAEHSADRTAATRQGTAESLKAARAAVKQARADRKAAMAAVAEQAKPKIQALGDDRDAEIKD
LYRRFCQDGVLLPRCGRCAGDLRSDGDCTDCGAAHEPRKLYWATYNAIREDHQTAVKLVEAKRKAGQPARLRF
RRWTGDGTLTVQLQRMHGPACRCVTCAEKLTRRARKTDPQAPAVAADPAYPPTDPPRDPALLASGQGKWRNVL
QLGTWIPPGEWSAMSRAERRRVGRSHIGWQLGGGRQLTLPVQLHRQMPADADVAMAQLTRVRVGGRHRMSVAL
TAKLPDPPQVQGLPPVALHLGWRQRPDGSLRVATWACPQPLDLPPAVADVVVSHGGRWGEVIMPARWLADAEV
PPRLLGRRDKAMEPVLEALADWLEAHTEACTARMTPALVRRWRSQGRLAGLTNRWRGQPPTGSAEILTYLEAW
RIQDKLLWERESHLRRRLAARRDDAWRRVASWLARHAGVLVVDDADIAELRRRDDPADTDPTMPASAAQAARA
RAALAAPGRLRHLATITATRDGLGVHTVASAGLTRLHRKCGHQAQPDPRYAASAVVTCPGCGNGYDQDYNAAM
LMLDRQQQP (SEQ ID NO: 258)
>3300012210|Ga0137378_10000107_47
[terrestrial-soil-vadose zone soil]
MENNITVMRYGARVPITGLESVDRQLRLSKKYRNALCEIERRRRDGIARVQHGTRAGASPLDADELLAPLILK
IDELEASISEMRKQTKLTHAGGGNPAARASLRDQIAAVKADLAVLRWLRGWSKSRLRLSDDERAAIAMCDGEK
MSAWMIHRVKPWWALGVAVHATMNGSAALRLRCEYMIIDETAKYERRQARAVAGLSPGTYLLIEAAADKWRQN
PEQPRFMRYDGTGRVGVQVQGGCTVAELEGGQDTRMRLLPATEIDPPVAPTSSRQIARAIAYGVVHFCRAGNM
APRDTYRILQLRVETMGRAPVWASIPIVYHRPLPADGVIVAAWLQRKKIGVRSVYDAQLVVRAAMTPQSHRPT
TGTIAVDIGSRDIPSTGETRVAYSLDSSGAHAAMILPLFRLSSATSRGTGRRRIVPDDEKKIDDIKSIRSRHL
DEIRDQITAYKVSVGAAASSPHPVPAIEWLRAATDRITSWRSPARIVWLRRQWQHHTGDEKIFSSIEAYIRQD
RHLLDWQSREMRRRLGRRRELYRTAAMRLARTYDTIILAARDYRREEWVPEDAPSTRAHESRSIMRGAAPGEF
REIIRRSAKKYGTTLIEMPLEGDTAWALDYRVCQRMLASTEVVDVQAAPLASASRSNHYGTDESFHRRRLGTD
ERIDPLARIDVSG (SEQ ID NO: 259)
>3300012532|Ga0137373_10000316_4
[terrestrial-soil-vadose zone soil]
MATLVYRYGVRAHGSARQQDAVVSDPAMLEQLRLGHELRNALVGVQHRYEDGKRAVWSGFASVAAADHRVTTG
ETAVAELEKQARAEHSADRTAATRQGTAESLKAARAAVKQARADRKAAMAAVAEQAKPKIQALGDDRDAEIKD
LYRRFCQDGVLLPRCGRCAGDLRSDGDCTDCGAAHEPRKLYWATYNAIREDHQTAVKLVEAKRKAGQPARLRF
RRWTGDGTLTVQLQRMHGPACRCVTCAEKLTRRARKTDPQAPAVAADPAYPPTDPPRDPALLASGQGKWRNVL
QLGTWIPPGEWSAMSRAERRRVGRSHIGWQLGGGRQLTLPVQLHRQMPADADVAMAQLTRVRVGGRHRMSVAL
TAKLPDPPQVQGLPPVALHLGWRQRPDGSLRVATWACPQPLDLPPAVADVVVSHGGRWGEVIMPARWLADAEV
PPRLLGRRDKAMEPVLEALADWLEAHTEACTARMTPALVRRWRSQGRLAGLTNRWRGQPPTGSAEILTYLEAW
RIQDKLLWERESHLRRRLAARRDDAWRRVASWLARHAGVLVVDDADIAELRRRDDPADTDPTMPASAAQAARA
RAALAAPGRLRHLATITATRDGLGVHTVASAGLTRLHRKCGHQAQPDPRYAASAVVTCPGCGNGYDQDYNAAM
LMLDRQQQP (SEQ ID NO: 258)
>3300012532|Ga0137373_10000407_43
[terrestrial-soil-vadose zone soil]
MIVYRYGALKPTEGFDLLLTQLRLACRYRNALVELLNWRIIAEQSGVERSAAKLVHAEMSCWLRSRCGLGWGT
YQAIEADVRRAAKSPYRAPRKVSGRARWFAQVRQIKIQRPPDADGNQDAQVREIGLDPTKFRARFRRFDGTGR
LGANIQACSGATTDDVLSGRGSLRLSAPEGRVTARLYLGLGIHVTLPVIAHRPLPPGVQVVRALICVERVGDR
YVYSVHVTMRHERPERQYGSGRAAINFGWRSLGDRGVRIAYVATDEGSTDELILPRRLIDKLRHSESLRGLAD
DAAVAYLGDARGRTRARREALRDPSATHRELGRVPIEGEPISAEHWARRDRHLYQWERDEYAKVLRQRREIYR
LWVRSLAAKYGSVVMEDYDLPTLISRDQPTEIPEARHVRFLVAPGSLRAEVQSVFGERATLATIKRRTMVCSV
CGCELTGDRVRDVVLYCEQCDAQRDQDANNAANQLIDTAAE (SEQ ID NO: 260)
>3300012930|Ga0137407_10020190_4
[terrestrial-soil-vadose zone soil]
MSTIVYRFGVHGAPLDNLELVKQQMRAAHNYANDLVAIERGRRTALHAIDDVPDVRNTIETVRQSTKSTRKAA
ITALRLARKAARAAAEEELARVQALDESIRRDARAITVCYWGSYLTIEMSAQQQRSQPLYEDDAITPNLPRFR
GWREEGQIGIQIQKGLPTSAVRACTDTRARLDRSDRKKQGSKGRSVEYADLWIRIGSDGRAPIWTRVRVIMHR
QIPDAAQWKWVRLSYRREGRTFAWSVEISVDVDRPKRTLDTTLRGAIAIEPQWSENADGSIVCATYRTEDGSS
GEIELSPRIVGALRKADSIRAVRDMILNEARKDIHRALVEAGPALPVWLKDARNTMHLWKSQERFYRLANHWR
RERCDAAREAYDRLQEWELRDDHLWRYEAGSRGQAIRSRNDFYHVIASQFARKHRFVIVPKRDYSREARFGAE
SDLRFIVSPSSLVSALDCSFDHGESAYVCPWVRPDGDGDSAEWPAIAIERFCAGDSAMIARKIRKENDSEEKQ
ESAWARRKRLKREKEMRLATARITDGNGTKSLGQ (SEQ ID NO: 261)
>3300005987|1071089|scaffold14955_2
[wastewater-nutrient removal-wastewater effluent]
MSTLVYRFGLLPPHENDALVRSQMRLAHRYRNDLVQIERARRAAVRDVARAAPEVARLELEAARTDAACAKVG
GQIKAARAAGRTRKDSVELIEQLKALRIEHKEVVLALREARKTARVAAEPVLAEIEERVAAMRRGARAICGVY
WGTYLMIEDADQAMRKMPLYDKDAEPSDPRFVPWTGDGSVGVQIQGGMTGEDTADDTRLRIESAAPPPGADPN
SKRSLRRRYCVLAMRVGSEGRDPVWARWRMVMERPLPADARIKRAAVKLRRVGPREEWSVTITLETAERDRRV
SDQVGMVGIDLGWRLMPDGLRVAAWHGSDGASGFLTLPDTRPTFHTTPGGRTRSSALGVVDAARKVEDLSSKR
DKAFNEARSAIARIEGAPPWFELATKTISQWKSQGRLAALVRRWRDARWDGDAEAYEEAEKWRYHDHHLWAWE
TSQQAKALRARREVFRLFAADMAKRYARLAIEGLDLRAFARKTDDDTNETARRNRVVVAPSKLREALLLAFGS
AINHWAETPKGDRVVVVEAAGTTMVHHECGSVERWDQATHVSHLCSSCGEIFDQDANAAKNILAAGERLGGPV
LSGAARNDENVSNLSQVREGRWAKAKRMKAEKDARLEAARKAAPSAAE (SEQ ID NO: 262)
>3300005988|1071091|scaffold06014_8
[wastewater-nutrient removal-wastewater effluent]
MSTLVYRFGLLPPHENDALVRSQMRLAHRYRNDLVQIERARRAAVRDVARAAPEVARLELEAARTDAACAKVG
GQIKAARAAGRTRKDSVELIEQLKALRIEHKEVVLALREARKTARVAAEPVLAEIEERVAAMRRGARAICGVY
WGTYLMIEDADQAMRKMPLYDKDAEPSDPRFVPWTGDGSVGVQIQGGMTGEDTADDTRLRIESAAPPPGADPN
SKRSLRRRYCVLAMRVGSEGRDPVWARWRMVMERPLPADARIKRAAVKLRRVGPREEWSVTITLETAERDRRV
SDQVGMVGIDLGWRLMPDGLRVAAWHGSDGASGFLTLPDTRPTFHTTPGGRTRSSALGVVDAARKVEDLSSKR
DKAFNEARSAIARIEGAPPWFELATKTISQWKSQGRLAALVRRWRDARWDGDAEAYEEAEKWRYHDHHLWAWE
TSQQAKALRARREVFRLFAADMAKRYARLAIEGLDLRAFARKTDDDTNETARRNRVVVAPSKLREALLLAFGS
AINHWAETPKGDRVVVVEAAGTTMVHHECGSVERWDQATHVSHLCSSCGEIFDQDANAAKNILAAGERLGGPV
LSGAARNDENVSNLSQVREGRWAKAKRMKAEKDARLEAARKAAPSAAE (SEQ ID NO: 262)
>3300006056|1071094|scaffold118627_2
[wastewater-nutrient removal-wastewater effluent]
MSTLVYRFGLLPPHENDALVRSQMRLAHRYRNDLVQIERARRAAVRDVARAAPEVARLELEAARTDAACAKVG
GQIKAARAAGRTRKDSVELIEQLKALRIEHKEVVLALREARKTARVAAEPVLAEIEERVAAMRRGARAICGVY
WGTYLMIEDADQAMRKMPLYDKDAEPSDPRFVPWTGDGSVGVQIQGGMTGEDTADDTRLRIESAAPPPGADPN
SKRSLRRRYCVLAMRVGSEGRDPVWARWRMVMERPLPADARIKRAAVKLRRVGPREEWSVTITLETAERDRRV
SDQVGMVGIDLGWRLMPDGLRVAAWHGSDGASGFLTLPDTRPTFHTTPGGRTRSSALGVVDAARKVEDLSSKR
DKAFNEARSAIARIEGAPPWFELATKTISQWKSQGRLAALVRRWRDARWDGDAEAYEEAEKWRYHDHHLWAWE
TSQQAKALRARREVFRLFAADMAKRYARLAIEGLDLRAFARKTDDDTNETARRNRVVVAPSKLREALLLAFGS
AINHWAETPKGDRVVVVEAAGTTMVHHECGSVERWDQATHVSHLCSSCGEIFDQDANAAKNILAAGERLGGPV
LSGAARNDENVSNLSQVREGRWAKAKRMKAEKDARLEAARKAAPSAAE (SEQ ID NO: 262)
*Effector proteins having identical amino acid sequences were identified from different sources and assigned the same sequence identifier.

TABLE 3
Nucleotide Sequences of Representative CLUST.018837 Direct Repeats

| Effector Accession | Direct Repeat Nucleotide Sequence |
|---|---|
| WP_081130164.1 (SEQ ID NO: 1) | GTTTCATCGGCCATCGCGGCGGCCTCGTAGCTGCGAC (SEQ |
| | ID NO: 27) |
| WP_018079340.1 (SEQ ID NO: 2) | GTTTCATCAGCCATTGCAGGGGCTTTG (SEQ ID NO: 28) |
| WP_064217851.1 (SEQ ID NO: 3) | GTGTTATGCCCATCTCAGCGGGCTGGTTGCTGAGAC (SEQ ID |
| | NO: 29) |
| JMEB01000165_11 (SEQ ID NO: 4) | GTTATAGTGGCCATTGTAGGGGCTT (SEQ ID NO: 30) |
| WP_051690567.1 (SEQ ID NO: 5) | GTTATAGTGGCCATTGTAGGGGCTT (SEQ ID NO: 30) |
| OJW42488.1 (SEQ ID NO: 6) | GGTTTAAGGCGATCACGGCCGCCTAGTTGCCGCGAC (SEQ ID |
| | NO: 31) |
| LNFM01018448_6 (SEQ ID NO: 7) | GGTGCTCAAGCCATCGCAGCGGCATCGTTGCTGCGAC (SEQ |
| | ID NO: 32) |
| 3300004774\|Ga0007794_10001723_8 | GAGCGGTTAACAGGGTGTCGATATAGATT (SEQ ID NO: |
| (SEQ ID NO: 8) | 33) |
| 3300004776\|Ga0007800_10001775_2 | GAGCGGTTAACAGGGTGTCGATATAGATT (SEQ ID NO: |
| (SEQ ID NO: 8) | 33) |
| 3300009004\|Ga0100377_1000348_44 | GTAACAACAGCCATTACCCTGGCTTAGTAAGGGTGAC (SEQ |
| (SEQ ID NO: 9) | ID NO: 34) |
| 3300004236\|Ga0066449_1000007_83 | AGTAGAACCCTGTCGCTTGGGCGGTAAAGCGAAC (SEQ ID |
| (SEQ ID NO: 10) | NO: 35) |
| 3300009432\|Ga0115005_10004282_5 | GATTAAGGCCCCTGTGCATTGGGGTGTAAATGCAAC (SEQ ID |
| (SEQ ID NO: 11) | NO: 36) |
| 3300009436\|Ga0115008_10017733_3 | GTCTAAGGCCCCTGTGCATTGGGGTGTAAATGCAAC (SEQ ID |
| (SEQ ID NO: 12) | NO: 37) |
| 3300009436\|Ga0115008_10017733_4 | GTCTAAGGCCCCTGTGCATTGGGGTGTAAATGCAAC (SEQ ID |
| (SEQ ID NO: 13) | NO: 37) |
| 3300001351\|JGI20153J14318_10007490_6 | CTTTAAGGACCCTGTACGTTGGGGTGTAAACGTAAC (SEQ ID |
| (SEQ ID NO: 14) | NO: 38) |
| 3300009447\|Ga0115560_1022222_2 | CTTTAAGGACCCTGTACGTTGGGGTGTAAACGTAAC (SEQ ID |
| (SEQ ID NO: 15) | NO: 38) |
| 3300009505\|Ga0115564_10016546_3 | CTTTAAGGACCCTGTACGTTGGGGTGTAAACGTAAC (SEQ ID |
| (SEQ ID NO: 16) | NO: 38) |
| 3300020165\|Ga0206125_10004811_3 | CTTTAAGGACCCTGTACGTTGGGGTGTAAACGTAAC (SEQ ID |
| (SEQ ID NO: 17) | NO: 38) |
| 3300010313\|Ga0116211_1004493_2 | GGTATCATGACCCTACGGGTGGGGGG (SEQ ID NO: 39) |
| (SEQ ID NO: 18) | |
| 3300009784\|Ga0123357_10002363_9 | AGCCATCGCAGGGGCTTGGTGCTTGCGAC (SEQ ID NO: |
| (SEQ ID NO: 19) | 40) |
| ADIG01000806_20 (SEQ ID NO: 20) | ATTCCAAGGCGATCACAGCCGCCTAGTAGTTGTGAC (SEQ ID |
| | NO: 41) |
| CXWL01128655_18 (SEQ ID NO: 21) | GTTTTAGAACCCTGTTGAGTGGGCATAAACTCAAACT (SEQ |
| | ID NO: 42) |
| OGCL01001770_13 (SEQ ID NO: 18) | GGTATCATGACCCTACGGATGGGGGG (SEQ ID NO: 43) |
| LNAP01002847_16 (SEQ ID NO: 22) | GATTCATAGCCCTGTCGGTTGGGCGGTAAACCGAAC (SEQ ID |
| | NO: 44) |
| 3300007533\|Ga0102944_1000048_72 | GTCTCAATAGCGATCGGCGCCGCTTAGTAGGGTCGAC (SEQ |
| (SEQ ID NO: 23) | ID NO: 45) |
| 3300007533\|Ga0102944_1003721_10 | GGTAACAATAGCGATCGGCGCCGCTTGGTAGTGTCGAC (SEQ |
| (SEQ ID NO: 24) | ID NO: 46) |
| 3300007533\|Ga0102944_1003721_8 | GGTAACAATAGCGATCGGCGCCGCTTGGTAGTGTCGAC (SEQ |
| (SEQ ID NO: 25) | ID NO: 46) |
| APMI01033782_24 (SEQ ID NO: 26) | ATCTCAATGGCCATCGTCGGGGCTTTGTACCGGCGAC (SEQ |
| | ID NO: 47) |
| NZ_JQKL01000024_23 (SEQ ID NO: 48) | GTTGCAATGCCTAGCTCAGAGGTTTAAAGACTGAGAC (SEQ |
| | ID NO: 263) |
| WP_081908191.1 (SEQ ID NO: 49) | GTTGCAATGCCTAGCTCAGAGGTTTAAAGACTGAGAC (SEQ |
| | ID NO: 263) |
| GAB36148.1 (SEQ ID NO: 50) | GTGTCAACGCCAGCGCGGAGGCGTCAAATCCGCGAC (SEQ ID |
| | NO: 264) |
| BAFB01000202_4 (SEQ ID NO: 51) | GTGTCAACGCCAGCGCGGAGGCGTCAAATCCGCGAC (SEQ ID |
| | NO: 264) |
| WP_039994403.1 (SEQ ID NO: 52) | GTGTCAACGCCAGCGCGGAGGCGTCAAATCCGCGAC (SEQ ID |
| | NO: 264) |
| WP_013159911.1 (SEQ ID NO: 53) | GTAGCAATGCCTAGCTCAGGGGCTT (SEQ ID NO: 265) |
| WP_096876841.1 (SEQ ID NO: 54) | GTTACAAACCCTGCTCATTGGGTTGGTTAATGAGAC (SEQ ID |
| | NO: 266) |
| WP_048895525.1 (SEQ ID NO: 55) | GTGAGAATGACCAGCGCACCGGTCGAAAGGTGCGAC (SEQ ID |
| | NO: 267) |
| WP_061006603.1 (SEQ ID NO: 56) | GTGTCATAGCCCAGCTTGGCGGGCGAAGGCCAAGAC (SEQ ID |
| | NO: 268) |
| WP_011733919.1 (SEQ ID NO: 57) | GTTGACATGCGTGCTCCGCCGCTTTGTAGTGGAGAC (SEQ ID |
| | NO: 269) |
| WP_018234394.1 (SEQ ID NO: 58) | TTGATAATGCCCGCTCTGCGGCCTCGTAGTAGAGAC (SEQ ID |
| | NO: 270) |
| 3300000944\|BBAY81_10000005_89 | CAACGCCTACAGTGGGCTTCGTACATTGTGAC (SEQ ID NO: |
| (SEQ ID NO: 59) | 271) |
| LSQX01035253_23 (SEQ ID NO: 60) | CAGCGGCCCACGCACGCGAGGGACGGTC (SEQ ID NO: |
| | 272) |
| 3300013131\|Ga0172373_10056063_2 | CAAGCAAGCCTTGCCGAAGAGGCTCGATCTTCGGACG (SEQ |
| (SEQ ID NO: 61) | ID NO: 273) |
| 3300013136\|Ga0172370_10027535_4 | ATGATAAGAGTTTCTCGATACTCTATAAATCGAGAC (SEQ ID |
| (SEQ ID NO: 62) | NO: 274) |
| 3300013137\|Ga0172375_10012175_6 | CAAGCAAGCCTTGCCGAAGAGGCTCGATCTTCGGAC (SEQ ID |
| (SEQ ID NO: 63) | NO: 275) |
| 3300010293\|Ga0116204_1010874_1 | GGTTGAAGCGCCCGCGCAAGGGCTTTGTACTTGCGAC (SEQ |
| (SEQ ID NO: 64) | ID NO: 276) |
| 3300010293\|Ga0116204_1010874_2 | GGTTGAAGCGCCCGCGCAAGGGCTTTGTACTTGCGAC (SEQ |
| (SEQ ID NO: 65) | ID NO: 276) |
| 3300008255\|Ga0100403_1011992_3 | GTCGCAATGCCAGCCGAACGGCTTGGAAGTTCGGAC (SEQ ID |
| (SEQ ID NO: 66) | NO: 277) |
| 3300014155\|Ga0181524_10003409_23 | GTGGCTACGGCATCGCGGCGCCTCGGAGATCGCGAC (SEQ ID |
| (SEQ ID NO: 67) | NO: 278) |
| 3300014156\|Ga0181518_10000096_28 | GTGGCTACGGCATCGCGGCGCCTCGGAGATCGCGAC (SEQ ID |
| (SEQ ID NO: 67) | NO: 278) |
| 3300014158\|Ga0181521_10000063_92 | GTGGCTACGGCATCGCGGCGCCTCGGAGATCGCGAC (SEQ ID |
| (SEQ ID NO: 67) | NO: 278) |
| 3300014159\|Ga0181530_10000119_98 | GTGGCTACGGCATCGCGGCGCCTCGGAGATCGCGAC (SEQ ID |
| (SEQ ID NO: 67) | NO: 278) |
| 3300014201\|Ga0181537_10003972_13 | GTCGCAGAGGAAGCTCGACGGATCGAGCGTCGAGAG (SEQ ID |
| (SEQ ID NO: 68) | NO: 279) |
| 3300014201\|Ga0181537_10021284_1 | GTCGCGATCGAAGCTCCATCGGCTGGCGATGGAGAC (SEQ ID |
| (SEQ ID NO: 69) | NO: 280) |
| 3300014201\|Ga0181537_10040512_3 | CCAGAAAGGGCAGCGCATCGCCCAGGAGAATGCGAC (SEQ ID |
| (SEQ ID NO: 70) | NO: 281) |
| 3300014654\|Ga0181525_10000532_4 | GTAGCAACGCCAGCTCGGTTGGTTCTGAGCCGAGACA (SEQ |
| (SEQ ID NO: 71) | ID NO: 282) |
| 3300014657\|Ga0181522_10000394_52 | GTGGAAACGTCAATGTCCGGCGGACGACGCTGGAAC (SEQ ID |
| (SEQ ID NO: 72) | NO: 283) |
| 3300014657\|Ga0181522_10000394_53 | GTGGAAACGTCAATGTCCGGCGGACGACGCTGGAAC (SEQ ID |
| (SEQ ID NO: 73) | NO: 283) |
| 3300009175\|Ga0073936_10014029_2 | CGCTGCAGGGCCTCAAACGACTAGACGAGGCACTGGCAAGCTAC |
| (SEQ ID NO: 74) | GACAAGG (SEQ ID NO: 284) |
| 3300015360\|Ga0163144_10020017_5 | CCAACAACGGCTGCTGAGCGCCTT (SEQ ID NO: 285) |
| (SEQ ID NO: 75) | |
| 3300015360\|Ga0163144_10020017_4 | CCAACAACGGCTGCTGAGCGCCTT (SEQ ID NO: 285) |
| (SEQ ID NO: 76) | |
| 3300015360\|Ga0163144_10033243_8 | GGCTGCTGAGCGCCTTACAAGCTCAGAC (SEQ ID NO: |
| (SEQ ID NO: 77) | 286) |
| 3300015360\|Ga0163144_10033243_7 | GGCTGCTGAGCGCCTTACAAGCTCAGAC (SEQ ID NO: |
| (SEQ ID NO: 78) | 286) |
| 3300015360\|Ga0163144_10062707_6 | GTAACAACCCCTAGCGCAAGAGGGAAAGCTTGCGAC (SEQ ID |
| (SEQ ID NO: 79) | NO: 287) |
| 3300015360\|Ga0163144_10062707_6 | GTAACAACCCCTAGCGCAAGAGGGAAAGCTTGCGAC (SEQ ID |
| (SEQ ID NO: 80) | NO: 287) |
| 3300020057\|Ga0163151_10006104_16 | CCAACAGCGGCTGCTGAACGCCTTACAAGTTCAGAC (SEQ ID |
| (SEQ ID NO: 75) | NO: 288) |
| 3300020186\|Ga0163153_10017638_7 | CTCACAGTGCCTGCGCAGCGGCTTCGTAGCTGCGAC (SEQ ID |
| (SEQ ID NO: 81) | NO: 289) |
| 3300020195\|Ga0163150_10003396_14 | CTCACAGTGCCTGCGCAGCGGCTTCGTAGCTGCGAC (SEQ ID |
| (SEQ ID NO: 82) | NO: 289) |
| 3300020203\|Ga0163148_10001247_2 | GTAACAACCCCTAGCGCAAGAGGGAAAGCTTGCGAC (SEQ ID |
| (SEQ ID NO: 83) | NO: 287) |
| 3300020203\|Ga0163148_10001247_2 | GTAACAACCCCTAGCGCAAGAGGGAAAGCTTGCGAC (SEQ ID |
| (SEQ ID NO: 84) | NO: 287) |
| 3300020213\|Ga0163152_10009495_14 | GTAACAACCCCTAGCGCAAGAGGGAAAGCTTGCGAC (SEQ ID |
| (SEQ ID NO: 85) | NO: 287) |
| 3300020213\|Ga0163152_10009495_14 | GTAACAACCCCTAGCGCAAGAGGGAAAGCTTGCGAC (SEQ ID |
| (SEQ ID NO: 86) | NO: 287) |
| 3300020219\|Ga0163146_10006198_18 | CCAACAGCGGCTGCTGAACGCCTTACAAGTTCAGAC (SEQ ID |
| (SEQ ID NO: 75) | NO: 288) |
| 3300020596\|Ga0163149_10010333_13 | CCAACAACGGCTGCTGAACGCCTTACAAGTTCAGAC (SEQ ID |
| (SEQ ID NO: 87) | NO: 290) |
| 3300020596\|Ga0163149_10010333_12 | CCAACAACGGCTGCTGAACGCCTTACAAGTTCAGAC (SEQ ID |
| (SEQ ID NO: 88) | NO: 290) |
| 3300004174\|Ga0066406_1000030_21 | GTTGCAATGCCTGCTCATAGGCTTGGTTTATGAGAC (SEQ ID |
| (SEQ ID NO: 89) | NO: 291) |
| 3300004200\|Ga0066422_1000628_7 | GTTGCAATGCCTGCTCATAGGCTTGGTTTATGAGAC (SEQ ID |
| (SEQ ID NO: 89) | NO: 291) |
| 3300004205\|Ga0066415_1000057_23 | GTTGCAATGCCTGCTCATAGGCTTGGTTTATGAGAC (SEQ ID |
| (SEQ ID NO: 89) | NO: 291) |
| 3300004565\|Ga0066503_104695_4 | GTTGCAATGCCTGCTCATAGGCTTGGTTTATGAGAC (SEQ ID |
| (SEQ ID NO: 89) | NO: 291) |
| 3300009686\|Ga0123338_10029047_2 | GTAGCAATACCCTAGCTCGAGGGGGGTTTGTCGAGAC (SEQ |
| (SEQ ID NO: 90) | ID NO: 292) |
| 3300001242\|C687J13896_1000006_134 | GTTTCAGTATCCTGCTCAGAGGAGTCGTTTCTGAGAC (SEQ |
| (SEQ ID NO: 91) | ID NO: 293) |
| 3300005236\|Ga0066636_10020712_3 | GTCGCAATGCCAGCCGAACGGCTTGGAAGTTCGGAC (SEQ ID |
| (SEQ ID NO: 92) | NO: 277) |
| 3300014208\|Ga0172379_10007070_15 | GCGACGACAACGGCAGCTCATCGCCACGAAAGATGAGAC (SEQ |
| (SEQ ID NO: 93) | ID NO: 294) |
| 3300014208\|Ga0172379_10014650_2 | GTGCAGGGCTGTCGAACCTGCGA (SEQ ID NO: 295) |
| (SEQ ID NO: 94) | |
| 3300014613\|Ga0180008_1000021_8 | GCTGGGATGTTTAGTGATC (SEQ ID NO: 296) |
| (SEQ ID NO: 95) | |
| 3300014613\|Ga0180008_1000021_9 | GCTGGGATGTTTAGTGATC (SEQ ID NO: 296) |
| (SEQ ID NO: 96) | |
| 3300014656\|Ga0180007_10000195_44 | GCTGGGAGGTTTAGTGATCCCAGAC (SEQ ID NO: 297) |
| (SEQ ID NO: 95) | |
| 3300014656\|Ga0180007_10000195_48 | GCTGGGAGGTTTAGTGATCCCAGAC (SEQ ID NO: 297) |
| (SEQ ID NO: 96) | |
| 3300014656\|Ga0180007_10004731_7 | GTCACAATGCCTGCGCAGAGGCTTTGTTTCTGCGACG (SEQ |
| (SEQ ID NO: 97) | ID NO: 298) |
| 3300014656\|Ga0180007_10004731_5 | GTCACAATGCCTGCGCAGAGGCTTTGTTTCTGCGACG (SEQ |
| (SEQ ID NO: 98) | ID NO: 298) |
| 3300015370\|Ga0180009_10002661_7 | GTATCAATGCCTGCTCAAGGGCTTTGTGCTTGAGAC (SEQ ID |
| (SEQ ID NO: 99) | NO: 299) |
| 3300009760\|Ga0116131_1003961_2 | CTCGCAATGCCAGCCCAGAGGCGGATGTTCTGGGAC (SEQ ID |
| (SEQ ID NO: 100) | NO: 300) |
| 3300018019\|Ga0187874_10017489_1 | CTTTCAAGACGAGCGGAAGCGTCTGCTTCTTCCGAC (SEQ ID |
| (SEQ ID NO: 101) | NO: 301) |
| 3300018025\|Ga0187885_10005575_2 | ATCGAAGAGCCTGCTCAGGGGCTTTGTTCTTGAGAC (SEQ ID |
| (SEQ ID NO: 102) | NO: 302) |
| 3300018025\|Ga0187885_10005575_1 | ATCGAAGAGCCTGCTCAGGGGCTTTGTTCTTGAGAC (SEQ ID |
| (SEQ ID NO: 103) | NO: 302) |
| 3300018057\|Ga0187858_10035455_2 | CTTTCAAGACGAGCGGAAGCGTCTGCTTCTTCCG (SEQ ID |
| (SEQ ID NO: 104) | NO: 303) |
| 3300012183\|Ga0136624_1011435_1 | TAAGTGTCAGTGCCTGCGCACCGG (SEQ ID NO: 304) |
| (SEQ ID NO: 105) | |
| 3300012682\|Ga0136611_10000100_4 | CAAACAATGCCCGCGCAGTGGGCTTCGTCACTGCGAC (SEQ |
| (SEQ ID NO: 106) | ID NO: 305) |
| 3300013127\|Ga0172365_10004082_5 | GTAGTAACGCCCGCGAACAGGCTTCGTTTGTTCGAC (SEQ ID |
| (SEQ ID NO: 107) | NO: 306) |
| 3300013127\|Ga0172365_10004082_3 | GTAGTAACGCCCGCGAACAGGCTTCGTTTGTTCGAC (SEQ ID |
| (SEQ ID NO: 108) | NO: 306) |
| 3300013127\|Ga0172365_10033732_1 | CGTTGCAAGGGCTGCGCGACACCCTGGAAGTCGCGAC (SEQ |
| (SEQ ID NO: 109) | ID NO: 307) |
| 3300013128\|Ga0172366_10016188_4 | GTAGTAACGCCCGCGAACAGGCTTCGTTTGTTCGAC (SEQ ID |
| (SEQ ID NO: 107) | NO: 306) |
| 3300013128\|Ga0172366_10018111_5 | GTCGCAATGGGGCGCTCACCCCCCTTGTAAGTGAGAC (SEQ |
| (SEQ ID NO: 110) | ID NO: 308) |
| 3300013129\|Ga0172364_10001281_26 | GTGGCAAAGGCATCGAATCGCCTGAGAAGATTCGAC (SEQ ID |
| (SEQ ID NO: 111) | NO: 309) |
| 3300013129\|Ga0172364_10017363_4 | AACGCCTGCGAACAGGCTTCGTTTGTTCGAC (SEQ ID NO: |
| (SEQ ID NO: 107) | 310) |
| 3300013129\|Ga0172364_10018773_2 | GTCGCAATGGGGCGCTCACCCCCTTTGTAAGTGAGAC (SEQ |
| (SEQ ID NO: 112) | ID NO: 311) |
| 3300013129\|Ga0172364_10045136_2 | GTTGCAAGGGCTGCGCGACACCCTGGAAGTCGCGAC (SEQ ID |
| (SEQ ID NO: 113) | NO: 312) |
| 3300013130\|Ga0172363_10000480_22 | GTGGCAAAGGCATCGAATCGCCTGAGAAGATTCGAC (SEQ ID |
| (SEQ ID NO: 111) | NO: 309) |
| 3300013130\|Ga0172363_10009486_8 | AACGCCTGCGAACAGGCTTCGTTTGTTCGAC (SEQ ID NO: |
| (SEQ ID NO: 114) | 310) |
| 3300013130\|Ga0172363_10014785_2 | GGGTGGCAGTGCCTGCTCAGAGGCTTAGTATCTGTGACA (SEQ |
| (SEQ ID NO: 115) | ID NO: 313) |
| 3300013133\|Ga0172362_10012573_3 | AACGCCTGCGAACAGGCTTCGTTTGTTCGAC (SEQ ID NO: |
| (SEQ ID NO: 107) | 310) |
| 3300013133\|Ga0172362_10022806_8 | TGTGGCAGTGCCTGCTCAGAGGCTTAGTATCTGTGACA (SEQ |
| (SEQ ID NO: 115) | ID NO: 314) |
| 3300013133\|Ga0172362_10025871_2 | GTTGCAAGGGTTGCGCGACACCCTGGAAGTCGCGAC (SEQ ID |
| (SEQ ID NO: 113) | NO: 315) |
| 3300010155\|Ga0098047_10009758_2 | GTGCAAATGCCCGCACAGAGGCTTAGTGTCTGTGAC (SEQ ID |
| (SEQ ID NO: 116) | NO: 316) |
| 3300006805\|Ga0075464_10026824_2 | GTCACAACGCCCGCGCAGGGGCTTGGTATCTGCGAC (SEQ ID |
| (SEQ ID NO: 117) | NO: 317) |
| 3300006805\|Ga0075464_10026824_2 | GTCACAACGCCCGCGCAGGGGCTTGGTATCTGCGAC (SEQ ID |
| (SEQ ID NO: 118) | NO: 317) |
| 3300009149\|Ga0114918_10020022_2 | CTTTCAAAGCTTGCTCGTTAGCTTTATGAACGAGAC (SEQ ID |
| (SEQ ID NO: 119) | NO: 318) |
| 3300006083\|Ga0081762_1007854_6 | GTTTGAAAGCAGCTATAGAGGGCAGAAAC (SEQ ID NO: |
| (SEQ ID NO: 120) | 319) |
| 3300010354\|Ga0129333_10000304_8 | GCAAGGGCTGCTCAGGGCCCTGGAATCTGAGAC (SEQ ID |
| (SEQ ID NO: 121) | NO: 320) |
| 3300010354\|Ga0129333_10000304_10 | GCAAGGGCTGCTCAGGGCCCTGGAATCTGAGAC (SEQ ID |
| (SEQ ID NO: 122) | NO: 320) |
| 3300009507\|Ga0115572_10029017_2 | ATTTAATGACCCTGCGTGTTGGGGTGTGAACACGAC (SEQ ID |
| (SEQ ID NO: 123) | NO: 321) |
| 3300017963\|Ga0180437_10000100_151 | CTTACAATACCTGCGAGACGGTTTAGAAGTCTCGAC (SEQ ID |
| (SEQ ID NO: 124) | NO: 322) |
| 3300017963\|Ga0180437_10000153_25 | CCTTCAATCCCTGCGAGACGGGTTAGAAGTCTCGAC (SEQ ID |
| (SEQ ID NO: 125) | NO: 323) |
| 3300017963\|Ga0180437_10000488_78 | GTAACAACACCAGTCCAAAGGTTTATGATTTGGAAC (SEQ ID |
| (SEQ ID NO: 126) | NO: 324) |
| 3300017963\|Ga0180437_10000692_13 | CTCGCGGTCCCATCGGAACGGGTTGTGGTTCCGACA (SEQ ID |
| (SEQ ID NO: 127) | NO: 325) |
| 3300017963\|Ga0180437_10006965_20 | GTCTCAACGCCTACTCAGGGGCTTTGT (SEQ ID NO: 326) |
| (SEQ ID NO: 128) | |
| 3300017963\|Ga0180437_10006965_20 | GTCTCAACGCCTACTCAGGGGCTTTGT (SEQ ID NO: 326) |
| (SEQ ID NO: 129) | |
| 3300017963\|Ga0180437_10073069_2 | CTTGCAACGGCAGCGTACCGCCTTCAAGTGTGCGAC (SEQ ID |
| (SEQ ID NO: 130) | NO: 327) |
| 3300017971\|Ga0180438_10000090_91 | CCTTCAATCCCTGCGAGACGGGTTAGAAGTCTCGAC (SEQ ID |
| (SEQ ID NO: 125) | NO: 323) |
| 3300017971\|Ga0180438_10000124_114 | CTCGCGGTCCCATCGGAACGGGTTGTGGTTCCGACA (SEQ ID |
| (SEQ ID NO: 127) | NO: 325) |
| 3300017971\|Ga0180438_10000195_144 | CTTACAATACCTGCGAGACGGTTTAGAAGTCTCGAC (SEQ ID |
| (SEQ ID NO: 124) | NO: 322) |
| 3300017971\|Ga0180438_10013386_7 | GTAACAACACCAGTCCAAAGGTTTATGATTTGGAAC (SEQ ID |
| (SEQ ID NO: 126) | NO: 324) |
| 3300017971\|Ga0180438_10021273_1 | GTCTCAACGCCTACTCAGGGGCTTTGT (SEQ ID NO: 326) |
| (SEQ ID NO: 131) | |
| 3300017971\|Ga0180438_10044179_5 | CCTTGCAACGGCAGCGTACCGCCTTCAAGTGTGCGAC (SEQ |
| (SEQ ID NO: 132) | ID NO: 328) |
| 3300017971\|Ga0180438_10056790_2 | CCGGGAACAGCCGCGCAGGGGCTTGGTGCCTGCGAC (SEQ ID |
| (SEQ ID NO: 133) | NO: 329) |
| 3300017971\|Ga0180438_10072596_2 | CGTCGCAACGCCTGCGGAGAGGCCTTGTTTCTCCGACGG (SEQ |
| (SEQ ID NO: 134) | ID NO: 330) |
| 3300017987\|Ga0180431_10022214_3 | GTTTCAGACCCATCGCAAGGGGTTATAGCTTGCGAC (SEQ ID |
| (SEQ ID NO: 135) | NO: 331) |
| 3300017987\|Ga0180431_10041976_5 | GTCGCAACGCCTGCGGAGAGGCCTTGTTTCTCCGAC (SEQ ID |
| (SEQ ID NO: 136) | NO: 332) |
| 3300017989\|Ga0180432_10002388_5 | GTTTCAGACCCATCGCAAGGGGTTATAGCTTGCGAC (SEQ ID |
| (SEQ ID NO: 135) | NO: 331) |
| 3300017989\|Ga0180432_10021155_3 | GTTGCAAAGCCATCTCCAGGGTTTGGTGCTGGAGAC (SEQ ID |
| (SEQ ID NO: 137) | NO: 333) |
| 3300017989\|Ga0180432_10021155_5 | GTTGCAAAGCCATCTCCAGGGTTTGGTGCTGGAGAC (SEQ ID |
| (SEQ ID NO: 138) | NO: 333) |
| 3300017989\|Ga0180432_10043261_1 | GTTGGAATGCCTGTGGAAAGGCTTTGTATTTCCAAC (SEQ ID |
| (SEQ ID NO: 139) | NO: 334) |
| 3300017989\|Ga0180432_10045094_6 | AGCAATGCGAGCGCAGACGCTTCGTATCTGCGAC (SEQ ID |
| (SEQ ID NO: 140) | NO: 335) |
| 3300017991\|Ga0180434_10002646_1 | GTTTCAGACCCATCGCAAGGGGTTATAGCTTGCGAC (SEQ ID |
| (SEQ ID NO: 135) | NO: 331) |
| 3300017991\|Ga0180434_10013735_9 | GTCACAACGCCTGCGCAAGGGCTTTGTTATTGCGAC (SEQ ID |
| (SEQ ID NO: 141) | NO: 336) |
| 3300017992\|Ga0180435_10018121_11 | CTTCCAATACCTGCGAGACGGTTTAGAAGTCTCGACG (SEQ |
| (SEQ ID NO: 142) | ID NO: 337) |
| 3300018065\|Ga0180430_10011859_2 | GGAGCAATGCCTGCACGAGGGCTTTGTGCTCGTGAC (SEQ ID |
| (SEQ ID NO: 143) | NO: 338) |
| 3300018065\|Ga0180430_10038979_3 | GTCGCAACGCCTGCGCGGAGGCTTTGTTTCCGCGAC (SEQ ID |
| (SEQ ID NO: 144) | NO: 339) |
| 3300018080\|Ga0180433_10006034_17 | ATGGAAATGCCATTACAAAGGTTTAGGATTTGTAAC (SEQ ID |
| (SEQ ID NO: 145) | NO: 340) |
| 3300018080\|Ga0180433_10006034_18 | ATGGAAATGCCATTACAAAGGTTTAGGATTTGTAAC (SEQ ID |
| (SEQ ID NO: 146) | NO: 340) |
| 3300018080\|Ga0180433_10012134_6 | TGTCGCAAAGCCATCTCCAAGGCTTGGTG (SEQ ID NO: |
| (SEQ ID NO: 147) | 341) |
| 3300018080\|Ga0180433_10012134_6 | TGTCGCAAAGCCATCTCCAAGGCTTGGTG (SEQ ID NO: |
| (SEQ ID NO: 148) | 341) |
| 3300018080\|Ga0180433_10020043_6 | CTCGCAATCGAAGCTCCGCCCGTTGTAGGCGGAGAC (SEQ ID |
| (SEQ ID NO: 149) | NO: 342) |
| 3300018080\|Ga0180433_10021337_5 | GTCACAACGCCTGCGCAAGGGCTTTGTTATTGCGAC (SEQ ID |
| (SEQ ID NO: 150) | NO: 336) |
| 3300018080\|Ga0180433_10021840_7 | TGGAAGAGCCATCGCAATGGCTTCGGATTGCGAC (SEQ ID |
| (SEQ ID NO: 151) | NO: 343) |
| 3300018080\|Ga0180433_10021840_7 | TGGAAGAGCCATCGCAATGGCTTCGGATTGCGAC (SEQ ID |
| (SEQ ID NO: 152) | NO: 343) |
| 3300001256\|JGI12210J13797_10495608_9 | CACGAAAAGGCAGCTCGATGCCTTACAAATCGAGAC (SEQ ID |
| (SEQ ID NO: 153) | NO: 344) |
| 3300001256\|JGI12210J13797_10495610_14 | CACGAAAAGGCAGCTCGATGCCTTACAAATCGAGAC (SEQ ID |
| (SEQ ID NO: 153) | NO: 344) |
| 3300005917\|Ga0075115_10002831_4 | AGAAATGGTTGTGAAATGCCTTTAAAATTTCAAC (SEQ ID |
| (SEQ ID NO: 154) | NO: 345) |
| 3300005918\|Ga0075116_10002890_7 | ACAGAAATGGTAGTGGAATGCCTTTAAAATT (SEQ ID NO: |
| (SEQ ID NO: 155) | 346) |
| 3300011414\|Ga0137442_1000121_10 | GCCACAGCACCTGCTCGAACGGTTCGAGTTCGAGAC (SEQ ID |
| (SEQ ID NO: 156) | NO: 347) |
| 3300011431\|Ga0137438_1001223_2 | GCCACAGCACCTGCTCGAACGGTTCGAGTTCGAGAC (SEQ ID |
| (SEQ ID NO: 156) | NO: 347) |
| 3300011441\|Ga0137452_1000071_9 | CTGAAAACGCCAGCGCGAAGGCTTCATATTCGCGAC (SEQ ID |
| (SEQ ID NO: 157) | NO: 348) |
| 3300006855\|Ga0079044_1002244_2 | GTGCAAGAGCCTGCGCCGAGGCGTCGTATCGGCGACA (SEQ |
| (SEQ ID NO: 158) | ID NO: 349) |
| 3300006855\|Ga0079044_1002244_2 | GTGCAAGAGCCTGCGCCGAGGCGTCGTATCGGCGACA (SEQ |
| (SEQ ID NO: 159) | ID NO: 349) |
| 3300009503\|Ga0123519_10000481_19 | GTGGAAAGGGTATCTCTGGACCTTACAATCAGAGAC (SEQ ID |
| (SEQ ID NO: 160) | NO: 350) |
| 3300009503\|Ga0123519_10000481_22 | GTGGAAAGGGTATCTCTGGACCTTACAATCAGAGAC (SEQ ID |
| (SEQ ID NO: 161) | NO: 350) |
| 3300006865\|Ga0073934_10032691_1 | GCCGCAACGCCTGTGGAAGGGC (SEQ ID NO: 351) |
| (SEQ ID NO: 162) | |
| 3300001340\|JGI20133J14441_1002607_2 | GGTGCAGCGGTTGCTCAGCACCGTAGAAGCTGAGAG (SEQ ID |
| (SEQ ID NO: 163) | NO: 352) |
| 3300009784\|Ga0123357_10000018_105 | CCCGTGCCCGTCTCCGTCTCC (SEQ ID NO: 353) |
| (SEQ ID NO: 164) | |
| 3300009784\|Ga0123357_10000074_42 | GATCGCGACGGCGACGGTATCC (SEQ ID NO: 354) |
| (SEQ ID NO: 165) | |
| 3300009784\|Ga0123357_10000076_32 | GGCGTCCGCCAGCGTCGGGGCAACCACACCGACGAC (SEQ ID |
| (SEQ ID NO: 166) | NO: 355) |
| BBPF01004549_6 (SEQ ID NO: 167) | GTTGCAGTGCCCAGCTCAGGGGCTTGATAACTGAGAC (SEQ |
| | ID NO: 356) |
| BBPG01001333_4 (SEQ ID NO: 167) | GTTGCAGTGCCCAGCTCAGGGGCTTGATAACTGAGAC (SEQ |
| | ID NO: 356) |
| OGZV01009429_1 (SEQ ID NO: 168) | TAGTGAGTTTAGCACATTTCTAAAAC (SEQ ID NO: 357) |
| OKWZ01000119_10 (SEQ ID NO: 169) | GTTTAAGTAGTAGTGAGTTTAGCACATCTCTAAAAC (SEQ ID |
| | NO: 358) |
| ODGR01000476_16 (SEQ ID NO: 170) | TTAAGTAGTAGTGAGTTTAGCATATTTCTAAAACG (SEQ ID |
| | NO: 359) |
| ODIG01000268_14 (SEQ ID NO: 169) | GTTTAAGTAGTAGTGAGTTTAGCACATCTCTAAAAC (SEQ ID |
| | NO: 358) |
| ODIP01002140_2 (SEQ ID NO: 171) | TAAGTAATAGTGAGTTTAGCACATCTCTAAAAC (SEQ ID |
| | NO: 360) |
| ODIW01000227_18 (SEQ ID NO: 171) | TTAAGTAATAGTGAGTTTAGCACATCTCTAAAAC (SEQ ID |
| | NO: 361) |
| ODJA01000260_38 (SEQ ID NO: 169) | GTTTAAGTAGTAGTGAGTTTAGCACATCTCTAAAAC (SEQ ID |
| | NO: 358) |
| ODJP01000229_55 (SEQ ID NO: 169) | GTTTAAGTAGTAGTGAGTTTAGCACATCTCTAAAAC (SEQ ID |
| | NO: 358) |
| ODKZ01007116_1 (SEQ ID NO: 172) | TTAAGTAGTAGTGAGTTTAGCATATTTCTAAAACG (SEQ ID |
| | NO: 359) |
| ODMO01000523_12 (SEQ ID NO: 169) | GTTTAAGTAGTAGTGAGTTTAGCACATCTCTAAAAC (SEQ ID |
| | NO: 358) |
| ODTN01000195_35 (SEQ ID NO: 169) | GTTTAAGTAGTAGTGAGTTTAGCACATCTCTAAAAC (SEQ ID |
| | NO: 358) |
| ODTP01000194_18 (SEQ ID NO: 172) | GGTTAAGTAGTAGTGAGTTTAGCACATCTCTAAAAC (SEQ ID |
| | NO: 362) |
| ODWI01002981_3 (SEQ ID NO: 169) | GTTTAAGTAGTAGTGAGTTTAGCACATTTCTA (SEQ ID NO: |
| | 363) |
| ODZZ01005262_2 (SEQ ID NO: 169) | GTTTAAGTAGTAGTGAGTTTAGCACATCTCTAAAAC (SEQ ID |
| | NO: 358) |
| OEED01000500_25 (SEQ ID NO: 173) | TAAGTAATAGTGAGTTTAGCACATCTCTAAAAC (SEQ ID |
| | NO: 360) |
| OEFT01000529_3 (SEQ ID NO: 169) | GTTTAAGTAGTAGTGAGTTTAGCACATCTCTAAAAC (SEQ ID |
| | NO: 358) |
| LAZR01002400_15 (SEQ ID NO: 174) | GTAGCAATGGCAGCGCATCGCCTTTTAAGATGCGAC (SEQ ID |
| | NO: 364) |
| LAZR01002400_19 (SEQ ID NO: 175) | GTAGCAATGGCAGCGCATCGCCTTTTAAGATGCGAC (SEQ ID |
| | NO: 364) |
| FLSK01003024_2 (SEQ ID NO: 169) | GTTTAAGTAGTAGTGAGTTTAGCACATCTCTAAAAC (SEQ ID |
| | NO: 358) |
| OFLM01000072_9 (SEQ ID NO: 176) | TAATGAGTTTAGCATATCTCTAAAAC (SEQ ID NO: 365) |
| OFLO01000090_50 (SEQ ID NO: 176) | TGAGTTTAGCATATCTCTAAAAC (SEQ ID NO: 366) |
| OFLU01000140_22 (SEQ ID NO: 177) | GGTTAAGTAGTAGTGAGTTTAGCATATCTCTAAAAC (SEQ ID |
| | NO: 367) |
| OFLV01000230_3 (SEQ ID NO: 177) | GGTTAAGTAGTAGTGAGTTTAGCATATCTCTAAAAC (SEQ ID |
| | NO: 367) |
| OGCY01000078_30 (SEQ ID NO: 178) | GGTTAAGTAGTAGTGAGTTTAGCACATCTCTAAAAC (SEQ ID |
| | NO: 362) |
| OGJO01000473_2 (SEQ ID NO: 169) | GTTTAAGTAGTAGTGAGTTTAGCACATCTCTAAAAC (SEQ ID |
| | NO: 358) |
| OGJT01000109_37 (SEQ ID NO: 179) | GGTTAAATAGTAGTGAGTTTAGCACATCTCTAAAAC (SEQ ID |
| | NO: 368) |
| OGJZ01005194_5 (SEQ ID NO: 180) | GGTTAAGTAGTAGTGAGTTTAGCATATCTCTAAAAC (SEQ ID |
| | NO: 367) |
| OGKO01001669_8 (SEQ ID NO: 181) | GGTTAAGTAGTAGTGAGTTTAGCACATCTCTAAAAC (SEQ ID |
| | NO: 362) |
| OFCI01000292_37 (SEQ ID NO: 182) | GTAACAGAGGCTGCTCAATGCCTTTGAAATTGAGAC (SEQ ID |
| | NO: 369) |
| 3300006048\|Ga0075363_100000001_25 | GTAACAATCCCAGTGCAACGGGTTAGTAGTTGCAAC (SEQ ID |
| (SEQ ID NO: 183) | NO: 370) |
| 3300006048\|Ga0075363_100000001_20 | GTAACAATCCCAGTGCAACGGGTTAGTAGTTGCAAC (SEQ ID |
| (SEQ ID NO: 184) | NO: 370) |
| 3300006048\|Ga0075363_100000020_49 | GTGTGGAGGCAGCTCGGTGCCGATGGAAACTGAGAC (SEQ ID |
| (SEQ ID NO: 185) | NO: 371) |
| 3300006178\|Ga0075367_10000108_6 | GTAACAATCCCAGTGCAACGGGTTAGTAGTTGCAAC (SEQ ID |
| (SEQ ID NO: 183) | NO: 370) |
| 3300006178\|Ga0075367_10000108_6 | GTAACAATCCCAGTGCAACGGGTTAGTAGTTGCAAC (SEQ ID |
| (SEQ ID NO: 184) | NO: 370) |
| 3300006195\|Ga0075366_10000160_13 | GTAACAATCCCAGTGCAACGGGTTAGTAGTTGCAAC (SEQ ID |
| (SEQ ID NO: 183) | NO: 370) |
| 3300009500\|Ga0116229_10010095_9 | CTTGCAATGGCTGCGCAGGGCCTTGGACGCTGCGAC (SEQ ID |
| (SEQ ID NO: 186) | NO: 372) |
| 3300009701\|Ga0116228_10018148_5 | TCGAATTCGGTTGACGCT (SEQ ID NO: 373) |
| (SEQ ID NO: 187) | |
| 3300005577\|Ga0068857_100000008_197 | GTTGGCAATAGAAGCTAACCTCTATAAGCGTTAGACC (SEQ |
| (SEQ ID NO: 188) | ID NO: 374) |
| 3300005338\|Ga0068868_100030384_5 | GTGGAGAGGCCAGCGCAGGGGCTTTGTGCCTGCGAC (SEQ ID |
| (SEQ ID NO: 189) | NO: 375) |
| 3300005841\|Ga0068863_100041042_2 | GTTGAAAACCCCATCGATTCGGGGTAGTG (SEQ ID NO: |
| (SEQ ID NO: 190) | 376) |
| 3300013306\|Ga0163162_10000022_153 | CTCGCAAGCGTTGCTCGACACGCTAGGTGTCGAGAC (SEQ ID |
| (SEQ ID NO: 191) | NO: 377) |
| 3300009148\|Ga0105243_10000126_60 | TCGTCCTGCAGCGTGATCCCGCC (SEQ ID NO: 378) |
| (SEQ ID NO: 192) | |
| 3300006846\|Ga0075430_100000057_67 | GCCGTCGAAATGCCTGCTCGGGGGCTTCGTACCTGAGAC (SEQ |
| (SEQ ID NO: 193) | ID NO: 379) |
| 3300006853\|Ga0075420_100000070_3 | GCCGTCGAAATGCCTGCTCGGGGGCTTCGTACCTGAGAC (SEQ |
| (SEQ ID NO: 193) | ID NO: 379) |
| 3300006854\|Ga0075425_100000037_57 | GTGTGGAGGCAGCTCGGTGCCGATGGAAACTGAGAC (SEQ ID |
| (SEQ ID NO: 185) | NO: 371) |
| 3300006903\|Ga0075426_10000611_28 | AAGAGCATCCCGGGCGCGAA (SEQ ID NO: 380) |
| (SEQ ID NO: 194) | |
| 3300006914\|Ga0075436_100000782_9 | AAGAGCATCCCGGGCGCGAA (SEQ ID NO: 380) |
| (SEQ ID NO: 194) | |
| 3300007076\|Ga0075435_100000061_47 | AAGAGCATCCCGGGCGCGAA (SEQ ID NO: 380) |
| (SEQ ID NO: 194) | |
| 3300007076\|Ga0075435_100000750_29 | GTGTGGAGGCAGCTCGGTGCCGATGGAAACTGAGAC (SEQ ID |
| (SEQ ID NO: 185) | NO: 371) |
| 3300009100\|Ga0075418_10076301_2 | GTAGGAACCCCTAGTGTCCTGGGTGGAGAGGACAAC (SEQ ID |
| (SEQ ID NO: 195) | NO: 381) |
| 3300009100\|Ga0075418_10076301_2 | GTAGGAACCCCTAGTGTCCTGGGTGGAGAGGACAAC (SEQ ID |
| (SEQ ID NO: 196) | NO: 381) |
| 3300009156\|Ga0111538_10081463_8 | GCGCGGGGGCTTGGTTCCTGCGAC (SEQ ID NO: 382) |
| (SEQ ID NO: 197) | |
| 3300005548\|Ga0070665_100000073_173 | GAAACAAAGCGTGCTCTGCCGCTTGGAAGCAGAGAC (SEQ ID |
| (SEQ ID NO: 198) | NO: 383) |
| OBLM01000011_1 (SEQ ID NO: 199) | CGTGACGATGGTTGCTCG (SEQ ID NO: 384) |
| OCTA010000646_37 (SEQ ID NO: 200) | AATCCCTGCTCAAGAGGGTGTGTCTTGAGAC (SEQ ID NO: |
| | 385) |
| ODAK010001378_33 (SEQ ID NO: 201) | CTCGCAATCGTTGCTGGACGGACTTCTCGTTCAGAC (SEQ ID |
| | NO: 386) |
| ODAK010029943_5 (SEQ ID NO: 202) | GTCGAAATGCCCGCTCAGCGGCTTAGTTGCTGAGAC (SEQ ID |
| | NO: 387) |
| ODAK010029943_6 (SEQ ID NO: 203) | GTCGAAATGCCCGCTCAGCGGCTTAGTTGCTGAGAC (SEQ ID |
| | NO: 387) |
| 3300005602\|Ga0070762_10000001_34 | GTCGAGATCAGGCTCCATGAGATCGACCATGGAGAC (SEQ ID |
| (SEQ ID NO: 204) | NO: 388) |
| 3300005602\|Ga0070762_10000001_32 | GTCGAGATCAGGCTCCATGAGATCGACCATGGAGAC (SEQ ID |
| (SEQ ID NO: 205) | NO: 388) |
| 3300006796\|Ga0066665_10000988_15 | GGTGACAAAGCCCTGTGCAGCGGGCTCAAAGCTGCGAC (SEQ |
| (SEQ ID NO: 206) | ID NO: 389) |
| 3300018429\|Ga0190272_10000030_113 | GTCGCAACGCCTGCGTCGGGGCCTCGTGCCGACGAC (SEQ ID |
| (SEQ ID NO: 207) | NO: 390) |
| 3300018432\|Ga0190275_10000082_154 | GCAATGGCTGCTCAGCGCCCTTGAAGCTGAGAC (SEQ ID |
| (SEQ ID NO: 208) | NO: 391) |
| 3300018481\|Ga0190271_10027355_3 | AGCCTGCGCGGTGGCTGAGGACCGCGAC (SEQ ID NO: |
| (SEQ ID NO: 209) | 392) |
| 3300019874\|Ga0193744_1000265_21 | GTGTGGAGGCTGCTCAAGGCCGATGGAACTTGAGAC (SEQ ID |
| (SEQ ID NO: 210) | NO: 393) |
| 3300020021\|Ga0193726_1013919_1 | GGTGGCACGAGTTGCTCGGCGCTCTACGAGCCGAGAC (SEQ |
| (SEQ ID NO: 211) | ID NO: 394) |
| 3300020021\|Ga0193726_1013919_1 | GGTGGCACGAGTTGCTCGGCGCTCTACGAGCCGAGAC (SEQ |
| (SEQ ID NO: 212) | ID NO: 394) |
| 3300020034\|Ga0193753_10002988_10 | AACATCTGCTCGATCGATCCGAGATCGAGCC (SEQ ID NO: |
| (SEQ ID NO: 213) | 395) |
| 3300020034\|Ga0193753_10002988_9 | AACATCTGCTCGATCGATCCGAGATCGAGCC (SEQ ID NO: |
| (SEQ ID NO: 214) | 395) |
| 3300020156\|Ga0196970_1000866_40 | TGGAAACCCTGCGCAGGGGGTTAAAGCCTGCGAC (SEQ ID |
| (SEQ ID NO: 215) | NO: 396) |
| 3300020579\|Ga0210407_10000200_14 | GTCTCAATGGCTGCGACGAGCCGTGCAATCGTCGAC (SEQ ID |
| (SEQ ID NO: 216) | NO: 397) |
| 3300020580\|Ga0210403_10000550_35 | GTCTCAATGGCTGCGACGAGCCGTGCAATCGTCGAC (SEQ ID |
| (SEQ ID NO: 216) | NO: 397) |
| 3300020580\|Ga0210403_10001296_17 | CGCAAGCATTCGTCACAATCA (SEQ ID NO: 398) |
| (SEQ ID NO: 217) | |
| 3300020581\|Ga0210399_10010852_9 | GTTGTAGAGGTAACGAAGCACCTGAAAGACTTCGAG (SEQ ID |
| (SEQ ID NO: 218) | NO: 399) |
| 3300020583\|Ga0210401_10033176_5 | GGTAGCGAAACACCTTGAAG (SEQ ID NO: 400) |
| (SEQ ID NO: 217) | |
| 3300005435\|Ga0070714_100002341_12 | GTGGAAAAGAGCAGCGCGGGGCTCCAACGCCGCGAC (SEQ ID |
| (SEQ ID NO: 219) | NO: 401) |
| 3300009095\|Ga0079224_100000262_28 | GCTGCAAAGGACGTGGCGTTCCTTGAACACGCCAAC (SEQ ID |
| (SEQ ID NO: 220) | NO: 402) |
| 3300009095\|Ga0079224_100170797_3 | GTTGCAATGCCGACTCCGCGGCTTGGTTGCGGAGAGG (SEQ |
| (SEQ ID NO: 221) | ID NO: 403) |
| 3300010343\|Ga0074044_10013672_1 | GTCGCAGGTGATGCTCTGAGGCTTTGGATCGGAGAC (SEQ ID |
| (SEQ ID NO: 222) | NO: 404) |
| 3300010343\|Ga0074044_10041345_4 | GTGGAAAGGGCAGCGCAGAGCCCGTGATGCTGCGAC (SEQ ID |
| (SEQ ID NO: 223) | NO: 405) |
| 3300005468\|Ga0070707_100000083_12 | CGGCGTGCCCTCGTCCTGCACCGTGATCCCGGC (SEQ ID |
| (SEQ ID NO: 224) | NO: 406) |
| 3300006163\|Ga0070715_10000067_44 | GCGACTGCGTTTGCTCGGTGACGGGTTCACCGAGAC (SEQ ID |
| (SEQ ID NO: 225) | NO: 407) |
| 3300014498\|Ga0182019_10003703_1 | GTAACAATGCCAGCCCAGAGGCAAAGGTTCTGGGAC (SEQ ID |
| (SEQ ID NO: 226) | NO: 408) |
| 3300001131\|JGI12631J13338_1000296_13 | GTGCAGCCCCTGGGCACGTGGGCGACGG (SEQ ID NO: |
| (SEQ ID NO: 227) | 409) |
| 3300001593\|JGI12635J15846_10002852_1 | GTGCAGCCCCTGGGCACGTGGGCGACGG (SEQ ID NO: |
| (SEQ ID NO: 227) | 409) |
| 3300009813\|Ga0105057_1000075_5 | GATCACACGGCGACCTCCGCCGCCTCGAAGCGGAGAC (SEQ |
| (SEQ ID NO: 228) | ID NO: 410) |
| 3300009813\|Ga0105057_1000075_5 | GATCACACGGCGACCTCCGCCGCCTCGAAGCGGAGAC (SEQ |
| (SEQ ID NO: 229) | ID NO: 410) |
| 3300014489\|Ga0182018_10031574_1 | CTCACAACGGCAGCGAGGCGCCTTGGAGTCCTCGAC (SEQ ID |
| (SEQ ID NO: 230) | NO: 411) |
| 3300014501\|Ga0182024_10047267_8 | GTAGCAAGAGGTGCTCATGCCTCTAGAATATGAGAC (SEQ ID |
| (SEQ ID NO: 231) | NO: 412) |
| 3300014501\|Ga0182024_10150440_2 | GTAGCAAAGGCAGCTTGACGCCTTAAAGATCAAGAC (SEQ ID |
| (SEQ ID NO: 232) | NO: 413) |
| 3300001356\|JGI12269J14319_10001968_12 | CTCGCAACGCCAGCGCAGGGGCCATGACGCTGCGAC (SEQ ID |
| (SEQ ID NO: 233) | NO: 414) |
| 3300007533\|Ga0102944_1012316_2 | GCATCAAAGGCAGTCCGATGCCTCTCAAATCGGAAC (SEQ ID |
| (SEQ ID NO: 234) | NO: 415) |
| 3300005903\|Ga0075279_10000001_30 | GTTGGAAAGGCTGCTCGAACGCCTTCAAGTCGAGAG (SEQ ID |
| (SEQ ID NO: 235) | NO: 416) |
| 3300005524\|Ga0070737_10002282_10 | CCGTGCGCAGCCGGATAACGCTGCGAC (SEQ ID NO: 417) |
| (SEQ ID NO: 236) | |
| 3300005524\|Ga0070737_10031205_1 | GTCGCGAAGCTAGCGCAGAAGCTTGGTATCTGCGAG (SEQ ID |
| (SEQ ID NO: 237) | NO: 418) |
| 3300005524\|Ga0070737_10031205_1 | GTCGCGAAGCTAGCGCAGAAGCTTGGTATCTGCGAG (SEQ ID |
| (SEQ ID NO: 238) | NO: 418) |
| 3300005534\|Ga0070735_10023967_5 | GCATCAAGTCTTATCTCGC (SEQ ID NO: 419) |
| (SEQ ID NO: 239) | |
| 3300005542\|Ga0070732_10013271_3 | GGCAGCGAAACGCCTTGAAG (SEQ ID NO: 420) |
| (SEQ ID NO: 240) | |
| 3300010373\|Ga0134128_10000310_109 | GTGTAATGCCCGGCAGAAGGCTTTGGATTCTGCGAC (SEQ ID |
| (SEQ ID NO: 241) | NO: 421) |
| 3300010373\|Ga0134128_10011458_1 | GTAGCAATGCCTACCAAGAGGCTTTGTATCTTGTGAG (SEQ |
| (SEQ ID NO: 242) | ID NO: 422) |
| 3300010373\|Ga0134128_10096594_3 | GGCGCAAGCCCTGCGCGGGAGGGCAAGATCCTGCGAC (SEQ |
| (SEQ ID NO: 243) | ID NO: 423) |
| 3300010400\|Ga0134122_10000107_57 | CAAGCCACGCTCGGGTGGC (SEQ ID NO: 424) |
| (SEQ ID NO: 244) | |
| 3300010401\|Ga0134121_10002041_17 | GGAGAGGGATCGTTTCTTCGACC (SEQ ID NO: 425) |
| (SEQ ID NO: 245) | |
| 3300004633\|Ga0066395_10000027_32 | CCGACAACGCCTGCGCAGGGGCGTGGTTTCTGCGAC (SEQ ID |
| (SEQ ID NO: 246) | NO: 426) |
| 3300005332\|Ga0066388_100004304_4 | GGTGCAACGTGCCTTGCGAGGGCTTGATACTCGCGAC (SEQ |
| (SEQ ID NO: 247) | ID NO: 427) |
| 3300005332\|Ga0066388_100004304_2 | GGTGCAACGTGCCTTGCGAGGGCTTGATACTCGCGAC (SEQ |
| (SEQ ID NO: 248) | ID NO: 427) |
| 3300005764\|Ga0066903_100000051_27 | CCGACAACGCCTGCGCAGGGGCGTGGTTTCTGCGAC (SEQ ID |
| (SEQ ID NO: 246) | NO: 426) |
| 3300010047\|Ga0126382_10001209_14 | GGTGCAACGTGCCTTGCGAGGGCTTGATACTCGCGAC (SEQ |
| (SEQ ID NO: 249) | ID NO: 427) |
| 3300010047\|Ga0126382_10001209_12 | GGTGCAACGTGCCTTGCGAGGGCTTGATACTCGCGAC (SEQ |
| (SEQ ID NO: 248) | ID NO: 427) |
| 3300010048\|Ga0126373_10000093_102 | GTGGTAATGGCAGCGCAGCGCCTTTGAGACTGCGAC (SEQ ID |
| (SEQ ID NO: 250) | NO: 428) |
| 3300010366\|Ga0126379_10001683_10 | GTTGCAGTACCCTGCTCACGGGGGAGACAAGTGAGAG (SEQ |
| (SEQ ID NO: 251) | ID NO: 429) |
| 3300010376\|Ga0126381_100020658_4 | GGTGAAATGGCATCGGGAGGCCACAAACGTTCCGAC (SEQ ID |
| (SEQ ID NO: 252) | NO: 430) |
| 3300010398\|Ga0126383_10032213_5 | GTGATAGTGCCTGCTCAGTGGCTTAGT (SEQ ID NO: 431) |
| (SEQ ID NO: 253) | |
| 3300017961\|Ga0187778_10004454_1 | GTGACAAGAGCAGCGCGGCGCTCTGCGAGCCGCGAC (SEQ ID |
| (SEQ ID NO: 254) | NO: 432) |
| 3300017970\|Ga0187783_10000008_23 | CGAGTAGGCTTAGTTTGCTCGAC (SEQ ID NO: 433) |
| (SEQ ID NO: 255) | |
| 3300017972\|Ga0187781_10019688_5 | GGCGAAGCGTCAGCGCAGCCGTCTCGAGGCTGCGAC (SEQ ID |
| (SEQ ID NO: 256) | NO: 434) |
| 3300018064\|Ga0187773_10011230_2 | GTCGCGATGGCTGCTCGACGCCAGGAAGATCGAGAC (SEQ ID |
| (SEQ ID NO: 257) | NO: 435) |
| 3300012204\|Ga0137374_10001132_4 | GTCGAAATGCCCGCGCGGGGGCGTCGTACCCGCGAC (SEQ ID |
| (SEQ ID NO: 258) | NO: 436) |
| 3300012210\|Ga0137378_10000107_47 | GCGTGTAGTAAGAGCAGCGGTGTCGCTCTGAGATGCCGAC |
| (SEQ ID NO: 259) | (SEQ ID NO: 437) |
| 3300012532\|Ga0137373_10000316_4 | GTCGAAATGCCCGCGCGGGGGCGTCGTACCCGCGAC (SEQ ID |
| (SEQ ID NO: 258) | NO: 436) |
| 3300012532\|Ga0137373_10000407_43 | GCCCAAAGGCAGCTCGGCGCCTACAGAAGCCGAGAC (SEQ ID |
| (SEQ ID NO: 260) | NO: 438) |
| 3300012930\|Ga0137407_10020190_4 | GGTCGAAATGCCTGCGCAGGGGCTTCAACGCTGCGAC (SEQ |
| (SEQ ID NO: 261) | ID NO: 439) |
| 3300005987\|1071089\|scaffold14955_2 | ATCGAAGAGCCTGCGCAGAGGCTTTTGATCTGCGAT (SEQ ID |
| (SEQ ID NO: 262) | NO: 440) |
| 3300005988\|1071091\|scaffold06014_8 | ATCGAAGAGCCTGCGCAGAGGCTTTTGATCTGCGAT (SEQ ID |
| (SEQ ID NO: 262) | NO: 440) |
| 3300006056\|1071094\|scaffold118627_2 | ATCGAAGAGCCTGCGCAGAGGCTTTTGATCTGCGAT (SEQ ID |
| (SEQ ID NO: 262) | NO: 440) |

### REFERENCES

D. A. Benson et al., GenBank. Nucleic Acids Res. 41, D36-42 (2013).
K. D. Pruitt, T. Tatusova, G. R. Brown, D. R. Maglott, NCBI Reference Sequences (RefSeq): current status, new features and genome annotation policy. Nucleic Acids Res. 40, D130-135 (2012).
V. M. Markowitz et al., IMG: the Integrated Microbial Genomes database and comparative analysis system. Nucleic Acids Res. 40, D115-122 (2012).

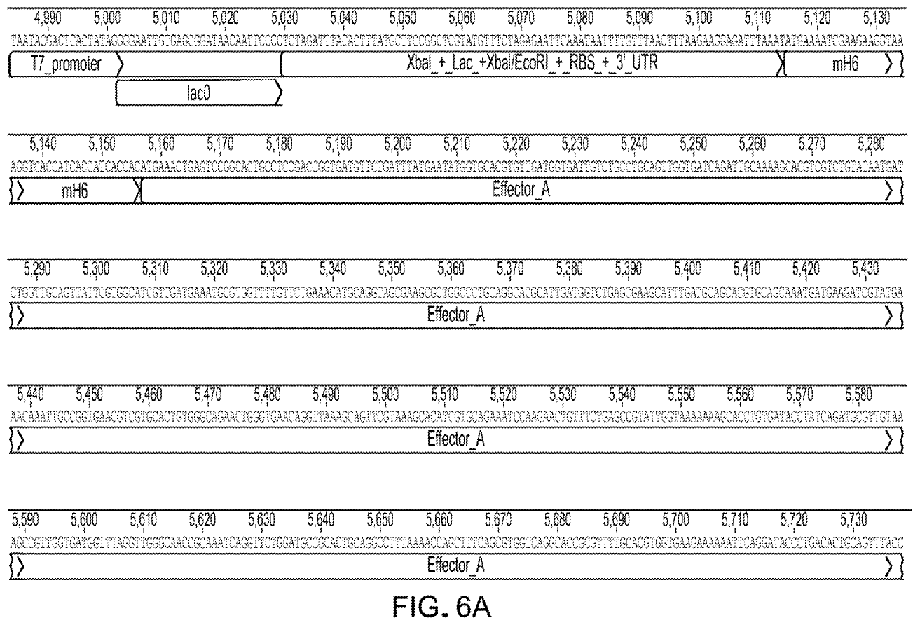

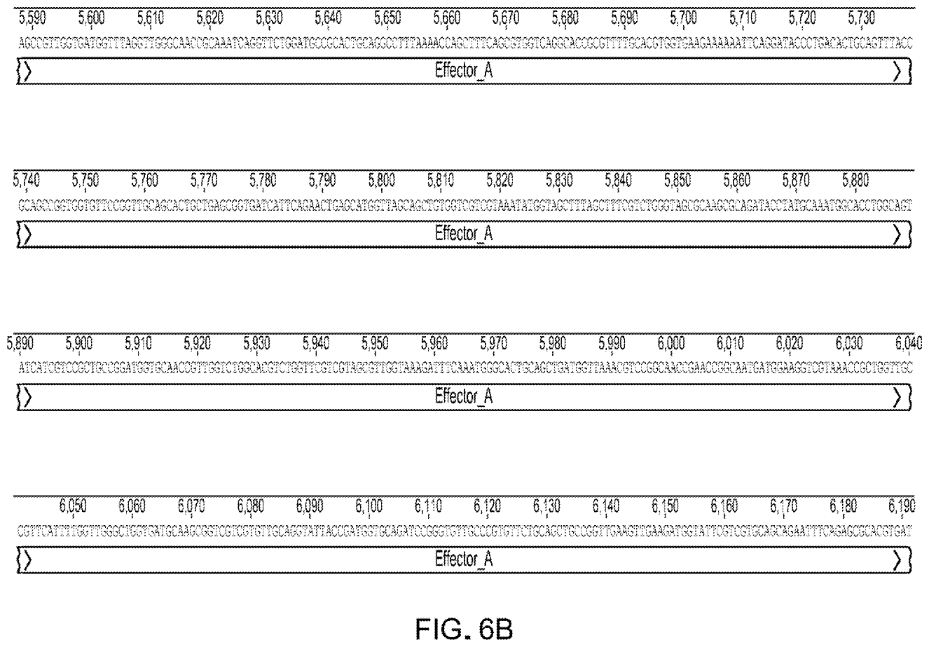

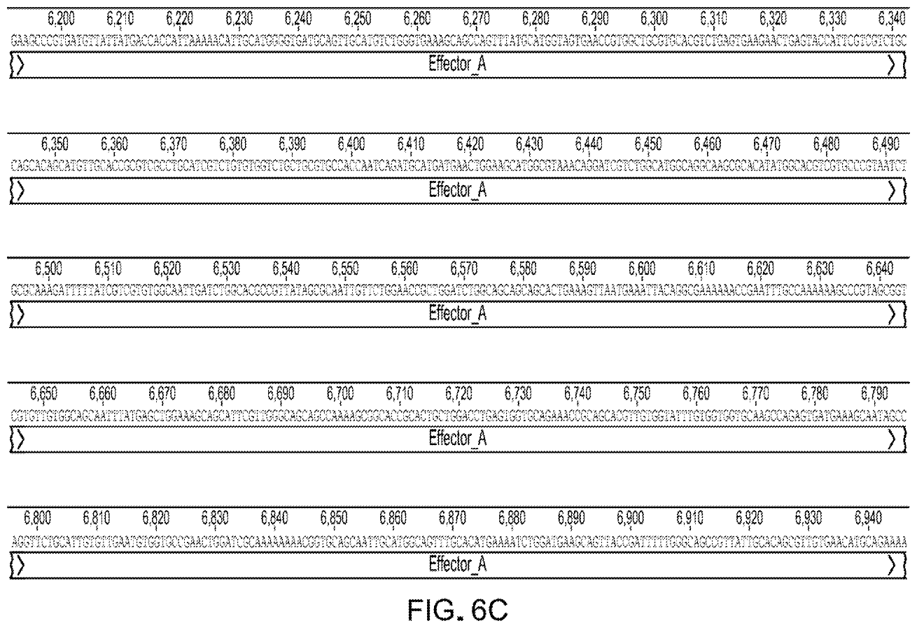

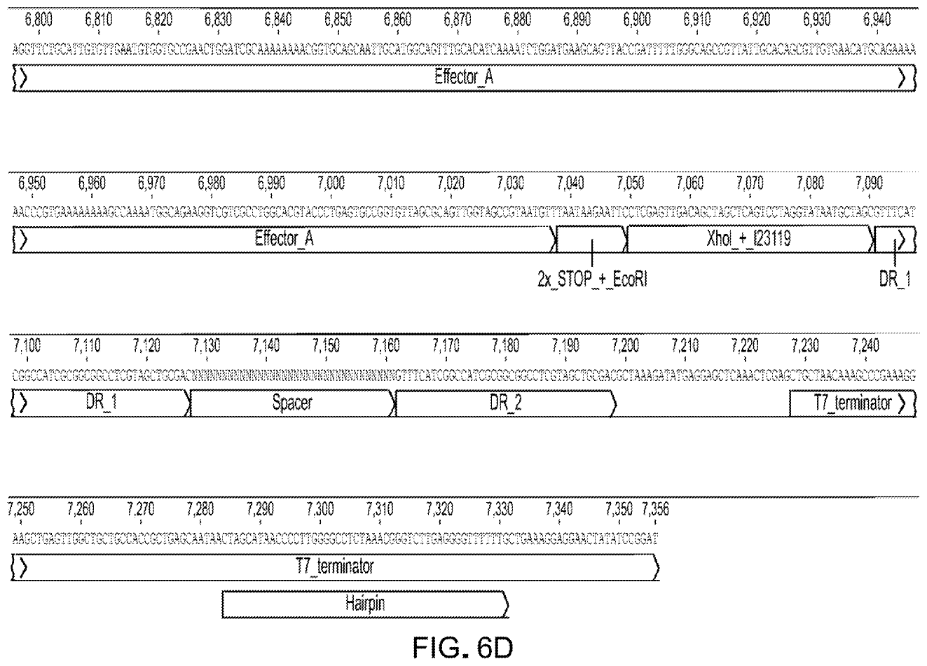

### Example 2—Functional Validation of Engineered CLUST.018837 CRISPR-Cas Systems (FIGS. 6-11)

Having identified the minimal components of CLUST.018837 CRISPR-Cas systems, multiple example systems were selected for functional validation, from the sources designated NZ_LDOS01000005 (SEQ ID NO: 1), 3300009004 (SEQ ID NO: 9), APMI01033782 (SEQ ID NO: 26), NZ_LVXZ01000012 (SEQ ID NO: 3), and ADIG01000806 (SEQ ID NO: 20).

### DNA Synthesis and Effector Library Cloning

To test the activity of an exemplary CLUST.018837 CRISPR-Cas system, systems containing the pET28a(+) vector were designed and synthesized. The E. coli codon-optimized nucleic acid sequences encoding the selected CLUST.018837 effector proteins (amino acid sequence provided in TABLE 2) were synthesized (Genscript) and cloned into a custom expression system derived from the pET-28a(+) (EMD-Millipore) to create the Effector Plasmid. The engineered, non-naturally occurring vector included a nucleic acid encoding the CLUST.018837 effector protein under the control of a lac promoter and an E. coli ribosome binding sequence. The vector also included an acceptor site for a CRISPR array library driven by a J23119 promoter following the open reading frame for the CLUST.018837 effector protein (FIGS. 6A-D).

For the minimal CRISPR array, oligonucleotide library synthesis (OLS) pools comprising two direct repeats flanking natural-length spacer sequences targeting the pACYC184 plasmid, select E. coli essential genes, and non-targeting negative control spacers were designed for a total of 8900 elements in the array library. The spacer length was determined by the mode of the spacer lengths found in the endogenous CRISPR array. Flanking the minimal CRISPR array were unique PCR priming sites that enabled amplification of a specific library from a larger pool of oligo synthesis. These sequences were placed under the control of a J23119 promoter and cloned into the Effector Plasmid in both the forward and reverse orientations for a total library of ˜18,000 plasmid elements

The minimal CRISPR array library was next cloned into the Effector Plasmid using the Golden Gate assembly method. Briefly, each minimal CRISPR array from the OLS pool (Agilent Genomics) was first amplified using unique PCR primers, and pre-linearized the plasmid backbone using BsaI to reduce potential background. Both DNA fragments were purified with Ampure® XP (Beckman Coulter) prior to addition to Golden Gate Assembly Master Mix (New England Biolabs) and incubated per the manufacturer's instructions. The Golden Gate reaction was further purified and concentrated to enable maximum transformation efficiency in the subsequent steps of the bacterial screen.

The plasmid library containing the distinct minimal CRISPR array and CLUST.018837 effector sequence was electroporated into E. Cloni® electrocompetent E. coli (Lucigen) using a Gene Pulser Xcell® (BioRad) following the protocol recommended by Lucigen. The library was co-transformed with purified pACYC184 plasmid, plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested and plasmid DNA extracted using a QIAprep Spin Miniprep® Kit (Qiagen) to create an “output library.” By performing a PCR using custom primers containing barcodes and sites compatible with Illumina sequencing chemistry, a barcoded next generation sequencing library was generated from both the pre-transformation “input library” and the post-harvest “output library,” which were then pooled and loaded onto a Nextseq 550 (Illumina) to evaluate the effectors. At least two independent biological replicates were performed for each screen to ensure consistency.

### Bacterial Screen Sequencing Analysis

Next generation sequencing (NGS) data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. coli essential genes) or negative control sequence (GFP) to determine the corresponding target.

To identify specific parameters resulting in enzymatic activity and bacterial cell death, NGS was used to quantify and compare the representation of individual CRISPR arrays (i.e., repeat-spacer-repeat) in the PCR product of the input and output plasmid libraries. The array depletion ratio was defined as the normalized output read count divided by the normalized input read count. An array was considered to be “strongly depleted” if the depletion ratio was less than 0.33 (more than 3-fold depletion). When calculating the array depletion ratio across biological replicates, the maximum depletion ratio value for a given CRISPR array across all experiments (i.e., a strongly depleted array must be strongly depleted in all biological replicates) was taken. A matrix including array depletion ratios and the following features for each spacer target: target strand, transcript targeting, ORI targeting, target sequence motifs, flanking sequence motifs, and target secondary structure were generated. The degree to which different features in this matrix explained target depletion for CLUST.018837 systems was investigated, thereby yielding a broad survey of functional parameters within a single screen.

A matrix including array depletion ratios and the following features for each spacer target: target strand, transcript targeting, ORI targeting, target sequence motifs, flanking sequence motifs, and target secondary structure were generated. The degree to which different features in this matrix explained target depletion for CLUST.018837 systems was investigated, thereby yielding a broad survey of functional parameters within a single screen.

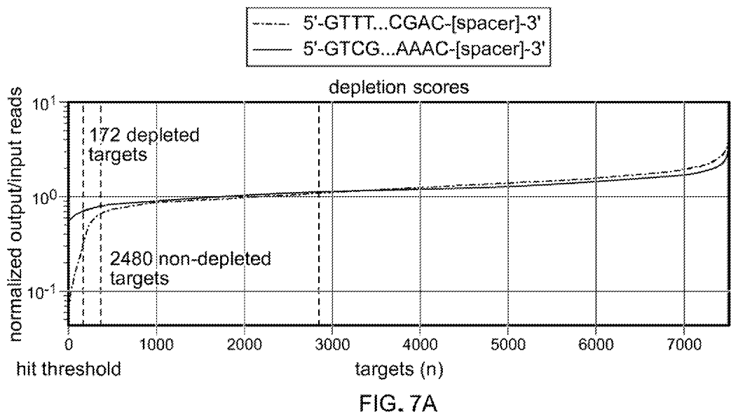

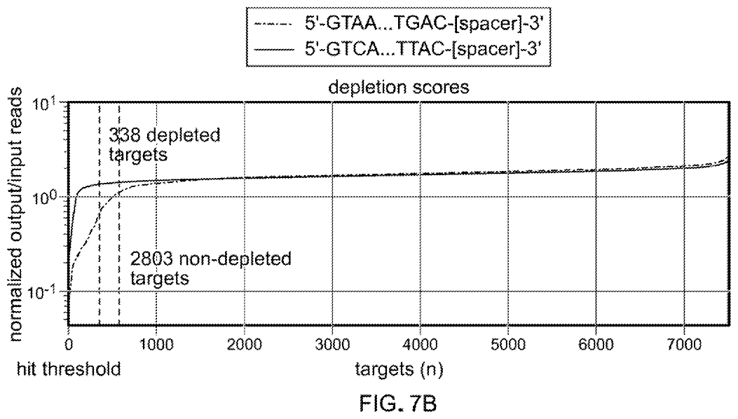

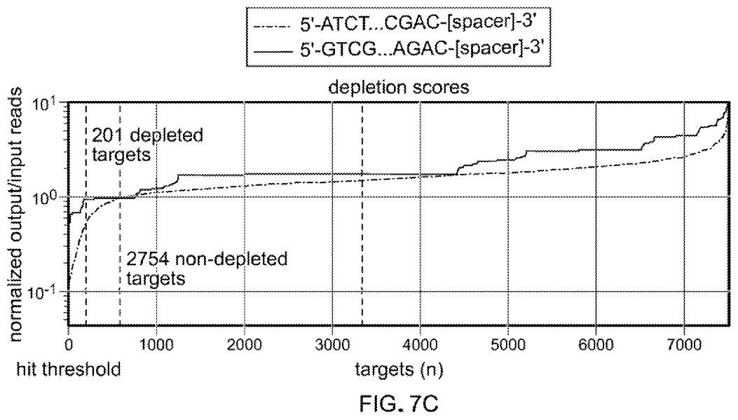

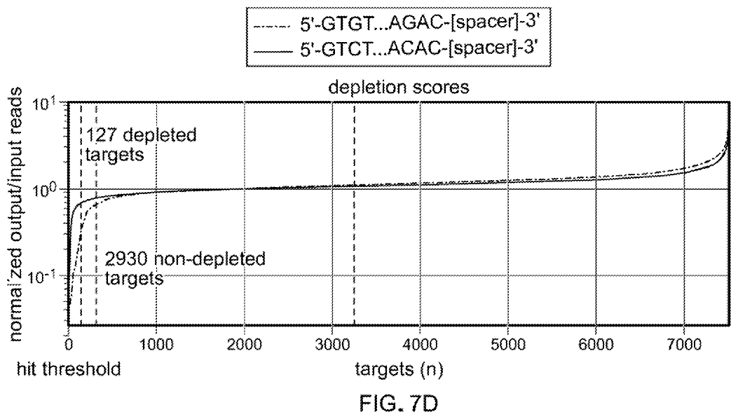

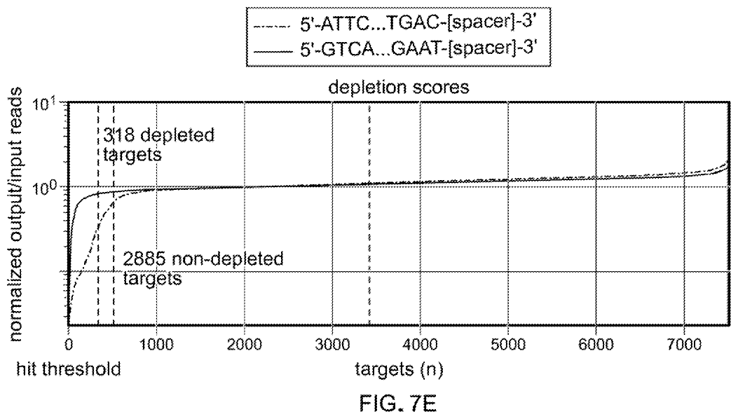

FIGS. 7A-E show the degree of depletion activity of the engineered compositions by plotting for a given target the normalized ratio of sequencing reads in the screen output versus the screen input.

To quantify depletion activity, an enrichment ratio was calculated as R_{treated}/Rᵢₙₚᵤₜ for each direct repeat and spacer. The normalized input read count was computed as:

Rᵢₙₚᵤₜ=# reads containing DR+spacer/total reads

where the reads counts were obtained from next-generation sequencing of the plasmid DNA library expressing a CLUST.018837 effector and associated crRNA prior to transformation. The normalized treated read count was computed as:

R_{treated}=(1+# reads containing DR+spacer)/total reads

where the read counts were obtained from next-generation sequencing of the plasmid DNA extracted from the surviving cells expressing CLUST.018837 effector and associated crRNA after antibiotic screening. A strongly depleted target had an enrichment less than ⅓, which was marked by the first vertical dashed line. Each CLUST.018837 effector was paired with a CRISPR array that took the form 5′-DR-[spacer]-DR-3′ or 5′-reverse_complement(DR)-[spacer]-reverse_complement(DR)-3′, and the depletion activity of both orientations of the DR are shown in the figure as indicated in the legend.

The results are plotted for each DR transcriptional orientation. In the functional screen for each composition, an active effector complexed with an active crRNA (expressed as a DR::spacer::DR) interferes with the ability of the pACYC184 to confer E coli resistance to chloramphenicol and tetracycline, resulting in cell death and depletion of the spacer element within the pool. Comparing the results of deep sequencing the initial DNA library (screen input) versus the surviving transformed E. coli (screen output) suggest specific target sequences and DR transcriptional orientation that enable an active, programmable CRISPR-Cas system. The screen also indicates that the effector complex is only active with one orientation of the DR.

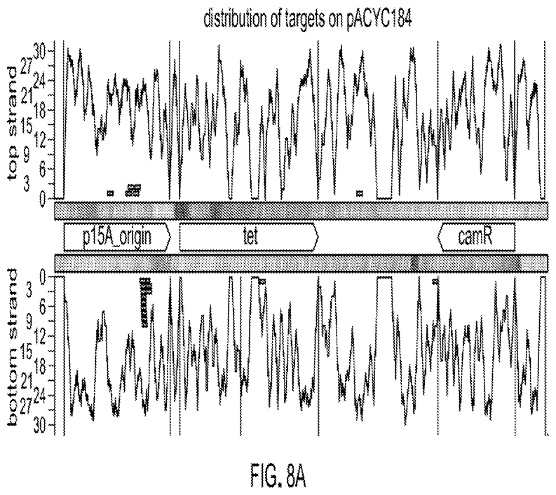

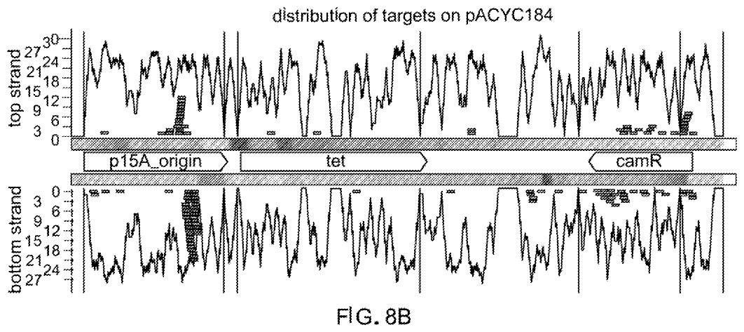

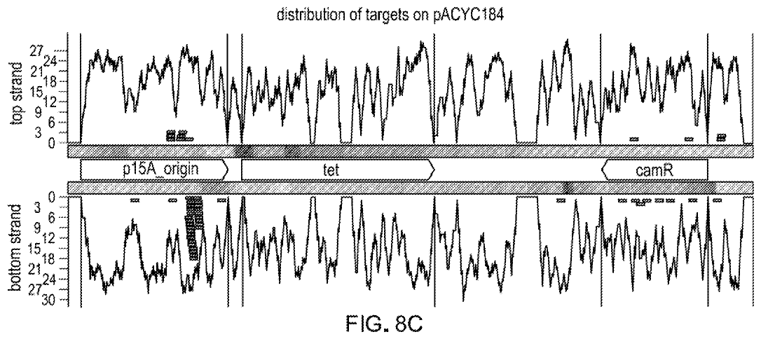

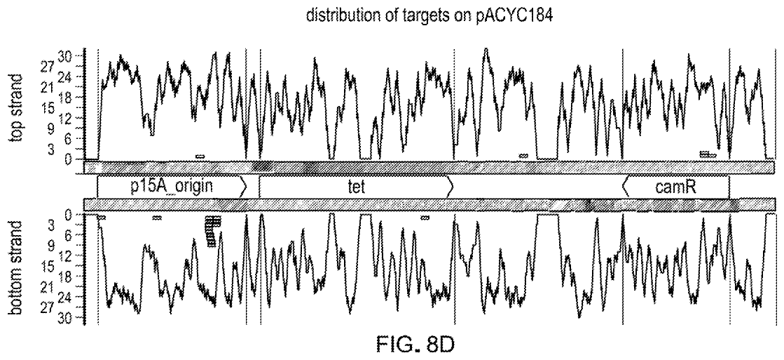

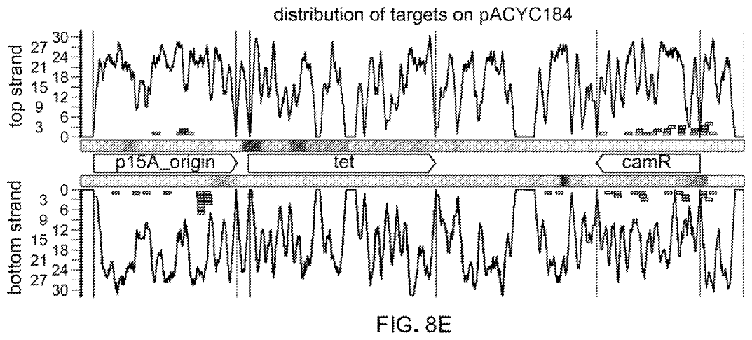

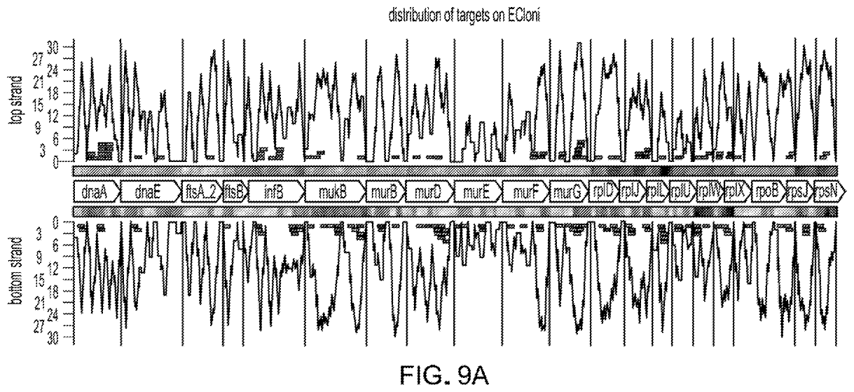

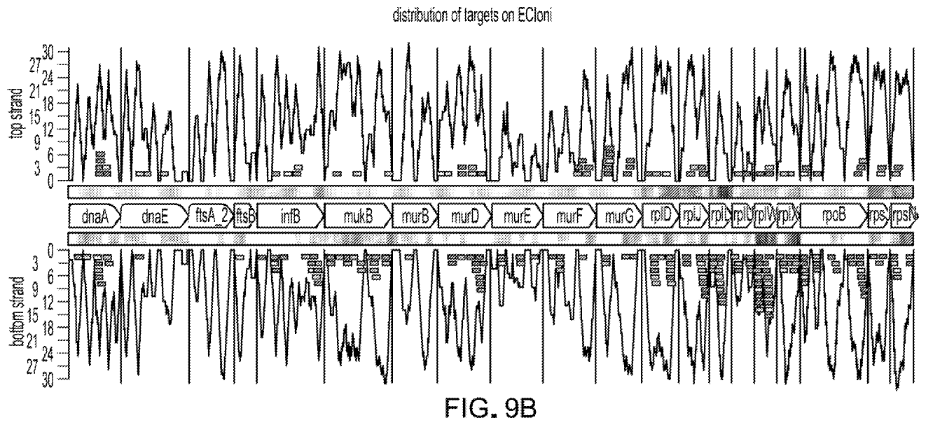

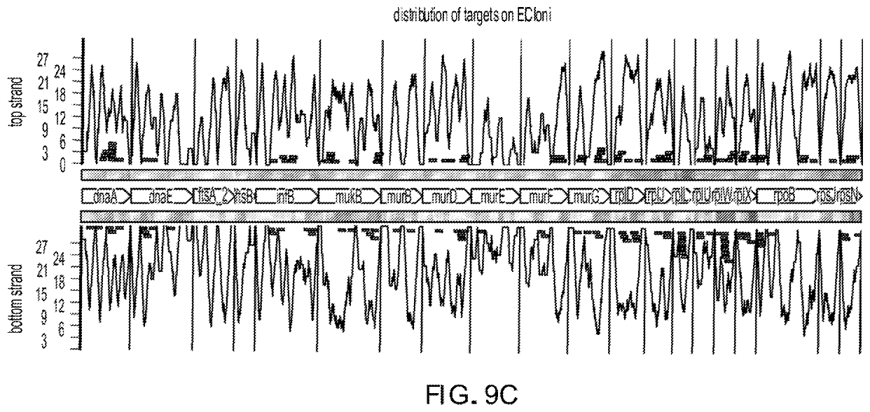

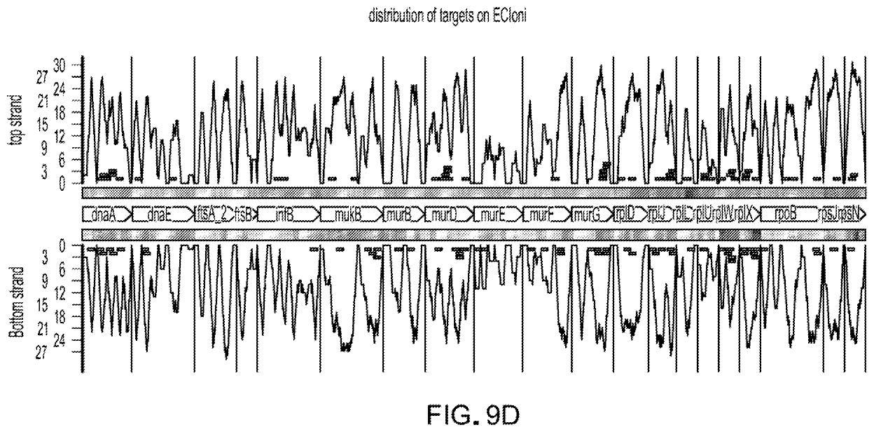

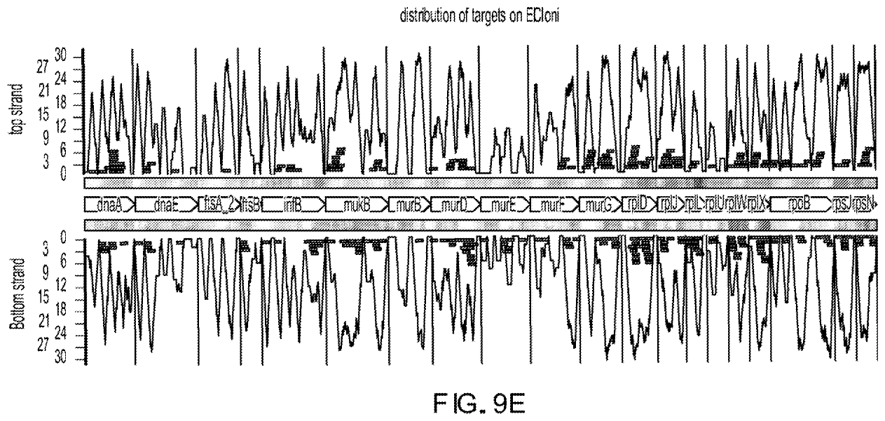

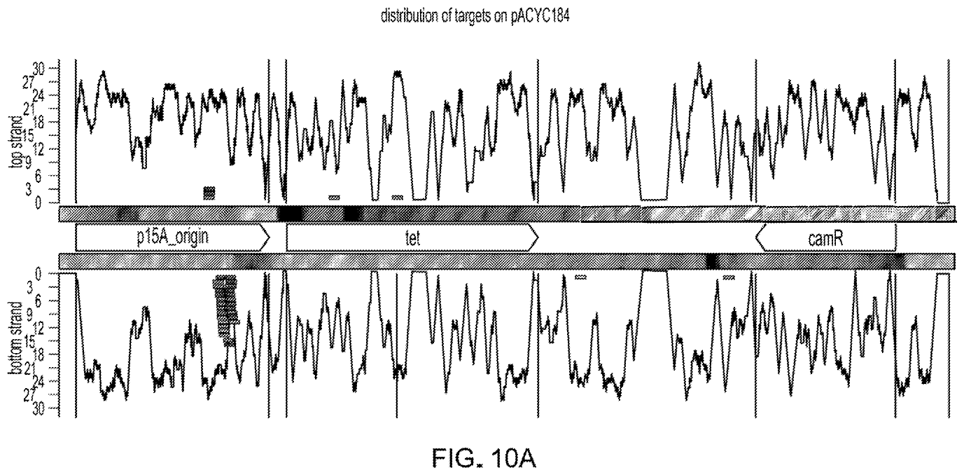

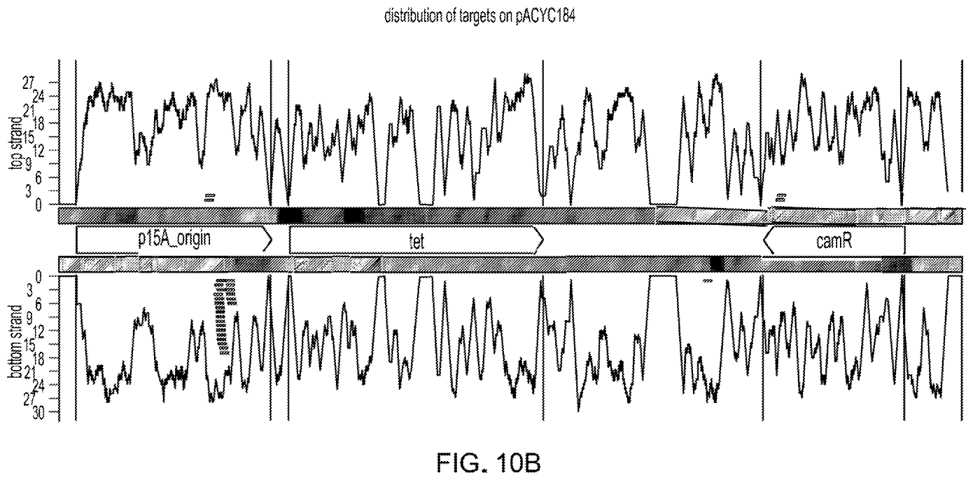

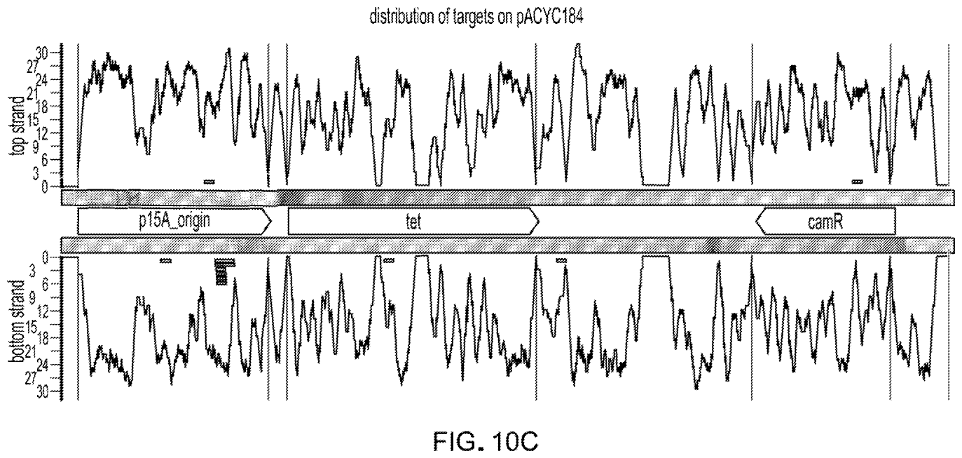

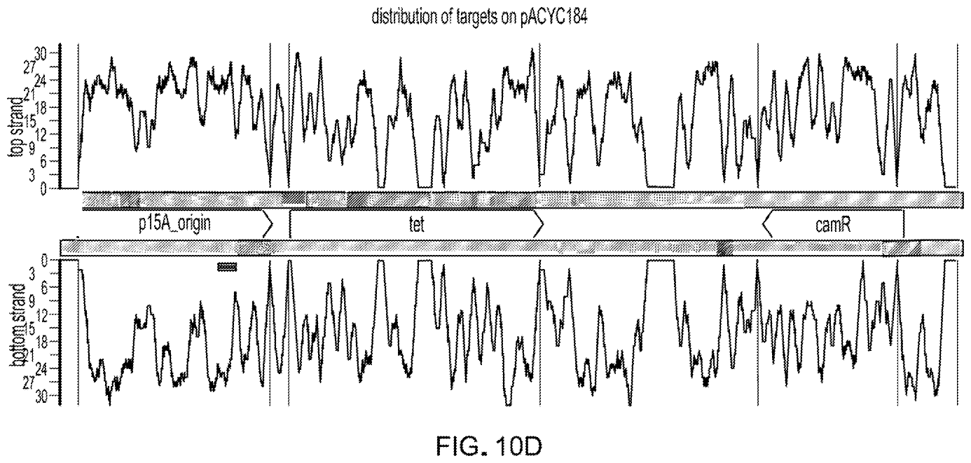

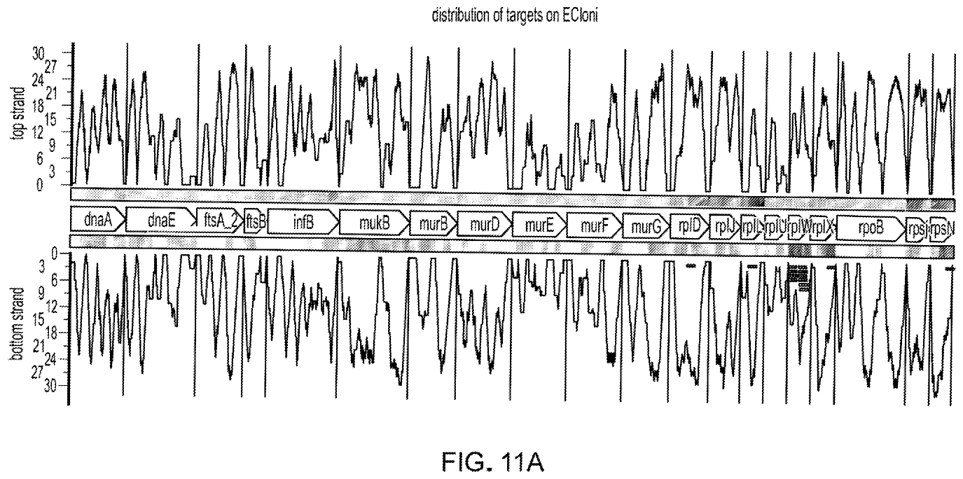

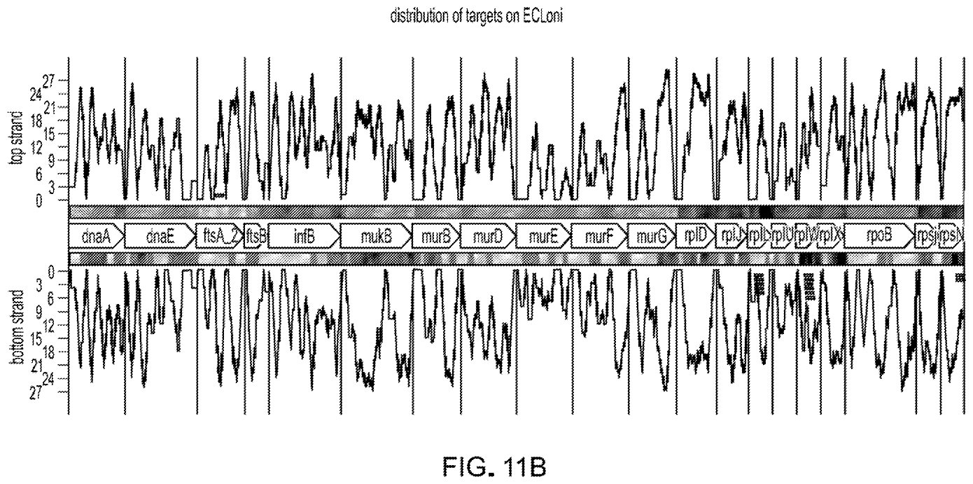

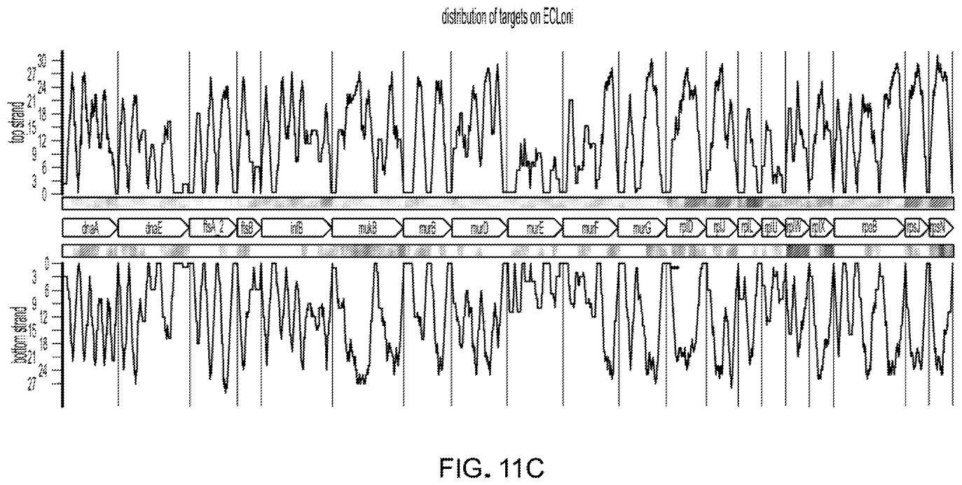

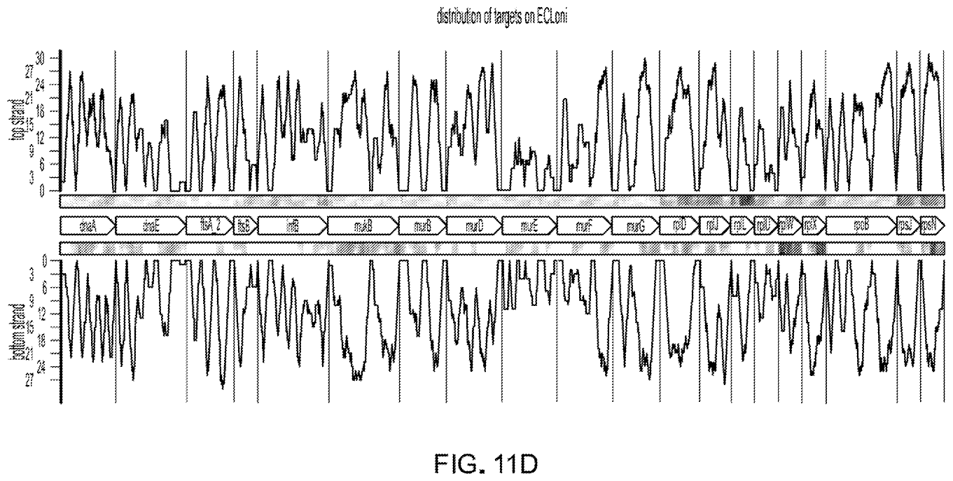

FIGS. 8A-E depicts the location of strongly depleted targets for CLUST.018837 systems targeting pACYC184, and FIGS. 9A-E depicts the location of strongly depleted targets for CLUST.018837 systems targeting E. coli essential genes. FIGS. 10A-E and FIGS. 11A-E depict strongly depleted targets for the negative control, whereby the nucleotide sequence encoding the CLUST.018837 effector has been deleted from the construct being screened. Notably, the presence of many strongly depleted targets in FIGS. 9A-E without corresponding activity in FIGS. 11A-E indicates interference activity that is dependent upon the expression of the CLUST.018837 effector and programmed by the RNA guide. Conversely, the appearance of strongly depleted spacers in the region of the pACYC184 origin of replication in both FIGS. 8A-E and FIGS. 10A-E (particularly prominent in the case of 3300009004) suggests that the observed depletion activity in the origin of replication is not related to the CLUST.018837 effector activity.

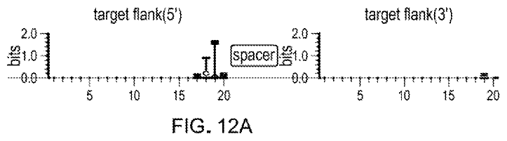

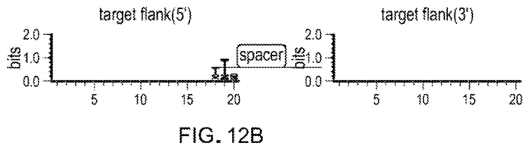

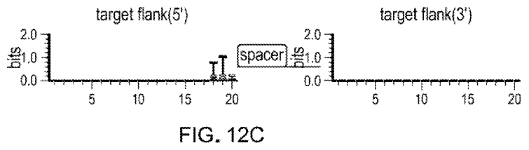

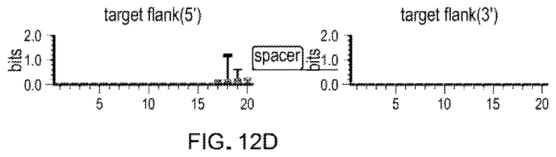

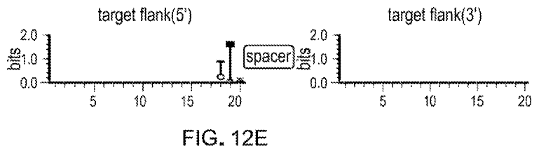

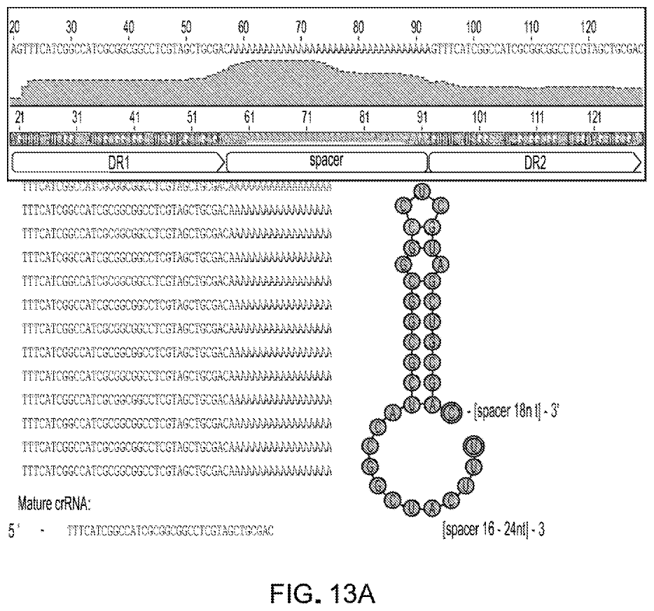

FIGS. 12A-E depict the sequences flanking targets strongly depleted by CLUST.018837 CRISPR-Cas systems, indicating a prominent 5′ PAM of 5′-TTN-3′ or 5′-YTN-3′.

RNA-Sequencing Mature crRNA from In Vivo Bacterial Screen

Sequencing the small RNA from the in vivo bacterial screen began by extracting total RNA from harvested screen bacteria using the Direct-zol RNA MiniPrep® Plus w/ TRI Reagent (Zymo Research). Ribosomal RNA was removed using a Ribo-Zero® rRNA Removal Kit for Bacteria, followed by cleanup using an RNA Clean and Concentrator-5 kit. The resultant ribosomal RNA depleted total RNA was treated with T4 PNK, RNA 5′ polyphosphatase, prepared for sequencing using the NEBNext® Small RNA Library Prep Set.

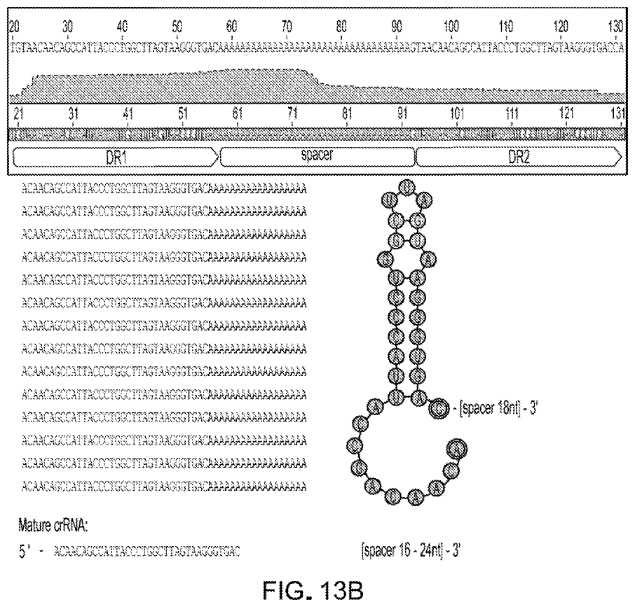

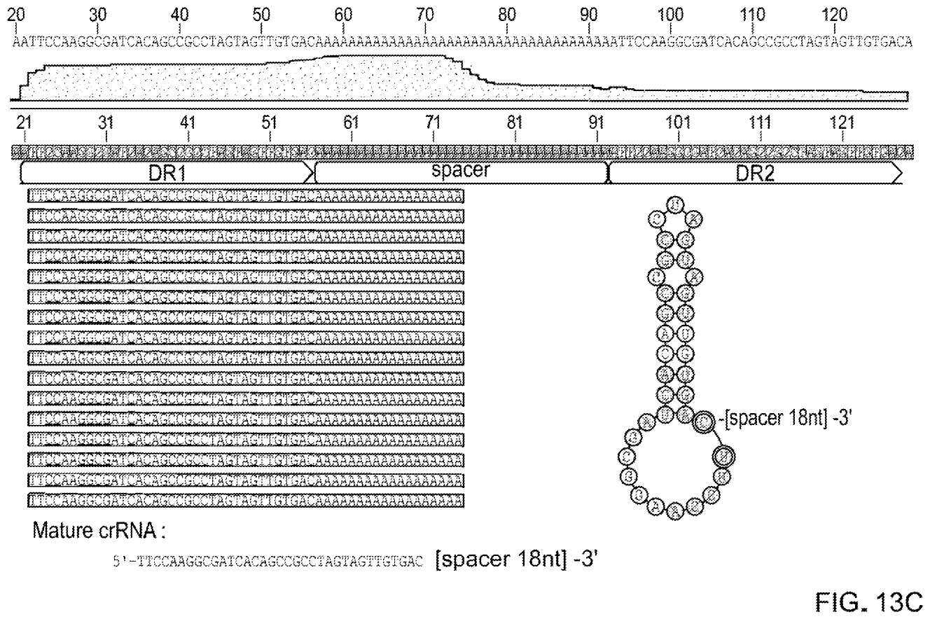

The pre-crRNA processing in the screen output samples for the direct repeat orientation that demonstrated successful targeting of pACYC184 and E. coli essential genes was investigated. FIGS. 13A-C depict the alignment of extracted RNA against the input minimal CRISPR arrays, revealing the form of the mature crRNA. Mature crRNA sequences for example CLUST.018837 CRISPR-Cas systems are given in Table 4.

TABLE 4
Nucleotide Sequences of Mature crRNA of
Representative CLUST.018837 CRISPR-Cas systems

| CLUST.018837 Effector | |
|---|---|
| Protein Accession | Mature crRNA Sequence |
| WP_081130164.1 | TTTCATCGGCCATCGCGGCGG |
| (SEQ ID NO: 1) | CCTCGTAGCTGCGACNNNNNN |
| | NNNNNNNNNNNN |
| | (SEQ ID NO: 1001) |
| WP_081130164.1 | TTCATCGGCCATCGCGGCGGC |
| (SEQ ID NO: 1) | CTCGTAGCTGCGACNNNNNNN |
| | NNNNNNNNNNN |
| | (SEQ ID NO: 1002) |
| 3300009004\|Ga0100377_ | ACAACAGCCATTACCCTGGCT |
| 1000348_44 | TAGTAAGGGTGACNNNNNNNN |
| (SEQ ID NO: 9) | NNNNNNNNNN |
| | (SEQ ID NO: 1003) |
| ADIG01000806_20 | TTCCAAGGCGATCACAGCCGC |
| (SEQ ID NO: 20) | CTAGTAGTTGTGACNNNNNNN |
| | NNNNNNNNNNN |
| | (SEQ ID NO: 1004) |

### In Vitro Pre-crRNA Processing

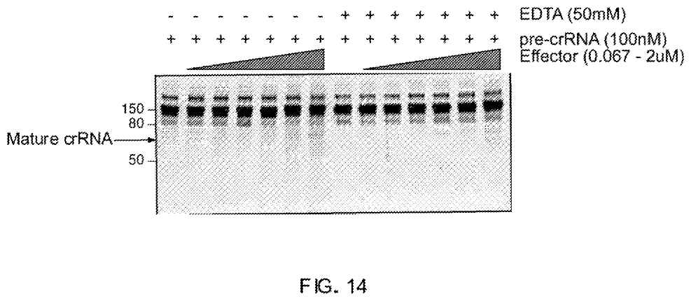

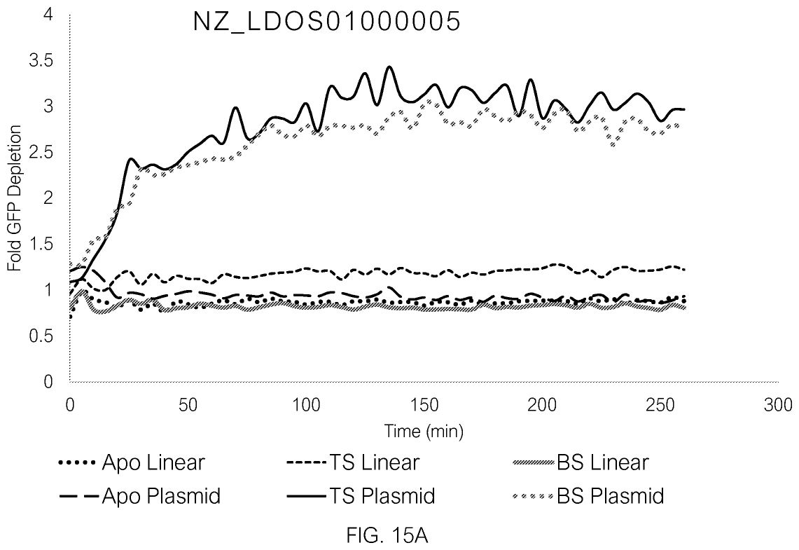

In an effort to reconstitute processing of the NZ_LDOS0100005 pre-crRNA into a mature crRNA in vitro, a pre-crRNA oligonucleotide template containing a T7 promoter followed the sequence, direct repeat (DR)-spacer1-DR-spacer2-DR, was synthesized. The purified oligonucleotide template was PCR amplified to select for full-length products and expressed the pre-crRNA using T7 in vitro transcription. The in vitro transcribed pre-crRNA was incubated with 0.0675 uM-1 μM of purified NZ_LDOS0100005 in 1×NEB Buffer2 with or without magnesium for 30 min. at 37° C. The resulting product was treated with proteinase K, supplemented with EDTA, denatured at 65° C. for 3 min., and run out on a 15% TBE-urea PAGE gel for analysis by SYBR-gold staining. FIG. 14 shows pre-crRNA treated with effector protein is processed into a mature crRNA in a dose-dependent manner without a dependence on magnesium.

### Example 3—Adaptation of CLUST.018837 CRISPR-Cas System Effectors for Eukaryotic and Mammalian Activity

DNA-modifying CRISPR-Cas systems such as CLUST.018837, systems described herein have important applications in eukaryotic cells such as therapeutic modification of the genome, with example modifications including but not limited to; genotype correction, gene knockout, genetic sequence insertion/deletion (by homology directed repair or otherwise), single nucleotide modification, or gene regulation. These gene modification modalities can utilize either natural or engineered activities of the CLUST.018837 CRISPR-Cas systems.

Without wishing to be limited, the applications in eukaryotic cells for the CLUST.018837 CRISPR-Cas system can be divided up into those utilizing nuclease and non-nuclease (also known as nuclease-dead) functionalities. For nucleases, in some embodiments, the natural nuclease activity of the CLUST.018837 CRISPR effector may be sufficient for applications such as gene modification, while in other embodiments, the targeted nuclease activity can be augmented by the fusion of additional nuclease domains (such as FokI) to either a nuclease-weak or nuclease-inactivated CLUST.018837 CRISPR effector. For non-nuclease functionalities, such nuclease-weak or nuclease inactivated CLUST.018837 CRISPR effectors can either be used directly or be fused to other functional domains. Both nuclease and non-nuclease functionalities are subsequently described in greater detail.

To develop CLUST.018837 CRISPR Cas systems for eukaryotic applications, the constructs encoding the protein effectors and/or their fusions are first codon-optimized for expression in mammalian cells, and specific localization tags are optionally appended to either or both the N-terminus or C-terminus of the effector protein. These localization tags can include sequences such as nuclear localization signal (NLS) sequences, which localize the effector to the nucleus for modification of genomic DNA. Other accessory proteins, such as fluorescent proteins, may be further appended. It has been demonstrated that the addition of robust, “superfolding” proteins such as superfolding green fluorescent protein (GFP) can increase the activity of CRISPR enzymes in mammalian cells when appended to the effector (Abudayyeh et al. (2017) Nature 550(7675): 280-4, and Cox et al. (2017) Science 358(6366): 1019-27).

The codon-optimized sequence coding for the CLUST.018837 effector and appended accessory proteins, fusion proteins, and/or localization signals is then cloned into a eukaryotic expression vector with the appropriate 5′ Kozak eukaryotic translation initiation sequence, eukaryotic promoters, and polyadenylation signals. In mammalian expression vectors, these promoters can include, e.g., general promoters such as CMV, EF1a, EFS, CAG, SV40, and cell-type specific RNA polymerase II promoters such as Syn and CamKIIa for neuronal expression, and thyroxine binding globulin (TBG) for hepatocyte expression to name a few. Similarly, useful polyadenylation signals include, but are not limited to, SV40, hGH, and BGH. For expression of the pre-crRNA or mature crRNA, RNA polymerase III promoters such as H1 or U6 can be used.

Delivery of the complete effector and RNA guide to the eukaryotic cells or tissues of choice can come in many different forms. For delivery to cells, in some embodiments. Transfection or nucleofection can deliver DNA or RNA from which the protein and/or RNA guide(s) is/are synthesized and assembled by the cellular machinery into active protein complexes, or the ribonucleoproteins (RNPs) themselves can be pre-formed extracellularly and delivered as a complete complex. Other applications may require the use of viral delivery, in which case the eukaryotic expression vector can be a lentiviral plasmid backbone, adeno-associated viral (AAV) plasmid backbone, or similar plasmid backbone capable of use in recombinant viral vector production. In particular, the small size of the CLUST.018837 CRISPR effectors make them ideally make them ideally suited for packaging, even when fused with other functional domains, along with its crRNA and appropriate control sequences into a single adeno-associated virus particle; the packaging size limit of 4.7 kb for AAV may preclude the use of larger effectors, particularly if large cell-type specific promoters are used for expression control.

After adapting the sequences, delivery vectors, and methods for eukaryotic and mammalian use, the different constructs as described herein are characterized for performance. For nuclease-based applications, in some instances, for testing of the mammalian nuclease activity of various constructs, a genomic dsDNA cleavage assay was used with either NGS or Surveyor nuclease readout to quantify the efficiency of indel formation (Hsu et al. (2013). In addition to testing various construct configurations and accessory sequences on individual targets, pooled library-based approaches are used to determine 1) any targeting dependency of specific constructs in mammalian cells as well as 2) the effect of mismatch locations and combinations along the length of the targeting crRNA. Briefly, the pooled library includes a selection plasmid that expresses a target DNA containing different flanking sequences as well as mismatches to the guide or guides used in the screening experiment, such that the successful target recognition and cleavage results in depletion of the sequence from the library. Furthermore, targeted indel sequencing or unbiased genome-wide cleavage assays can be used to evaluate the specificity of the CLUST.018837 nuclease constructs (Hsu et al. (2013), Tsai et al. (2015), Kim et al. (2015), Tsai et al. (2017)).

In addition to nuclease-based genome editing using CLUST.018837 effectors and a crRNA, additional template DNA sequences can be co-delivered either in a vector, such as an AAV viral vector, or as linear single stranded or double stranded DNA fragments. For insertion of template DNA by homology directed repair (HDR), template sequences are designed containing a payload sequence to be inserted into the locus of interest as well as flanking sequences that are homologous to endogenous sequences flanking the desired insertion site. In some instances, for insertion of short DNA payloads less than (for example: less than 1 kb in length), flanking homologous sequences can be short (for example: ranging from 15 to 200 nt in length). In other instances, for the insertion of long DNA payloads (for example: 1 kb or greater in length), long homologous flanking sequences are required to facilitate efficient HDR (for example: greater than 200 nt in length). Cleavage of target genomic loci for HDR between sequences homologous to template DNA flanking regions can significantly increase the frequency of HDR. CLUST.018837 effector cleavage events facilitating HDR include, but are not limited to dsDNA cleavage, double nicking, and single strand nicking activity.

Applications can also be based on non-nuclease functionalities of the CLUST.018837 effector and constructs from the fusion of the effector with a functional domain. In this context, the CLUST.018837 effector refers to both the natural effector amino acid sequence as well as any functional modifications to reduce or eliminate its nuclease activity. CLUST.018837 effectors have programmable DNA binding activity, which can be directly used in applications such as DNA immunoprecipitation, or other domains can be appended onto the effector to provide further functionality. Activities of these domains include, but are not limited to, DNA base modification (ex: ecTAD and its evolved forms, APOBEC), DNA methylation (m⁶A methyltransferases and demethylases), localization factors (KDEL retention sequence, mitochondrial targeting signal), transcription modification factors (ex: KRAB, VP64). Additionally, domains can be appended to provide additional control, such as light-gated control (cryptochromes) and chemically inducible components (FKBP-FRB chemically inducible dimerization).

Optimizing the activity of such fusion proteins requires a systematic way of comparing linkers that connect the CLUST.018837 effector with the appended domain. These linkers may include, but are not limited to, flexible glycine-serine (GS) linkers in various combinations and lengths, rigid linkers such as the alpha-helix forming EAAAK sequence, XTEN linker (Schellenberger V, et al. Nat. Biotechnol. 2009; 27:1186-1190), as well as different combinations thereof (see TABLE 5). The various designs are then assayed in parallel over the same crRNA target complex and functional readout to determine which one yields the desired properties.

For adapting CLUST.018837 effectors for use in targeted DNA base modification (see, e.g., Gaudelli et al. (2017) “Programmable base editing of A•T to G•C in genomic DNA without DNA cleavage” Science 25 Oct. 2017), one begins with a panel of CLUST.018837 effectors that yielded strong interference activity in in vivo E. coli bacterial screens. These effectors, whether with nuclease-inactivating mutations or in their natural forms, are mammalian codon optimized and tested for specific and programmable dsDNA binding in an in vitro environment such as using an electrophoretic mobility shift assay (EMSA).

Next, a linker is used to create the fusion protein between CLUST.018837 effector and the base editing domain. Initially, this domain consists of the ecTadA(wt)/ecTadA*(7.10) heterodimer (hereafter referred to as the dCas12i-TadA heterodimer) engineered previously for hyperactivity and modification of dsDNA A•T dinucleotides to G•C (TABLE 7). Given the structural differences between the smaller CLUST.018837 effectors versus the previously characterized Cas9 effectors, alternate linker designs and lengths may yield the optimal design of the base editing fusion protein. Further optimization of the location of the nuclear localization sequence may also be required.

To evaluate the activity of the CLUST.018837-derived base editors, the HEK 293T cells are transiently transfected with the CLUST.018837 effector-TadA heterodimer construct, a plasmid expressing the crRNA, and optionally, a reporter plasmid if targeting the reporter and not an endogenous locus. The cells are harvested 48 hours after transient transfection, and the DNA is extracted and prepared for next generation sequencing. Analysis of the base composition of loci of samples containing the targeting vs. negative control non-targeting crRNAs provide information about the editing efficiency, and analysis of the sequences at computationally predicted sites of close sequence similarity yields information about the off-target activity.

One particular advantage of developing a DNA base editing system using CLUST.018837 effectors is that the small size, smaller than the existing Cas9 and Cas12a effectors, enables more ready packaging in AAV of CLUST.018837 effector-TadA heterodimer along with its crRNA and control elements without the need for protein truncations. This all-in-one AAV vector enables greater efficacy of in vivo base editing in tissues, which is particularly relevant as a path towards therapeutic applications of CLUST.018837 effectors.

TABLE 5
Amino Acid Sequences of Motifs and Functional
Domains in Engineered Variants
of CLUST.018837 CRISPR-Cas Effector Proteins

>LINKER_1
(SEQ ID NO: 1005)
GS
>LINKER_2
(SEQ ID NO: 1006)
GSGGGGS
>LINKER_3
(SEQ ID NO: 1007)
GGGGSGGGGSGGGGS
>LINKER_4
(SEQ ID NO: 1008)
GGSGGSGGSGGSGGSGGS
>LINKER 5 (Gaudelli et al., 2017)
(SEQ ID NO: 1009)
SGGSSGGSSGSETPGTSESATPESSGGSSGGS
>ecTadA(wt) (Gaudelli et al., 2017) [N-term fusion
to ecTadA*(7.10)]
(SEQ ID NO: 1010)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQK KAQSSTD
>ecTadA*(7.10) (Gaudelli et al., 2017) [N-term
fusion to CRISPR nuclease]
(SEQ ID NO: 1011)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQK KAQSSTD
[Cytidine deaminase, AID, APOBEC1: N-term fusion
(or optionally C-term)]
>AID-APOBEC1 (Dickerson et al., 2003, Komor et
al., 2017)
(SEQ ID NO: 1012)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
>Lamprey_AID-APOBEC1 (Rogozin et al., 2007, Komor
et al., 2017)
(SEQ ID NO: 1013)
MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADC
AEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKIL
HTTKSPAV
>APOBEC1_BE1 (Komor et al., 2016)
(SEQ ID NO: 1014)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

### Example 4—Minimal Type V-U1 System Interferes with Gene Expression from Supercoiled dsDNA In Vitro

### In Vitro Interference Activity

To recapitulate interference activity seen in in vivo screens, effectors were targeted against GFP in an in vitro transcription-translation assay. Pre-crRNAs under a 17 promoter containing direct repeat (DR)-spacer-direct repeat (DR), with a spacer targeting GFP, were PCR amplified to select for full-length product. Effector and sigma28 templates also under a T7 promoter, and RFP and GFP templates under a fliC promoter were PCR amplified as well. All templates were then incubated together in an in vitro transcription-translation assay at 37° C. GFP and RFP fluorescence were read every 10 minutes by a TECAN Infinite M Plex plate reader for 12 hours.

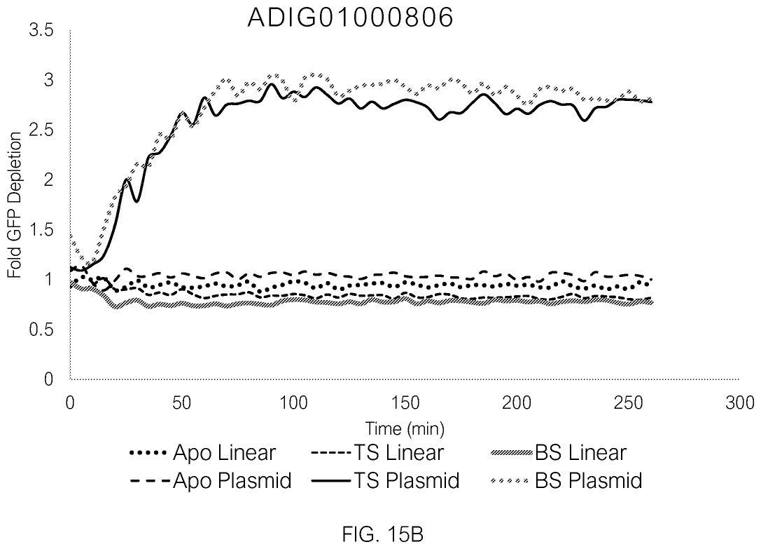

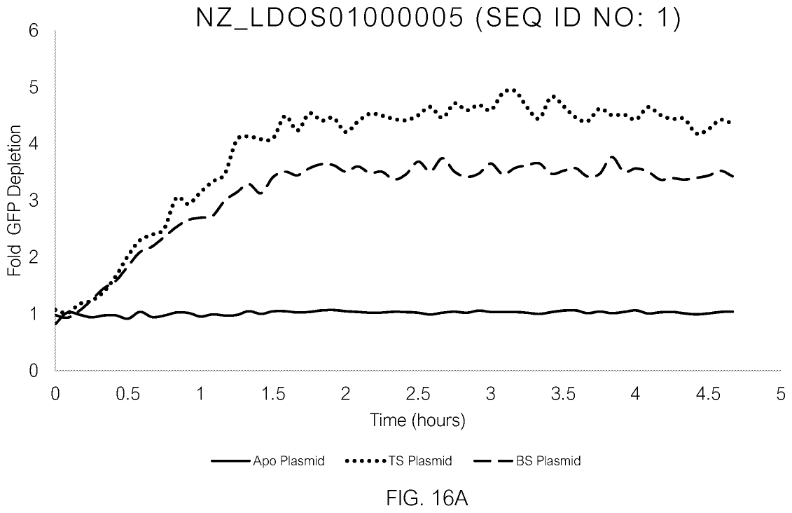

To calculate the fold depletion of GFP fluorescence, GFP signal was normalized to RFP signal at each time point, then the average fluorescence of two technical replicates was taken. GFP fluorescence depletion was then calculated by dividing the GFP signal of an effector incubated with a non-GFP targeting pre-crRNA by the GFP signal of an effector incubated with a GFP targeting pre-crRNA. Depletion of the GFP signal indicates that the effector has formed a functional RNP and interfered with the production of GFP. FIGS. 15A and 15B show the fold depletion of GFP over time by systems NZ_LDOS01000005 (SEQ ID NO: 1) and ADIG01000806 (SEQ ID NO: 20). Each effector demonstrated interference activity only when targeted against supercoiled plasmid expressing GFP, with a maximum depletion of GFP fluorescence around three-fold.

Pre-crRNA 17 template DNA sequences and primers used in the in vitro transcription-translation assay are listed in Table 6. Sequences for the GFP linear DNA and plasmid target DNA used for the in vitro transcription-translation assay are set forth in SEQ ID NO: 1075 and SEQ ID NO: 1076, respectively.

To assess the roles of the conserved putative RuvC domains found in NZ_LDOS01000005 and ADIG01000806 systems, point mutants were generated within putative RuvC I (H297A, D303A for NZ_LDOS01000005, H300A, D306A for ADIG01000806), RuvC II (E311A, D504A for NZ_LDOS01000005 and E332A. E516A for ADIG01000806), and RuvC III (D559A for NZ_LDOS01000005 and D569A for ADIG01000806) motifs and the resultant variants were evaluated for in vitro interference activity. FIGS. 16A-L demonstrate that mutation of amino acids within the putative RuvC domains of NZ_LDOS01000005 and ADIG01000806 systems result in an changes in in vitro interference activity.

TABLE 6
Pre-crRNA T7 template DNA used in
NZ_LDOS01000005 and ADIG01000806
(Type V-U1) for vitro biochemistry

| | | Fwd | Rev | |
|---|---|---|---|---|
| Name | Template Sequence | Primer | Primer | FIGS. |
| NZ_LDOS01000005 | CCCAACAGAAGCAAACC | AAGGGAACT | CTATGCGTC | FIGs. |
| TS pre-crRNA | TCGGTCGCAGCTACGAG | TGACAATGT | CCTAATGGA | 15A-B |
| | GCCGCCGCGATGGCCGA | CA | GT | |
| | TGAAACCCTTCCAGTTC | (SEQ ID | (SEQ ID | |
| | GGTGATATACGGGATCT | NO: 1071) | NO: 1072) | |
| | CTTTCTGTCGCAGCTAC | | | |
| | GAGGCCGCCGCGATGGC | | | |
| | CGATGAAACCCCTATAG | | | |
| | TGAGTCGTATTA | | | |
| | (SEQ ID NO: 1067) | | | |
| NZ.LDOS01000005 | CCCAACAGAAGCAAACC | AAGGGAACT | CTATGCGTC | FIGs. |
| BS pre-crRNA | TCGGTCGCAGCTACGAG | TGACAATGT | CCTAATGGA | 15A-B |
| | GCCGCCGCGATGGCCGA | CA | GT | |
| | TGAAACTATCCCGCGTT | (SEQ ID | (SEQ ID | |
| | ATCATCACATTACCTAC | NO: 1071) | NO: 1072) | |
| | CACACCGTCGCAGCTAC | | | |
| | GAGGCCGCCGCGATGGC | | | |
| | CGATGAAACCCCTATAG | | | |
| | TGAGTCGTATTA | | | |
| | (SEQ ID NO: 1068) | | | |
| ADIG01000806 | CCCAACAGAAGCAAACC | AAGGGAACT | CTATGCGTC | FIGs. |
| TS pre-crRNA | TCGGTCACAACTACTAG | TGACAATGT | CCTAATGGA | 15A-B |
| | GCGGCTGTGATCGCCTT | CA | GT | |
| | GGAATCGCCTTCCAGTT | (SEQ ID | (SEQ ID | |
| | CGGTGATATACGGGATC | NO: 1073) | NO: 1074) | |
| | TCTTTCTGTCACAACTA | | | |
| | CTAGGCGGCTGTGATCG | | | |
| | CCTTGGAATCCCTATAG | | | |
| | TGAGTCGTATTA | | | |
| | (SEQ ID NO: 1069) | | | |
| ADIG01000806 | CCCAACAGAAGCAAACC | AAGGGAACT | CTATGCGTC | FIGs. |
| BS pre-crRNA | TCGGTCACAACTACTAG | TGACAATGT | CCTAATGGA | 15A-B |
| | GCGGCTGTGATCGCCTT | CA | GT | |
| | GGAATCATATCCCGCGT | (SEQ ID | (SEQ ID | |
| | TATCATCACATTACCTA | NO: 1073) | NO: 1074) | |
| | CCACACCGTCACAACTA | | | |
| | CTAGGCGGCTGTGATCG | | | |
| | CCTTGGAATCCCTATAG | | | |
| | TGAGTCGTATTA | | | |
| | (SEQ ID NO: 1070) | | | |

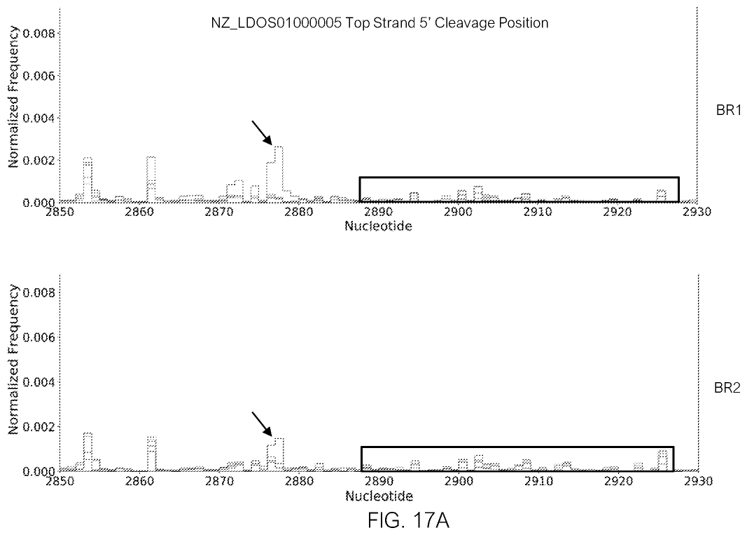

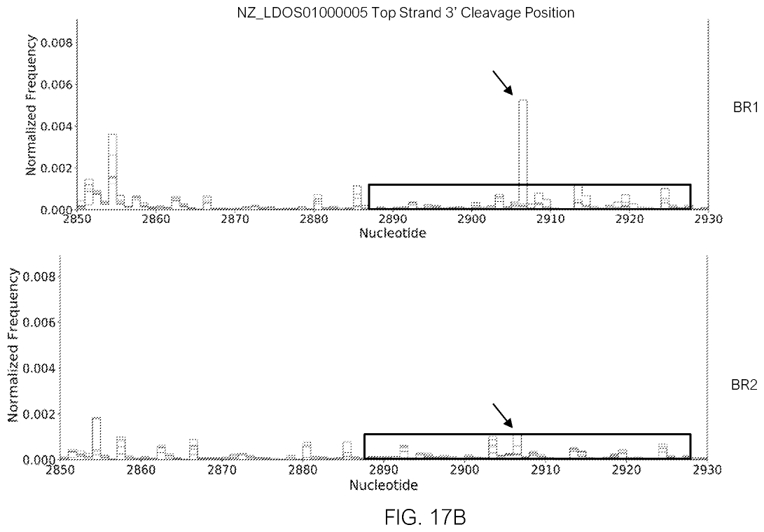

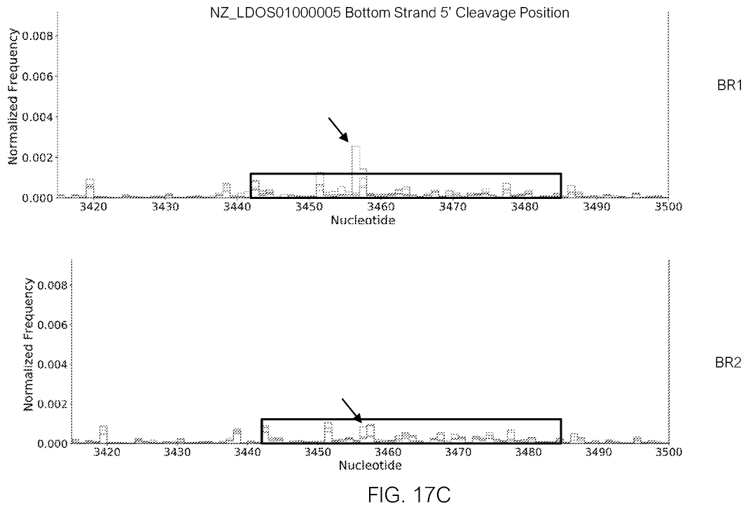

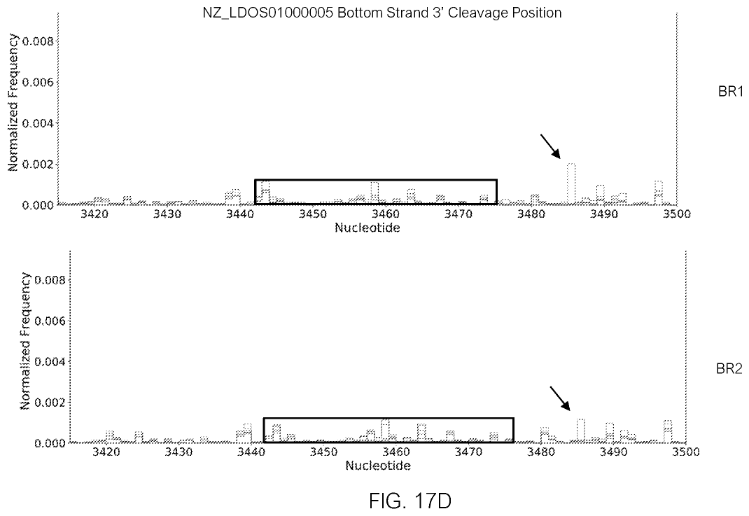

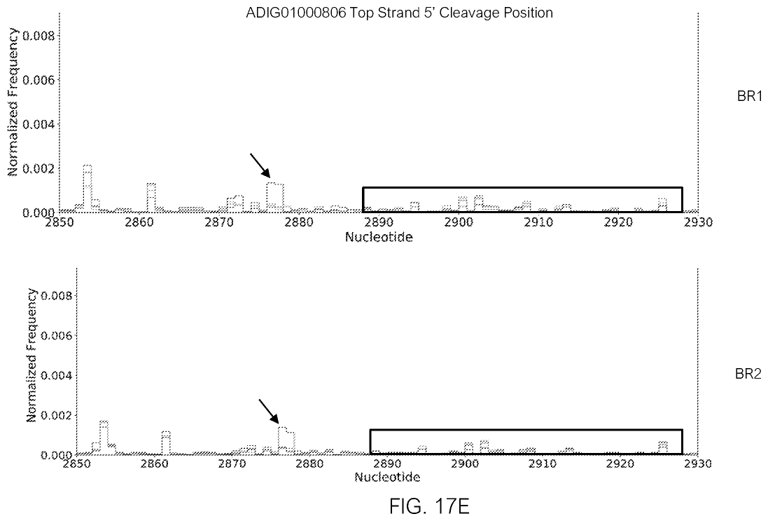

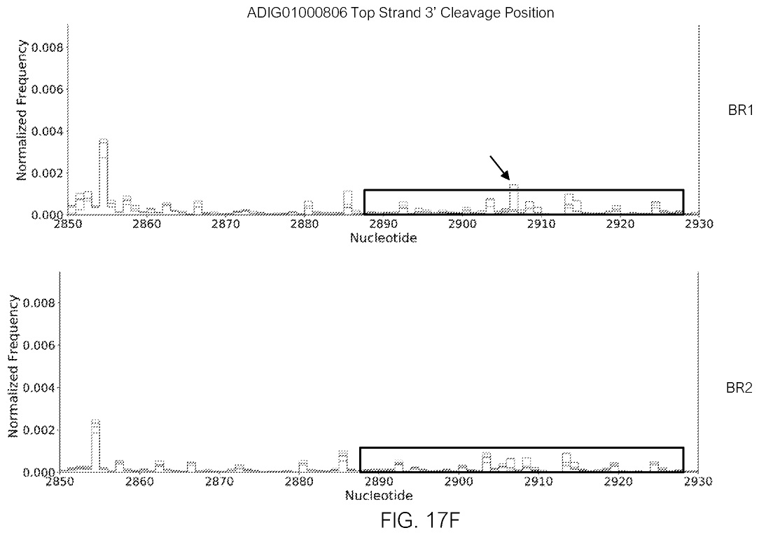

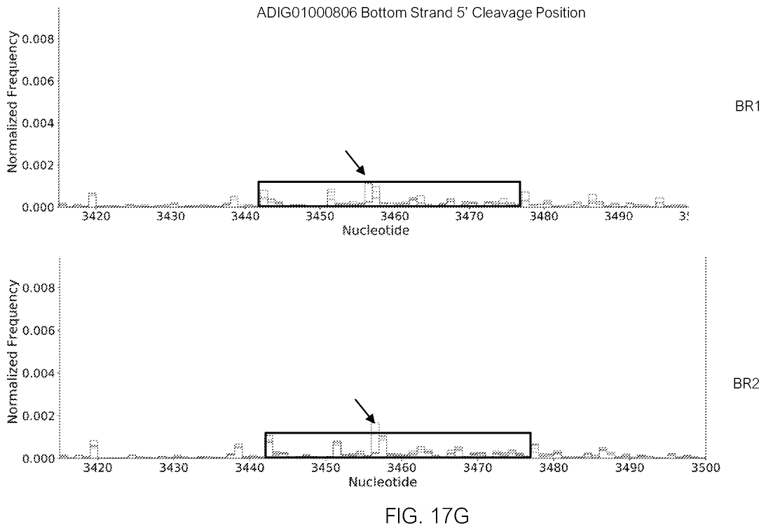

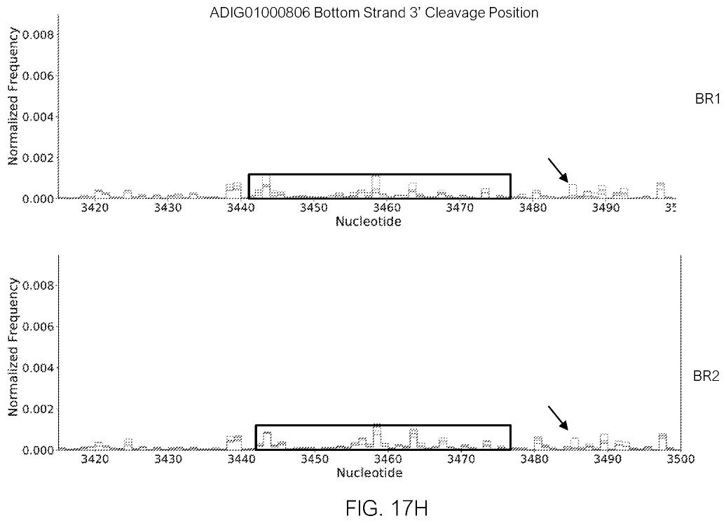

### Example 5—Minimal Type V-U1 System Cleaves Supercoiled dsDNA In Vitro (FIGS. 17A-H)

Detection of Supercoiled dsDNA Cleavage by Type V-UI CRISPR Effectors

To elucidate the molecular basis of interference of gene expression by the NZ_LDOS01000005 and ADIG01000806 systems, reactions from the above in vitro GFP interference assay were prepared for next generation sequencing (NGS). Reactions were quenched with EDTA and RNAse, then SPRI purified to isolate plasmid DNA. The plasmid DNA was then treated with NEBNext dsDNA Fragmentase (NEB) to generate fragments of DNA of ˜75 or 150 base pairs (bio-replicate 1 and 2, respectively) in length.

Fragments were then prepared for Illumiina next generation sequencing using New England Biolab's NEBNext Ultra DNA Library Prep Kit for Illumina. Sequencing was performed on an Illumina NextSeq 550 using. Reads were aligned to the sequence of GFP plasmid and normalized to total reads. Both NZ_LDOS01000005 and ADIG01000806 displayed nuclease activity when either the top or bottom strand was targeted across two biological replicates, with NZ_LDOS01000005 having a higher average cleavage frequency than ADIG01000806.

FIGS. 17A-H show that the cleavage by NZ_LDOS01000005 and ADIG01000806 systems appear to result in a nick at position 19 within the crRNA:target DNA duplex and a nick on the reciprocal DNA strand 10 bp downstream of the crRNA:target DNA duplex. These nicks are 28 bp downstream from one another within the crRNA:target DNA duplex.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a CRISPR-Cas effector protein or a nucleic acid encoding the CRISPR-Cas effector protein, wherein the CRISPR-Cas effector protein has at least 80% identity to an amino acid sequence set forth in any one of SEQ ID NOs: 1057-1066.

2. The composition of claim 1, wherein the CRISPR-Cas effector protein has an amino acid sequence set forth in any one of SEQ ID NOs: 1057-1066.

3. A composition comprising a CRISPR-Cas effector protein or a nucleic acid encoding the CRISPR-Cas effector protein, wherein the CRISPR-Cas effector protein comprises a mutation in a RuvC motif.

4. The composition of any previous claim, wherein the CRISPR-Cas effector protein comprises a mutation in a catalytic residue of a RuvC motif.

5. The composition of any previous claim, wherein the RuvC motif is a RuvC I, RuvC II, and/or RuvC III motif.

6. The composition of any previous claim, wherein the CRISPR-Cas effector protein comprises at least 10% less nuclease activity than a reference composition.

7. The composition of any previous claim, wherein the CRISPR-Cas effector protein lacks nuclease activity.

8. The composition of any previous claim, wherein the CRISPR-Cas effector protein comprises at least 10% greater nuclease activity than a reference composition.

9. The composition of any previous claim, wherein the composition further comprises an RNA guide or a nucleic acid encoding the RNA guide, wherein the RNA guide comprises a direct repeat sequence and a spacer sequence, wherein the CRISPR-Cas effector protein binds to the RNA guide, and wherein the spacer sequence binds to a target nucleic acid.

10. The composition of any previous claim, wherein the spacer sequence comprises between 15 and 24 nucleotides in length.

11. The composition of any previous claim, wherein the spacer sequence comprises between 16 and 22 nucleotides in length.

12. The composition of any previous claim, wherein the target nucleic acid comprises a sequence complementary to a nucleotide sequence in the spacer sequence.

13. The composition of any previous claim, wherein the CRISPR-Cas effector protein recognizes a protospacer adjacent motif (PAM) sequence in the target nucleic acid, wherein the PAM sequence comprises a nucleotide sequence set forth as 5′-TTN-3′ or 5′-YTN-3′, wherein N is any nucleotide and Y is cytosine or thymine.

14. The composition of any previous claim, wherein the target nucleic acid is DNA.

15. The composition of any previous claim, wherein the target nucleic acid is supercoiled (e.g., plasmid) DNA.

16. The composition of any previous claim, wherein the CRISPR-Cas effector protein further comprises at least one nuclear localization signal (NLS), at least one nuclear export signal (NES), or at least one NLS and at least one NES.

17. The composition of any previous claim, wherein the nucleic acid encoding the CRISPR-Cas effector protein is codon-optimized for expression in a cell.

18. The composition of any previous claim, wherein the nucleic acid encoding the CRISPR-Cas effector protein is operably linked to a promoter.

19. The composition of any previous claim, wherein the nucleic acid encoding the CRISPR-Cas effector protein is in a vector.

20. The composition of any previous claim, wherein the vector comprises a retroviral vector, a lentiviral vector, a phage vector, an adenoviral vector, an adeno-associated vector, or a herpes simplex vector.

21. The composition of any previous claim, wherein the composition is present in a delivery system comprising a nanoparticle, a liposome, an exosome, a microvesicle, or a gene-gun.

22. A cell comprising the composition of any previous claim.

23. The cell of any previous claim, wherein the cell is a eukaryotic cell.

24. The cell of any previous claim, wherein the cell is a prokaryotic cell.

25. A method of expressing the composition in a cell, wherein the method comprises:
(a) providing the composition; and
(b) delivering the composition to the cell.
